(19) United States
(12) Patent Application Publication
(10) Pub. No.: US 2020/0095641 A1
(43) Pub. Date: Mar. 26, 2020

(54) MEANS AND METHODS FOR ANTI-VEGF THERAPY

(71) Applicants: VIB VZW, Gent (BE); KATHOLIEKE UNIVERSITEIT LEUVEN, K.U.LEUVEN R&D, Leuven (BE); ROYAL COLLEGE OF SURGEONS IN IRELAND, Dublin (IE)

(72) Inventors: Diether Lambrechts, Kessel-Lo (BE); Dominiek Smeets, Wintershoven (BE); Annette Byrne, Dublin (IE)

(21) Appl. No.: 16/095,197

(22) PCT Filed: Apr. 21, 2017
(86) PCT No.: PCT/EP2017/059559

§ 371 Date: Oct. 19, 2018

(30) Foreign Application Priority Data
Apr. 21, 2016 (GB) 1606923.9

(51) Int. Cl. C12Q 1/6886; G16H 50/30; G16H 50/20; G16B 20/10

(52) U.S. Cl. CPC C12Q 1/6886; G16H 50/30; C12Q 2600/156; G16B 20/10; C12Q 2600/106; G16H 50/20

(57) ABSTRACT

The present application relates to the field of cancer, particularly to colorectal cancer (CRC). A panel of biomarkers is presented herein that can be used to cluster CRC samples into distinct genetic subtypes. It further relates to the use of the clustering method on patients treated with an anti-VEGF therapy and the identification of anti-VEGF responsive genetic subtypes.

### FIELD OF THE INVENTION

The present application relates to the field of cancer, particularly to colorectal cancer (CRC). A panel of biomarkers is presented herein that can be used to cluster CRC samples into distinct genetic subtypes. It further relates to the use of the clustering method on patients treated with an anti-VEGF therapy and the identification of anti-VEGF responsive genetic subtypes.

### BACKGROUND

Colorectal cancer (CRC) is the third most commonly diagnosed cancer in both men and women and an important contributor to cancer mortality and morbidity. CRC develops through an ordered series of events beginning with the transformation of normal colonic epithelium to an adenomatous intermediate and then ultimately adenocarcinoma, the so-called “adenoma-carcinoma sequence” (Pino and Chung, 2010). It is now generally accepted that multiple genetic events are required for tumor progression and that the temporal acquisition of these genetic changes matters. Recent genome-wide sequencing efforts have calculated as many as 80 mutated genes per colorectal tumor, but a smaller group of mutations (<15) were considered to be the true “drivers” of tumorigenesis (Wood et al 2007; Leary et al 2008). Genomic instability is recognized as an essential cellular feature that accompanies the acquisition of these mutations. In colorectal cancer, at least 3 distinct pathways of genomic instability have been described: the chromosomal instability (CIN), microsatellite instability (MSI), and CpG island methylator phenotype (CIMP) pathways. The CIN pathway underlies the majority of all colorectal cancers. CIN is observed in 65%-70% of sporadic colorectal cancers; the term refers to an accelerated rate of gains or losses of whole or large portions of chromosomes that results in karyotypic variability from cell to cell (Lengauer et al 1998). The consequence of CIN is an imbalance in chromosome number (aneuploidy), sub-chromosomal genomic amplifications, and a high frequency of loss of heterozygosity (LOH).

Although the rich history of investigations and the identification of numerous genetic changes that are causative for CRC development, CRC is still a frequently lethal disease with heterogeneous outcomes and heterogeneous drug responses. To move to personalized medicine and thus to more effective treatment strategies, it would be advantageous to identify clinically relevant and molecularly homogeneous subtyping of CRC tumors. However subclassification perse, even when built on what are believed to be relevant features of cancer cells (such as expression of cancer pathway components or driver gene mutations), may still not be predictive of differential drug responses. This can be due to the drugs themselves, with promiscuous mechanisms of action that may not track well with single pathway descriptors, or to our inability to properly define pathway engagement or cross-talk using static ‘omics’ data. Recently a consensus gene expression-based subtyping classification system for CRC was identified by the CRC Subtyping Consortium (Guinney et al 2015). The published gene expression-based subtyping classification makes use of six independent classification systems to categorize CRC samples into one of the four consensus molecular subtypes (CMS). However, this classification can only sort 87% of the CRC samples. Still 13% of the samples do not fall within one of the four CMS groups and should be considered separately as indeterminate subtypes, of yet unknown biological and clinical behavior. Moreover none of the currently available gene expression based CRC sub-classification methods is predictive of one or more differential drug responses. To solve this problem we developed a new DNA sub-classification method based on copy number alterations (CNA) of specific DNA regions. The use of CNAs to classify cancer has been shown previously for e.g. non-small lung cancer (Li et al 2014), melanoma (WO2010/051319) and colorectal cancer (WO2010/051318). However, with the method described in this application we are not only using distinct DNA regions but we were also able to classify 100% of all tested metastatic CRC (mCRC) tumor samples in one of three different subgroups. Moreover the subgroups defined by this new DNA-based classification method are related with the patients' response to Avastin therapy. Avastin or bevacizumab is a frequently used anti-VEGF antibody for treating cancer (Ferrara et al 2004).

### SUMMARY

Using copy number aberrations of specific DNA regions in a mCRC sample and subsequent unsupervised clustering we were able to classify mCRC tumors in 3 different subgroups. These subgroups are related with the patients' response to chemotherapy and outcome. Tumors that are classified in clusters 2 and 3 show additional benefit from Avastin treatment when compared to patients from the same clusters that received chemotherapy only. Hypermutator phenotypes, such as tumors with POLE or POLD1 mutations or micro-satellite instable tumors show no additional benefit from Avastin treatment. Copy number instability of specifically selected DNA regions is thus a biomarker for Avastin response. Tumors with a high proportion of the genome affected by CNAs have a significantly better response when treated with Avastin compared to copy number stable tumors.

It is an object of the invention to provide a colorectal cancer biomarker panel comprising at least 5 genomic DNA regions or fragments thereof selected from Table 1.

Another aspect of the invention is the use of said biomarker panel to determine the copy number alteration status of a colorectal cancer sample. The biomarker panel can also be used to determine the copy number instability of a colorectal cancer sample. According to particular embodiments, the biomarker panel comprising at least 5 genomic DNA regions or fragments thereof listed in Table 1, is used to cluster colorectal cancer samples in 3 distinct genetic subtypes wherein said subtypes are characterized by the copy number alteration specifications depicted in Tables 2, 3 and 4. According to particular embodiments, the said biomarker panel of the invention is used to predict the responsiveness of a colorectal cancer patient to anti-VEGF therapy.

According to another aspect a method is provided for determining the genetic subtype of a colorectal cancer sample, comprising determining the copy number alteration status of a colorectal cancer sample of a colorectal cancer patient using a biomarker panel, comprising at least 5 genomic DNA regions or fragments thereof selected from Table 1; and classifying said colorectal cancer sample in one of 3 distinct genetic subtypes wherein said subtypes are characterized by the copy number alteration specifications depicted in Tables 2, 3 and 4. According to particular embodiments, said method for determining the genetic subtype of a colorectal cancer sample can also be used to identify a patient responsive to anti-VEGF therapy, wherein classification of said patient in genetic subtypes 2 or 3 respectively depicted in Table 3 or 4 is indicative for said patient to be responsive to anti-VEGF therapy.

According to another aspect, a method is provided for the identification of a patient responsive to anti-VEGF therapy comprising determining the copy number instability of a CRC sample of a CRC patient using the biomarker panel comprising at least 5 genomic DNA regions or fragments thereof selected from Table 1, wherein a copy number instability of 15% or more is indicative for said patient to be responsive to anti-VEGF therapy.

### BRIEF DESCRIPTION OF THE FIGURES

FIG. 1. Recurrent CNAs in primary and metastatic colorectal cancer. Recurrent amplifications (red) and deletions (blue) are represented. Focal amplifications are presented in (a), focal deletions in (b) and whole-arm amplifications and deletions in (c). The green lines represent the significance threshold at q<0.25. In total 43 recurrent focal amplifications and 59 focal deletions were identified.

FIG. 2. Unsupervised hierarchical consensus clustering of copy number profiles of primary and metastatic colorectal cancer (n=880). (a) Unsupervised hierarchical clustering classified tumors into 3 consensus CNA subgroups termed clusters 1-3 based on recurrent CNAs as determined by GISTIC. Presence of recurrent amplifications (red) and deletions (blue) for each sample are represented. (b-f) Genomic characterization of the 3 clusters revealed that cluster 1 was enriched for MSI tumors and hypermutators as well as tumors with mutations in BRAF and PIK3CA. In contrast, Clusters 2 and 3 were enriched for tumors with mutations in TP53, a high copy number instability and a higher number of chromosomal breakpoints. Mutations in KRAS and APC are found across all clusters.

FIG. 3. Kaplan Meier plots and univariate and multivariate correlation of the different clusters with PFS in primary and metastatic colorectal cancer. (a) Cluster 1 correlates with better survival compared to clusters 2 and 3. (b) However, multivariate analysis correcting for relevant covariates showed that not the cluster but primary tumor, regional lymph nodes and distant metastases staging are the main contributors to worse prognosis.

FIG. 4. Clinical characteristics of the different clusters in primary and metastatic colorectal cancer. Characterizing of the clusters on a clinical level revealed that clusters 2 and 3 were enriched for tumors with higher regional lymph nodes and distant metastases staging.

FIG. 5. Unsupervised hierarchical Ward consensus clustering of copy number profiles of metastatic colorectal cancer (n=444). (a) Unsupervised hierarchical clustering performed on the metastatic tumors only classified tumors into 3 consensus CNA subgroups termed clusters 1-3 based on recurrent CNAs as determined by GISTIC. Presence of recurrent amplifications (red) and deletions (blue) for each sample are represented. (b-f) Genomic characterization of the 3 clusters revealed that the characteristics of the clusters are almost identical to the clusters determined in primary and metastatic colorectal cancer combined. Cluster 1 was enriched for MSI tumors and hypermutators as well as tumors with mutations in BRAF and PIK3CA. In contrast, Clusters 2 and 3 were enriched for tumors with mutations in TP53, a high copy number instability and a higher number of chromosomal breakpoints. Mutations in KRAS and APC are found across all clusters.

FIG. 6. Kaplan Meier plots and univariate and multivariate cox-regression of progression free and overall survival of the different clusters. Kaplan Meier plots (a) and univariate and multivariate analysis (b) for progression free survival and Kaplan Meier plots (c) and univariate and multivariate analysis (d) for overall survival are presented. Clusters 1 correlates with worse survival, clusters 2 and 3 with better survival. This effect is independent of clinical factors such as age, gender and TNM-staging.

FIG. 7. Clinical characteristics of the different clusters in metastatic colorectal cancer. Characterisation of the clusters revealed no enrichment for particular clinical characteristics.

FIG. 8. Comparison of patients treated with Avastin to those not treated with Avastin for each of the clusters and the effect on PFS. Patients from clusters 2 (b) and 3 (c) show additional benefit when treated with Avastin compared to patients not treated with Avastin. No such effect is observed for patients from cluster 1 (a). Similar results were obtained when combining patients from clusters 2 and 3 in one group (d).

FIG. 9. Comparison of patients treated with Avastin to those not treated with Avastin for each of the clusters and the effect on OS. Patients from cluster 3 (c) show additional benefit when treated with Avastin compared to patients not treated with Avastin. No such effect is observed for patients from cluster 1 (a) and 2 (b). When combining patients from cluster 2 and 3 an additional benefit is observed albeit less pronounced than patients from cluster 3 alone (d).

FIG. 10. Comparison of CNA-high with CNA-low tumors. Patients were stratified in CNA-high and CNA-low tumors based on the proportion of genomic regions that are affected by CNAs. CNA-high tumors are defined as having more affected regions than the first quartile, CAN-low is defined as equal or less. CNA-high patients that are treated with Avastin have a significantly better progression free survival and overall compared to CNA-high patients treated with standard-of-care chemotherapy (a). This effect is not observed for CNA-low tumors (b). For the Avastin treated tumors, a higher proportion of the genome affected by CNAs correlates with a higher progression-free survival and overall survival compared to to tumors with a lower proportion of the genome (c). This effect is not present for tumors not treated with Avastin (d).

FIG. 11. Unsupervised hierarchical Ward consensus clustering of copy number profiles of primary and metastatic colorectal cancer (n=880) using only the focal amplifications and deletions. Unsupervised hierarchical clustering using only the focal regions classified tumors into 3 consensus CNA subgroups that are nearly identical compared to clustering using all 180 regions. (a) Kaplan Meier plots of overall survival for the 3 clusters. (b) Multivariate analysis correcting for relevant covariates again showed that not the cluster but primary tumor, regional lymph nodes and distant metastases staging are the main contributors to worse prognosis. (c-g) Genomic characterization of the 3 clusters revealed that cluster 1 was enriched for MSI tumors and hypermutators as well as tumors with mutations in BRAF and PIK3CA. In contrast, Clusters 2 and 3 were enriched for tumors with mutations in TP53, a high copy number instability and a higher number of chromosomal breakpoints. Mutations in KRAS and APC are found across all clusters.

FIG. 12. Kaplan Meier plots and univariate and multivariate cox-regression of progression free and overall survival of the different clusters in metastatic colorectal cancer using only the focal amplifications and deletions. Clusters 1 correlates with worse survival, clusters 2 and 3 with better survival. This effect is independent of clinical factors such as age, gender and TNM-staging.

FIG. 13. Comparison of patients treated with Avastin to those not treated with Avastin for each of the clusters and the effect on PFS using only focal amplifications and deletions. Patients from clusters 2 (b,f) and 3 (c,g) show additional benefit when treated with Avastin compared to patients not treated with Avastin. No such effect is observed for patients from cluster 1 (a,e). Similar results were obtained when combining patients from clusters 2 and 3 in one group (d,h).

FIG. 14. Comparison of patients treated with Avastin to those not treated with Avastin for each of the clusters and the effect on OS using only focal amplifications and deletions. Patients from cluster 3 (c,g) show additional benefit when treated with Avastin compared to patients not treated with Avastin. No such effect is observed for patients from cluster 1 (a,e) and 2 (b,f). When combining patients from cluster 2 and 3 an additional benefit is observed albeit less pronounced than patients from cluster 3 alone (d,h).

FIG. 15. Accuracy of the different tiers identified using recursive partitioning analysis. (a) Boxplot of the accuracies of all trees generated using the different tiers starting from all 180 genomic regions. (b) Boxplot of the accuracies of all tree generated using the different tiers starting from the 102 focal genomic regions.

FIG. 16. K-nearest neighbors classification and random forest classification results on the replication cohort generated using all 180 genomic regions. Application of both the k-nearest neighbors classification model (a) and the random forest classification model (b) to the replication cohort classified the samples in 3 different clusters with very similar characteristics as the original clustered obtained from hierarchical clustering in terms of proportion of the genome affected by CNAs and number of breakpoints. Similar as the original clustering results cluster 2 and 3 show improved progression free survival.

FIG. 17. K-nearest neighbors classification and random forest classification results on the replication cohort generated using the 102 focal genomic regions. Application of both the k-nearest neighbors classification model (a) and the random forest classification model (b) to the replication cohort classified the samples in 3 different clusters with very similar characteristics as the original clustered obtained from hierarchical clustering in terms of proportion of the genome affected by CNAs and number of breakpoints. Similar as the original clustering results cluster 2 and 3 show improved progression free survival.

FIG. 18. Comparison of CNA-high with CNA-low tumors using only the 102 focal regions. Similar as the analysis for FIG. 10 we stratified patients in CNA-high and low and determined the relation with response to Avastin therapy. CNA-high tumors of patients treated with Avastin show a significant increase in progression free (a) and overall survival (c) compared to patients treated with standard-of-care chemotherapy. No such effect was noted for CNA-low tumors (b,d).

FIG. 19. Comparison of CNA-high with CNA-low tumors using the tier 1 and tier 2 regions from the recursive partitioning applied on the 102 focal regions. Similar as the analysis for FIG. 10 we stratified patients in CNA-high and low and determined the relation with response to Avastin therapy. CNA-high tumors of patients treated with Avastin show a significant increase in progression free (a) and overall survival (c) compared to patients treated with standard-of-care chemotherapy. No such effect was noted for CNA-low tumors (b,d).

FIG. 20. Comparison of CNA-high with CNA-low tumors using the tier 1 and tier 2 regions from the recursive partitioning applied all 180 regions. Similar as the analysis for FIG. 10 we stratified patients in CNA-high and low and determined the relation with response to Avastin therapy. CNA-high tumors of patients treated with Avastin show a significant increase in progression free (a) and overall survival (c) compared to patients treated with standard-of-care chemotherapy. No such effect was noted for CNA-low tumors (b,d).

FIG. 21. Comparison of CNA-high with CNA-low tumors using the top 50 ranked regions from the random forest classification model built with the 102 focal regions. Similar as the analysis for FIG. 10 we stratified patients in CNA-high and low and determined the relation with response to Avastin therapy. CNA-high tumors of patients treated with Avastin show a significant increase in progression free (a) and overall survival (c) compared to patients treated with standard-of-care chemotherapy. No such effect was noted for CNA-low tumors (b,d).

FIG. 22. Comparison of CNA-high with CNA-low tumors using the top 50 ranked regions from the random forest classification model built with the 180 focal regions. Similar as the analysis for FIG. 10 we stratified patients in CNA-high and low and determined the relation with response to Avastin therapy. CNA-high tumors of patients treated with Avastin show a significant increase in progression free (a) and overall survival (c) compared to patients treated with standard-of-care chemotherapy. No such effect was noted for CNA-low tumors (b,d).

FIG. 23. Classification of tumors in CNA-high or CNA-low for the avastin-treated replication cohort. Similar as the analysis for FIG. 10 we stratified patients in CNA-high and CNA-low (with the thresholds set to 30% and 25% for the top 50 ranking regions from the random forest classifiers (a,b) and the tier 1 and 2 regions (c,d) for both only focal regions (b,c) and all 180 regions (a,c) respectively. For each of the different subsets of genomic regions CNA-high tumors showed an increased progression free survival.

FIG. 24. The effect is independent from MSI status. To investigate whether the irresponsiveness of patients from cluster 1 to Avastin therapy was caused by tumors that show microsatellite instability we stratified patients from cluster 1 in MSI (a) and microsatellite stable (MSS) patients (b) and determined the relation with response to Avastin therapy. No difference between the samples was observed indicating that copy number stable samples that are MSS show no improved response on Avastin therapy and the effect is not solely dependent on MSI.

### DETAILED DESCRIPTION

### Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, N.Y. (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

In a first aspect, the invention relates to a colorectal cancer biomarker panel for determining the copy number alteration status of a colorectal cancer sample, comprising at least 5 genomic DNA regions or fragments thereof selected from Table 1. It also relates to a colorectal cancer biomarker panel for determining the copy number instability of a CRC sample comprising at least 5 genomic DNA regions or fragments thereof selected from Table 1. In a particular embodiment, said biomarker panel comprises at least 5 genomic DNA regions or fragments thereof selected from Table 5. In a more particular embodiment, said biomarker panel comprises at least 5 genomic DNA regions or fragments thereof selected from Table 6. In an even more particular embodiment, said biomarker panel comprises at least 5 genomic DNA regions or fragments thereof selected from Table 10.

The term “colorectal cancer biomarker panel” as used herein, and from hereon also referred to as “biomarker panel”, means a limited list of genomic DNA regions which can be used to determine the copy number alteration status or the copy number instability of a CRC sample. Importantly, although all genomic DNA regions listed in Table 1 are valuable and can be used as markers to evaluate copy number instability of CRC samples, it does not imply that all regions are needed to classify a CRC sample as copy number stable or unstable. Depending on the classification method used and the % accuracy the practitioner aims for, subselections of the listed genomic DNA regions can be used. In this application, Applicant teaches that a selection of 5 from the 180 genomic DNA regions listed in Table 1 can be enough to cluster CRC samples and thus to determine whether a CRC sample is copy number stable or instable with a high accuracy. Accordingly, CRC biomarker panel comprising “at least 5 genomic DNA regions” is envisaged in the embodiments described above. In alternative embodiments, the colorectal cancer biomarker panel of the application comprises at least 6, at least 7, at least 8, at least 9, at least 10, at least 11, at least 12 or at least 20 genomic DNA regions or fragments thereof selected from Table 1 or from Table 5 or from Table 6 or from Table 10. In other alternative embodiments, a colorectal cancer biomarker panel is provided comprising at least 5, at least 6, at least 7, at least 8, at least 9, at least 10, at least 11, at least 12 or at least 20 genomic DNA regions or fragments thereof selected from the genomic DNA regions listed in Table 6 and Table 7 or from the genomic DNA regions listed in Table 10 and Table 11.

The term “genomic DNA region” as used herein means a DNA sequence that is part of the genome of a cell or organism, as distinguished from extrachromosomal DNA, such as plasmids. The genomic DNA regions which are used within the scope of this invention are listed in Table 1. For every genomic DNA region within the scope of this application, Table 1 and Table 5 show the “wide peak limits” and the “peak limits”. The “wide peak limits” indicate the full sequence which contains the most comprehensive information. However, the invention also relates to smaller fragments of the listed genomic DNA regions. The “peak limits” for example indicate a subregion within the “wide peak limits” which contains the most condensed information to determine the copy number alteration status or copy number instability. Even smaller fragments within the “peak limits” can be used to cluster CRC samples according to the methods of the invention or can be used to determine the genetic subtype of CRC samples according to the methods of the invention or can be used to determine copy number instability of CRC samples according to the methods of the invention. Thus also smaller fragments which are part of the listed genomic DNA regions of Table 1 or Table 5 and which for example can be amplified by PCR or detected with probes related to DNA detection techniques (e.g. Southern blot) fall within the scope of the invention. Hence, the “biomarker panel” has to be read as comprising at least 5 genomic DNA regions listed in Table 1 or Table 5 or fragments thereof. Said fragments are thus smaller DNA fragments that are part of said genomic DNA regions. However, the said fragments of the at least 5 genomic DNA regions still need to have predictive power to determine the CNA status of a CRC sample, to determine the CIN of a CRC sample, to be useful to cluster CRC samples into the 3 genetic subtypes of the invention, to predict the responsiveness of a CRC patient to anti-VEGF therapy, to determine the genetic subtypes of the invention and/or to identify patients responsive to anti-VEGF therapy.

The term “copy number alterations” or “copy number aberrations”, both abbreviated as CNAs and interchangeably used in this application, are changes in copy number of specific DNA regions whereby the changes have arisen in somatic tissue, for example, only in a tumor. These changes can be amplifications or deletions. The “copy number alteration status” is thus the level or number of changes in copy number of a predefined list of DNA regions. For example, tumors can be categorized in CNA-high tumors and CNA-low tumors.

The relative number of regions affected by CNAs can be seen as a measure for copy number instability. Using different thresholds to define tumors as copy number unstable and stratify the patients accordingly we were able to observe beneficial responses to Avastin treatment for tumor instabilities ranging from 10% to 40% of regions affected by CNAs. We performed this analysis on 6 different subsets (1) using only the 102 focal regions, (2) using the top 50 ranked regions from the random forest classification model built with the 102 focal regions, (3) using the tier 1 and tier 2 regions from the recursive partitioning applied on the 102 focal regions, (4) using all 180 genomic regions (5) using the tier 1 and tier 2 regions from the recursive partitioning applied all 180 regions and (6) using the top 50 ranked regions from the random forest classification model built with the 180 focal regions.

In this application, CNA-high tumors are defined as tumors in which preferably 10% or more, more preferably 15% or more of the DNA region consisting of the biomarker panel used for the analysis (i.e. the genomic regions selected from Table 1 or Table 5 or fragments thereof that were used to determine the CNAs) is affected by CNAs, more preferably in which 20% or more of the DNA region consisting of the biomarker panel used for the analysis (i.e. the genomic regions selected from Table 1 or Table 5 or fragments thereof that were used to determine the CNAs) is affected by CNAs, and most preferably in which 26% or more of the DNA region consisting of the biomarker panel used for the analysis (i.e. the genomic regions selected from Table 1 or Table 5 or fragments thereof that were used to determine the CNAs) is affected by CNAs. For example, if 6 of the 180 genomic regions listed in Table 1 or Table 5 or fragments thereof are used to classify a CRC sample into one of the three genetic subtypes, then “x % or more of the DNA sequence consisting of the biomarker panel used for the analysis” means x % or more of the DNA sequence consisting of the 6 used genomic regions or fragments thereof. Similarly, if 10 of the 180 genomic regions listed in Table 1 or Table 5 or fragments thereof are used to classify a CRC sample into one of the three genetic subtypes, then “x % or more of the DNA sequence consisting of the biomarker panel used for the analysis” means x % or more of the DNA sequence consisting of the 10 used genomic regions or fragments thereof. A CNA-high tumor is thus copy number instable and therefore also referred to as “copy number instability high tumor” or CIN-high tumor. CNA-low tumors as used herein are tumors in which less than 15% of the DNA sequence consisting of the biomarker panel used for the analysis (i.e. the genomic regions selected from Table 1 or Table 5 or fragments thereof that were used to determine the CNAs) is affected by CNAs. Thus, if 6 of the 180 genomic regions listed in Table 1 or Table 5 or fragments thereof are used to classify a CRC sample into one of the three genetic subtypes, than less than 15% of the DNA sequence consisting of the 6 used genomic regions or fragments thereof is affected by CNA's to categorize the tumor as CNA-low. In alternative embodiments, CNA-low tumors as used herein are tumors in which less than 10% of the DNA sequence consisting of the biomarker panel used for the analysis (i.e. the genomic regions selected from Table 1 or Table 5 or fragments thereof that were used to determine the CNAs) is affected by CNAs. A CNA-low tumor has thus a low copy number instability and therefore also referred to as “copy number instability low tumor” or CIN-low tumor. Copy number alterations or copy number aberrations are not the same as copy number variations (CNVs). CNVs originate from changes in copy number in germline cells (and are thus in all cells of the organism).

The term “colorectal cancer” as used herein is meant to include malignant neoplasms of colon (C18 in ICD-10), malignant neoplasms of rectosigmoid junction (C19 in ICD-10), malignant neoplasms of rectum (C20 in ICD-10) and malignant neoplasms of anus and anal canal (C21 in ICD-10). A “colorectal cancer sample” refers to a biological sample of a “colorectal cancer patient”. A “colorectal cancer patient” refers to a living subject diagnosed with colorectal cancer or suspected to have colorectal cancer. In case that colorectal cancer is diagnosed with a living subject, a CRC sample comprises at least one colorectal cancer cell.

The 180 genomic DNA regions or fragments thereof which are listed in Table 1 or the 102 genomic DNA regions of fragments thereof which are listed in Table 5 are DNA regions which can be used to evaluate the copy number alteration status of a CRC sample and thus whether a colorectal cancer sample is copy number stable or instable or which can be used to cluster CRC samples (explained below). Although all 180 genomic DNA regions or fragments thereof are all informative and as valuable, some have a larger impact on the outcome of the analysis. In first instance, the impact of deletions or amplifications of specific genomic DNA regions on the evaluation of the copy number instability or of the copy number alteration status of a CRC sample depends on the classification method used. In current application, Applicant has confirmed the relevance of all 180 genomic DNA regions listed in Table 1 and of all 102 genomic DNA regions listed in Table 5 with three different methods, i.e. using regression trees, using the random forest classification and using the K-nearest neighbour classification (see Example 7). Another reason for the genomic DNA region dependent impact, is that some mutations affecting the copy number of specific genomic DNA regions occur early in colorectal tumor development, while other mutations occur at a later stage. However, the observation that copy number alterations of some genomic DNA regions have more or less impact does not mean that selecting genomic DNA regions with less impact is not useful to cluster CRC samples. All the genomic DNA regions listed in Table 1 are as valuable. The impact of selecting DNA regions with less strength might be that more of these regions will have to be used in the analysis to achieve the same level of accuracy.

Depending on the analysis method, different subselections can be made from the 180 genomic DNA regions or fragments thereof listed in Table 1 or from the 102 genomic DNA regions or fragments thereof listed in Table 5.

Therefore, in a particular embodiment of the first aspect, said colorectal cancer biomarker panel comprises at least 5 genomic DNA regions or fragments thereof selected from Tables 6, 7, 8 and/or 9, wherein at least 2 genomic DNA regions or fragments thereof are selected from Table 6. In another particular embodiment, said colorectal cancer biomarker panel comprises at least 5 genomic DNA regions or fragments thereof selected from Tables 6, 7, 8 and/or 9, wherein at least 3, at least 4 or at least 5 genomic DNA regions or fragments thereof are selected from Table 6. In another particular embodiment, said colorectal cancer biomarker panel comprises at least 6 genomic DNA regions or fragments thereof selected from Tables 6, 7, 8 and/or 9, wherein at least 2, at least 3, at least 4, at least 5 or at least 6 genomic DNA regions or fragments thereof are selected from Table 6.

In another particular embodiment of the first aspect, said colorectal cancer biomarker panel comprises at least 5 genomic DNA regions or fragments thereof selected from Tables 6, 7 and/or 8, wherein at least 2 genomic DNA regions or fragments thereof are selected from Table 6. In another particular embodiment, said colorectal cancer biomarker panel comprises at least 5 genomic DNA regions or fragments thereof selected from Tables 6, 7 and/or 8, wherein at least 3, at least 4 or at least 5 genomic DNA regions or fragments thereof are selected from Table 6. In another particular embodiment, said colorectal cancer biomarker panel comprises at least 6 genomic DNA regions or fragments thereof selected from Tables 6, 7 and/or 8, wherein at least 2, at least 3, at least 4, at least 5 or at least 6 genomic DNA regions or fragments thereof are selected from Table 6.

In another particular embodiment of the first aspect, said colorectal cancer biomarker panel comprises at least 5 genomic DNA regions or fragments thereof selected from Tables 6 and/or 7, wherein at least 2 genomic DNA regions or fragments thereof are selected from Table 6. In another particular embodiment, said colorectal cancer biomarker panel comprises at least 5 genomic DNA regions or fragments thereof selected from Tables 6 and/or 7, wherein at least 3, at least 4 or at least 5 genomic DNA regions or fragments thereof are selected from Table 6. In another particular embodiment, said colorectal cancer biomarker panel comprises at least 6 genomic DNA regions or fragments thereof selected from Tables 6 and/or 7, wherein at least 2, at least 3, at least 4, at least 5 or at least 6 genomic DNA regions or fragments thereof are selected from Table 6.

In another particular embodiment of the first aspect, said colorectal cancer biomarker panel comprises at least 5 genomic DNA regions or fragments thereof selected from Tables 6 and/or 7. In another particular embodiment, said colorectal cancer biomarker panel comprises at least 6, at least 7, at least 8, at least 9, at least 10, at least 11 or at least 12 genomic DNA regions or fragments thereof selected from Tables 6 and/or 7.

In yet another particular embodiment of the first aspect, said colorectal cancer biomarker panel comprises at least 5 genomic DNA regions or fragments thereof selected from Tables 10, 11 and/or 12, wherein at least 2 genomic DNA regions or fragments thereof are selected from Table 10. In another particular embodiment, said colorectal cancer biomarker panel comprises at least 5 genomic DNA regions or fragments thereof selected from Tables 10, 11 and/or 12, wherein at least 3, at least 4 or at least 5 genomic DNA regions or fragments thereof are selected from Table 10. In another particular embodiment, said colorectal cancer biomarker panel comprises at least 6 genomic DNA regions or fragments thereof selected from Tables 10, 11 and/or 12, wherein at least 2, at least 3, at least 4, at least 5 or at least 6 genomic DNA regions or fragments thereof are selected from Table 10.

In another particular embodiment of the first aspect, said colorectal cancer biomarker panel comprises at least 5 genomic DNA regions or fragments thereof selected from Tables 10 and/or 11, wherein at least 2 genomic DNA regions or fragments thereof are selected from Table 10. In another particular embodiment, said colorectal cancer biomarker panel comprises at least 5 genomic DNA regions or fragments thereof selected from Tables 10 and/or 11, wherein at least 3, at least 4 or at least 5 genomic DNA regions or fragments thereof are selected from Table 10. In another particular embodiment, said colorectal cancer biomarker panel comprises at least 6 genomic DNA regions or fragments thereof selected from Tables 10 and/or 11, wherein at least 2, at least 3, at least 4, at least 5 or at least 6 genomic DNA regions or fragments thereof are selected from Table 10.

In another particular embodiment of the first aspect, said colorectal cancer biomarker panel comprises at least 5 genomic DNA regions or fragments thereof selected from Tables 10 and/or 11. In another particular embodiment, said colorectal cancer biomarker panel comprises at least 6, at least 7, at least 8, at least 9, at least 10, at least 11 or at least 12 genomic DNA regions or fragments thereof selected from Tables 10 and/or 11.

Using the random forest classification method, a contribution value could be determined for every genomic DNA region listed in Table 1 or Table 5. The contribution value illustrates the importance of a CNA for correct classification of a sample. For each tree, the prediction error rate on the out-of-bag portion of the data is recorded. Then the same is done after permuting each predictor variable. The difference between the two are then averaged over all trees, and normalized by the standard deviation of the differences.

Therefore, in a particular embodiment of the first aspect, said colorectal cancer biomarker panel comprises at least 5 genomic DNA regions selected from Table 13, wherein said at least 5 genomic DNA regions have a contribution value of at least 1, at least 2, at least 3, at least 4 or at least 5 as listed in Table 13. In another particular embodiment, said colorectal cancer biomarker panel comprises at least 6 genomic DNA regions selected from Table 13, wherein said at least 6 genomic DNA regions have a contribution value of at least 1, at least 2, at least 3, at least 4 or at least 5 as listed in Table 13.

In another particular embodiment, said colorectal cancer biomarker panel comprises at least 5 genomic DNA regions selected from Table 13, wherein at least 2, at least 3, at least 4 or at least 5 genomic DNA regions from said at least 5 genomic DNA regions have a contribution value between 1 and 6 or between 2 and 6 or between 3 and 6 or between 4 and 6 or between 1 and 5 or between 2 and 5 or between 3 and 5 or between 2 and 4 as listed in Table 13.

In another particular embodiment, said colorectal cancer biomarker panel comprises at least 6 genomic DNA regions selected from Table 13, wherein at least 2, at least 3, at least 4, at least 5 or at least 6 genomic DNA regions from said at least 6 genomic DNA regions have a contribution value between 1 and 6 or between 2 and 6 or between 3 and 6 or between 4 and 6 or between 1 and 5 or between 2 and 5 or between 3 and 5 or between 2 and 4 as listed in Table 13.

In yet another particular embodiment, said colorectal cancer biomarker panel comprises at least 5 genomic DNA regions selected from Table 14, wherein said at least 5 genomic DNA regions have a contribution value of at least 1, at least 2, at least 3, at least 4, at least 7, at least 8 or at least 9 as listed in Table 14. In another particular embodiment, said colorectal cancer biomarker panel comprises at least 6 genomic DNA regions selected from Table 14, wherein said at least 6 genomic DNA regions have a contribution value of at least 1, at least 2, at least 3, at least 4, at least 7, at least 8 or at least 9 as listed in Table 14.

In another particular embodiment, said colorectal cancer biomarker panel comprises at least 5 genomic DNA regions selected from Table 14, wherein at least 2, at least 3, at least 4 or at least 5 genomic DNA regions from said at least 5 genomic DNA regions have a contribution value between 2 and 10 or between 3 and 10 or between 4 and 10 or between 7 and 10 or between 2 and 8 or between 3 and 8 or between 4 and 8 as listed in Table 14.

In another particular embodiment, said colorectal cancer biomarker panel comprises at least 6 genomic DNA regions selected from Table 14, wherein at least 2, at least 3, at least 4, at least 5 or at least 6 genomic DNA regions from said at least 6 genomic DNA regions have a contribution between 2 and 10 or between 3 and 10 or between 4 and 10 or between 7 and 10 or between 2 and 8 or between 3 and 8 or between 4 and 8 as listed in Table 14.

In yet other embodiments, said colorectal cancer biomarker panel comprises at least 20, at least 30, at least 40, at least 50, at least 60, at least 70, at least 80, at least 90, at least 100, at least 120, at least 140 or at least 160 genomic DNA regions selected from Table 1. In a most particular embodiment, said colorectal cancer biomarker panel consist of the genomic DNA regions depicted in Table 1.

In yet other embodiments, said colorectal cancer biomarker panel comprises at least 20, at least 30, at least 40, at least 50, at least 60, at least 70, at least 80, at least 90 or at least 100 genomic DNA regions selected from Table 5.

In most particular embodiments, said colorectal cancer biomarker panel consist of the genomic DNA regions depicted in Table 10, in Table 10 and Table 11, in Table 6 or in Table 6 and Table 7. In another most particular embodiment, said colorectal cancer biomarker panel consist of the genomic DNA regions depicted in Table 5 or in Table 1.

The colorectal cancer biomarker panels disclosed above in the first aspect of this application are from here on referred as “one of the colorectal cancer biomarker panels disclosed in one of the embodiments of the first aspect of the application” or as “one of the colorectal cancer biomarker panels of the application”.

In a second aspect, the invention relates to the use of a biomarker panel comprising at least 5 or at least 6 genomic DNA regions or fragments thereof selected from Table 1 to determine the copy number alteration status of a colorectal cancer sample. The invention also relates to the use of said biomarker panel to predict copy number instability of a colorectal cancer sample of a CRC patient. In particular embodiments, said Table 1 is Table 5, Table 6 or Table 10. In other particular embodiments, the use of a biomarker panel is provided for determining the copy number alteration status of a CRC sample or the copy number instability of a CRC sample, wherein said biomarker panel is one of the colorectal cancer biomarker panels disclosed in one of the embodiments of the first aspect of the application described above.

“Copy number instability” as used herein is defined as the gain and/or loss of copies of a specific set of genomic DNA regions. In a more particular embodiment, the invention relates to the use of one of the colorectal cancer biomarker panels of the application to classify a colorectal cancer sample of a CRC patient in a copy number instability (CIN) high or copy number instability low group. Copy number instability-high sample is defined as a sample in which 10%, preferable 15% or more of the DNA region consisting of the biomarker panel used for the analysis (e.g. the genomic regions selected from Table 1 or fragments thereof that were used to determine the CNAs) is affected by copy number alterations, more preferably 20% or more of the DNA region consisting of the biomarker panel used for the analysis (e.g. the genomic regions selected from Table 1 or fragments thereof that were used to determine the CNAs) is affected by CNAs and most preferably 26% or more of the DNA region consisting of the biomarker panel used for the analysis (e.g. the genomic regions selected from Table 1 or fragments thereof that were used to determine the CNAs) is affected by CNAs. This is especially important since our data surprisingly revealed that patients' samples with a high copy number instability are responsive to anti-VEGF therapy. To investigate whether the irresponsiveness of patients from cluster 1 to Avastin therapy was caused by tumors that show microsatellite instability we stratified patients from cluster 1 in MSI (a) and microsatellite stable (MSS) patients (b) and determined the relation with response to Avastin therapy. No difference between the samples was observed indicating that copy number stable samples that are MSS show no improved response on Avastin therapy and the effect is not solely dependent on MSI. In an alternative embodiment, a CIN-low sample is defined as a sample in which less than 10% of the DNA region consisting of the biomarker panel used for the analysis (e.g. the genomic regions selected from Table 1 or fragments thereof that were used to determine the CNAs) is affected by CNAs.

In a third aspect, the invention relates to the use of a biomarker panel comprising at least 5 or at least 6 genomic DNA regions or fragments thereof selected from Table 1 to cluster colorectal cancer samples in distinct genetic subtypes, wherein said subtypes are constructed using a dataset of multiple CRC samples. In a particular embodiment, said Table 1 is Table 5, Table 6 or Table 10. In another particular embodiment, the use of a biomarker panel to cluster colorectal cancer samples in distinct genetic subtypes is provided, wherein said subtypes are constructed using a dataset of multiple CRC samples and wherein said biomarker panel is one of the colorectal cancer biomarker panels disclosed in one of the embodiments of the first aspect of the application described above. More particular said biomarker panel comprises at least 20, at least 30, at least 40, at least 50, at least 60, at least 70, at least 80, at least 90 or at least 100 genomic regions or fragments thereof selected from Table 1 or Table 5. Even more particularly, said biomarker panel comprises at least 120, at least 130, at least 140, at least 150, at least 160 or at least 170 genomic regions or fragments thereof selected from Table 1. Most particularly, said biomarker panel consists of the genomic regions or fragments thereof selected from Table 1.

Dataset of multiple CRC samples are free available and are accessible to the person skilled in the art. In a preferred embodiment, said dataset consist of at least 100 CRC samples, more preferably at least 200 CRC samples, more preferably at least 300 CRC samples, most preferably at least 400 CRC samples.

In a particular embodiment, the use of a biomarker panel to cluster colorectal cancer samples in distinct genetic subtypes is provided, wherein said subtypes are constructed using a dataset of multiple CRC samples and wherein said biomarker panel is one of the colorectal cancer biomarker panels disclosed in one of the embodiments of the first aspect of the application described above, and wherein said subtypes are constructed using unsupervised hierarchical clustering. In a more particular embodiment, said subtypes are characterized by the copy number alteration specifications depicted in Tables 2, 3 and 4.

Using the means and methods of current application, 3 distinct genetic subtypes were determined, however depending on the clustering method and preferences of the practitioner more or less genetic subtypes can be constructed using the biomarker panel of the invention. In another particular embodiment, the use of a biomarker panel to cluster colorectal cancer samples in 3 distinct genetic subtypes is provided, wherein said subtypes are characterized by the copy number alteration specifications depicted in Tables 2, 3 and 4, wherein said biomarker panel is one of the colorectal cancer biomarker panels disclosed in one of the embodiments of the first aspect of the application described above.

The term “genetic subtype” as used herein means a category of CRC samples having common genetic characteristics, more precisely having a common copy number alteration status. “Distinct” means different, separate or diverse. In the application “genetic subtype” refers to a specific cluster. Cluster 1, 2, 3 are thus respectively the same as genetic subtype 1, 2, 3. The term “copy number alteration specifications” as used herein means the conditions to which a genetic sample must comply to fall into one of the genetic subtypes described in this application.

In another embodiment, the use of a biomarker panel is provided to predict the responsiveness of a colorectal cancer patient to anti-VEGF therapy, wherein said biomarker panel is one of the colorectal cancer biomarker panels disclosed in one of the embodiments of the first aspect of the application described above. The invention thus also relates to the use of the biomarker panel comprising at least 5 or at least 6 genomic DNA regions or fragments thereof selected from Table 1 or Table 5 to predict the responsiveness of a colorectal cancer patient to anti-VEGF therapy. In a more particular embodiment, said anti-VEGF therapy is bevacizumab therapy.

This is equivalent as saying that the biomarker panels described in the first aspect of the application or more particularly the biomarker panel comprising at least 5 genomic DNA regions or fragments thereof selected from Table 1 or Table 5 are provided for use in diagnosis of a CRC patient with responsiveness to anti-VEGF therapy, where in a particular embodiment said anti-VEGF therapy is bevacizumab therapy.

“Responsiveness” is defined in this application as the reaction or response of a CRC patient to an anti-VEGF treatment, more precisely to bevacizumab therapy. The response is positive or the patient is responsive to anti-VEGF therapy if the treatment clinically improves the situation of the patient. The term “anti-VEGF therapy” as used herein refers to an anti-angiogenic therapy, i.e. a therapy for example a medicament that inhibits angiogenesis or the growth of new blood vessels. VEGF stands for vascular endothelial growth factor and is a signal protein produced by cells that stimulates vasculogenesis and angiogenesis. Bevacizumab or avastin is a frequently used anti-VEGF antibody for treating cancer.

Bevacizumab and avastin are interchangeably used in this application. “Bevacizumab therapy” thus refers to the treatment of a patient that comprises bevacizumab administration. Bevacizumab can be administered as monotherapy or as combination therapy. Typically, monotherapy is used to describe the use of a single medication, while combination therapy or polytherapy uses more than one medication. A pharmacological therapy (i.e. a therapy that consists of one or more medicament against a single disease) can also be combined with other non-pharmacological therapies as radiation therapy and surgery.

In a fourth aspect, a method is provided to determine the genetic subtype of a colorectal cancer sample from a colorectal cancer patient, said method comprises:
a. Clustering a dataset of multiple CRC samples in distinct genetic subtypes using one of the CRC biomarker panels disclosed in one of the embodiments of the first aspect of current application;
b. Classifying said CRC sample from said CRC patient in one of said distinct genetic subtypes using one of the CRC biomarker panels disclosed in one of the embodiments of the first aspect of current application.

In particular embodiments, said clustering of step a) is done using a CRC biomarker panel comprising at least 5 genomic DNA regions or fragments thereof selected from Table 1 or Table 5 while said classification of step b) is done using the same said CRC biomarker panel.

The invention also provides a method for determining the genetic subtype of a colorectal cancer sample, comprising determining the copy number alteration status of a colorectal cancer sample of a colorectal cancer patient using one of the colorectal cancer biomarker panels described in the first aspect of the application (e.g. comprising at least 5 genomic DNA regions or fragments thereof selected from Table 1 or Table 5); classifying said colorectal cancer sample in one of 3 distinct genetic subtypes wherein said subtypes are characterized by the copy number alteration specifications depicted in Tables 2, 3 and 4; to determine the genetic subtype of said colorectal cancer sample.

“Classifying” means arranging a sample in a specific category (e.g. genetic subtype) according to shared qualities or characteristics with the other subjects of the specific category.

In a fifth aspect, the invention provides a method for the identification of a patient responsive to anti-VEGF therapy comprising determining the copy number alteration status of a colorectal cancer sample of a colorectal cancer patient using one of the colorectal cancer biomarker panels disclosed in one of the embodiments of the first aspect of this application (e.g. comprising at least 5 genomic DNA regions or fragments thereof selected from Table 1 or Table 5); classifying said colorectal cancer sample in one of 3 distinct genetic subtypes wherein said subtypes are characterized by the copy number alteration specifications depicted in Tables 2, 3 and 4 to determine the genetic subtype of said colorectal cancer sample, wherein classification of said patient in genetic subtypes 2 or 3 respectively depicted in Table 3 or 4 is indicative for said patient to be responsive to anti-VEGF therapy. In a more particular embodiment, the anti-VEGF therapy is bevacizumab therapy.

In a sixth aspect, the invention provides a method for the identification of a patient responsive to anti-VEGF therapy comprising:
Determining the copy number alteration status of a colorectal cancer sample of said subject using a biomarker panel, wherein said biomarker panel is one of the CRC biomarker panels disclosed in the first aspect of the application;
Classifying said patient as responsive to anti-VEGF therapy, if the copy number alteration status of the CRC sample of said patient classifies the CRC sample as genetic subtype 2 or 3 of which the copy number alteration specification are respectively depicted in Table 3 or 4.

In a particular embodiment, the invention provides a method for the identification of a patient responsive to anti-VEGF therapy comprising:
Determining the copy number alteration status of a colorectal cancer sample of said subject using a biomarker panel comprising at least 5 or at least 6 genomic DNA regions or fragments thereof selected from Table 1 or Table 5 or Table 6 or Table 10;
Classifying said patient as responsive to anti-VEGF therapy, if the copy number alteration status of the CRC sample of said patient classifies the CRC sample as genetic subtype 2 or 3 of which the copy number alteration specification are respectively depicted in Table 3 or 4.

In a more particular embodiment, the anti-VEGF therapy is bevacizumab therapy.

The term “indicative” as used herein means that a patient is predicted to be responsive to a therapy.

In another embodiment the invention provides a method for the identification of a patient responsive to anti-VEGF therapy, said method comprising determining the copy number instability of the genome of a CRC sample of a CRC patient, wherein a high copy number instability is indicative for said patient to be responsive to anti-VEGF therapy. The invention also provides methods for the identification of a patient responsive to anti-VEGF therapy, said methods comprising determining the copy number instability of a CRC sample of a CRC patient using one of the CRC biomarker panels disclosed in the first aspect of the application, wherein a high copy number instability is indicative for said CRC patient to be responsive to anti-VEGF therapy. The invention also provides methods for the identification of a patient responsive to anti-VEGF therapy, said methods comprising determining the copy number instability of a CRC sample of a CRC patient using a biomarker panel comprising at least 5 or at least 6 genomic DNA regions or fragments thereof selected from Table 1 or Table 5 or Table 6 or Table 10 wherein a high copy number instability is indicative for said CRC patient to be responsive to anti-VEGF therapy. In a more particular embodiment, the anti-VEGF therapy is bevacizumab therapy.

A high copy number instability means 10% or more, 15% or more, more preferably 20% or more, most preferably 26% or more of the DNA sequence consisting of the genomic regions or fragments thereof used for the analysis (e.g. those selected from Table 1 or Table 5) is affected by copy number alterations. The invention thus also provides methods for the identification of a patient responsive to anti-VEGF therapy comprising determining the copy number instability of a CRC sample of a CRC patient using one of the biomarker panels disclosed in the first aspect of the application or using a biomarker panel comprising at least 5 genomic DNA regions or fragments thereof selected from Table 1 or Table 5 or Table 6 or Table 10, wherein a copy number instability of 15% or more is indicative for said CRC patient to be responsive to anti-VEGF therapy or more particularly to bevacizumab therapy. The invention also provides methods for the identification of a patient responsive to anti-VEGF therapy comprising determining the copy number instability of the genome of a CRC sample of a CRC patient using one of the biomarker panels disclosed in the first aspect of the application or using a biomarker panel comprising at least 5 genomic DNA regions or fragments thereof selected from Table 1 or Table 5 or Table 6 or Table 10, wherein a copy number instability of 20% or more is indicative for said patient to be responsive to anti-VEGF therapy or more particularly to bevacizumab therapy. The invention also provides methods for the identification of a patient responsive to anti-VEGF therapy comprising determining the copy number instability of a CRC sample of a CRC patient using one of the biomarker panels disclosed in the first aspect of the application or using a biomarker panel comprising at least 5 genomic DNA regions or fragments thereof selected from Table 1 or Table 5 or Table 6 or Table 10, wherein a copy number instability of 26% or more is indicative for said CRC patient to be responsive to anti-VEGF therapy or more particularly to bevacizumab therapy.

In another embodiment, the invention provides a method for treating colorectal cancer in a subject in need thereof, comprising:
Determining the genetic subtype of a colorectal cancer sample of said subject according to the method of the invention for determining the genetic subtype of a colorectal cancer sample;
Administering anti-VEGF therapy to a subject, if the genetic subtype of said sample of said subject is classified as genetic subtype 2 or 3 of which the copy number alteration specifications are respectively depicted in Table 3 or 4.

In a more particular embodiment, the anti-VEGF therapy is bevacizumab therapy.

In another embodiment, the invention provides a method of treating colorectal cancer in a subject in need thereof, comprising:
Determining the copy number alteration status of a colorectal cancer sample of said subject using one of the biomarker panels disclosed in the first aspect of this application or using a biomarker panel comprising at least 5 genomic DNA regions or fragments thereof selected from Table 1 or Table 5 or Table 6 or Table 10;
Administering anti-VEGF therapy to said subject, if the copy number alteration status of the CRC sample of said subject classifies the CRC sample as genetic subtype 2 or 3 of which the copy number alteration specifications are respectively depicted in Table 3 or 4.

In a more particular embodiment, the anti-VEGF therapy is bevacizumab therapy.

“Respectively depicted” as used in the application means that the specifications of subtype 2 are shown in Table 3 and that of subtype 3 are presented in Table 4.

In another embodiment the invention provides a method of treating colorectal cancer in a subject in need thereof, comprising:
Determining the copy number instability of the genome of a CRC sample of said subject wherein said sample comprises at least one colorectal cancer cell;
Administering anti-VEGF therapy to a subject, if said sample of said subject is characterized by a high copy number instability, wherein a high copy number instability means that 15% or more of said genome is affected by copy number alterations, wherein said genome comprises the genomic regions used for the analysis and selected from Table 1 or Table 5 or Table 6 or Table 10.

In a more particular embodiment, the anti-VEGF therapy is bevacizumab therapy.

In another embodiment the invention provides a method of treating colorectal cancer in a subject in need thereof, comprising:
Determining the copy number instability of the genome of a CRC sample of said subject wherein said sample comprises at least one colorectal cancer cell;
Administering anti-VEGF therapy to a subject, if said sample of said subject is characterized by a high copy number instability, wherein a high copy number instability means that 20% or more of said genome is affected by copy number alterations, wherein said genome comprises the genomic regions used for the analysis and selected from Table 1 or Table 5 or Table 6 or Table 10.

In a more particular embodiment, the anti-VEGF therapy is bevacizumab therapy.

In another embodiment the invention provides a method of treating colorectal cancer in a subject in need thereof, comprising:
Determining the copy number instability of the genome of a CRC sample of said subject wherein said sample comprises at least one colorectal cancer cell;
Administering anti-VEGF therapy to a subject, if said sample of said subject is characterized by a high copy number instability, wherein a high copy number instability means that 26% or more of said genome is affected by copy number alterations, wherein said genome comprises the genomic regions used for the analysis and selected from Table 1 or Table 5 or Table 6 or Table 10.

In a more particular embodiment, the anti-VEGF therapy is bevacizumab therapy.

In another embodiment the invention provides a method of treating colorectal cancer in a subject in need thereof, comprising:
Determining the copy number instability of a CRC sample of said subject using one of the biomarker panels disclosed in the first aspect of the application or using a biomarker panel comprising at least 5 genomic regions or fragments thereof selected from Table 1 or Table 5 or Table 6 or Table 10;
Administering anti-VEGF therapy to said subject, if 15% or more of said CRC sample of said subject is affected by copy number alterations.

In a more particular embodiment, the anti-VEGF therapy is bevacizumab therapy.

In another embodiment the invention provides a method of treating colorectal cancer in a subject in need thereof, comprising:
Determining the copy number instability of a CRC sample of said subject using one of the biomarker panels disclosed in the first aspect of the application or using a biomarker panel comprising at least 6 genomic regions or fragments thereof selected from Table 1 or Table 5 or Table 6 or Table 10;
Administering anti-VEGF therapy to a said subject, if 20% or more of said CRC sample of said subject is affected by copy number alterations.

In a more particular embodiment, the anti-VEGF therapy is bevacizumab therapy.

In another embodiment the invention provides a method of treating colorectal cancer in a subject in need thereof, comprising:
Determining the copy number instability of a CRC sample of said subject using one of the biomarker panels disclosed in the first aspect of the application or using a biomarker panel comprising at least 6 genomic regions or fragments thereof selected from Table 1 or Table 5 or Table 6 or Table 10;
Administering anti-VEGF therapy to said subject, if 26% or more of said CRC sample of said subject is affected by copy number alterations.

In a more particular embodiment, the anti-VEGF therapy is bevacizumab therapy.

In another embodiment the invention provides a kit to determine the copy number alteration status in a colorectal cancer sample, comprising primers or probes for detection of at least 5 genomic DNA regions or fragments thereof selected from Table 1, Table 5, Table 6 or Table 10. In another embodiment the invention provides a kit to determine the copy number instability of a colorectal cancer sample, comprising primers or probes for detection of at least 5 genomic DNA regions selected from Table 1, Table 5, Table 6 or Table 10.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1: Study Population

We collected tumor biopsies from 278 metastatic colorectal cancer (mCRC) patients. After confirming the histopathology and assessing tumor content as described in the methods, we successfully performed whole-exome sequencing (WES) on paired biopsies from 194 patients (Coverage 57.3±41.6×). Low-coverage whole-genome sequencing was performed on 238 patients and copy number profiles were generated as described in the methods section. Subsequently, we manually assessed each profile to check whether tumor content was sufficiently high resulting in 176 profiles that were used for further downstream analysis. In total 157 patients were treated with combination-bevacizumab (bvz) therapy, 2 with monotherapy bvz and 15 with chemotherapy backbone only. For the survival analyses, we excluded the 2 patients with mono-therapy for the survival analyses and furthermore excluded 16 patients that received combination-bvz therapy in 2nd, 3rd or 4th line (n=13, 2 and 1 respectively) for which no information about first-line therapy was available. Another 2 patients were excluded since no clinical information about the therapy was available. Furthermore we performed whole-exome sequencing on 128 out of the 156 patients. Additionally we downloaded publicly available copy number data of a cohort of 205 patients from the CAIRO trial that were treated with Irinotecan-Capecitabine (CAPIRI) or capecitabine (CAP) only (Agilent oligonucleotide hybridization arrays; GSE36864) (Haan et al 2014) and a cohort of 499 patients from the TCGA network (http://gdac.broadinstitute.org/). Furthermore, a second cohort 106 of combination bevacizumab treated metastatic colorectal tumors and accompanying normal tissue from the MOMA clinical trial was provided by The University of Pisa in Italy (NCT02271464). For this replication cohort, the same procedure was followed, resulting in 78 patients selected for further analysis. All patients were treated with combination-bvz therapy.

### Example 2: Unsupervised Consensus Clustering of Copy Number Profiling Reveals 3 Clusters

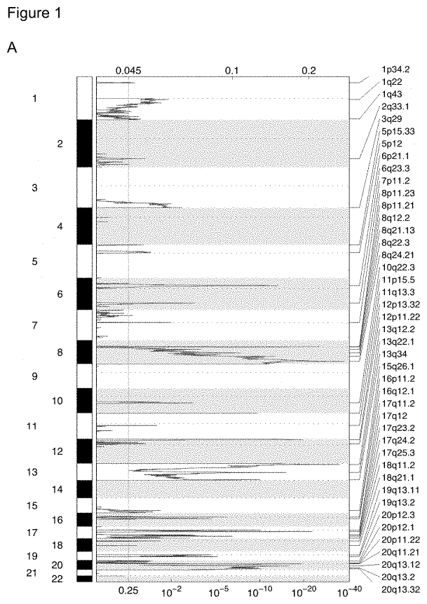

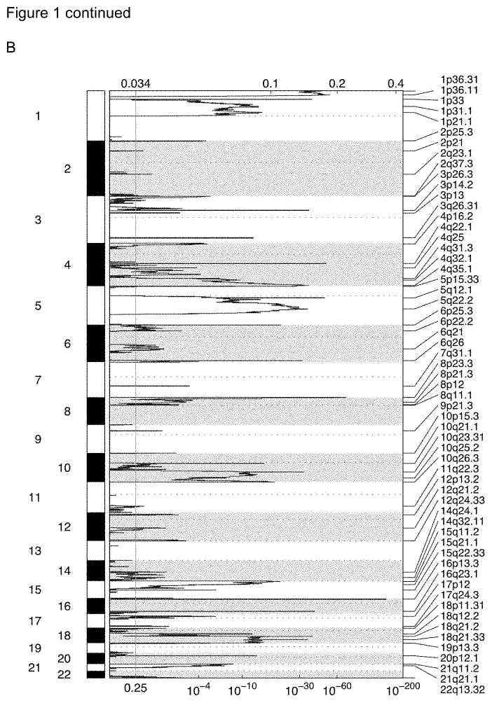

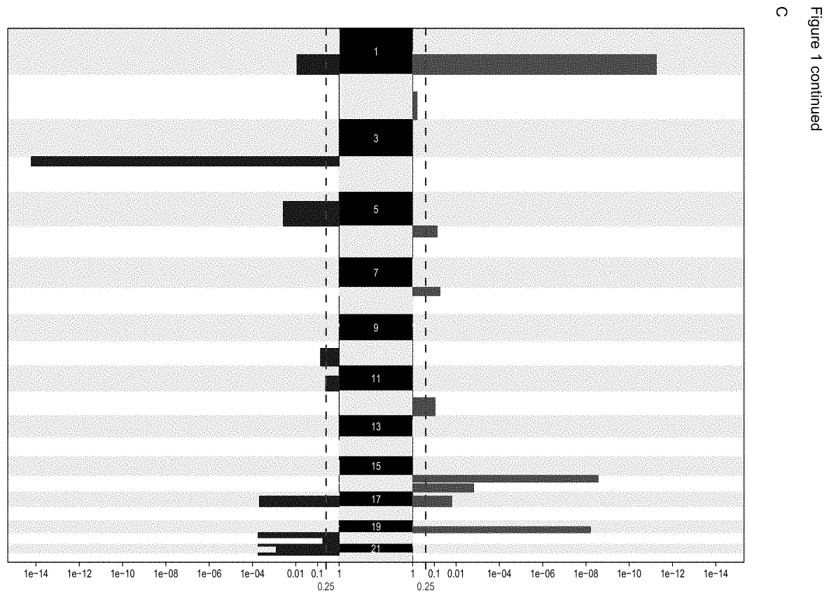

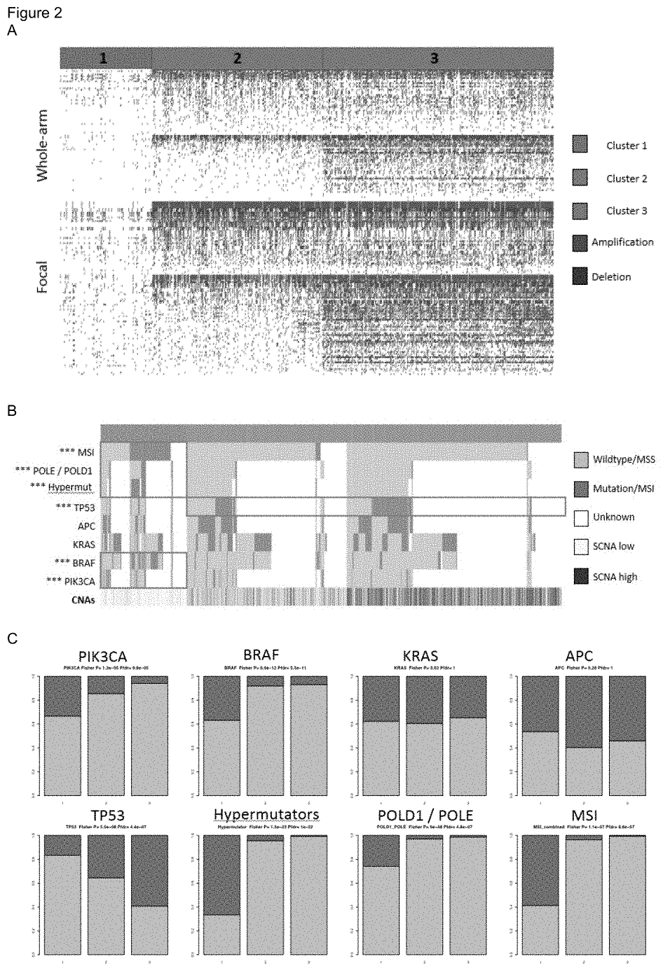

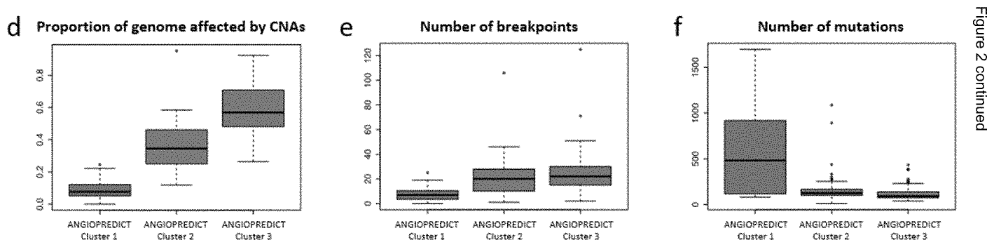

GISTIC analysis was performed on all 880 tumors to identify the most frequent and overrepresented somatic copy number aberrations (SCNAs) in the tumors (hereafter referred to as recurrent SCNAs). This analysis revealed the presence of 43 recurrent focal amplifications and 59 recurrent focal deletions as well as whole-arm aberrations in every chromosome (Table 1; FIG. 1). We then performed unsupervised hierarchical clustering in an iterative manner using the status of each of the 102 regions and 78 whole-arms, resulting in 3 clusters to which patients could be assigned (FIG. 2; Table 2). Table 2 shows the specifications of a cluster 1 sample. The specifications of a CRC sample that is categorized in cluster 2 are shown in Table 3. The specifications which should be fulfilled by a CRC sample that is characterized as a cluster 3 sample are shown in Table 4.

Characterisation of the clusters on the somatic mutation and copy number level revealed that patients in clusters 2 and 3 had substantially more chromosomal breakpoints and a higher proportion of the genome was affected by copy number aberrations, while cluster 1 showed almost no CNAs or breakpoints (FIG. 2). We then focused on the somatic mutations that were detected using whole-exome sequencing (WES) for the 142 patients for which WES data was available and mutational data available for the TCGA samples. Cluster 1 was enriched for MSI-tumors (P=8.8×10⁻⁵⁷) and tumors with POLE and POLD1 mutations (P=4.8×10⁻⁰⁷), which are related to a hypermutator phenotype (P=1×10⁻²²) but also for BRAF (P=9.8×10⁻⁰⁵) and PIK3CA (P=5.5×10⁻¹¹) mutations. TP53 mutations were predominantly present in clusters 2 and 3 (P=4.4×10⁻⁰⁷), while APC and KRAS were evenly distributed among all 3 clusters. Cluster 1 showed a very high mutation rate confirming that this cluster is enriched for hypermutators, which is in line with the increased rate of POLE, POLD1 mutations and increased MSI tumors (FIG. 2).

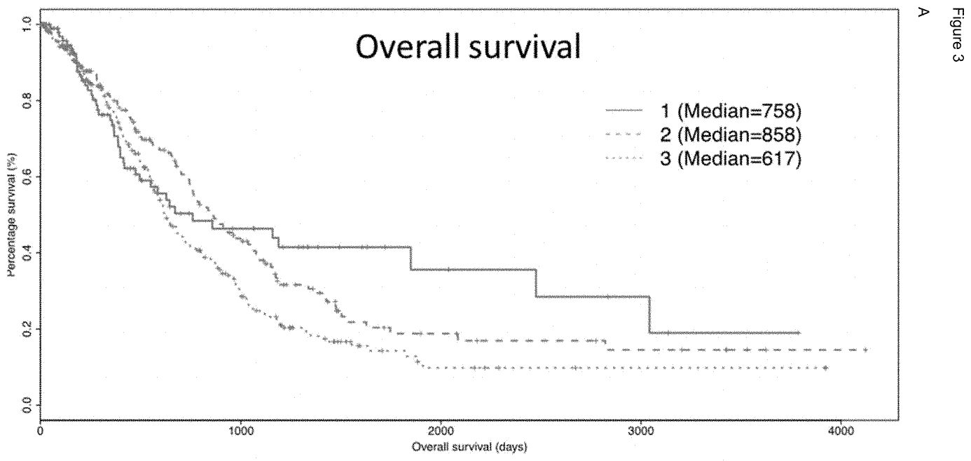

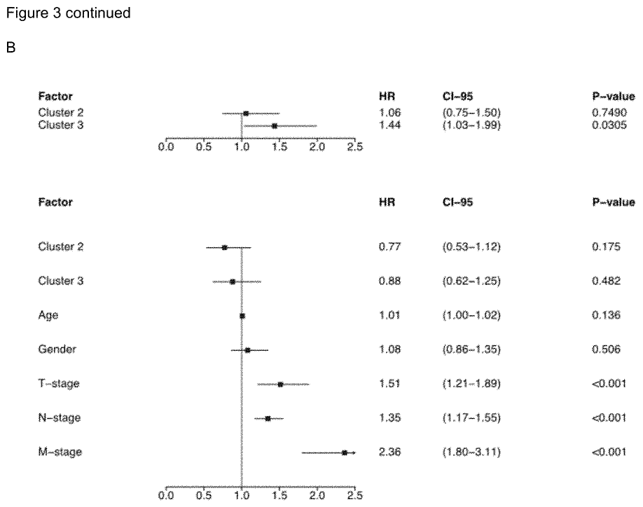

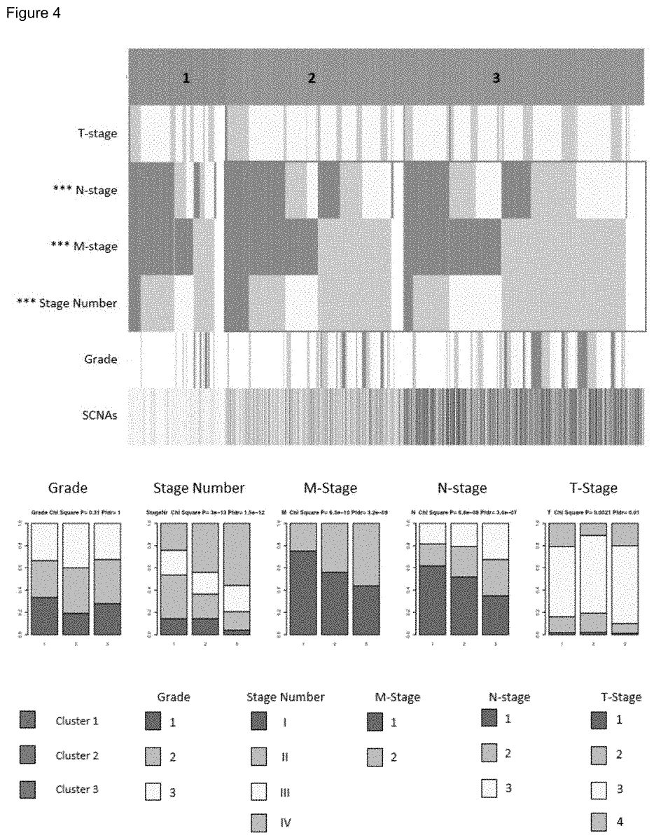

Next, we performed Kaplan-Meier analysis to determine the relationship between the different clusters and the patients' progression free survival and overall survival. Univariate COX-regression revealed that cluster 3 correlated with slightly worse overall survival compared to cluster 1 (P=0.3×10⁻²; H R=1.44 CI95=1.03-1.99). However, multivariate analysis using a COX-regression with age, TNM-staging as numerical factors and age as categorical factor showed that not the cluster but primary tumor, regional lymph nodes and distant metastases staging are the main contributors to worse prognosis (P=2.53×10⁻⁴, HR=1.51, CI95=1.21-1.89; P=2.25×10⁻⁵, HR=1.35, CI95=1.17-1.55 and P=7.63×10⁻¹⁰, HR=2.36, CI95=1.80-3.11 respectively) (FIG. 3). Characterizing of the clusters on a clinical level revealed that clusters 2 and 3 were enriched for tumors with higher regional lymph nodes (P=3.4×10⁻⁷) and distant metastases staging (3.2×10⁻⁹) and higher stage numbers (P=1.5×10⁻¹²) (FIG. 4).

### Example 3: Unsupervised Consensus Clustering of Metastatic Colorectal Cancer Patients

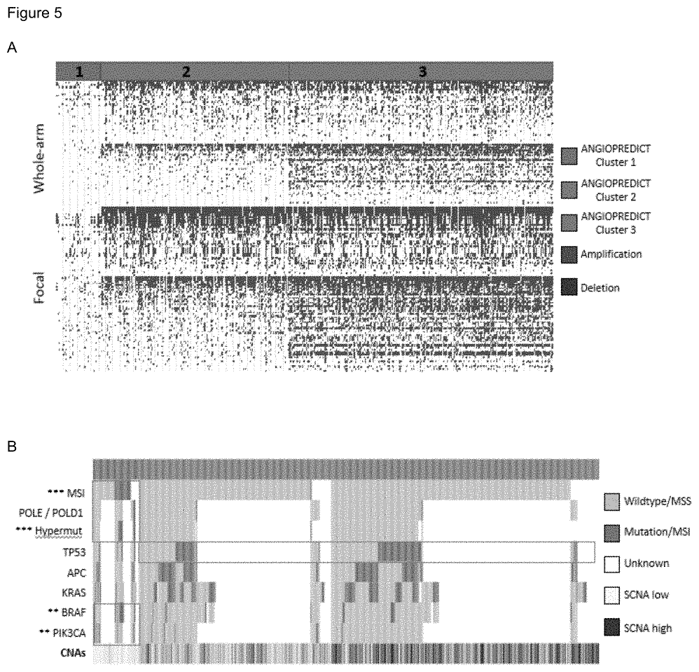

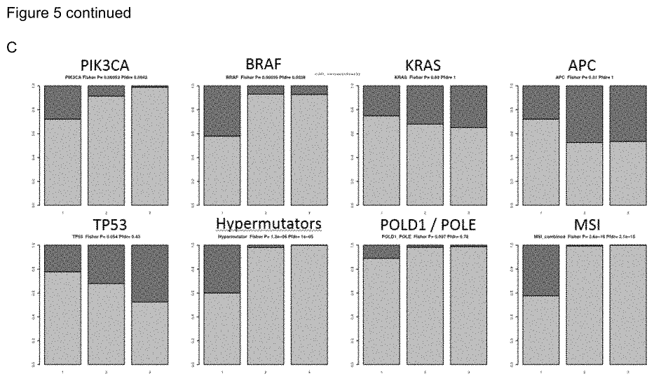

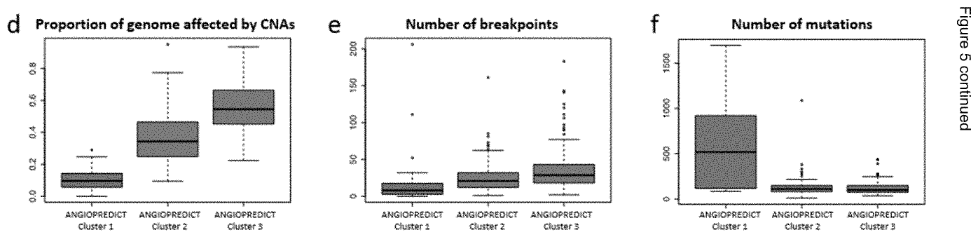

In a next step, we selected all the stage IV tumors, repeated the hierarchical clustering and performed the same characterization on clinical and genomic level. Similar as for all the colorectal samples, the subset of mCRC tumors showed an enrichment for TP53 mutations (P=5.2×10⁻⁴) and a substantially increased number of CNAs and breakpoints in clusters 2 and 3, while cluster 1 showed almost no CNAs and was enriched for MSI-tumors (P=2.1×10⁻¹⁵) and hypermutator phenotypes (P=1×10⁻⁵) as well as BRAF (P=2.6×10⁻³) and PIK3CA mutations (P=4.2×10³)(FIG. 5).

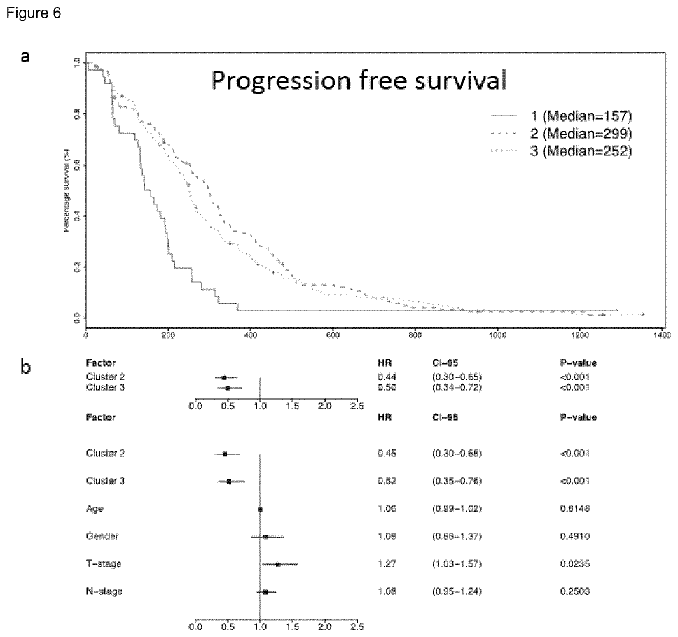

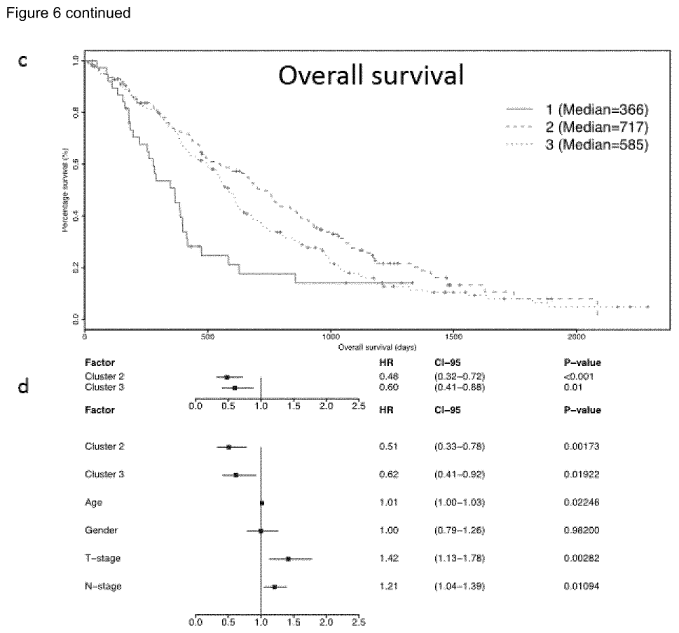

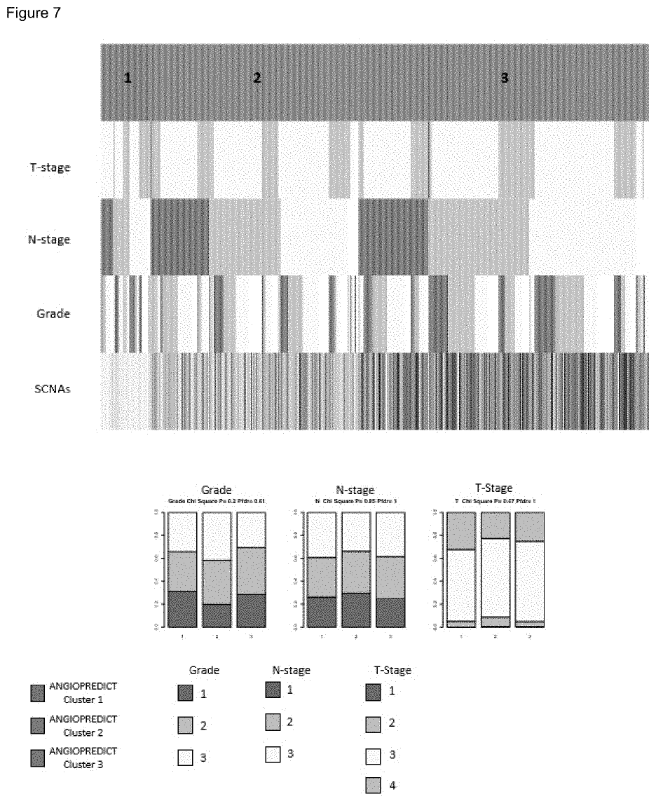

However, when assessing progression free and overall survival it became apparent that, compared to cluster 1, cluster 2 and 3 showed significantly better PFS (P=2.63×10⁻⁵, HR=0.44, CI95=0.30-0.65 and P=1.85×10⁻⁴, HR=0.50, CI95=0.34-0.72 for cluster 2 and 3 respectively) and OS (P=3.85×10⁻⁴, HR=0.48, CI95=0.32-0.72 and P=9.73×10⁻³, HR=0.60, CI95=0.41-0.88 for cluster 2 and 3 respectively). Multi-variate analysis correcting for relevant covariates confirmed that clusters 2 and 3 were significantly correlated with improved PFS (P=1.22×10⁻⁴, HR=0.45, CI95=0.30-0.68 and P=7.47×10⁻⁴, HR=0.52, CI95=0.35-0.76 for cluster 2 and 3 respectively) and OS (P=1.73×10³, HR=0.51, CI95=0.33-0.78 and P=1.92×10⁻², HR=0.62, CI95=0.41-0.92 for cluster 2 and 3 respectively), while primary tumor (P=2.35×10⁻², HR=1.27, CI95=1.03-1.57 and P=2.82×10⁻³, HR=1.42, CI95=1.13-1.78 for clusters 2 and 3) and regional lymph node staging contributed negatively (P=2.50×10⁻¹, HR=1.08, CI95=0.95-1.24 and P=1.09×10⁻², HR=1.21, CI95=1.04-1.39 for clusters 2 and 3)(FIG. 6). Characterization on a clinical level revealed no enrichments for particular clinical stages. (FIG. 7)

### Example 4: Patients from Clusters 2 and 3 Show Additional Benefit from Combination-Avastin Therapy Compared to Patients Treated with Chemotherapy Only

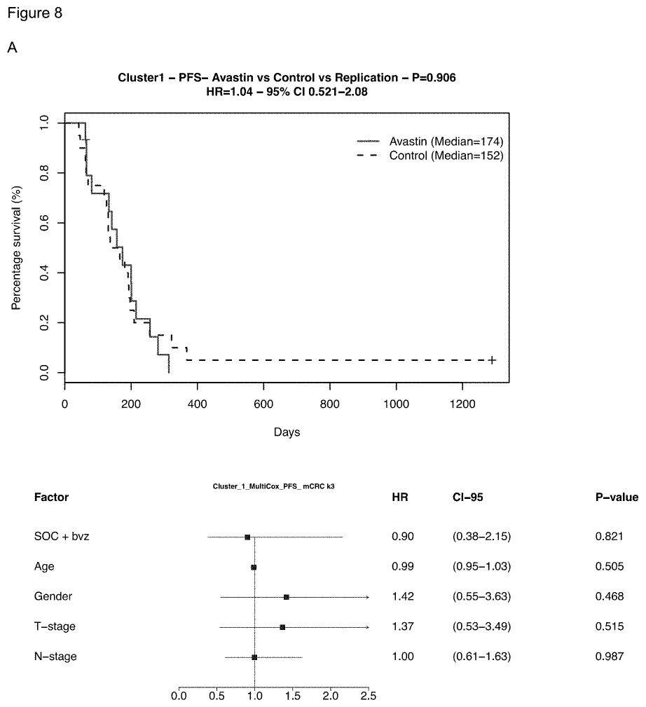

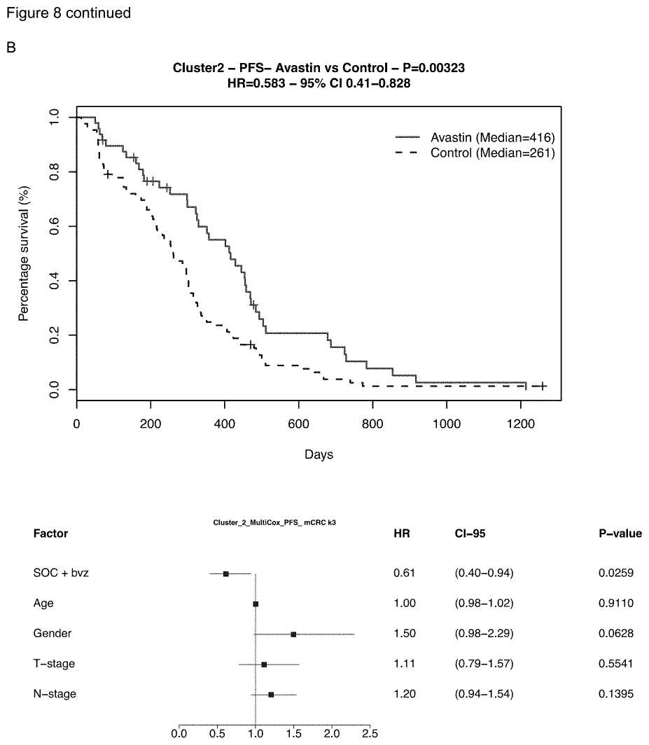

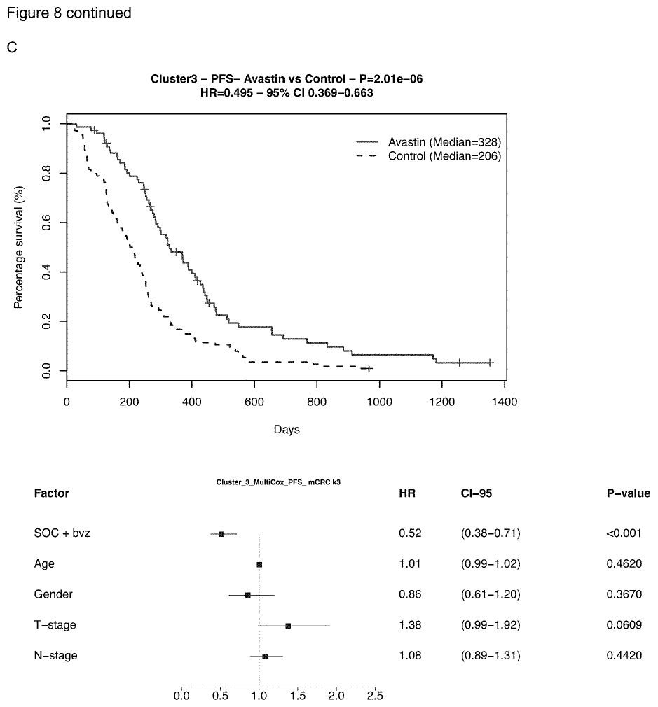

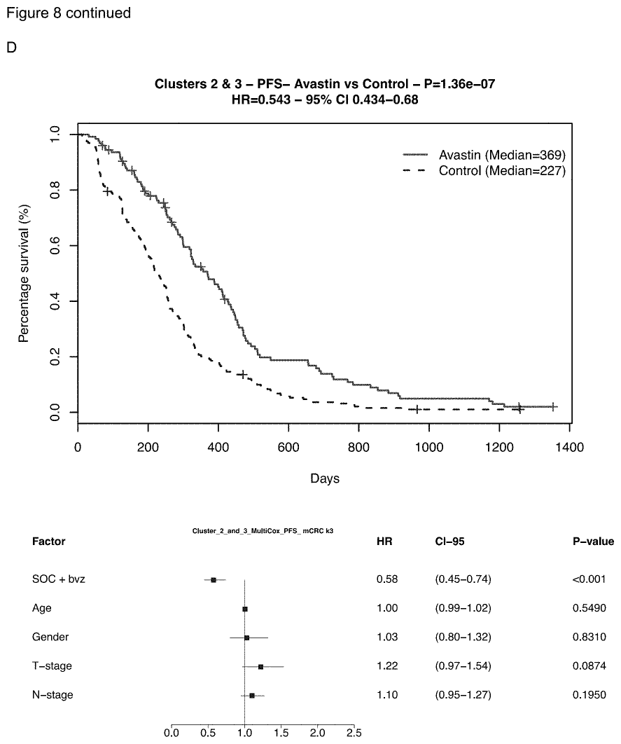

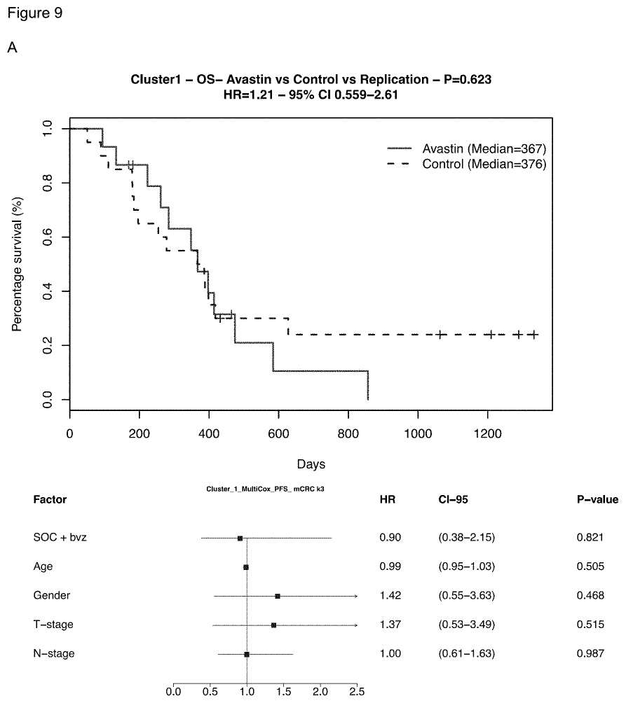

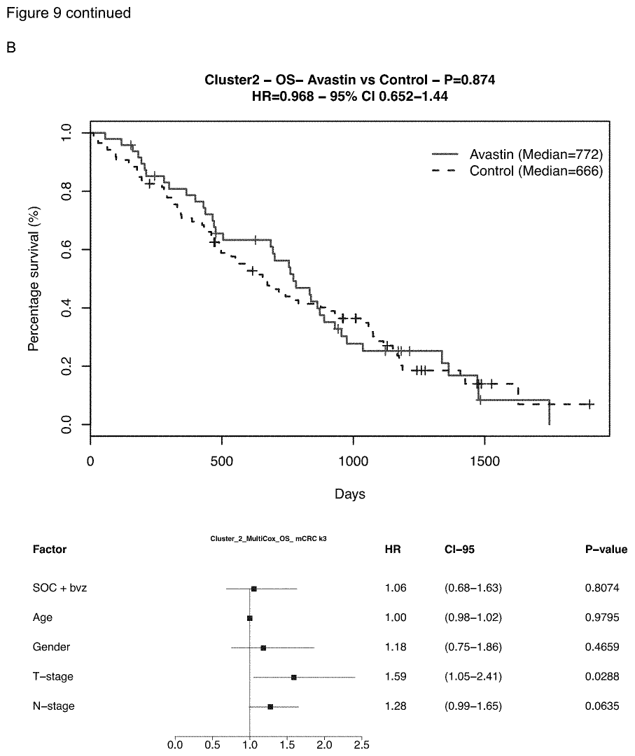

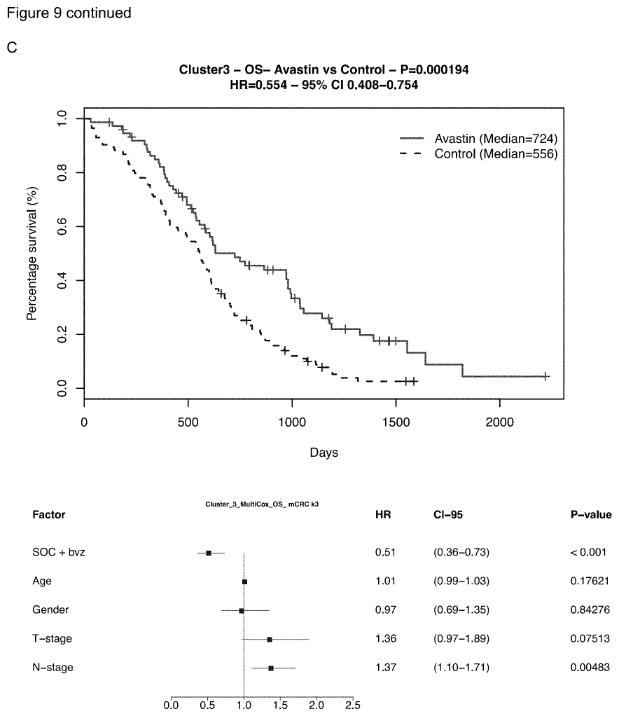

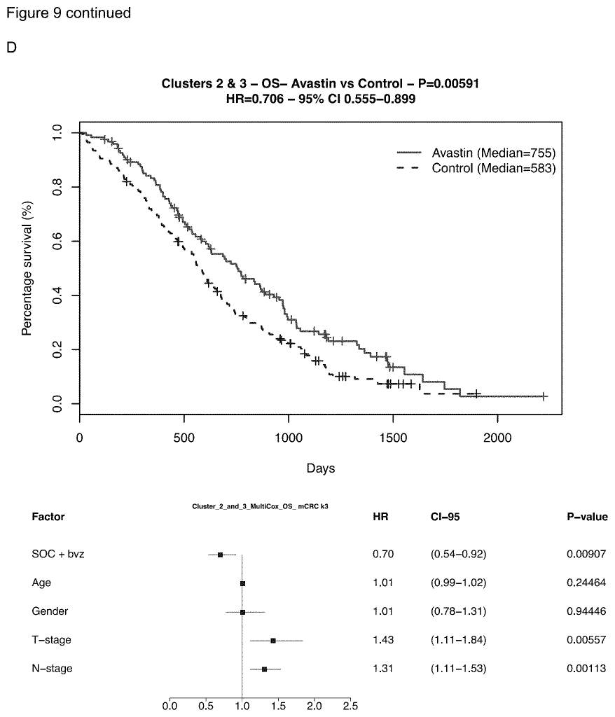

We stratified patients in two groups, namely those that received combination-Avastin therapy (n=141) and a control group receiving combination chemotherapy only (n=220). For each cluster we used the Kaplan Meier method with a log-rank test to evaluate the correlation with progression-free survival and overall survival. For progression free survival, patients from clusters 2 and 3 showed a significant benefit when comparing patients treated with Avastin to the control group (P=3.23×10⁻³, HR=0.58, CI95=0.41-0.83 and P=2.01×10⁻⁶, HR=0.495, CI=0.37-66 for cluster 2 and 3 respectively). No difference was noted for the patients in cluster 1. Similar results were obtained when combining the patients from clusters 2 and 3 in one group (P=1.36×10⁻⁷, HR=0.54, CI95=0.43-0.68) (FIG. 8). Furthermore, multivariate analysis using COX-regression correcting for relevant covariables confirmed that clusters 2 and 3 were significantly correlated with improved PFS and OS for patients treated with Avastin compared to patients treated with standard-of-care therapy. Similar results were obtained for overall survival (FIG. 9).

### Example 5: Relation Copy Number Instability (CIN) and Response to Avastin

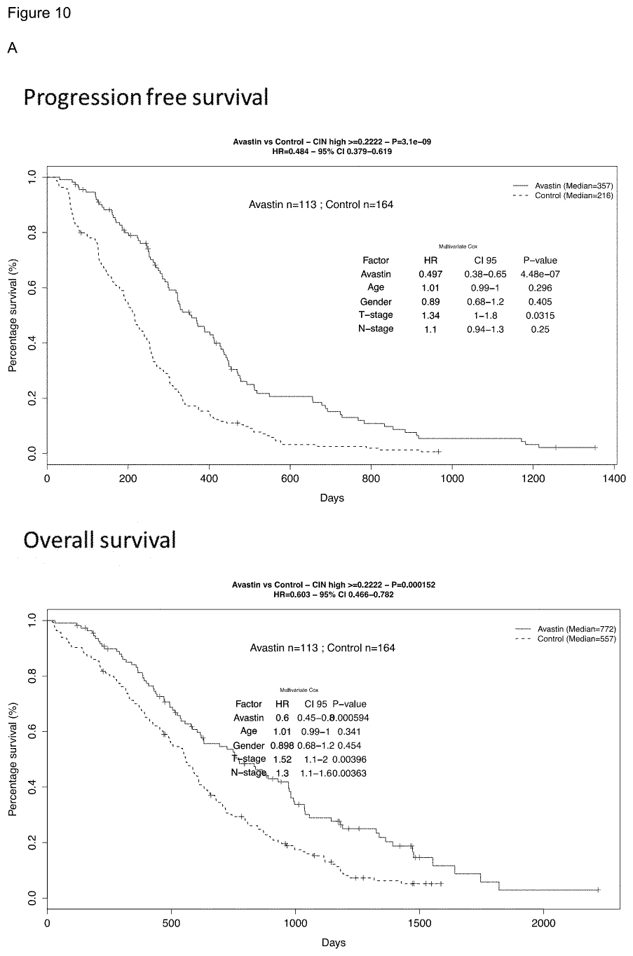

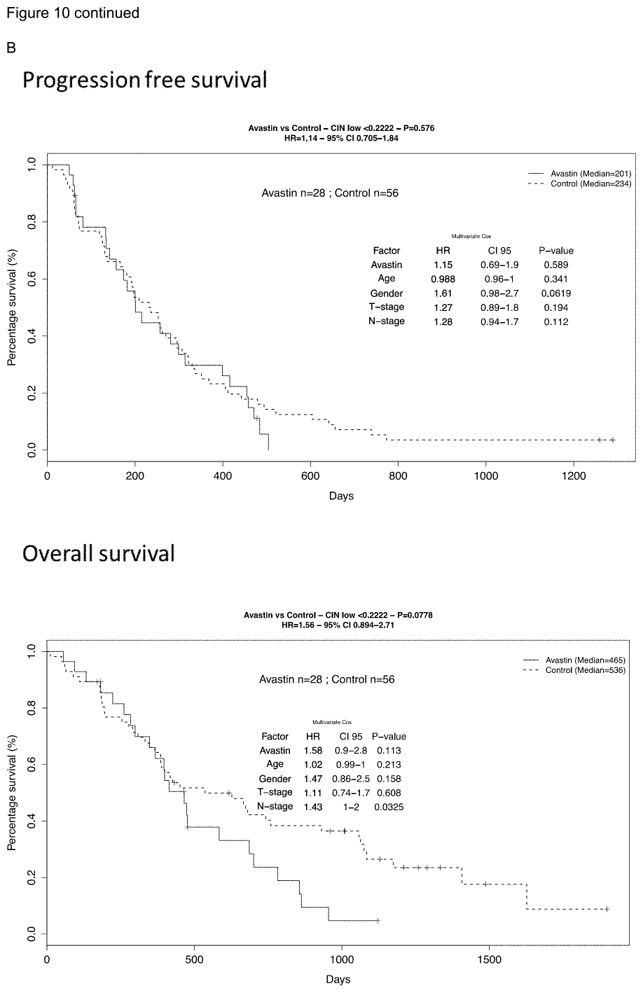

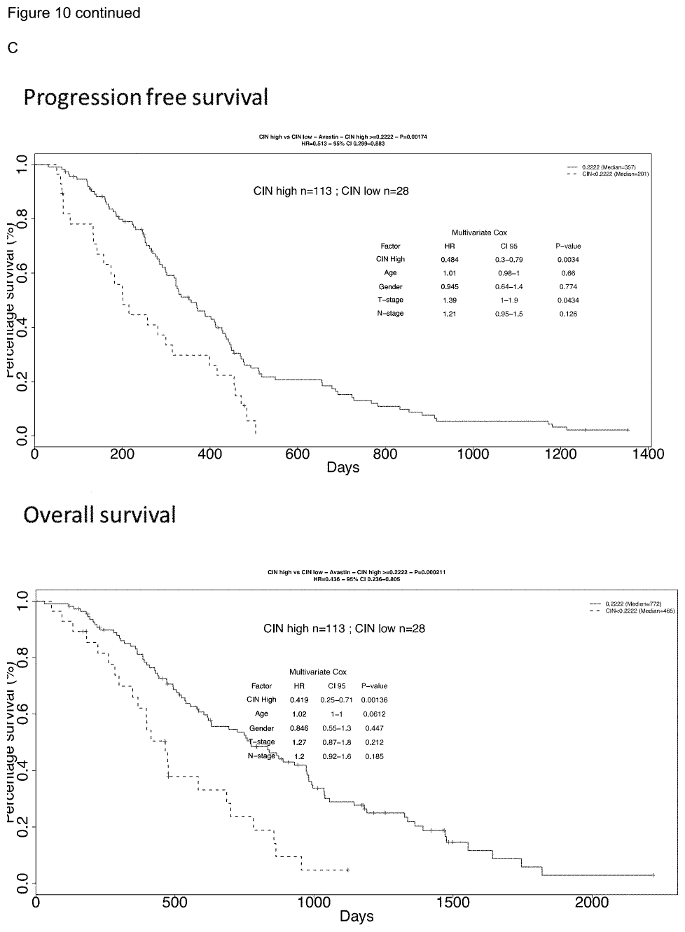

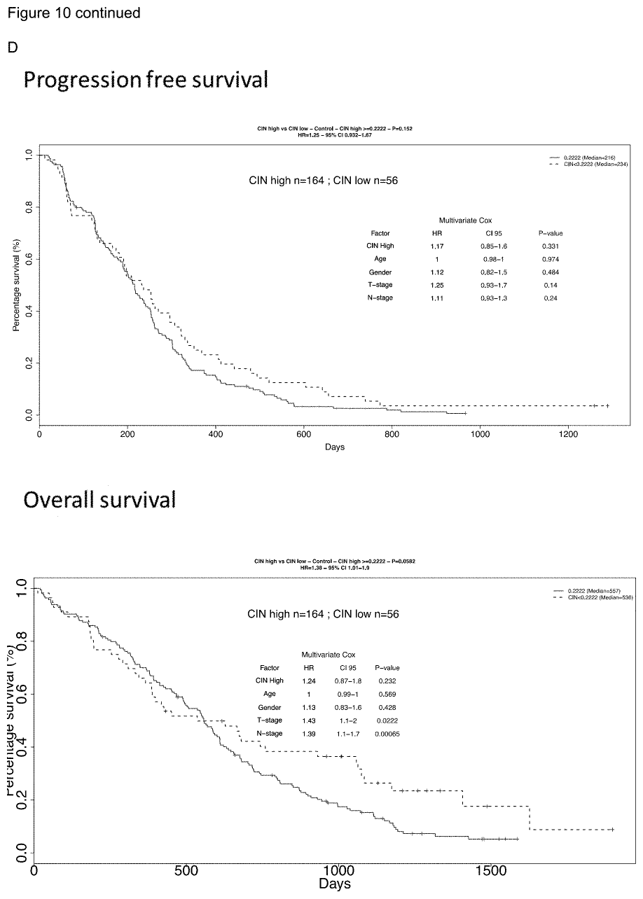

Since patients from clusters 2 and 3 showed a good response to Avastin and these clusters were characterized by a higher CIN we hypothesized that tumors with CIN would have a better response to Avastin. We therefore divided the patients in two groups based on the proportion of the regions that are affected by CNAs (i.e. CNA-high tumors which have more affected regions the first quartile limit and CNA-low tumors with less than or equal to the first quartile limit)(Guinney et al 2015 Nature Medicine 21:1350-1356). When comparing CNA-high with CNA-low tumors within the group of patients that were treated with Avastin, CNA-high tumors showed a significant better progression free survival (P=1.74×10³; HR=0.513; CI95=0.30-0.88). Multivariate analysis using a COX-regression with age, TNM-staging as numerical factors and age as categorical factor revealed that this effect was also observed independent from clinical factors (P=3.4×10⁻³; HR=0.484; CI95=0.30-0.79). In contrast, this correlation was not observed when comparing CNA-high and CNA-low tumors in the control group (FIG. 10). Additionally we compared CNA-high tumors treated with Avastin to those that were not treated with Avastin. We observed a very strong association with increased survival for CNA-high patients treated with Avastin (P=3.1×10-9; HR=0.484; CI95=0.38-0.61). This correlation was confirmed using multivariate analysis (P=4.4×10-7; HR=0.497; CI95=0.38-0.65), again this correlation was observed independent from clinical factors. No such correlation was observed when assessing CNA-low patients treated with Avastin versus CNA-low patients not treated with Avastin (FIG. 10). Similar results were obtained when analysing overall survival.

### Example 6: Hierarchical Clustering Using Only Focal Amplifications

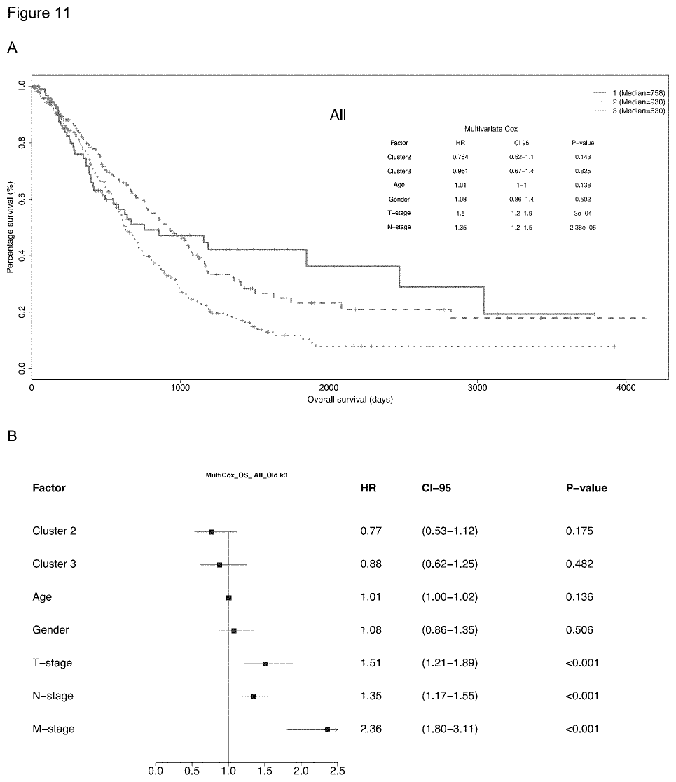

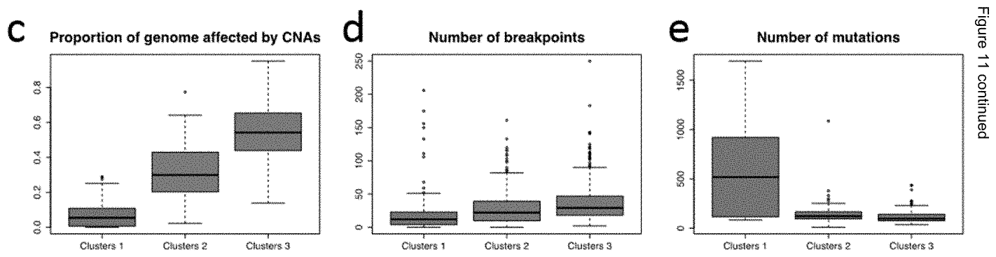

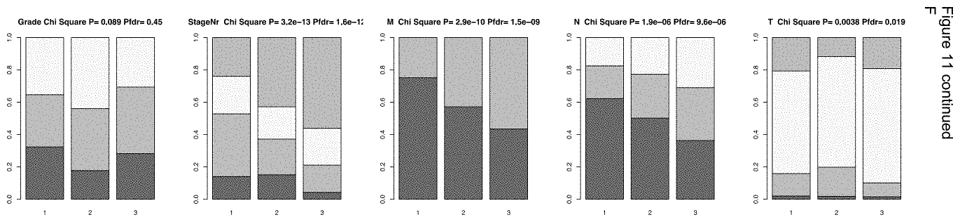

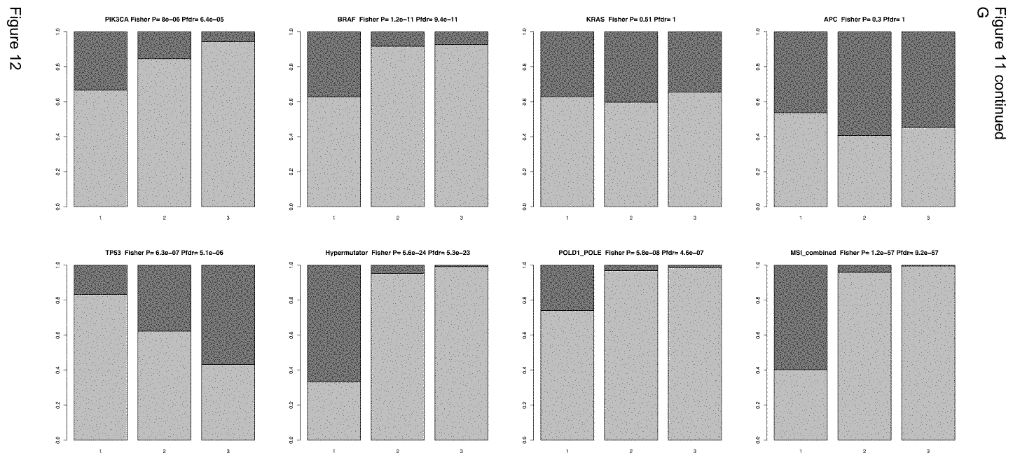

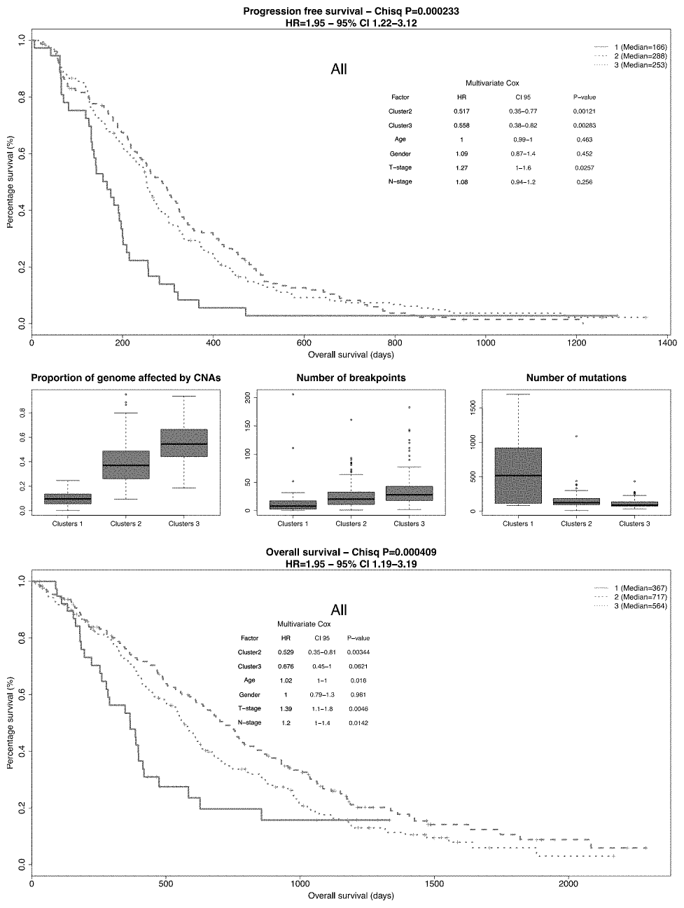

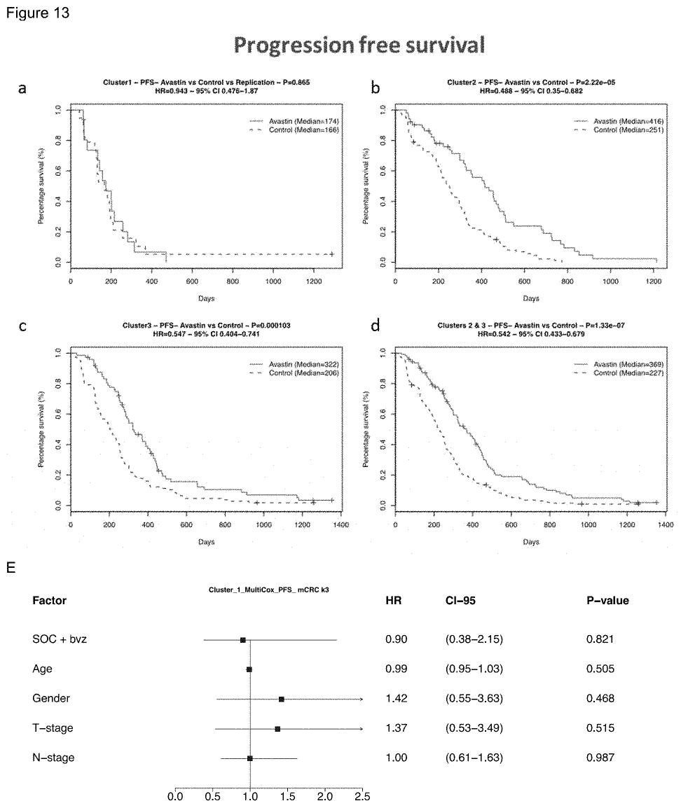

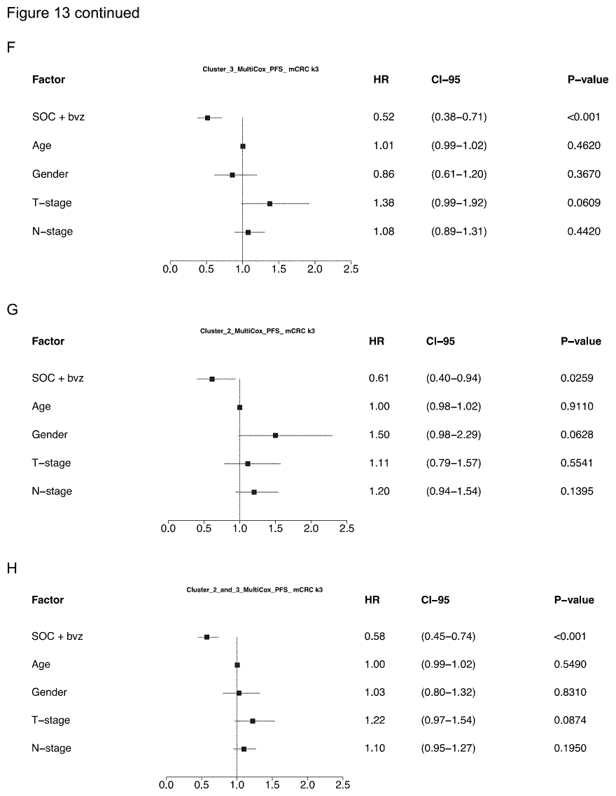

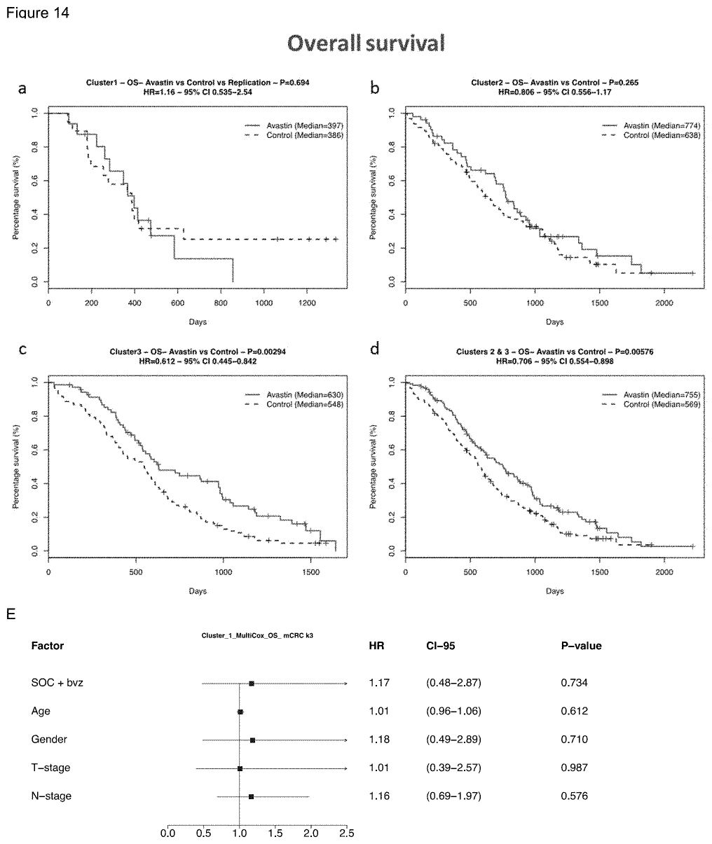

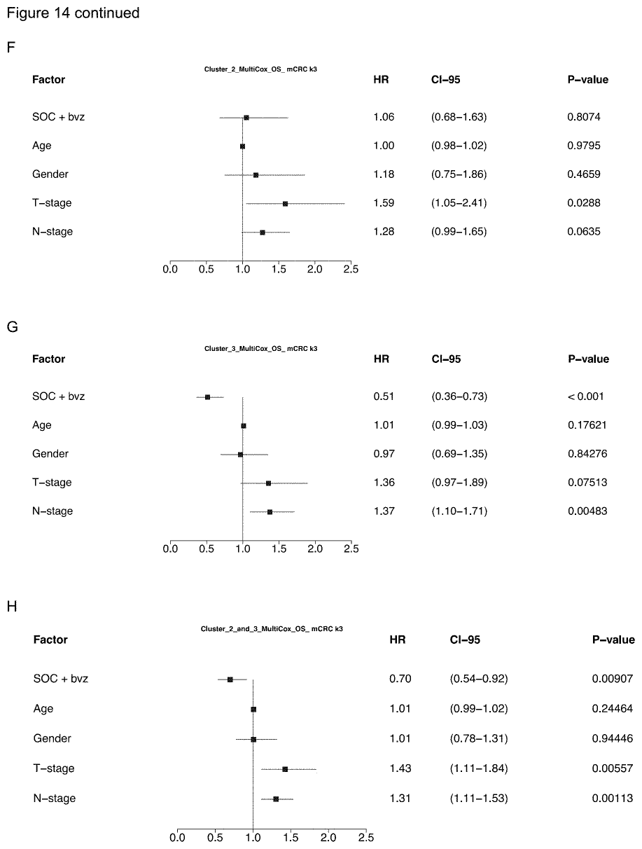

To determine whether our clustering technique could also be performed using only focal CNAs we repeated the clustering using only focal CNAs as input (Table 5). This resulted in the identification of 3 very similar clusters. Characteristics of the three clusters when performing clustering on all CRC samples (n=883) are nearly identical to the clusters created with all 180 CNAs (FIG. 11). Similarly, in mCRC samples, cluster 1 is correlated with a worse prognosis compared to cluster 2 and 3 (FIG. 12) and clusters 2 and 3 are furthermore predictive for a beneficial response to bevacizumab (FIG. 13, 14).

### Example 7: Methods for Single Sample Classification

In order to be able to classify a single sample to one of the three clusters and evaluate how many genomic regions are needed to classify a given CRC sample into one of the three predefined clusters we used 3 different techniques: recursive partitioning, random forest classification and k-nearest neighbour classification (Breiman et al 1984 Classification and regression trees, Wadsworth 368; Breiman 2001 Random Forest, Machine Learning 45:5-32; Venables and Ripley 2002 Modern Applied Statistics with S, Springer).

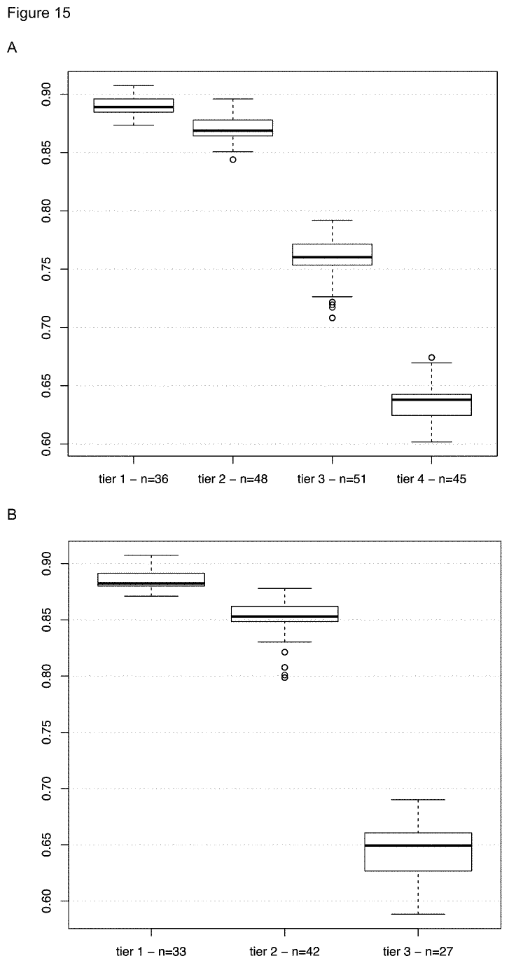

Recursive partitioning revealed that as little as 5 regions can be used to classify samples in one of the three defined clusters with an accuracy as high as 90.7% (FIG. 15). At first instance, we performed this analysis on all 180 regions. We were able to identify 36 regions that yielded the highest accuracy (Table 6; tier 1; average accuracy 89.1+−0.8% for 2789 generated trees using 5-11 regions per tree), a second set of 48 regions (Table 7; tier 2; n=5073 trees generated using 6-12 regions per tree) allowed to assign samples to the predefined clusters with an average accuracy of 87.0±0.9%, a third and fourth set of respectively 51 and 45 regions allowed to assign samples with an average accuracy of respectively 76.0±1.6% and 63.5±1.4% (Table 8 and 9; tier 3 and 4; n=7597 and 3555 trees using 5-16 and 4-13 regions per tree respectively). Similarly, using only the 102 regions that are affected by focal CNAs we were able to identify 33 regions that on average yielded the highest accuracy (Table 10; tier 1; 88.6±0.8% for 2378 generated trees using 5-13 regions per tree), a second set of 42 regions (Table 11; tier 2; n=3726 trees generated using 5-13 regions per tree) allowed to assign samples to the predefined clusters with an average accuracy of 85.4±1.3% and a third set of 27 regions allowed to assign samples with an average accuracy of 64.4±1.9% (Table 12; tier 3; n=9142 trees using 2-17 regions per tree).

In a second approach, we used the random forest classification algorithm to build a classification model using the predefined clusters from the hierarchical clustering as golden standard. In a first step, we performed a 10-fold cross-validation on the original dataset to determine the accuracy of the model. Hereto, we divided the 442 mCRC samples used for the original clustering 10 times at random, each time in a training set (90% of the samples) and validation set (10% of the samples) in such a manner that each sample is presented only once in the whole of 10 validation sets. Next a random forest classifier was generated from 500 balanced bootstraps of the training data. When we applied this classifier to the validation data the classifier demonstrated robust performance with high overall accuracy (94.1% and 91.5% for all 180 regions and only the 102 focal regions respectively) with a >90% balanced accuracy across all 3 clusters (Table 17). In a last step, we built a final model using the complete dataset as input and subsequently also calculated the contribution of each region to the final model. These contributions are represented in Table 13. Similarly, for the analysis using the 102 regions affected by focal CNAs only, the contribution of each region to this model is represented in Table 14.

It is of interest to note that the tier 1 and 2 from the recursive partitioning are also the highest ranking regions when comparing them with the random test contribution (Table 15 and table 16).

In a third approach, we used the k-nearest neighbors algorithm to build a classification model. Similar as the random forest classification we used a 10-fold cross validation to determine the model accuracy which was 86.6% and 87.3% for all 180 regions and only the 102 focal regions respectively with a >88% balanced accuracy across all 3 clusters. In a next step we applied these models to an independent dataset.

### Example 8. Replication on an Independent Dataset Using Knn and Randomforest Classification

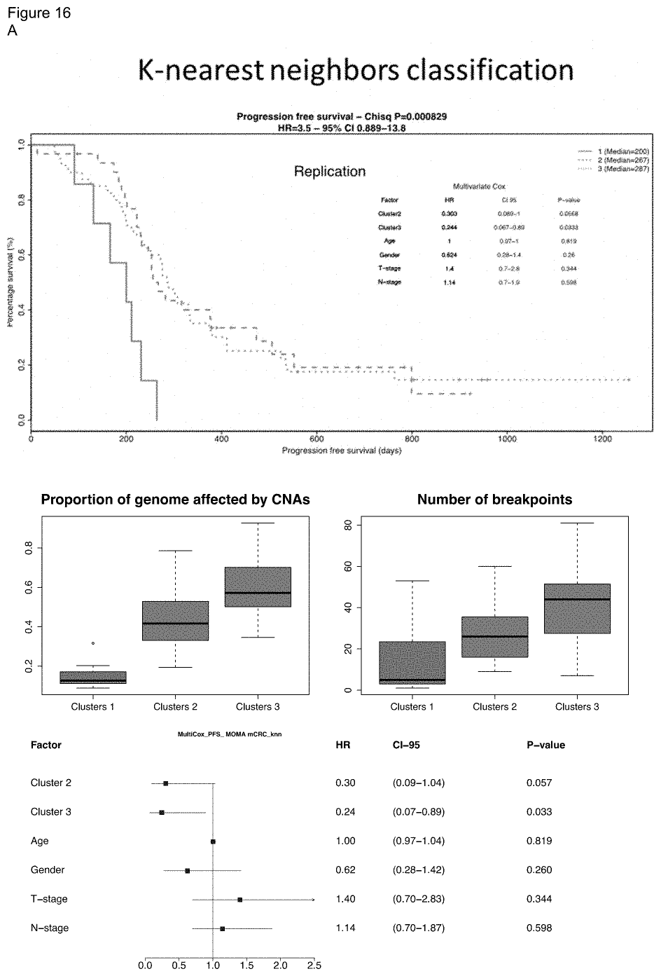

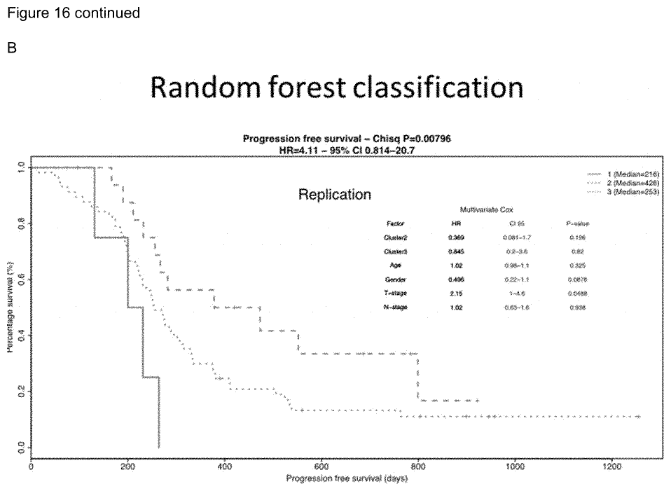

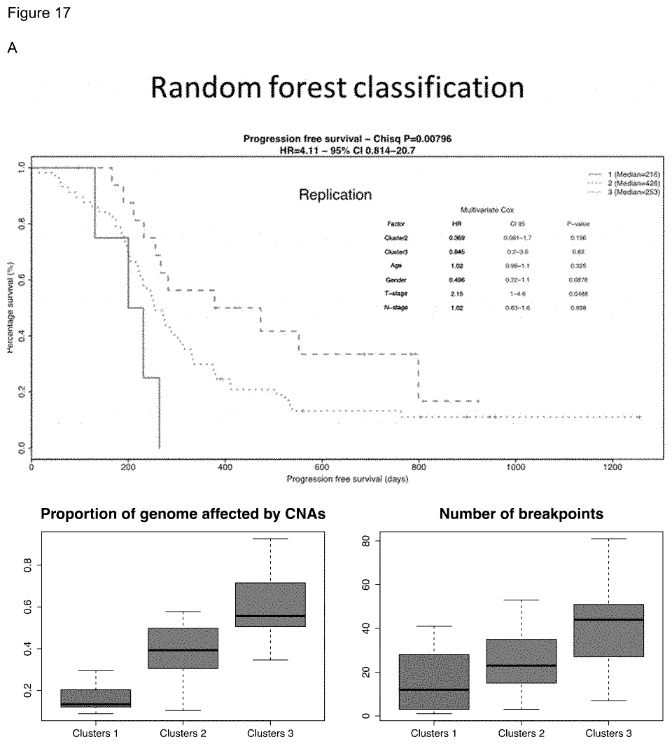

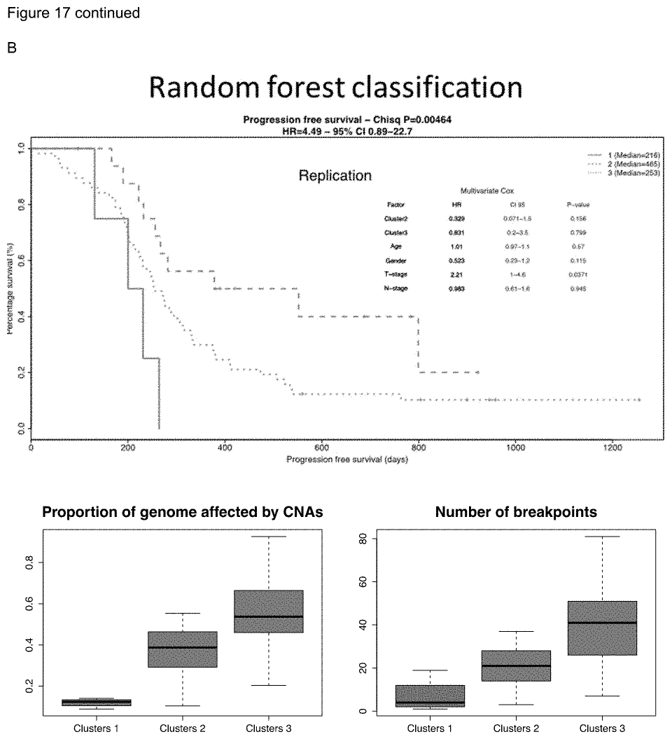

We applied both the k-nearest neighbors and random forest models to the additional dataset of 78 mCRC samples. Using both the k-nearest neighbouring model and the random forest classification we were able to classify the samples in 3 different clusters with very similar characteristics as the predefined clusters that arose from the hierarchical clustering. Further survival analysis again showed that patients from cluster 1 have worse PFS compared to patients from clusters 2 and 3 both in the analysis with all 180 regions as well as using only the 102 regions affected by focal CNAs (FIGS. 16 and 17 respectively).

### Example 9. The Relation of Copy Number Instability in the Different Subsets of Regions and the Response to Avastin

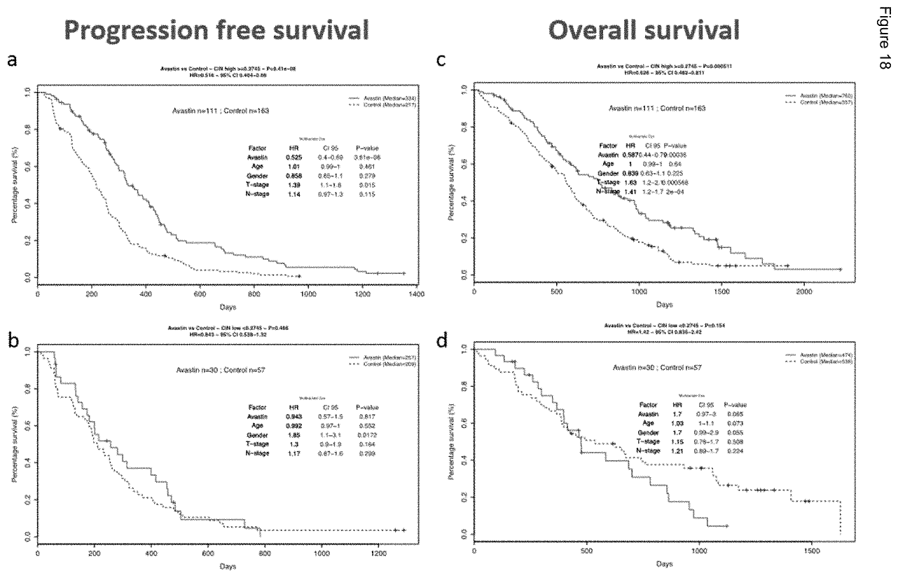

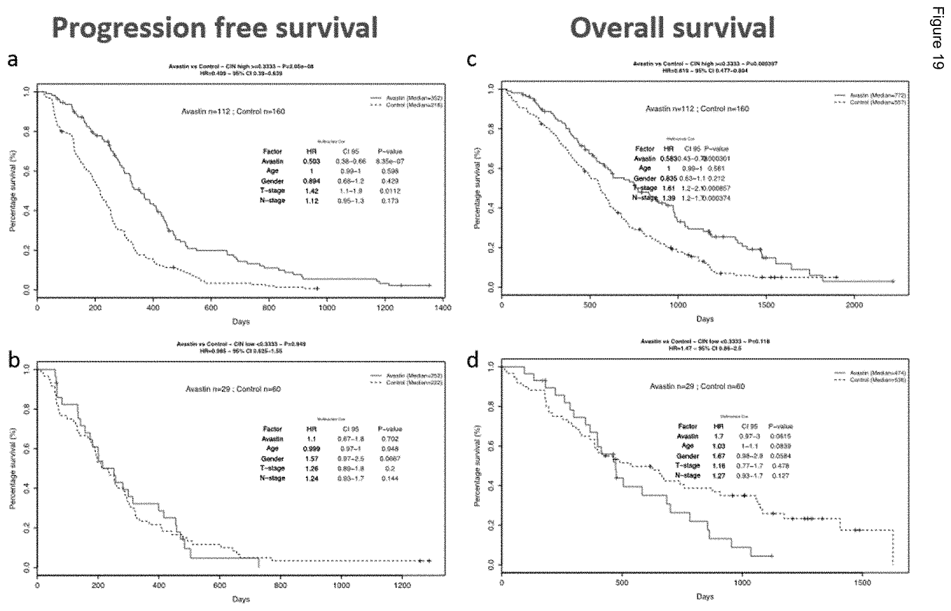

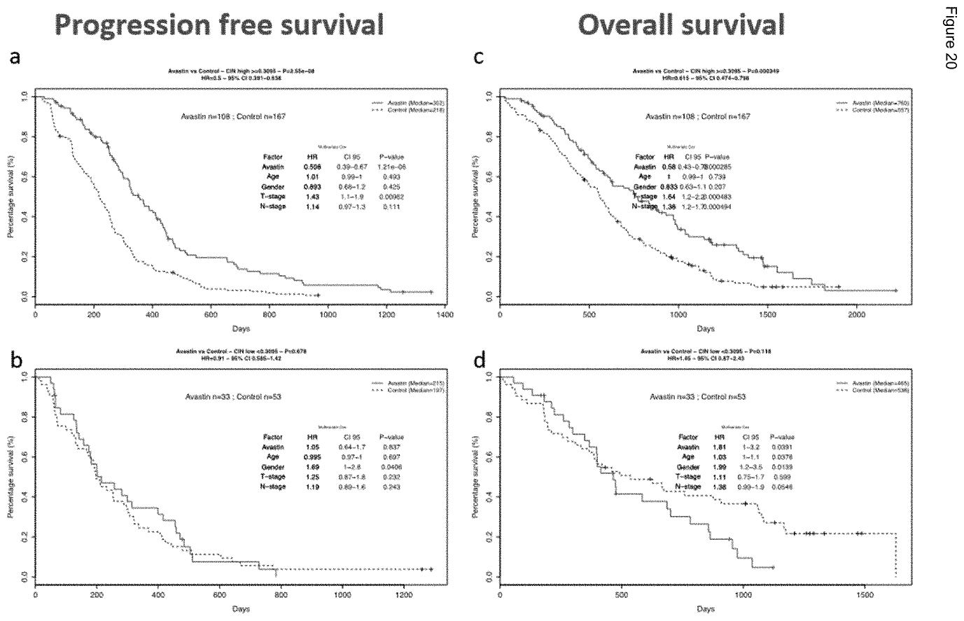

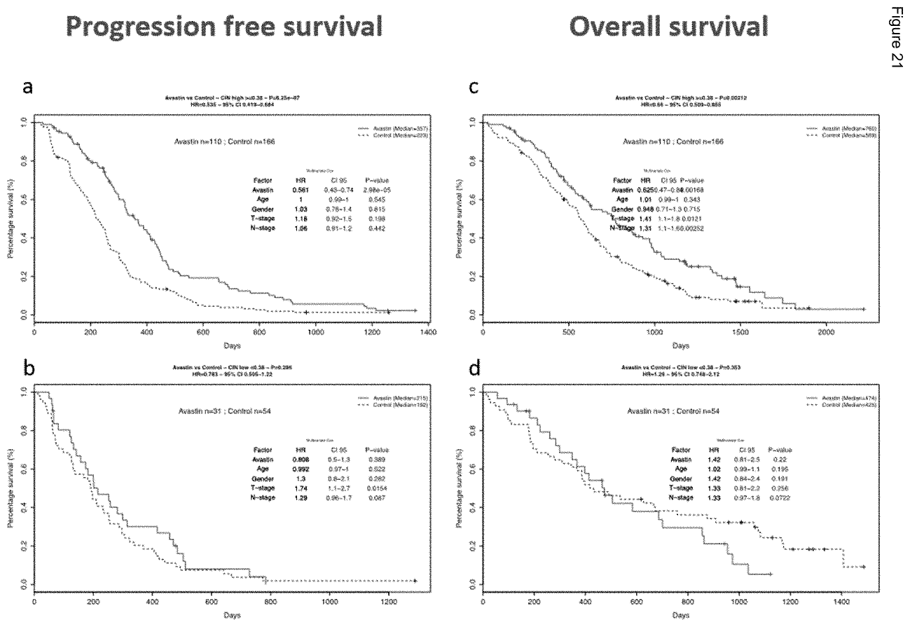

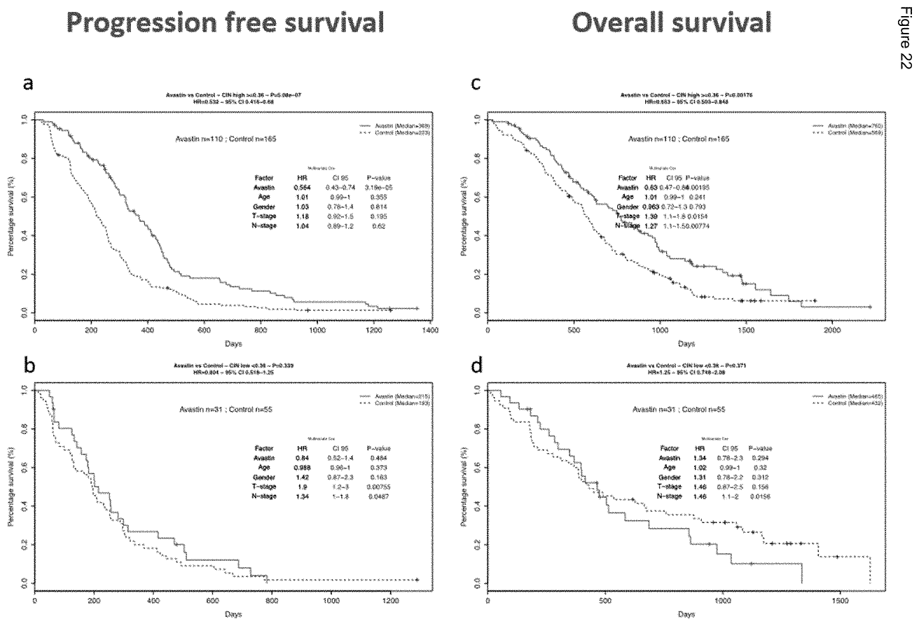

Similar as in example 5 we divided the patients in two groups based on the proportion of the regions that are affected by CNAs (i.e. CNA-high tumors which have more affected regions the first quartile limit and CNA-low tumors with less than or equal to the first quartile limit) and performed this for 6 different subsets (1) using only the 102 focal regions, (2) using the top 50 ranked regions from the random forest classification model built with the 102 focal regions, (3) using the tier 1 and tier 2 regions from the recursive partitioning applied on the 102 focal regions, (4) using all 180 genomic regions (5) using the tier 1 and tier 2 regions from the recursive partitioning applied all 180 regions and (6) using the top 50 ranked regions from the random forest classification model built with the 102 focal regions (FIGS. 18-22, Table 18). For each of the subsets we observed that CNA-high tumors of patients treated with Avastin show a significant increase in progression free and overall survival compared to patients treated with standard-of-care chemotherapy. No such effect was noted for CNA-low tumors.

### Example 10. Replication of the Relation Between CIN and Response to Avastin

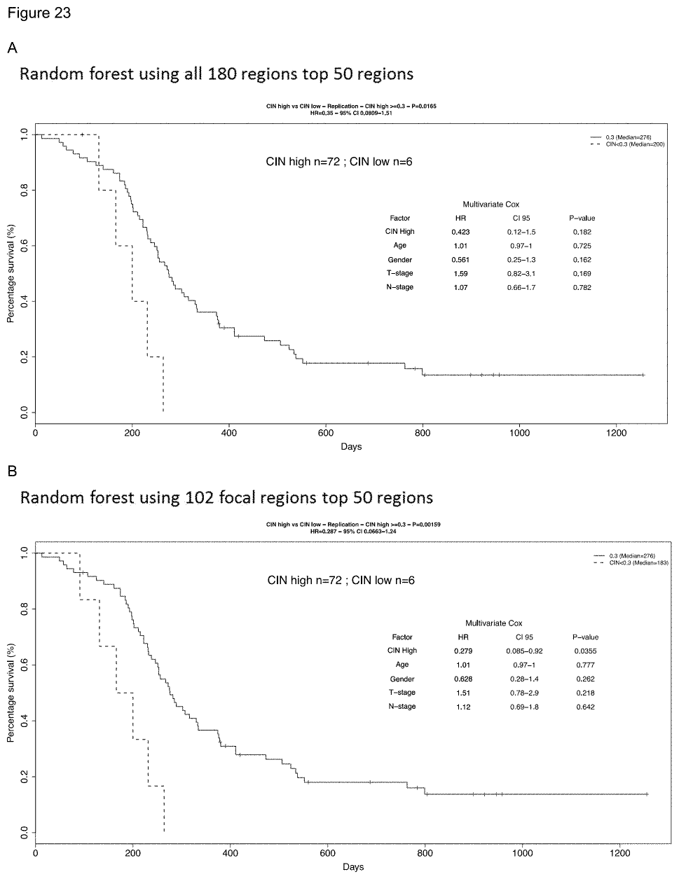

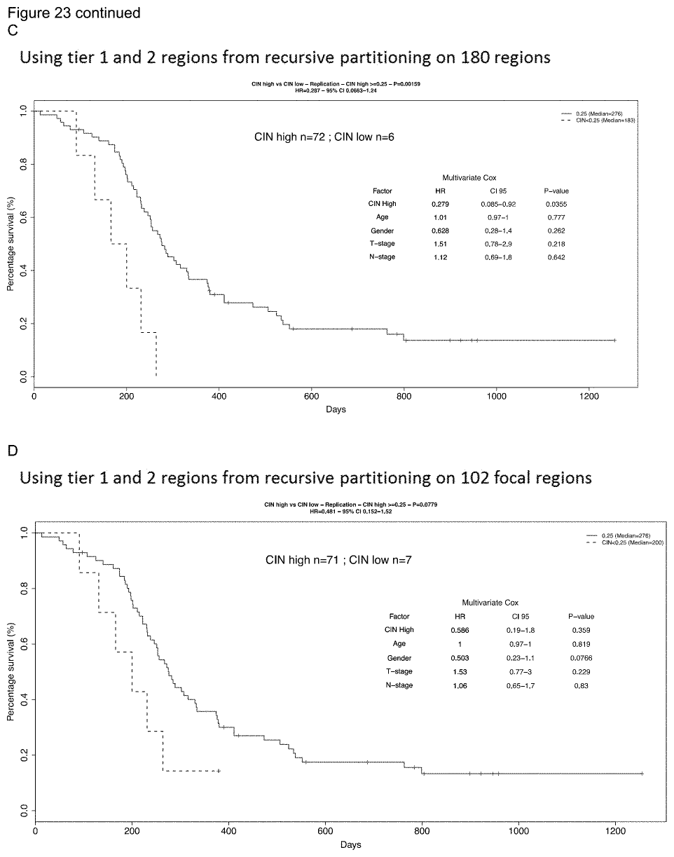

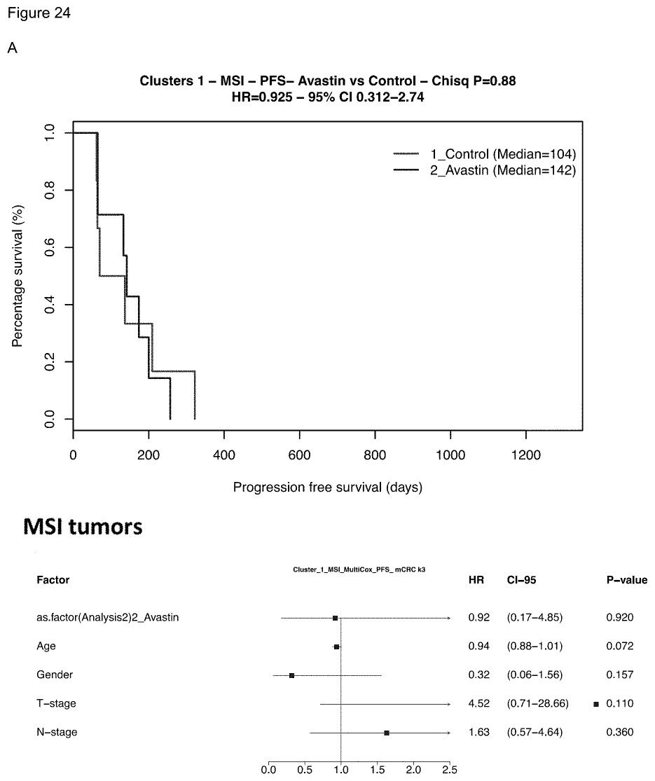

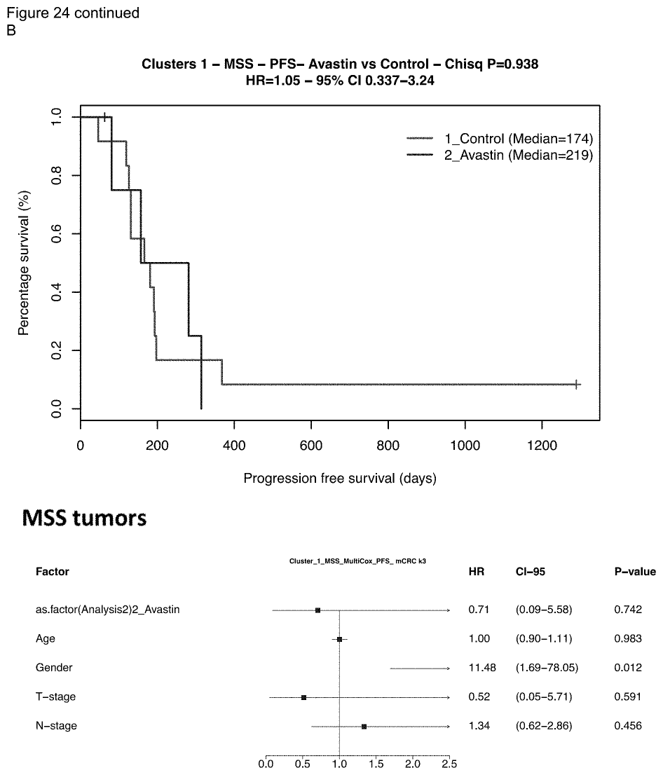

Similar as the analysis in example 5 we stratified patients in CNA-high and CNA-low (with the thresholds set to 30% and 25% for the top 50 ranking regions from the random forest classifiers and the tier 1 and 2 regions for both only focal regions and all 180 regions respectively. For each of the different subsets of genomic regions CNA-high tumors showed an increased progression free survival (FIG. 23).

TABLE 1
Biomarker panel listing the 180 genomic regions used to cluster the studied CRC samples in 3
genomic subtypes. The panel of genomic regions consists of 43 focal amplifications, 59 focal deletions,
39 whole-arm amplifications and 39 whole-arm deletions.

| Unique Name | Descriptor | Wide Peak Limits | Peak Limits | Region Limits |
|---|---|---|---|---|
| Amplification | 1p34.2 | chr1: 39144054-44367347 | chr1: 42834925-43114106 | chr1: 42692914-43456374 |
| Peak 1 | | (probes 8327:9928) | (probes 9441:9533) | (probes 9397:9635) |
| Amplification | 1q22 | chr1: 120494739-199253746 | chr1: 155143717-155177826 | chr1: 120497533-175439985 |
| Peak 2 | | (probes 33660:51419) | (probes 35410:35412) | (probes 33661:42337) |
| Amplification | 1q43 | chr1: 201615295-249250621 | chr1: 242413158-242638099 | chr1: 242270003-249250621 |
| Peak 3 | | (probes 52203:66664) | (probes 65295:65368) | (probes 65251:66664) |
| Amplification | 2q33.1 | chr2: 181446594-243199373 | chr2: 199603526-199649638 | chr2: 199525178-199824262 |
| Peak 4 | | (probes 121383:140439) | (probes 127082:127089) | (probes 127052:127139) |
| Amplification | 3q29 | chr3: 174745992-198022430 | chr3: 195922177-195991311 | chr3: 174746836-198022430 |
| Peak 5 | | (probes 195590:202834) | (probes 202552:202570) | (probes 195591:202834) |
| Amplification | 5p15.33 | chr5: 1-6418038 | chr5: 1-949725 | chr5: 1-2807521 |
| Peak 6 | | (probes 260156:261588) | (probes 260156:260170) | (probes 260156:260460) |
| Amplification | 5p12 | chr5: 33430780-50470114 | chr5: 43822799-43929095 | chr5: 37334807-44266311 |
| Peak 7 | | (probes 269602:272992) | (probes 272538:272572) | (probes 270584:272613) |
| Amplification | 6p21.1 | chr6: 43545080-44164949 | chr6: 43765716-44163738 | chr6: 36748055-52028937 |
| Peak 8 | | (probes 323466:323553) | (probes 323497:323552) | (probes 321584:325929) |
| Amplification | 6q23.3 | chr6: 135342981-135632150 | chr6: 135513416-135593890 | chr6: 133418393-138096856 |
| Peak 9 | | (probes 350621:350776) | (probes 350670:350763) | (probes 350049:351529) |
| Amplification | 7p11.2 | chr7: 54949302-55950640 | chr7: 55248172-55271747 | chr7: 54949902-55955413 |
| Peak 10 | | (probes 380333:380688) | (probes 380546:380547) | (probes 380334:380690) |
| Amplification | 8p11.23 | chr8: 38162916-38237532 | chr8: 38165117-38236937 | chr8: 34342520-48854434 |
| Peak 11 | | (probes 417907:417912) | (probes 417908:417911) | (probes 417738:418611) |
| Amplification | 8p11.21 | chr8: 41760295-42054539 | chr8: 41767516-41796226 | chr8: 34342520-48854434 |
| Peak 12 | | (probes 418156:418183) | (probes 418157:418159) | (probes 417738:418611) |
| Amplification | 8q12.2 | chr8: 60847395-62906825 | chr8: 61658252-61874896 | chr8: 54646329-146364022 |
| Peak 13 | | (probes 421733:422344) | (probes 421947:422037) | (probes 420141:444693) |
| Amplification | 8q21.13 | chr8: 80636493-82552412 | chr8: 81853752-81935079 | chr8: 54646329-146364022 |
| Peak 14 | | (probes 427272:427752) | (probes 427579:427595) | (probes 420141:444693) |
| Amplification | 8q22.3 | chr8: 101853171-101878037 | chr8: 101853464-101876083 | chr8: 54646329-146364022 |
| Peak 15 | | (probes 432850:432855) | (probes 432851:432854) | (probes 420141:444693) |
| Amplification | 8q24.21 | chr8: 128574277-128592142 | chr8: 128574768-128591041 | chr8: 54646329-146364022 |
| Peak 16 | | (probes 440457:440461) | (probes 440458:440460) | (probes 420141:444693) |
| Amplification | 10q22.3 | chr10: 79956009-83248579 | chr10: 80445049-80732930 | chr10: 77952482-83583616 |
| Peak 17 | | (probes 504622:505397) | (probes 504807:504859) | (probes 503919:505484) |
| Amplification | 11p15.5 | chr11: 2091739-2302637 | chr11: 2095340-2301396 | chr11: 2095340-2305324 |
| Peak 18 | | (probes 520960:520971) | (probes 520961:520970) | (probes 520961:520973) |
| Amplification | 11q13.3 | chr11: 68746750-69827851 | chr11: 69311828-69824706 | chr11: 68748468-70760456 |
| Peak 19 | | (probes 539709:539869) | (probes 539796:539868) | (probes 539710:540034) |
| Amplification | 12p13.32 | chr12: 3941807-4552801 | chr12: 4257035-4414967 | chr12: 1-8970181 |
| Peak 20 | | (probes 562075:562238) | (probes 562162:562219) | (probes 561076:563266) |
| Amplification | 12p11.22 | chr12: 24880798-28477058 | chr12: 27800441-27813180 | chr12: 25955987-28156868 |
| Peak 21 | | (probes 567897:568917) | (probes 568722:568722) | (probes 568181:568838) |
| Amplification | 13q12.2 | chr13: 28164386-28564331 | chr13: 28174614-28222659 | chr13: 1-115169878 |
| Peak 22 | | (probes 600542:600653) | (probes 600543:600550) | (probes 599000:624834) |
| Amplification | 13q22.1 | chr13: 73775176-74007122 | chr13: 73906681-74004816 | chr13: 1-115169878 |
| Peak 23 | | (probes 612853:612922) | (probes 612891:612921) | (probes 599000:624834) |
| Amplification | 13q34 | chr13: 110097815-111753132 | chr13: 110614601-110852082 | chr13: 1-115169878 |
| Peak 24 | | (probes 624037:624356) | (probes 624207:624235) | (probes 599000:624834) |
| Amplification | 15q26.1 | chr15: 84648780-102531392 | chr15: 90811423-90958538 | chr15: 90086447-91895585 |
| Peak 25 | | (probes 669559:675028) | (probes 671504:671507) | (probes 671145:671795) |
| Amplification | 16p11.2 | chr16: 30548665-30684980 | chr16: 30549334-30682578 | chr16: 28556705-46663588 |
| Peak 26 | | (probes 681497:681499) | (probes 681498:681498) | (probes 681456:681707) |
| Amplification | 16q12.1 | chr16: 52243935-52652774 | chr16: 52383686-52652198 | chr16: 51078632-53117566 |
| Peak 27 | | (probes 683896:684007) | (probes 683905:684006) | (probes 683397:684118) |
| Amplification | 17q11.2 | chr17: 27339261-27528113 | chr17: 27339592-27419750 | chr17: 20042949-40168023 |
| Peak 28 | | (probes 701849:701907) | (probes 701850:701866) | (probes 701549:704344) |
| Amplification | 17q12 | chr17: 37747533-38052599 | chr17: 37950674-38031986 | chr17: 20042949-40168023 |
| Peak 29 | | (probes 703891:703930) | (probes 703917:703929) | (probes 701549:704344) |
| Amplification | 17q23.2 | chr17: 58400463-58674819 | chr17: 58400997-58532661 | chr17: 57045336-62714320 |
| Peak 30 | | (probes 708991:709072) | (probes 708992:709031) | (probes 708646:710280) |
| Amplification | 17q24.2 | chr17: 65638523-65873408 | chr17: 65649475-65769071 | chr17: 63042470-68655121 |
| Peak 31 | | (probes 710976:711074) | (probes 710978:711037) | (probes 710378:712109) |
| Amplification | 17q25.3 | chr17: 77601682-77857881 | chr17: 77766436-77856811 | chr17: 75845339-81195210 |
| Peak 32 | | (probes 714127:714155) | (probes 714137:714154) | (probes 713778:714413) |
| Amplification | 18q11.2 | chr18: 19517253-20155059 | chr18: 19525020-20154578 | chr18: 19243919-21268418 |
| Peak 33 | | (probes 718275:718278) | (probes 718276:718277) | (probes 718239:718447) |
| Amplification | 18q21.1 | chr18: 46041609-46930382 | chr18: 46474762-46604295 | chr18: 46043163-46624631 |
| Peak 34 | | (probes 725325:725688) | (probes 725462:725478) | (probes 725326:725481) |
| Amplification | 19q13.11 | chr19: 32439834-33021877 | chr19: 32966965-32988344 | chr19: 19939350-41312227 |
| Peak 35 | | (probes 739770:739825) | (probes 739799:739805) | (probes 739769:742785) |
| Amplification | 19q13.2 | chr19: 39844313-40357920 | chr19: 39848086-40009185 | chr19: 19939350-41312227 |
| Peak 36 | | (probes 742339:742488) | (probes 742340:742365) | (probes 739769:742785) |
| Amplification | 20p12.3 | chr20: 5376354-5742957 | chr20: 5379141-5737687 | chr20: 5156552-5961498 |
| Peak 37 | | (probes 749386:749394) | (probes 749387:749393) | (probes 749320:749400) |
| Amplification | 20p12.1 | chr20: 16140291-16967195 | chr20: 16488303-16962609 | chr20: 16146669-16970141 |
| Peak 38 | | (probes 751931:752121) | (probes 751982:752120) | (probes 751932:752123) |
| Amplification | 20p11.22 | chr20: 22058560-22353663 | chr20: 22063756-22350551 | chr20: 20938975-24286806 |
| Peak 39 | | (probes 753204:753212) | (probes 753205:753211) | (probes 753103:753480) |
| Amplification | 20q11.21 | chr20: 29981165-30284236 | chr20: 30158807-30231055 | chr20: 24375411-63025520 |
| Peak 40 | | (probes 753711:753769) | (probes 753729:753748) | (probes 753519:762719) |
| Amplification | 20q13.12 | chr20: 42534268-43281676 | chr20: 42537604-42773258 | chr20: 24375411-63025520 |
| Peak 41 | | (probes 756743:756930) | (probes 756744:756753) | (probes 753519:762719) |
| Amplification | 20q13.2 | chr20: 52240629-52852159 | chr20: 52246659-52447690 | chr20: 24375411-63025520 |
| Peak 42 | | (probes 759885:759989) | (probes 759886:759947) | (probes 753519:762719) |
| Amplification | 20q13.32 | chr20: 56110288-57258354 | chr20: 57000076-57085518 | chr20: 24375411-63025520 |
| Peak 43 | | (probes 760955:761384) | (probes 761307:761325) | (probes 753519:762719) |
| Deletion Peak 1 | 1p36.31 | chr1: 4843384-6053964 | chr1: 5646446-6050774 | chr1: 1-37080378 |
| | | (probes 343:539) | (probes 537:538) | (probes 1:7694) |
| Deletion Peak 2 | 1p36.11 | chr1: 26898389-27219375 | chr1: 27139248-27185402 | chr1: 1-37080378 |
| | | (probes 5577:5639) | (probes 5629:5630) | (probes 1:7694) |
| Deletion Peak 3 | 1p33 | chr1: 49187432-50544677 | chr1: 48847112-50490440 | chr1: 49196205-51396500 |
| | | (probes 11383:11605) | (probes 11319:11598) | (probes 11385:11801) |
| Deletion Peak 4 | 1p31.1 | chr1: 68959091-82269682 | chr1: 79308665-79566142 | chr1: 54944113-149961894 |
| | | (probes 17617:22019) | (probes 20993:20994) | (probes 12746:33663) |
| Deletion Peak 5 | 1p21.1 | chr1: 102460262-107618224 | chr1: 104041213-104355050 | chr1: 54944113-149961894 |
| | | (probes 28724:30035) | (probes 29059:29060) | (probes 12746:33663) |
| Deletion Peak 6 | 2p25.3 | chr2: 1-4752017 | chr2: 1-488785 | chr2: 1-5260278 |
| | | (probes 66665:67509) | (probes 66665:66665) | (probes 66665:67543) |
| Deletion Peak 7 | 2p21 | chr2: 42587649-44009118 | chr2: 43440855-43455807 | chr2: 43159857-43470966 |
| | | (probes 79800:80265) | (probes 80094:80097) | (probes 79991:80104) |
| Deletion Peak 8 | 2q23.1 | chr2: 147341956-149499455 | chr2: 148437688-148750846 | chr2: 148444810-148756213 |
| | | (probes 110935:111344) | (probes 111211:111212) | (probes 111212:111213) |
| Deletion Peak 9 | 2q37.3 | chr2: 240321205-243199373 | chr2: 240940975-241060875 | chr2: 240589192-241997730 |
| | | (probes 139897:140439) | (probes 140116:140117) | (probes 139997:140352) |
| Deletion Peak | 3p26.3 | chr3: 1-8350135 | chr3: 1-2251569 | chr3: 1-9456413 |
| 10 | | (probes 140440:142376) | (probes 140440:140440) | (probes 140440:142660) |
| Deletion Peak | 3p14.2 | chr3: 58946448-61555632 | chr3: 59692189-61457375 | chr3: 59692189-61460946 |
| 11 | | (probes 159123:159363) | (probes 159317:159339) | (probes 159317:159341) |
| Deletion Peak | 3p13 | chr3: 70014100-71250049 | chr3: 70976616-71182883 | chr3: 70977475-71315576 |
| 12 | | (probes 162276:162774) | (probes 162708:162756) | (probes 162709:162793) |
| Deletion Peak | 3q26.31 | chr3: 173995229-175766885 | chr3: 174575326-175766885 | chr3: 174646449-174998725 |
| 13 | | (probes 195434:195620) | (probes 195555:195620) | (probes 195574:195593) |
| Deletion Peak | 4p16.2 | chr4: 1-9787265 | chr4: 5915497-5925651 | chr4: 1-11455571 |
| 14 | | (probes 202835:205184) | (probes 204117:204118) | (probes 202835:205810) |
| Deletion Peak | 4q22.1 | chr4: 91143530-93226628 | chr4: 91046982-92557520 | chr4: 91103554-92561908 |
| 15 | | (probes 229840:230295) | (probes 229814:230049) | (probes 229834:230051) |
| Deletion Peak | 4q25 | chr4: 109046573-109544048 | chr4: 109451604-109544048 | chr4: 109489936-109535239 |
| 16 | | (probes 235677:235829) | (probes 235801:235829) | (probes 235811:235826) |
| Deletion Peak | 4q31.3 | chr4: 152679194-153694102 | chr4: 153232273-153473069 | chr4: 152233433-191154276 |
| 17 | | (probes 249686:250016) | (probes 249909:249977) | (probes 249594:260155) |
| Deletion Peak | 4q32.1 | chr4: 156135292-162306004 | chr4: 159683040-159830757 | chr4: 152233433-191154276 |
| 18 | | (probes 250770:252941) | (probes 252055:252100) | (probes 249594:260155) |
| Deletion Peak | 4q35.1 | chr4: 184432080-185262191 | chr4: 184545375-185154488 | chr4: 152233433-191154276 |
| 19 | | (probes 259152:259216) | (probes 259180:259181) | (probes 249594:260155) |
| Deletion Peak | 5p15.33 | chr5: 1-2750686 | chr5: 1368344-2252637 | chr5: 1-3864653 |
| 20 | | (probes 260156:260443) | (probes 260268:260279) | (probes 260156:260838) |
| Deletion Peak | 5q12.1 | chr5: 58263825-59784640 | chr5: 58263825-59784640 | chr5: 50968806-135478060 |
| 21 | | (probes 275334:275642) | (probes 275334:275642) | (probes 273162:297537) |
| Deletion Peak | 5q22.2 | chr5: 111312546-112362638 | chr5: 111747052-111761475 | chr5: 50968806-135478060 |
| 22 | | (probes 290226:290685) | (probes 290365:290367) | (probes 273162:297537) |
| Deletion Peak | 6p25.3 | chr6: 1613114-2256643 | chr6: 1621843-2256643 | chr6: 1626114-2752758 |
| 23 | | (probes 311779:311876) | (probes 311784:311876) | (probes 311786:312043) |
| Deletion Peak | 6p22.2 | chr6: 25926929-26025173 | chr6: 25932794-26025173 | chr6: 25933563-26346501 |
| 24 | | (probes 319619:319631) | (probes 319620:319631) | (probes 319621:319690) |
| Deletion Peak | 6q21 | chr6: 91006553-129206367 | chr6: 111544567-112706964 | chr6: 108785353-116683959 |
| 25 | | (probes 337170:348684) | (probes 343210:343577) | (probes 342315:344882) |
| Deletion Peak | 6q26 | chr6: 161540781-163179430 | chr6: 161540781-163179430 | chr6: 160448048-171115067 |
| 26 | | (probes 359016:359155) | (probes 359016:359155) | (probes 358753:361110) |
| Deletion Peak | 7q31.1 | chr7: 109590674-111367757 | chr7: 110232248-111358792 | chr7: 110595801-110618403 |
| 27 | | (probes 392698:393070) | (probes 392932:393069) | (probes 393001:393002) |
| Deletion Peak | 8p23.3 | chr8: 1-1449850 | chr8: 1-623109 | chr8: 1-10801317 |
| 28 | | (probes 408036:408314) | (probes 408036:408036) | (probes 408036:410838) |
| Deletion Peak | 8p21.3 | chr8: 13423967-26607015 | chr8: 21547484-21647267 | chr8: 11646511-25738962 |
| 29 | | (probes 411497:416107) | (probes 414395:414425) | (probes 411142:415781) |
| Deletion Peak | 8p12 | chr8: 33445578-37452445 | chr8: 34944675-36494184 | chr8: 35186331-35207233 |
| 30 | | (probes 417581:417886) | (probes 417838:417861) | (probes 417850:417851) |
| Deletion Peak | 8q11.1 | chr8: 42874387-48101823 | chr8: 42931769-48061147 | chr8: 42932439-48062091 |
| 31 | | (probes 418403:418427) | (probes 418413:418414) | (probes 418414:418415) |
| Deletion Peak | 9p21.3 | chr9: 21558582-22452906 | chr9: 21995318-22021004 | chr9: 21711940-22331169 |
| 32 | | (probes 452306:452602) | (probes 452487:452491) | (probes 452378:452557) |
| Deletion Peak | 10p15.3 | chr10: 1-855610 | chr10: 1-418075 | chr10: 1-2055670 |
| 33 | | (probes 482107:482163) | (probes 482107:482107) | (probes 482107:482391) |
| Deletion Peak | 10q21.1 | chr10: 51561927-53481136 | chr10: 51561927-54065263 | chr10: 50763189-54253314 |
| 34 | | (probes 495717:495904) | (probes 495717:496053) | (probes 495699:496079) |
| Deletion Peak | 10q23.31 | chr10: 89502327-90051809 | chr10: 89574482-89607380 | chr10: 83248858-135534747 |
| 35 | | (probes 507191:507378) | (probes 507201:507204) | (probes 505400:520735) |
| Deletion Peak | 10q25.2 | chr10: 114197471-115353755 | chr10: 114708532-114929210 | chr10: 83248858-135534747 |
| 36 | | (probes 515003:515382) | (probes 515200:515269) | (probes 505400:520735) |
| Deletion Peak | 10q26.3 | chr10: 133107199-135534747 | chr10: 135221096-135534747 | chr10: 83248858-135534747 |
| 37 | | (probes 520190:520735) | (probes 520735:520735) | (probes 505400:520735) |
| Deletion Peak | 11q22.3 | chr11: 102958343-135006516 | chr11: 108251085-108350263 | chr11: 108179920-108470348 |
| 38 | | (probes 551128:561075) | (probes 552798:552832) | (probes 552783:552882) |
| Deletion Peak | 12p13.2 | chr12: 12412186-13039757 | chr12: 12525464-12721981 | chr12: 11739866-13828006 |
| 39 | | (probes 564161:564352) | (probes 564230:564257) | (probes 563986:564574) |
| Deletion Peak | 12q21.2 | chr12: 75602135-79819184 | chr12: 76423769-76523220 | chr12: 75648890-78593389 |
| 40 | | (probes 581594:583083) | (probes 581862:581874) | (probes 581614:582689) |
| Deletion Peak | 12q24.33 | chr12: 125048064-133851895 | chr12: 131256822-131362341 | chr12: 125246979-133851895 |
| 41 | | (probes 597584:598999) | (probes 598784:598790) | (probes 597646:598999) |
| Deletion Peak | 14q24.1 | chr14: 68280014-69351476 | chr14: 68284712-68948164 | chr14: 68343659-68985929 |
| 42 | | (probes 640423:640650) | (probes 640425:640565) | (probes 640446:640573) |
| Deletion Peak | 14q32.11 | chr14: 78347564-107349540 | chr14: 90999841-91286309 | chr14: 90170502-92315681 |
| 43 | | (probes 643482:652331) | (probes 647605:647693) | (probes 647318:648035) |
| Deletion Peak | 15q11.2 | chr15: 25436434-25466900 | chr15: 25438412-25459423 | chr15: 1-58781038 |
| 44 | | (probes 652975:652987) | (probes 652978:652984) | (probes 652332:661726) |
| Deletion Peak | 15q21.1 | chr15: 44850582-45321541 | chr15: 44851811-45052539 | chr15: 1-58781038 |
| 45 | | (probes 657156:657216) | (probes 657157:657198) | (probes 652332:661726) |
| Deletion Peak | 15q22.33 | chr15: 67053712-67697647 | chr15: 67337017-67551787 | chr15: 67245914-67753643 |
| 46 | | (probes 664346:664503) | (probes 664432:664448) | (probes 664397:664514) |
| Deletion Peak | 16p13.3 | chr16: 5141600-8053542 | chr16: 6056420-8053542 | chr16: 5279421-7736962 |
| 47 | | (probes 676035:676388) | (probes 676266:676388) | (probes 676069:676383) |
| Deletion Peak | 16q23.1 | chr16: 78131135-79628242 | chr16: 78131135-79286336 | chr16: 78424695-79295468 |
| 48 | | (probes 692486:692917) | (probes 692486:692739) | (probes 692585:692741) |
| Deletion Peak | 17p12 | chr17: 11466949-12461211 | chr17: 11872374-11906034 | chr17: 10231123-12752622 |
| 49 | | (probes 699252:699560) | (probes 699350:699356) | (probes 698870:699686) |
| Deletion Peak | 17q24.3 | chr17: 68174484-70599305 | chr17: 70337175-70590424 | chr17: 70549664-70552757 |
| 50 | | (probes 711906:712713) | (probes 712684:712712) | (probes 712699:712700) |
| Deletion Peak | 18p11.31 | chr18: 3277394-4265401 | chr18: 3441391-3481373 | chr18: 3398771-3729936 |
| 51 | | (probes 715148:715497) | (probes 715189:715190) | (probes 715178:715261) |
| Deletion Peak | 18q12.2 | chr18: 35136370-39061915 | chr18: 36781295-37333625 | chr18: 35827844-46846953 |
| 52 | | (probes 722194:723278) | (probes 722627:722752) | (probes 722377:725639) |
| Deletion Peak | 18q21.2 | chr18: 48472034-48707815 | chr18: 48547928-48660122 | chr18: 47049746-78077248 |
| 53 | | (probes 725965:726002) | (probes 725987:725988) | (probes 725727:734875) |
| Deletion Peak | 18q21.33 | chr18: 60645473-61013467 | chr18: 60788090-61013467 | chr18: 47049746-78077248 |
| 54 | | (probes 730126:730222) | (probes 730167:730222) | (probes 725727:734875) |
| Deletion Peak | 19p13.3 | chr19: 1488247-1660256 | chr19: 1488455-1660256 | chr19: 1-5957502 |
| 55 | | (probes 735045:735049) | (probes 735046:735049) | (probes 734876:735790) |
| Deletion Peak | 20p12.1 | chr20: 13955189-16350354 | chr20: 14416445-15462745 | chr20: 14096517-16064189 |
| 56 | | (probes 751741:751931) | (probes 751773:751774) | (probes 751766:751890) |
| Deletion Peak | 21q11.2 | chr21: 15555708-16334057 | chr21: 15854887-15919066 | chr21: 1-31258004 |
| 57 | | (probes 762725:762981) | (probes 762833:762869) | (probes 762720:767383) |
| Deletion Peak | 21q21.1 | chr21: 23106546-26219369 | chr21: 23248652-23494410 | chr21: 1-31258004 |
| 58 | | (probes 765049:765801) | (probes 765109:765118) | (probes 762720:767383) |
| Deletion Peak | 22q13.32 | chr22: 48649199-49178363 | chr22: 48906309-49158314 | chr22: 47983027-51304566 |
| 59 | | (probes 781080:781144) | (probes 781137:781140) | (probes 780856:781177) |
| Whole arm | 10p | NA | NA | chr10: 1-39254935 |
| amplification 1 | | | | |
| Whole arm | 10q | NA | NA | chr10: 42254935-135534747 |
| amplification 2 | | | | |
| Whole arm | 11p | NA | NA | chr11: 1-51644205 |
| amplification 3 | | | | |
| Whole arm | 11q | NA | NA | chr11: 54644205-135006516 |
| amplification 4 | | | | |
| Whole arm | 12p | NA | NA | chr12: 1-34856694 |
| amplification 5 | | | | |
| Whole arm | 12q | NA | NA | chr12: 37856694-133851895 |
| amplification 6 | | | | |
| Whole arm | 13q | NA | NA | chr13: 19000000-115169878 |
| amplification 7 | | | | |
| Whole arm | 14q | NA | NA | chr14: 19000000-107349540 |
| amplification 8 | | | | |
| Whole arm | 15q | NA | NA | chr15: 20000000-102531392 |
| amplification 9 | | | | |
| Whole arm | 16p | NA | NA | chr16: 1-35335801 |
| amplification 10 | | | | |
| Whole arm | 16q | NA | NA | chr16: 38335801-90354753 |
| amplification 11 | | | | |
| Whole arm | 17p | NA | NA | chr17: 1-22263006 |
| amplification 12 | | | | |
| Whole arm | 17q | NA | NA | chr17: 25263006-81195210 |
| amplification 13 | | | | |
| Whole arm | 18p | NA | NA | chr18: 1-15460898 |
| amplification 14 | | | | |
| Whole arm | 18q | NA | NA | chr18: 18460898-78077248 |
| amplification 15 | | | | |
| Whole arm | 19p | NA | NA | chr19: 1-24681782 |
| amplification 16 | | | | |
| Whole arm | 19q | NA | NA | chr19: 27681782-59128983 |
| amplification 17 | | | | |
| Whole arm | 1p | NA | NA | chr1: 1-121535434 |
| amplification 18 | | | | |
| Whole arm | 1q | NA | NA | chr1: 124535434-249250621 |
| amplification 19 | | | | |
| Whole arm | 20p | NA | NA | chr20: 1-26369569 |
| amplification 20 | | | | |
| Whole arm | 20q | NA | NA | chr20: 29369569-63025520 |
| amplification 21 | | | | |
| Whole arm | 21q | NA | NA | chr21: 14288129-48129895 |
| amplification 22 | | | | |
| Whole arm | 22q | NA | NA | chr22: 16000000-51304566 |
| amplification 23 | | | | |
| Whole arm | 2p | NA | NA | chr2: 1-92326171 |
| amplification 24 | | | | |
| Whole arm | 2q | NA | NA | chr2: 95326171-243199373 |
| amplification 25 | | | | |
| Whole arm | 3p | NA | NA | chr3: 1-90504854 |
| amplification 26 | | | | |
| Whole arm | 3q | NA | NA | chr3: 93504854-198022430 |
| amplification 27 | | | | |
| Whole arm | 4p | NA | NA | chr4: 1-49660117 |
| amplification 28 | | | | |
| Whole arm | 4q | NA | NA | chr4: 52660117-191154276 |
| amplification 29 | | | | |
| Whole arm | 5p | NA | NA | chr5: 1-46405641 |
| amplification 30 | | | | |
| Whole arm | 5q | NA | NA | chr5: 49405641-180915260 |
| amplification 31 | | | | |
| Whole arm | 6p | NA | NA | chr6: 1-58830166 |
| amplification 32 | | | | |
| Whole arm | 6q | NA | NA | chr6: 61830166-171115067 |
| amplification 33 | | | | |
| Whole arm | 7p | NA | NA | chr7: 1-58054331 |
| amplification 34 | | | | |
| Whole arm | 7q | NA | NA | chr7: 61054331-159138663 |
| amplification 35 | | | | |
| Whole arm | 8p | NA | NA | chr8: 1-43838887 |
| amplification 36 | | | | |
| Whole arm | 8q | NA | NA | chr8: 46838887-146364022 |
| amplification 37 | | | | |
| Whole arm | 9p | NA | NA | chr9: 1-47367679 |
| amplification 38 | | | | |
| Whole arm | 9q | NA | NA | chr9: 50367679-141213431 |
| amplification 39 | | | | |
| Whole arm | 10p | NA | NA | chr10: 1-39254935 |
| deletion 1 | | | | |
| Whole arm | 10q | NA | NA | chr10: 42254935-135534747 |
| deletion 2 | | | | |
| Whole arm | 11p | NA | NA | chr11: 1-51644205 |
| deletion 3 | | | | |
| Whole arm | 11q | NA | NA | chr11: 54644205-135006516 |
| deletion 4 | | | | |
| Whole arm | 12p | NA | NA | chr12: 1-34856694 |
| deletion 5 | | | | |
| Whole arm | 12q | NA | NA | chr12: 37856694-133851895 |
| deletion 6 | | | | |
| Whole arm | 13q | NA | NA | chr13: 19000000-115169878 |
| deletion 7 | | | | |
| Whole arm | 14q | NA | NA | chr14: 19000000-107349540 |
| deletion 8 | | | | |
| Whole arm | 15q | NA | NA | chr15: 20000000-102531392 |
| deletion 9 | | | | |
| Whole arm | 16p | NA | NA | chr16: 1-35335801 |
| deletion 10 | | | | |
| Whole arm | 16q | NA | NA | chr16: 38335801-90354753 |
| deletion 11 | | | | |
| Whole arm | 17p | NA | NA | chr17: 1-22263006 |
| deletion 12 | | | | |
| Whole arm | 17q | NA | NA | chr17: 25263006-81195210 |
| deletion 13 | | | | |
| Whole arm | 18p | NA | NA | chr18: 1-15460898 |
| deletion 14 | | | | |
| Whole arm | 18q | NA | NA | chr18: 18460898-78077248 |
| deletion 15 | | | | |
| Whole arm | 19p | NA | NA | chr19: 1-24681782 |
| deletion 16 | | | | |
| Whole arm | 19q | NA | NA | chr19: 27681782-59128983 |
| deletion 17 | | | | |
| Whole arm | 1p | NA | NA | chr1: 1-121535434 |
| deletion 18 | | | | |
| Whole arm | 1q | NA | NA | chr1: 124535434-249250621 |
| deletion 19 | | | | |
| Whole arm | 20p | NA | NA | chr20: 1-26369569 |
| deletion 20 | | | | |
| Whole arm | 20q | NA | NA | chr20: 29369569-63025520 |
| deletion 21 | | | | |
| Whole arm | 21q | NA | NA | chr21: 14288129-48129895 |
| deletion 22 | | | | |
| Whole arm | 22q | NA | NA | chr22: 16000000-51304566 |
| deletion 23 | | | | |
| Whole arm | 2p | NA | NA | chr2: 1-92326171 |
| deletion 24 | | | | |
| Whole arm | 2q | NA | NA | chr2: 95326171-243199373 |
| deletion 25 | | | | |
| Whole arm | 3p | NA | NA | chr3: 1-90504854 |
| deletion 26 | | | | |
| Whole arm | 3q | NA | NA | chr3: 93504854-198022430 |
| deletion 27 | | | | |
| Whole arm | 4p | NA | NA | chr4: 1-49660117 |
| deletion 28 | | | | |
| Whole arm | 4q | NA | NA | chr4: 52660117-191154276 |
| deletion 29 | | | | |
| Whole arm | 5p | NA | NA | chr5: 1-46405641 |
| deletion 30 | | | | |
| Whole arm | 5q | NA | NA | chr5: 49405641-180915260 |
| deletion 31 | | | | |
| Whole arm | 6p | NA | NA | chr6: 1-58830166 |
| deletion 32 | | | | |
| Whole arm | 6q | NA | NA | chr6: 61830166-171115067 |
| deletion 33 | | | | |
| Whole arm | 7p | NA | NA | chr7: 1-58054331 |
| deletion 34 | | | | |
| Whole arm | 7q | NA | NA | chr7: 61054331-159138663 |
| deletion 35 | | | | |
| Whole arm | 8p | NA | NA | chr8: 1-43838887 |
| deletion 36 | | | | |
| Whole arm | 8q | NA | NA | chr8: 46838887-146364022 |
| deletion 37 | | | | |
| Whole arm | 9p | NA | NA | chr9: 1-47367679 |
| deletion 38 | | | | |
| Whole arm | 9q | NA | NA | chr9: 50367679-141213431 |
| deletion 39 | | | | |

TABLE 2
The copy number alteration specifications of cluster1 (genetic subtype 1). All genomic regions
of the biomarker panel of Table 1 are listed together with the frequency that these regions are affected in cluster 1.

| | | | | | Frequency of |
|---|---|---|---|---|---|
| | | | | | region being affected in |
| Unique Name | Descriptor | Wide Peak Limits | Peak Limits | Region Limits | cluster 1 |
| Amplification | 1p34.2 | chr1: 39144054-44367347 | chr1: 42834925-43114106 | chr1: 42692914-43456374 | 0.00 |
| Peak 1 | | | | | |
| Amplification | 1q22 | chr1: 120494739-199253746 | chr1: 155143717-155177826 | chr1: 120497533-175439985 | 0.09 |
| Peak 2 | | | | | |
| Amplification | 1q43 | chr1: 201615295-249250621 | chr1: 242413158-242638099 | chr1: 242270003-249250621 | 0.09 |
| Peak 3 | | | | | |
| Amplification | 2q33.1 | chr2: 181446594-243199373 | chr2: 199603526-199649638 | chr2: 199525178-199824262 | 0.06 |
| Peak 4 | | | | | |
| Amplification | 3q29 | chr3: 174745992-198022430 | chr3: 195922177-195991311 | chr3: 174746836-198022430 | 0.03 |
| Peak 5 | | | | | |
| Amplification | 5p15.33 | chr5: 1-6418038 | chr5: 1-949725 | chr5: 1-2807521 | 0.03 |
| Peak 6 | | | | | |
| Amplification | 5p12 | chr5: 33430780-50470114 | chr5: 43822799-43929095 | chr5: 37334807-44266311 | 0.00 |
| Peak 7 | | | | | |
| Amplification | 6p21.1 | chr6: 43545080-44164949 | chr6: 43765716-44163738 | chr6: 36748055-52028937 | 0.06 |
| Peak 8 | | | | | |
| Amplification | 6q23.3 | chr6: 135342981-135632150 | chr6: 135513416-135593890 | chr6: 133418393-138096856 | 0.09 |
| Peak 9 | | | | | |
| Amplification | 7p11.2 | chr7: 54949302-55950640 | chr7: 55248172-55271747 | chr7: 54949902-55955413 | 0.34 |
| Peak 10 | | | | | |
| Amplification | 8p11.23 | chr8: 38162916-38237532 | chr8: 38165117-38236937 | chr8: 34342520-48854434 | 0.17 |
| Peak 11 | | | | | |
| Amplification | 8p11.21 | chr8: 41760295-42054539 | chr8: 41767516-41796226 | chr8: 34342520-48854434 | 0.17 |
| Peak 12 | | | | | |
| Amplification | 8q12.2 | chr8: 60847395-62906825 | chr8: 61658252-61874896 | chr8: 54646329-146364022 | 0.23 |
| Peak 13 | | | | | |
| Amplification | 8q21.13 | chr8: 80636493-82552412 | chr8: 81853752-81935079 | chr8: 54646329-146364022 | 0.23 |
| Peak 14 | | | | | |
| Amplification | 8q22.3 | chr8: 101853171-101878037 | chr8: 101853464-101876083 | chr8: 54646329-146364022 | 0.26 |
| Peak 15 | | | | | |
| Amplification | 8q24.21 | chr8: 128574277-128592142 | chr8: 128574768-128591041 | chr8: 54646329-146364022 | 0.29 |
| Peak 16 | | | | | |
| Amplification | 10q22.3 | chr10: 79956009-83248579 | chr10: 80445049-80732930 | chr10: 77952482-83583616 | 0.03 |
| Peak 17 | | | | | |
| Amplification | 11p15.5 | chr11: 2091739-2302637 | chr11: 2095340-2301396 | chr11: 2095340-2305324 | 0.00 |
| Peak 18 | | | | | |
| Amplification | 11q13.3 | chr11: 68746750-69827851 | chr11: 69311828-69824706 | chr11: 68748468-70760456 | 0.00 |
| Peak 19 | | | | | |
| Amplification | 12p13.32 | chr12: 3941807-4552801 | chr12: 4257035-4414967 | chr12: 1-8970181 | 0.20 |
| Peak 20 | | | | | |
| Amplification | 12p11.22 | chr12: 24880798-28477058 | chr12: 27800441-27813180 | chr12: 25955987-28156868 | 0.17 |
| Peak 21 | | | | | |
| Amplification | 13q12.2 | chr13: 28164386-28564331 | chr13: 28174614-28222659 | chr13: 1-115169878 | 0.14 |
| Peak 22 | | | | | |
| Amplification | 13q22.1 | chr13: 73775176-74007122 | chr13: 73906681-74004816 | chr13: 1-115169878 | 0.11 |
| Peak 23 | | | | | |
| Amplification | 13q34 | chr13: 110097815-111753132 | chr13: 110614601-110852082 | chr13: 1-115169878 | 0.17 |
| Peak 24 | | | | | |
| Amplification | 15q26.1 | chr15: 84648780-102531392 | chr15: 90811423-90958538 | chr15: 90086447-91895585 | 0.00 |
| Peak 25 | | | | | |
| Amplification | 16p11.2 | chr16: 30548665-30684980 | chr16: 30549334-30682578 | chr16: 28556705-46663588 | 0.11 |
| Peak 26 | | | | | |
| Amplification | 16q12.1 | chr16: 52243935-52652774 | chr16: 52383686-52652198 | chr16: 51078632-53117566 | 0.03 |
| Peak 27 | | | | | |
| Amplification | 17q11.2 | chr17: 27339261-27528113 | chr17: 27339592-27419750 | chr17: 20042949-40168023 | 0.03 |
| Peak 28 | | | | | |
| Amplification | 17q12 | chr17: 37747533-38052599 | chr17: 37950674-38031986 | chr17: 20042949-40168023 | 0.00 |
| Peak 29 | | | | | |
| Amplification | 17q23.2 | chr17: 58400463-58674819 | chr17: 58400997-58532661 | chr17: 57045336-62714320 | 0.11 |
| Peak 30 | | | | | |
| Amplification | 17q24.2 | chr17: 65638523-65873408 | chr17: 65649475-65769071 | chr17: 63042470-68655121 | 0.09 |
| Peak 31 | | | | | |
| Amplification | 17q25.3 | chr17: 77601682-77857881 | chr17: 77766436-77856811 | chr17: 75845339-81195210 | 0.09 |
| Peak 32 | | | | | |
| Amplification | 18q11.2 | chr18: 19517253-20155059 | chr18: 19525020-20154578 | chr18: 19243919-21268418 | 0.11 |
| Peak 33 | | | | | |
| Amplification | 18q21.1 | chr18: 46041609-46930382 | chr18: 46474762-46604295 | chr18: 46043163-46624631 | 0.09 |
| Peak 34 | | | | | |
| Amplification | 19q13.11 | chr19: 32439834-33021877 | chr19: 32966965-32988344 | chr19: 19939350-41312227 | 0.17 |
| Peak 35 | | | | | |
| Amplification | 19q13.2 | chr19: 39844313-40357920 | chr19: 39848086-40009185 | chr19: 19939350-41312227 | 0.23 |
| Peak 36 | | | | | |
| Amplification | 20p12.3 | chr20: 5376354-5742957 | chr20: 5379141-5737687 | chr20: 5156552-5961498 | 0.00 |
| Peak 37 | | | | | |
| Amplification | 20p12.1 | chr20: 16140291-16967195 | chr20: 16488303-16962609 | chr20: 16146669-16970141 | 0.00 |
| Peak 38 | | | | | |
| Amplification | 20p11.22 | chr20: 22058560-22353663 | chr20: 22063756-22350551 | chr20: 20938975-24286806 | 0.00 |
| Peak 39 | | | | | |
| Amplification | 20q11.21 | chr20: 29981165-30284236 | chr20: 30158807-30231055 | chr20: 24375411-63025520 | 0.00 |
| Peak 40 | | | | | |
| Amplification | 20q13.12 | chr20: 42534268-43281676 | chr20: 42537604-42773258 | chr20: 24375411-63025520 | 0.00 |
| Peak 41 | | | | | |
| Amplification | 20q13.2 | chr20: 52240629-52852159 | chr20: 52246659-52447690 | chr20: 24375411-63025520 | 0.00 |
| Peak 42 | | | | | |
| Amplification | 20q13.32 | chr20: 56110288-57258354 | chr20: 57000076-57085518 | chr20: 24375411-63025520 | 0.00 |
| Peak 43 | | | | | |
| Deletion Peak 1 | 1p36.31 | chr1: 4843384-6053964 | chr1: 5646446-6050774 | chr1: 1-37080378 | 0.14 |
| Deletion Peak 2 | 1p36.11 | chr1: 26898389-27219375 | chr1: 27139248-27185402 | chr1: 1-37080378 | 0.14 |
| Deletion Peak 3 | 1p33 | chr1: 49187432-50544677 | chr1: 48847112-50490440 | chr1: 49196205-51396500 | 0.03 |
| Deletion Peak 4 | 1p31.1 | chr1: 68959091-82269682 | chr1: 79308665-79566142 | chr1: 54944113-149961894 | 0.03 |
| Deletion Peak 5 | 1p21.1 | chr1: 102460262-107618224 | chr1: 104041213-104355050 | chr1: 54944113-149961894 | 0.03 |
| Deletion Peak 6 | 2p25.3 | chr2: 1-4752017 | chr2: 1-488785 | chr2: 1-5260278 | 0.11 |
| Deletion Peak 7 | 2p21 | chr2: 42587649-44009118 | chr2: 43440855-43455807 | chr2: 43159857-43470966 | 0.06 |
| Deletion Peak 8 | 2q23.1 | chr2: 147341956-149499455 | chr2: 148437688-148750846 | chr2: 148444810-148756213 | 0.03 |
| Deletion Peak 9 | 2q37.3 | chr2: 240321205-243199373 | chr2: 240940975-241060875 | chr2: 240589192-241997730 | 0.00 |
| Deletion Peak | 3p26.3 | chr3: 1-8350135 | chr3: 1-2251569 | chr3: 1-9456413 | 0.03 |
| 10 | | | | | |
| Deletion Peak | 3p14.2 | chr3: 58946448-61555632 | chr3: 59692189-61457375 | chr3: 59692189-61460946 | 0.09 |
| 11 | | | | | |
| Deletion Peak | 3p13 | chr3: 70014100-71250049 | chr3: 70976616-71182883 | chr3: 70977475-71315576 | 0.11 |
| 12 | | | | | |
| Deletion Peak | 3q26.31 | chr3: 173995229-175766885 | chr3: 174575326-175766885 | chr3: 174646449-174998725 | 0.00 |
| 13 | | | | | |
| Deletion Peak | 4p16.2 | chr4: 1-9787265 | chr4: 5915497-5925651 | chr4: 1-11455571 | 0.11 |
| 14 | | | | | |
| Deletion Peak | 4q22.1 | chr4: 91143530-93226628 | chr4: 91046982-92557520 | chr4: 91103554-92561908 | 0.03 |
| 15 | | | | | |
| Deletion Peak | 4q25 | chr4: 109046573-109544048 | chr4: 109451604-109544048 | chr4: 109489936-109535239 | 0.06 |
| 16 | | | | | |
| Deletion Peak | 4q31.3 | chr4: 152679194-153694102 | chr4: 153232273-153473069 | chr4: 152233433-191154276 | 0.09 |
| 17 | | | | | |
| Deletion Peak | 4q32.1 | chr4: 156135292-162306004 | chr4: 159683040-159830757 | chr4: 152233433-191154276 | 0.06 |
| 18 | | | | | |
| Deletion Peak | 4q35.1 | chr4: 184432080-185262191 | chr4: 184545375-185154488 | chr4: 152233433-191154276 | 0.11 |
| 19 | | | | | |
| Deletion Peak | 5p15.33 | chr5: 1-2750686 | chr5: 1368344-2252637 | chr5: 1-3864653 | 0.17 |
| 20 | | | | | |
| Deletion Peak | 5q12.1 | chr5: 58263825-59784640 | chr5: 58263825-59784640 | chr5: 50968806-135478060 | 0.03 |
| 21 | | | | | |
| Deletion Peak | 5q22.2 | chr5: 111312546-112362638 | chr5: 111747052-111761475 | chr5: 50968806-135478060 | 0.06 |
| 22 | | | | | |
| Deletion Peak | 6p25.3 | chr6: 1613114-2256643 | chr6: 1621843-2256643 | chr6: 1626114-2752758 | 0.06 |
| 23 | | | | | |
| Deletion Peak | 6p22.2 | chr6: 25926929-26025173 | chr6: 25932794-26025173 | chr6: 25933563-26346501 | 0.11 |
| 24 | | | | | |
| Deletion Peak | 6q21 | chr6: 91006553-129206367 | chr6: 111544567-112706964 | chr6: 108785353-116683959 | 0.09 |
| 25 | | | | | |
| Deletion Peak | 6q26 | chr6: 161540781-163179430 | chr6: 161540781-163179430 | chr6: 160448048-171115067 | 0.14 |
| 26 | | | | | |
| Deletion Peak | 7q31.1 | chr7: 109590674-111367757 | chr7: 110232248-111358792 | chr7: 110595801-110618403 | 0.00 |
| 27 | | | | | |
| Deletion Peak | 8p23.3 | chr8: 1-1449850 | chr8: 1-623109 | chr8: 1-10801317 | 0.23 |
| 28 | | | | | |
| Deletion Peak | 8p21.3 | chr8: 13423967-26607015 | chr8: 21547484-21647267 | chr8: 11646511-25738962 | 0.06 |
| 29 | | | | | |
| Deletion Peak | 8p12 | chr8: 33445578-37452445 | chr8: 34944675-36494184 | chr8: 35186331-35207233 | 0.00 |
| 30 | | | | | |
| Deletion Peak | 8q11.1 | chr8: 42874387-48101823 | chr8: 42931769-48061147 | chr8: 42932439-48062091 | 0.00 |
| 31 | | | | | |
| Deletion Peak | 9p21.3 | chr9: 21558582-22452906 | chr9: 21995318-22021004 | chr9: 21711940-22331169 | 0.00 |
| 32 | | | | | |
| Deletion Peak | 10p15.3 | chr10: 1-855610 | chr10: 1-418075 | chr10: 1-2055670 | 0.06 |
| 33 | | | | | |
| Deletion Peak | 10q21.1 | chr10: 51561927-53481136 | chr10: 51561927-54065263 | chr10: 50763189-54253314 | 0.03 |
| 34 | | | | | |
| Deletion Peak | 10q23.31 | chr10: 89502327-90051809 | chr10: 89574482-89607380 | chr10: 83248858-135534747 | 0.06 |
| 35 | | | | | |
| Deletion Peak | 10q25.2 | chr10: 114197471-115353755 | chr10: 114708532-114929210 | chr10: 83248858-135534747 | 0.09 |
| 36 | | | | | |
| Deletion Peak | 10q26.3 | chr10: 133107199-135534747 | chr10: 135221096-135534747 | chr10: 83248858-135534747 | 0.23 |
| 37 | | | | | |
| Deletion Peak | 11q22.3 | chr11: 102958343-135006516 | chr11: 108251085-108350263 | chr11: 108179920-108470348 | 0.00 |
| 38 | | | | | |
| Deletion Peak | 12p13.2 | chr12: 12412186-13039757 | chr12: 12525464-12721981 | chr12: 11739866-13828006 | 0.06 |
| 39 | | | | | |
| Deletion Peak | 12q21.2 | chr12: 75602135-79819184 | chr12: 76423769-76523220 | chr12: 75648890-78593389 | 0.00 |
| 40 | | | | | |
| Deletion Peak | 12q24.33 | chr12: 125048064-133851895 | chr12: 131256822-131362341 | chr12: 125246979-133851895 | 0.14 |
| 41 | | | | | |
| Deletion Peak | 14q24.1 | chr14: 68280014-69351476 | chr14: 68284712-68948164 | chr14: 68343659-68985929 | 0.09 |
| 42 | | | | | |
| Deletion Peak | 14q32.11 | chr14: 78347564-107349540 | chr14: 90999841-91286309 | chr14: 90170502-92315681 | 0.03 |
| 43 | | | | | |
| Deletion Peak | 15q11.2 | chr15: 25436434-25466900 | chr15: 25438412-25459423 | chr15: 1-58781038 | 0.09 |
| 44 | | | | | |
| Deletion Peak | 15q21.1 | chr15: 44850582-45321541 | chr15: 44851811-45052539 | chr15: 1-58781038 | 0.06 |
| 45 | | | | | |
| Deletion Peak | 15q22.33 | chr15: 67053712-67697647 | chr15: 67337017-67551787 | chr15: 67245914-67753643 | 0.06 |
| 46 | | | | | |
| Deletion Peak | 16p13.3 | chr16: 5141600-8053542 | chr16: 6056420-8053542 | chr16: 5279421-7736962 | 0.09 |
| 47 | | | | | |
| Deletion Peak | 16q23.1 | chr16: 78131135-79628242 | chr16: 78131135-79286336 | chr16: 78424695-79295468 | 0.06 |
| 48 | | | | | |
| Deletion Peak | 17p12 | chr17: 11466949-12461211 | chr17: 11872374-11906034 | chr17: 10231123-12752622 | 0.09 |
| 49 | | | | | |
| Deletion Peak | 17q24.3 | chr17: 68174484-70599305 | chr17: 70337175-70590424 | chr17: 70549664-70552757 | 0.03 |
| 50 | | | | | |
| Deletion Peak | 18p11.31 | chr18: 3277394-4265401 | chr18: 3441391-3481373 | chr18: 3398771-3729936 | 0.00 |
| 51 | | | | | |
| Deletion Peak | 18q12.2 | chr18: 35136370-39061915 | chr18: 36781295-37333625 | chr18: 35827844-46846953 | 0.06 |
| 52 | | | | | |
| Deletion Peak | 18q21.2 | chr18: 48472034-48707815 | chr18: 48547928-48660122 | chr18: 47049746-78077248 | 0.11 |
| 53 | | | | | |
| Deletion Peak | 18q21.33 | chr18: 60645473-61013467 | chr18: 60788090-61013467 | chr18: 47049746-78077248 | 0.17 |
| 54 | | | | | |
| Deletion Peak | 19p13.3 | chr19: 1488247-1660256 | chr19: 1488455-1660256 | chr19: 1-5957502 | 0.06 |
| 55 | | | | | |
| Deletion Peak | 20p12.1 | chr20: 13955189-16350354 | chr20: 14416445-15462745 | chr20: 14096517-16064189 | 0.06 |
| 56 | | | | | |
| Deletion Peak | 21q11.2 | chr21: 15555708-16334057 | chr21: 15854887-15919066 | chr21: 1-31258004 | 0.00 |
| 57 | | | | | |
| Deletion Peak | 21q21.1 | chr21: 23106546-26219369 | chr21: 23248652-23494410 | chr21: 1-31258004 | 0.00 |
| 58 | | | | | |
| Deletion Peak | 22q13.32 | chr22: 48649199-49178363 | chr22: 48906309-49158314 | chr22: 47983027-51304566 | 0.09 |
| 59 | | | | | |
| Whole arm | 10p | NA | NA | chr10: 1-39254935 | 0.00 |
| amplification 1 | | | | | |
| Whole arm | 10q | NA | NA | chr16: 1-35335801 | 0.00 |
| amplification 2 | | | | | |
| Whole arm | 11p | NA | NA | chr16: 38335801-90354753 | 0.00 |
| amplification 3 | | | | | |
| Whole arm | 11q | NA | NA | chr17: 1-22263006 | 0.00 |
| amplification 4 | | | | | |
| Whole arm | 12p | NA | NA | chr17: 25263006-81195210 | 0.20 |
| amplification 5 | | | | | |
| Whole arm | 12q | NA | NA | chr18: 1-15460898 | 0.17 |
| amplification 6 | | | | | |
| Whole arm | 13q | NA | NA | chr18: 18460898-78077248 | 0.14 |
| amplification 7 | | | | | |
| Whole arm | 14q | NA | NA | chr19: 1-24681782 | 0.03 |
| amplification 8 | | | | | |
| Whole arm | 15q | NA | NA | chr19: 27681782-59128983 | 0.00 |
| amplification 9 | | | | | |
| Whole arm | 16p | NA | NA | chr1: 1-121535434 | 0.09 |
| amplification | | | | | |
| 10 | | | | | |
| Whole arm | 16q | NA | NA | chr1: 124535434-249250621 | 0.09 |
| amplification | | | | | |
| 11 | | | | | |
| Whole arm | 17p | NA | NA | chr10: 42254935-135534747 | 0.00 |
| amplification | | | | | |
| 12 | | | | | |
| Whole arm | 17q | NA | NA | chr20: 1-26369569 | 0.03 |
| amplification | | | | | |
| 13 | | | | | |
| Whole arm | 18p | NA | NA | chr20: 29369569-63025520 | 0.09 |
| amplification | | | | | |
| 14 | | | | | |
| Whole arm | 18q | NA | NA | chr21: 14288129-48129895 | 0.09 |
| amplification | | | | | |
| 15 | | | | | |
| Whole arm | 19p | NA | NA | chr22: 16000000-51304566 | 0.17 |
| amplification | | | | | |
| 16 | | | | | |
| Whole arm | 19q | NA | NA | chr2: 1-92326171 | 0.20 |
| amplification | | | | | |
| 17 | | | | | |
| Whole arm | 1p | NA | NA | chr2: 95326171-243199373 | 0.00 |
| amplification | | | | | |
| 18 | | | | | |
| Whole arm | 1q | NA | NA | chr3: 1-90504854 | 0.09 |
| amplification | | | | | |
| 19 | | | | | |
| Whole arm | 20p | NA | NA | chr3: 93504854-198022430 | 0.00 |
| amplification | | | | | |
| 20 | | | | | |
| Whole arm | 20q | NA | NA | chr4: 1-49660117 | 0.00 |
| amplification | | | | | |
| 21 | | | | | |
| Whole arm | 21q | NA | NA | chr4: 52660117-191154276 | 0.00 |
| amplification | | | | | |
| 22 | | | | | |
| Whole arm | 22q | NA | NA | chr11: 1-51644205 | 0.00 |
| amplification | | | | | |
| 23 | | | | | |
| Whole arm | 2p | NA | NA | chr5: 1-46405641 | 0.03 |
| amplification | | | | | |
| 24 | | | | | |
| Whole arm | 2q | NA | NA | chr5: 49405641-180915260 | 0.03 |
| amplification | | | | | |
| 25 | | | | | |
| Whole arm | 3p | NA | NA | chr6: 1-58830166 | 0.06 |
| amplification | | | | | |
| 26 | | | | | |
| Whole arm | 3q | NA | NA | chr6: 61830166-171115067 | 0.06 |
| amplification | | | | | |
| 27 | | | | | |
| Whole arm | 4p | NA | NA | chr7: 1-58054331 | 0.00 |
| amplification | | | | | |
| 28 | | | | | |
| Whole arm | 4q | NA | NA | chr7: 61054331-159138663 | 0.00 |
| amplification | | | | | |
| 29 | | | | | |
| Whole arm | 5p | NA | NA | chr8: 1-43838887 | 0.00 |
| amplification | | | | | |
| 30 | | | | | |
| Whole arm | 5q | NA | NA | chr8: 46838887-146364022 | 0.00 |
| amplification | | | | | |
| 31 | | | | | |
| Whole arm | 6p | NA | NA | chr9: 1-47367679 | 0.06 |
| amplification | | | | | |
| 32 | | | | | |
| Whole arm | 6q | NA | NA | chr9: 50367679-141213431 | 0.06 |
| amplification | | | | | |
| 33 | | | | | |
| Whole arm | 7p | NA | NA | chr11: 54644205-135006516 | 0.34 |
| amplification | | | | | |
| 34 | | | | | |
| Whole arm | 7q | NA | NA | chr12: 1-34856694 | 0.34 |
| amplification | | | | | |
| 35 | | | | | |
| Whole arm | 8p | NA | NA | chr12: 37856694-133851895 | 0.14 |
| amplification | | | | | |
| 36 | | | | | |
| Whole arm | 8q | NA | NA | chr13: 19000000-115169878 | 0.23 |
| amplification | | | | | |
| 37 | | | | | |
| Whole arm | 9p | NA | NA | chr14: 19000000-107349540 | 0.06 |
| amplification | | | | | |
| 38 | | | | | |
| Whole arm | 9q | NA | NA | chr15: 20000000-102531392 | 0.06 |
| amplification | | | | | |
| 39 | | | | | |
| Whole arm | 10p | NA | NA | chr10: 1-39254935 | 0.03 |
| deletion 1 | | | | | |
| Whole arm | 10q | NA | NA | chr16: 1-35335801 | 0.00 |
| deletion 2 | | | | | |
| Whole arm | 11p | NA | NA | chr16: 38335801-90354753 | 0.00 |
| deletion 3 | | | | | |
| Whole arm | 11q | NA | NA | chr17: 1-22263006 | 0.00 |
| deletion 4 | | | | | |
| Whole arm | 12p | NA | NA | chr17: 25263006-81195210 | 0.00 |
| deletion 5 | | | | | |
| Whole arm | 12q | NA | NA | chr18: 1-15460898 | 0.00 |
| deletion 6 | | | | | |
| Whole arm | 13q | NA | NA | chr18: 18460898-78077248 | 0.00 |
| deletion 7 | | | | | |
| Whole arm | 14q | NA | NA | chr19: 1-24681782 | 0.00 |
| deletion 8 | | | | | |
| Whole arm | 15q | NA | NA | chr19: 27681782-59128983 | 0.03 |
| deletion 9 | | | | | |
| Whole arm | 16p | NA | NA | chr1: 1-121535434 | 0.06 |
| deletion 10 | | | | | |
| Whole arm | 16q | NA | NA | chr1: 124535434-249250621 | 0.03 |
| deletion 11 | | | | | |
| Whole arm | 17p | NA | NA | chr10: 42254935-135534747 | 0.09 |
| deletion 12 | | | | | |
| Whole arm | 17q | NA | NA | chr20: 1-26369569 | 0.03 |
| deletion 13 | | | | | |
| Whole arm | 18p | NA | NA | chr20: 29369569-63025520 | 0.03 |
| deletion 14 | | | | | |
| Whole arm | 18q | NA | NA | chr21: 14288129-48129895 | 0.06 |
| deletion 15 | | | | | |
| Whole arm | 19p | NA | NA | chr22: 16000000-51304566 | 0.06 |
| deletion 16 | | | | | |
| Whole arm | 19q | NA | NA | chr2: 1-92326171 | 0.06 |
| deletion 17 | | | | | |
| Whole arm | 1p | NA | NA | chr2: 95326171-243199373 | 0.03 |
| deletion 18 | | | | | |
| Whole arm | 1q | NA | NA | chr3: 1-90504854 | 0.00 |
| deletion 19 | | | | | |
| Whole arm | 20p | NA | NA | chr3: 93504854-198022430 | 0.03 |
| deletion 20 | | | | | |
| Whole arm | 20q | NA | NA | chr4: 1-49660117 | 0.00 |
| deletion 21 | | | | | |
| Whole arm | 21q | NA | NA | chr4: 52660117-191154276 | 0.00 |
| deletion 22 | | | | | |
| Whole arm | 22q | NA | NA | chr11: 1-51644205 | 0.09 |
| deletion 23 | | | | | |
| Whole arm | 2p | NA | NA | chr5: 1-46405641 | 0.00 |
| deletion 24 | | | | | |
| Whole arm | 2q | NA | NA | chr5: 49405641-180915260 | 0.00 |
| deletion 25 | | | | | |
| Whole arm | 3p | NA | NA | chr6: 1-58830166 | 0.00 |
| deletion 26 | | | | | |
| Whole arm | 3q | NA | NA | chr6: 61830166-171115067 | 0.00 |
| deletion 27 | | | | | |
| Whole arm | 4p | NA | NA | chr7: 1-58054331 | 0.00 |
| deletion 28 | | | | | |
| Whole arm | 4q | NA | NA | chr7: 61054331-159138663 | 0.03 |
| deletion 29 | | | | | |
| Whole arm | 5p | NA | NA | chr8: 1-43838887 | 0.03 |
| deletion 30 | | | | | |
| Whole arm | 5q | NA | NA | chr8: 46838887-146364022 | 0.03 |
| deletion 31 | | | | | |
| Whole arm | 6p | NA | NA | chr9: 1-47367679 | 0.03 |
| deletion 32 | | | | | |
| Whole arm | 6q | NA | NA | chr9: 50367679-141213431 | 0.03 |
| deletion 33 | | | | | |
| Whole arm | 7p | NA | NA | chr11: 54644205-135006516 | 0.00 |
| deletion 34 | | | | | |
| Whole arm | 7q | NA | NA | chr12: 1-34856694 | 0.00 |
| deletion 35 | | | | | |
| Whole arm | 8p | NA | NA | chr12: 37856694-133851895 | 0.03 |
| deletion 36 | | | | | |
| Whole arm | 8q | NA | NA | chr13: 19000000-115169878 | 0.00 |
| deletion 37 | | | | | |
| Whole arm | 9p | NA | NA | chr14: 19000000-107349540 | 0.00 |
| deletion 38 | | | | | |
| Whole arm | 9q | NA | NA | chr15: 20000000-102531392 | 0.00 |
| deletion 39 | | | | | |

TABLE 3
The copy number alteration specifications of cluster 2 (genetic subtype 2). All genomic regions
of the biomarker panel of Table 1 are listed together with the frequency that these regions are affected in cluster 2.

| | | | | | Frequency of |
|---|---|---|---|---|---|
| | | | | | region being affected in |
| Unique Name | Descriptor | Wide Peak Limits | Peak Limits | Region Limits | cluster 2 |
| Amplification | 1p34.2 | chr1: 39144054-44367347 | chr1: 42834925-43114106 | chr1: 42692914-43456374 | 0.04 |
| Peak 1 | | | | | |
| Amplification | 1q22 | chr1: 120494739-199253746 | chr1: 155143717-155177826 | chr1: 120497533-175439985 | 0.29 |
| Peak 2 | | | | | |
| Amplification | 1q43 | chr1: 201615295-249250621 | chr1: 242413158-242638099 | chr1: 242270003-249250621 | 0.30 |
| Peak 3 | | | | | |
| Amplification | 2q33.1 | chr2: 181446594-243199373 | chr2: 199603526-199649638 | chr2: 199525178-199824262 | 0.23 |
| Peak 4 | | | | | |
| Amplification | 3q29 | chr3: 174745992-198022430 | chr3: 195922177-195991311 | chr3: 174746836-198022430 | 0.19 |
| Peak 5 | | | | | |
| Amplification | 5p15.33 | chr5: 1-6418038 | chr5: 1-949725 | chr5: 1-2807521 | 0.20 |
| Peak 6 | | | | | |
| Amplification | 5p12 | chr5: 33430780-50470114 | chr5: 43822799-43929095 | chr5: 37334807-44266311 | 0.18 |
| Peak 7 | | | | | |
| Amplification | 6p21.1 | chr6: 43545080-44164949 | chr6: 43765716-44163738 | chr6: 36748055-52028937 | 0.23 |
| Peak 8 | | | | | |
| Amplification | 6q23.3 | chr6: 135342981-135632150 | chr6: 135513416-135593890 | chr6: 133418393-138096856 | 0.18 |
| Peak 9 | | | | | |
| Amplification | 7p11.2 | chr7: 54949302-55950640 | chr7: 55248172-55271747 | chr7: 54949902-55955413 | 0.63 |
| Peak 10 | | | | | |
| Amplification | 8p11.23 | chr8: 38162916-38237532 | chr8: 38165117-38236937 | chr8: 34342520-48854434 | 0.42 |
| Peak 11 | | | | | |
| Amplification | 8p11.21 | chr8: 41760295-42054539 | chr8: 41767516-41796226 | chr8: 34342520-48854434 | 0.55 |
| Peak 12 | | | | | |
| Amplification | 8q12.2 | chr8: 60847395-62906825 | chr8: 61658252-61874896 | chr8: 54646329-146364022 | 0.68 |
| Peak 13 | | | | | |
| Amplification | 8q21.13 | chr8: 80636493-82552412 | chr8: 81853752-81935079 | chr8: 54646329-146364022 | 0.74 |
| Peak 14 | | | | | |
| Amplification | 8q22.3 | chr8: 101853171-101878037 | chr8: 101853464-101876083 | chr8: 54646329-146364022 | 0.76 |
| Peak 15 | | | | | |
| Amplification | 8q24.21 | chr8: 128574277-128592142 | chr8: 128574768-128591041 | chr8: 54646329-146364022 | 0.77 |
| Peak 16 | | | | | |
| Amplification | 10q22.3 | chr10: 79956009-83248579 | chr10: 80445049-80732930 | chr10: 77952482-83583616 | 0.04 |
| Peak 17 | | | | | |
| Amplification | 11p15.5 | chr11: 2091739-2302637 | chr11: 2095340-2301396 | chr11: 2095340-2305324 | 0.10 |
| Peak 18 | | | | | |
| Amplification | 11q13.3 | chr11: 68746750-69827851 | chr11: 69311828-69824706 | chr11: 68748468-70760456 | 0.09 |
| Peak 19 | | | | | |
| Amplification | 12p13.32 | chr12: 3941807-4552801 | chr12: 4257035-4414967 | chr12: 1-8970181 | 0.31 |
| Peak 20 | | | | | |
| Amplification | 12p11.22 | chr12: 24880798-28477058 | chr12: 27800441-27813180 | chr12: 25955987-28156868 | 0.31 |
| Peak 21 | | | | | |
| Amplification | 13q12.2 | chr13: 28164386-28564331 | chr13: 28174614-28222659 | chr13: 1-115169878 | 0.82 |
| Peak 22 | | | | | |
| Amplification | 13q22.1 | chr13: 73775176-74007122 | chr13: 73906681-74004816 | chr13: 1-115169878 | 0.79 |
| Peak 23 | | | | | |
| Amplification | 13q34 | chr13: 110097815-111753132 | chr13: 110614601-110852082 | chr13: 1-115169878 | 0.78 |
| Peak 24 | | | | | |
| Amplification | 15q26.1 | chr15: 84648780-102531392 | chr15: 90811423-90958538 | chr15: 90086447-91895585 | 0.06 |
| Peak 25 | | | | | |
| Amplification | 16p11.2 | chr16: 30548665-30684980 | chr16: 30549334-30682578 | chr16: 28556705-46663588 | 0.36 |
| Peak 26 | | | | | |
| Amplification | 16q12.1 | chr16: 52243935-52652774 | chr16: 52383686-52652198 | chr16: 51078632-53117566 | 0.29 |
| Peak 27 | | | | | |
| Amplification | 17q11.2 | chr17: 27339261-27528113 | chr17: 27339592-27419750 | chr17: 20042949-40168023 | 0.24 |
| Peak 28 | | | | | |
| Amplification | 17q12 | chr17: 37747533-38052599 | chr17: 37950674-38031986 | chr17: 20042949-40168023 | 0.22 |
| Peak 29 | | | | | |
| Amplification | 17q23.2 | chr17: 58400463-58674819 | chr17: 58400997-58532661 | chr17: 57045336-62714320 | 0.21 |
| Peak 30 | | | | | |
| Amplification | 17q24.2 | chr17: 65638523-65873408 | chr17: 65649475-65769071 | chr17: 63042470-68655121 | 0.21 |
| Peak 31 | | | | | |
| Amplification | 17q25.3 | chr17: 77601682-77857881 | chr17: 77766436-77856811 | chr17: 75845339-81195210 | 0.22 |
| Peak 32 | | | | | |
| Amplification | 18q11.2 | chr18: 19517253-20155059 | chr18: 19525020-20154578 | chr18: 19243919-21268418 | 0.06 |
| Peak 33 | | | | | |
| Amplification | 18q21.1 | chr18: 46041609-46930382 | chr18: 46474762-46604295 | chr18: 46043163-46624631 | 0.04 |
| Peak 34 | | | | | |
| Amplification | 19q13.11 | chr19: 32439834-33021877 | chr19: 32966965-32988344 | chr19: 19939350-41312227 | 0.19 |
| Peak 35 | | | | | |
| Amplification | 19q13.2 | chr19: 39844313-40357920 | chr19: 39848086-40009185 | chr19: 19939350-41312227 | 0.18 |
| Peak 36 | | | | | |
| Amplification | 20p12.3 | chr20: 5376354-5742957 | chr20: 5379141-5737687 | chr20: 5156552-5961498 | 0.42 |
| Peak 37 | | | | | |
| Amplification | 20p12.1 | chr20: 16140291-16967195 | chr20: 16488303-16962609 | chr20: 16146669-16970141 | 0.46 |
| Peak 38 | | | | | |
| Amplification | 20p11.22 | chr20: 22058560-22353663 | chr20: 22063756-22350551 | chr20: 20938975-24286806 | 0.55 |
| Peak 39 | | | | | |
| Amplification | 20q11.21 | chr20: 29981165-30284236 | chr20: 30158807-30231055 | chr20: 24375411-63025520 | 0.94 |
| Peak 40 | | | | | |
| Amplification | 20q13.12 | chr20: 42534268-43281676 | chr20: 42537604-42773258 | chr20: 24375411-63025520 | 0.93 |
| Peak 41 | | | | | |
| Amplification | 20q13.2 | chr20: 52240629-52852159 | chr20: 52246659-52447690 | chr20: 24375411-63025520 | 0.95 |
| Peak 42 | | | | | |
| Amplification | 20q13.32 | chr20: 56110288-57258354 | chr20: 57000076-57085518 | chr20: 24375411-63025520 | 0.95 |
| Peak 43 | | | | | |
| Deletion Peak 1 | 1p36.31 | chr1: 4843384-6053964 | chr1: 5646446-6050774 | chr1: 1-37080378 | 0.40 |
| Deletion Peak 2 | 1p36.11 | chr1: 26898389-27219375 | chr1: 27139248-27185402 | chr1: 1-37080378 | 0.42 |
| Deletion Peak 3 | 1p33 | chr1: 49187432-50544677 | chr1: 48847112-50490440 | chr1: 49196205-51396500 | 0.19 |
| Deletion Peak 4 | 1p31.1 | chr1: 68959091-82269682 | chr1: 79308665-79566142 | chr1: 54944113-149961894 | 0.21 |
| Deletion Peak 5 | 1p21.1 | chr1: 102460262-107618224 | chr1: 104041213-104355050 | chr1: 54944113-149961894 | 0.22 |
| Deletion Peak 6 | 2p25.3 | chr2: 1-4752017 | chr2: 1-488785 | chr2: 1-5260278 | 0.13 |
| Deletion Peak 7 | 2p21 | chr2: 42587649-44009118 | chr2: 43440855-43455807 | chr2: 43159857-43470966 | 0.01 |
| Deletion Peak 8 | 2q23.1 | chr2: 147341956-149499455 | chr2: 148437688-148750846 | chr2: 148444810-148756213 | 0.04 |
| Deletion Peak 9 | 2q37.3 | chr2: 240321205-243199373 | chr2: 240940975-241060875 | chr2: 240589192-241997730 | 0.05 |
| Deletion Peak | 3p26.3 | chr3: 1-8350135 | chr3: 1-2251569 | chr3: 1-9456413 | 0.18 |
| 10 | | | | | |
| Deletion Peak | 3p14.2 | chr3: 58946448-61555632 | chr3: 59692189-61457375 | chr3: 59692189-61460946 | 0.15 |
| 11 | | | | | |
| Deletion Peak | 3p13 | chr3: 70014100-71250049 | chr3: 70976616-71182883 | chr3: 70977475-71315576 | 0.15 |
| 12 | | | | | |
| Deletion Peak | 3q26.31 | chr3: 173995229-175766885 | chr3: 174575326-175766885 | chr3: 174646449-174998725 | 0.02 |
| 13 | | | | | |
| Deletion Peak | 4p16.2 | chr4: 1-9787265 | chr4: 5915497-5925651 | chr4: 1-11455571 | 0.24 |
| 14 | | | | | |
| Deletion Peak | 4q22.1 | chr4: 91143530-93226628 | chr4: 91046982-92557520 | chr4: 91103554-92561908 | 0.10 |
| 15 | | | | | |
| Deletion Peak | 4q25 | chr4: 109046573-109544048 | chr4: 109451604-109544048 | chr4: 109489936-109535239 | 0.08 |
| 16 | | | | | |
| Deletion Peak | 4q31.3 | chr4: 152679194-153694102 | chr4: 153232273-153473069 | chr4: 152233433-191154276 | 0.06 |
| 17 | | | | | |
| Deletion Peak | 4q32.1 | chr4: 156135292-162306004 | chr4: 159683040-159830757 | chr4: 152233433-191154276 | 0.09 |
| 18 | | | | | |
| Deletion Peak | 4q35.1 | chr4: 184432080-185262191 | chr4: 184545375-185154488 | chr4: 152233433-191154276 | 0.13 |
| 19 | | | | | |
| Deletion Peak | 5p15.33 | chr5: 1-2750686 | chr5: 1368344-2252637 | chr5: 1-3864653 | 0.20 |
| 20 | | | | | |
| Deletion Peak | 5q12.1 | chr5: 58263825-59784640 | chr5: 58263825-59784640 | chr5: 50968806-135478060 | 0.12 |
| 21 | | | | | |
| Deletion Peak | 5q22.2 | chr5: 111312546-112362638 | chr5: 111747052-111761475 | chr5: 50968806-135478060 | 0.21 |
| 22 | | | | | |
| Deletion Peak | 6p25.3 | chr6: 1613114-2256643 | chr6: 1621843-2256643 | chr6: 1626114-2752758 | 0.06 |
| 23 | | | | | |
| Deletion Peak | 6p22.2 | chr6: 25926929-26025173 | chr6: 25932794-26025173 | chr6: 25933563-26346501 | 0.06 |
| 24 | | | | | |
| Deletion Peak | 6q21 | chr6: 91006553-129206367 | chr6: 111544567-112706964 | chr6: 108785353-116683959 | 0.12 |
| 25 | | | | | |
| Deletion Peak | 6q26 | chr6: 161540781-163179430 | chr6: 161540781-163179430 | chr6: 160448048-171115067 | 0.17 |
| 26 | | | | | |
| Deletion Peak | 7q31.1 | chr7: 109590674-111367757 | chr7: 110232248-111358792 | chr7: 110595801-110618403 | 0.02 |
| 27 | | | | | |
| Deletion Peak | 8p23.3 | chr8: 1-1449850 | chr8: 1-623109 | chr8: 1-10801317 | 0.59 |
| 28 | | | | | |
| Deletion Peak | 8p21.3 | chr8: 13423967-26607015 | chr8: 21547484-21647267 | chr8: 11646511-25738962 | 0.58 |
| 29 | | | | | |
| Deletion Peak | 8p12 | chr8: 33445578-37452445 | chr8: 34944675-36494184 | chr8: 35186331-35207233 | 0.46 |
| 30 | | | | | |
| Deletion Peak | 8q11.1 | chr8: 42874387-48101823 | chr8: 42931769-48061147 | chr8: 42932439-48062091 | 0.03 |
| 31 | | | | | |
| Deletion Peak | 9p21.3 | chr9: 21558582-22452906 | chr9: 21995318-22021004 | chr9: 21711940-22331169 | 0.09 |
| 32 | | | | | |
| Deletion Peak | 10p15.3 | chr10: 1-855610 | chr10: 1-418075 | chr10: 1-2055670 | 0.09 |
| 33 | | | | | |
| Deletion Peak | 10q21.1 | chr10: 51561927-53481136 | chr10: 51561927-54065263 | chr10: 50763189-54253314 | 0.07 |
| 34 | | | | | |
| Deletion Peak | 10q23.31 | chr10: 89502327-90051809 | chr10: 89574482-89607380 | chr10: 83248858-135534747 | 0.14 |
| 35 | | | | | |
| Deletion Peak | 10q25.2 | chr10: 114197471-115353755 | chr10: 114708532-114929210 | chr10: 83248858-135534747 | 0.09 |
| 36 | | | | | |
| Deletion Peak | 10q26.3 | chr10: 133107199-135534747 | chr10: 135221096-135534747 | chr10: 83248858-135534747 | 0.20 |
| 37 | | | | | |
| Deletion Peak | 11q22.3 | chr11: 102958343-135006516 | chr11: 108251085-108350263 | chr11: 108179920-108470348 | 0.08 |
| 38 | | | | | |
| Deletion Peak | 12p13.2 | chr12: 12412186-13039757 | chr12: 12525464-12721981 | chr12: 11739866-13828006 | 0.10 |
| 39 | | | | | |
| Deletion Peak | 12q21.2 | chr12: 75602135-79819184 | chr12: 76423769-76523220 | chr12: 75648890-78593389 | 0.02 |
| 40 | | | | | |
| Deletion Peak | 12q24.33 | chr12: 125048064-133851895 | chr12: 131256822-131362341 | chr12: 125246979-133851895 | 0.15 |
| 41 | | | | | |
| Deletion Peak | 14q24.1 | chr14: 68280014-69351476 | chr14: 68284712-68948164 | chr14: 68343659-68985929 | 0.24 |
| 42 | | | | | |
| Deletion Peak | 14q32.11 | chr14: 78347564-107349540 | chr14: 90999841-91286309 | chr14: 90170502-92315681 | 0.22 |
| 43 | | | | | |
| Deletion Peak | 15q11.2 | chr15: 25436434-25466900 | chr15: 25438412-25459423 | chr15: 1-58781038 | 0.30 |
| 44 | | | | | |
| Deletion Peak | 15q21.1 | chr15: 44850582-45321541 | chr15: 44851811-45052539 | chr15: 1-58781038 | 0.26 |
| 45 | | | | | |
| Deletion Peak | 15q22.33 | chr15: 67053712-67697647 | chr15: 67337017-67551787 | chr15: 67245914-67753643 | 0.21 |
| 46 | | | | | |
| Deletion Peak | 16p13.3 | chr16: 5141600-8053542 | chr16: 6056420-8053542 | chr16: 5279421-7736962 | 0.08 |
| 47 | | | | | |
| Deletion Peak | 16q23.1 | chr16: 78131135-79628242 | chr16: 78131135-79286336 | chr16: 78424695-79295468 | 0.05 |
| 48 | | | | | |
| Deletion Peak | 17p12 | chr17: 11466949-12461211 | chr17: 11872374-11906034 | chr17: 10231123-12752622 | 0.63 |
| 49 | | | | | |
| Deletion Peak | 17q24.3 | chr17: 68174484-70599305 | chr17: 70337175-70590424 | chr17: 70549664-70552757 | 0.13 |
| 50 | | | | | |
| Deletion Peak | 18p11.31 | chr18: 3277394-4265401 | chr18: 3441391-3481373 | chr18: 3398771-3729936 | 0.59 |
| 51 | | | | | |
| Deletion Peak | 18q12.2 | chr18: 35136370-39061915 | chr18: 36781295-37333625 | chr18: 35827844-46846953 | 0.81 |
| 52 | | | | | |
| Deletion Peak | 18q21.2 | chr18: 48472034-48707815 | chr18: 48547928-48660122 | chr18: 47049746-78077248 | 0.83 |
| 53 | | | | | |
| Deletion Peak | 18q21.33 | chr18: 60645473-61013467 | chr18: 60788090-61013467 | chr18: 47049746-78077248 | 0.82 |
| 54 | | | | | |
| Deletion Peak | 19p13.3 | chr19: 1488247-1660256 | chr19: 1488455-1660256 | chr19: 1-5957502 | 0.05 |
| 55 | | | | | |
| Deletion Peak | 20p12.1 | chr20: 13955189-16350354 | chr20: 14416445-15462745 | chr20: 14096517-16064189 | 0.33 |
| 56 | | | | | |
| Deletion Peak | 21q11.2 | chr21: 15555708-16334057 | chr21: 15854887-15919066 | chr21: 1-31258004 | 0.12 |
| 57 | | | | | |
| Deletion Peak | 21q21.1 | chr21: 23106546-26219369 | chr21: 23248652-23494410 | chr21: 1-31258004 | 0.14 |
| 58 | | | | | |
| Deletion Peak | 22q13.32 | chr22: 48649199-49178363 | chr22: 48906309-49158314 | chr22: 47983027-51304566 | 0.31 |
| 59 | | | | | |
| Whole arm | 10p | NA | NA | chr10: 1-39254935 | 0.14 |
| amplification 1 | | | | | |
| Whole arm | 10q | NA | NA | chr16: 1-35335801 | 0.04 |
| amplification 2 | | | | | |
| Whole arm | 11p | NA | NA | chr16: 38335801-90354753 | 0.06 |
| amplification 3 | | | | | |
| Whole arm | 11q | NA | NA | chr17: 1-22263006 | 0.07 |
| amplification 4 | | | | | |
| Whole arm | 12p | NA | NA | chr17: 25263006-81195210 | 0.29 |
| amplification 5 | | | | | |
| Whole arm | 12q | NA | NA | chr18: 1-15460898 | 0.20 |
| amplification 6 | | | | | |
| Whole arm | 13q | NA | NA | chr18: 18460898-78077248 | 0.78 |
| amplification 7 | | | | | |
| Whole arm | 14q | NA | NA | chr19: 1-24681782 | 0.06 |
| amplification 8 | | | | | |
| Whole arm | 15q | NA | NA | chr19: 27681782-59128983 | 0.03 |
| amplification 9 | | | | | |
| Whole arm | 16p | NA | NA | chr1: 1-121535434 | 0.29 |
| amplification | | | | | |
| 10 | | | | | |
| Whole arm | 16q | NA | NA | chr1: 124535434-249250621 | 0.28 |
| amplification | | | | | |
| 11 | | | | | |
| Whole arm | 17p | NA | NA | chr10: 42254935-135534747 | 0.02 |
| amplification | | | | | |
| 12 | | | | | |
| Whole arm | 17q | NA | NA | chr20: 1-26369569 | 0.13 |
| amplification | | | | | |
| 13 | | | | | |
| Whole arm | 18p | NA | NA | chr20: 29369569-63025520 | 0.05 |
| amplification | | | | | |
| 14 | | | | | |
| Whole arm | 18q | NA | NA | chr21: 14288129-48129895 | 0.01 |
| amplification | | | | | |
| 15 | | | | | |
| Whole arm | 19p | NA | NA | chr22: 16000000-51304566 | 0.15 |
| amplification | | | | | |
| 16 | | | | | |
| Whole arm | 19q | NA | NA | chr2: 1-92326171 | 0.18 |
| amplification | | | | | |
| 17 | | | | | |
| Whole arm | 1p | NA | NA | chr2: 95326171-243199373 | 0.01 |
| amplification | | | | | |
| 18 | | | | | |
| Whole arm | 1q | NA | NA | chr3: 1-90504854 | 0.23 |
| amplification | | | | | |
| 19 | | | | | |
| Whole arm | 20p | NA | NA | chr3: 93504854-198022430 | 0.50 |
| amplification | | | | | |
| 20 | | | | | |
| Whole arm | 20q | NA | NA | chr4: 1-49660117 | 0.90 |
| amplification | | | | | |
| 21 | | | | | |
| Whole arm | 21q | NA | NA | chr4: 52660117-191154276 | 0.06 |
| amplification | | | | | |
| 22 | | | | | |
| Whole arm | 22q | NA | NA | chr11: 1-51644205 | 0.01 |
| amplification | | | | | |
| 23 | | | | | |
| Whole arm | 2p | NA | NA | chr5: 1-46405641 | 0.18 |
| amplification | | | | | |
| 24 | | | | | |
| Whole arm | 2q | NA | NA | chr5: 49405641-180915260 | 0.19 |
| amplification | | | | | |
| 25 | | | | | |
| Whole arm | 3p | NA | NA | chr6: 1-58830166 | 0.05 |
| amplification | | | | | |
| 26 | | | | | |
| Whole arm | 3q | NA | NA | chr6: 61830166-171115067 | 0.14 |
| amplification | | | | | |
| 27 | | | | | |
| Whole arm | 4p | NA | NA | chr7: 1-58054331 | 0.03 |
| amplification | | | | | |
| 28 | | | | | |
| Whole arm | 4q | NA | NA | chr7: 61054331-159138663 | 0.02 |
| amplification | | | | | |
| 29 | | | | | |
| Whole arm | 5p | NA | NA | chr8: 1-43838887 | 0.17 |
| amplification | | | | | |
| 30 | | | | | |
| Whole arm | 5q | NA | NA | chr8: 46838887-146364022 | 0.11 |
| amplification | | | | | |
| 31 | | | | | |
| Whole arm | 6p | NA | NA | chr9: 1-47367679 | 0.16 |
| amplification | | | | | |
| 32 | | | | | |
| Whole arm | 6q | NA | NA | chr9: 50367679-141213431 | 0.15 |
| amplification | | | | | |
| 33 | | | | | |
| Whole arm | 7p | NA | NA | chr11: 54644205-135006516 | 0.63 |
| amplification | | | | | |
| 34 | | | | | |
| Whole arm | 7q | NA | NA | chr12: 1-34856694 | 0.54 |
| amplification | | | | | |
| 35 | | | | | |
| Whole arm | 8p | NA | NA | chr12: 37856694-133851895 | 0.20 |
| amplification | | | | | |
| 36 | | | | | |
| Whole arm | 8q | NA | NA | chr13: 19000000-115169878 | 0.69 |
| amplification | | | | | |
| 37 | | | | | |
| Whole arm | 9p | NA | NA | chr14: 19000000-107349540 | 0.16 |
| amplification | | | | | |
| 38 | | | | | |
| Whole arm | 9q | NA | NA | chr15: 20000000-102531392 | 0.17 |
| amplification | | | | | |
| 39 | | | | | |
| Whole arm | 10p | NA | NA | chr10: 1-39254935 | 0.04 |
| deletion 1 | | | | | |
| Whole arm | 10q | NA | NA | chr16: 1-35335801 | 0.07 |
| deletion 2 | | | | | |
| Whole arm | 11p | NA | NA | chr16: 38335801-90354753 | 0.01 |
| deletion 3 | | | | | |
| Whole arm | 11q | NA | NA | chr17: 1-22263006 | 0.04 |
| deletion 4 | | | | | |
| Whole arm | 12p | NA | NA | chr17: 25263006-81195210 | 0.04 |
| deletion 5 | | | | | |
| Whole arm | 12q | NA | NA | chr18: 1-15460898 | 0.01 |
| deletion 6 | | | | | |
| Whole arm | 13q | NA | NA | chr18: 18460898-78077248 | 0.02 |
| deletion 7 | | | | | |
| Whole arm | 14q | NA | NA | chr19: 1-24681782 | 0.21 |
| deletion 8 | | | | | |
| Whole arm | 15q | NA | NA | chr19: 27681782-59128983 | 0.21 |
| deletion 9 | | | | | |
| Whole arm | 16p | NA | NA | chr1: 1-121535434 | 0.03 |
| deletion 10 | | | | | |
| Whole arm | 16q | NA | NA | chr1: 124535434-249250621 | 0.02 |
| deletion 11 | | | | | |
| Whole arm | 17p | NA | NA | chr10: 42254935-135534747 | 0.57 |
| deletion 12 | | | | | |
| Whole arm | 17q | NA | NA | chr20: 1-26369569 | 0.05 |
| deletion 13 | | | | | |
| Whole arm | 18p | NA | NA | chr20: 29369569-63025520 | 0.62 |
| deletion 14 | | | | | |
| Whole arm | 18q | NA | NA | chr21: 14288129-48129895 | 0.79 |
| deletion 15 | | | | | |
| Whole arm | 19p | NA | NA | chr22: 16000000-51304566 | 0.02 |
| deletion 16 | | | | | |
| Whole arm | 19q | NA | NA | chr2: 1-92326171 | 0.01 |
| deletion 17 | | | | | |
| Whole arm | 1p | NA | NA | chr2: 95326171-243199373 | 0.17 |
| deletion 18 | | | | | |
| Whole arm | 1q | NA | NA | chr3: 1-90504854 | 0.01 |
| deletion 19 | | | | | |
| Whole arm | 20p | NA | NA | chr3: 93504854-198022430 | 0.19 |
| deletion 20 | | | | | |
| Whole arm | 20q | NA | NA | chr4: 1-49660117 | 0.00 |
| deletion 21 | | | | | |
| Whole arm | 21q | NA | NA | chr4: 52660117-191154276 | 0.10 |
| deletion 22 | | | | | |
| Whole arm | 22q | NA | NA | chr11: 1-51644205 | 0.21 |
| deletion 23 | | | | | |
| Whole arm | 2p | NA | NA | chr5: 1-46405641 | 0.01 |
| deletion 24 | | | | | |
| Whole arm | 2q | NA | NA | chr5: 49405641-180915260 | 0.01 |
| deletion 25 | | | | | |
| Whole arm | 3p | NA | NA | chr6: 1-58830166 | 0.10 |
| deletion 26 | | | | | |
| Whole arm | 3q | NA | NA | chr6: 61830166-171115067 | 0.02 |
| deletion 27 | | | | | |
| Whole arm | 4p | NA | NA | chr7: 1-58054331 | 0.12 |
| deletion 28 | | | | | |
| Whole arm | 4q | NA | NA | chr7: 61054331-159138663 | 0.05 |
| deletion 29 | | | | | |
| Whole arm | 5p | NA | NA | chr8: 1-43838887 | 0.03 |
| deletion 30 | | | | | |
| Whole arm | 5q | NA | NA | chr8: 46838887-146364022 | 0.06 |
| deletion 31 | | | | | |
| Whole arm | 6p | NA | NA | chr9: 1-47367679 | 0.02 |
| deletion 32 | | | | | |
| Whole arm | 6q | NA | NA | chr9: 50367679-141213431 | 0.06 |
| deletion 33 | | | | | |
| Whole arm | 7p | NA | NA | chr11: 54644205-135006516 | 0.00 |
| deletion 34 | | | | | |
| Whole arm | 7q | NA | NA | chr12: 1-34856694 | 0.00 |
| deletion 35 | | | | | |
| Whole arm | 8p | NA | NA | chr12: 37856694-133851895 | 0.39 |
| deletion 36 | | | | | |
| Whole arm | 8q | NA | NA | chr13: 19000000-115169878 | 0.02 |
| deletion 37 | | | | | |
| Whole arm | 9p | NA | NA | chr14: 19000000-107349540 | 0.03 |
| deletion 38 | | | | | |
| Whole arm | 9q | NA | NA | chr15: 20000000-102531392 | 0.03 |
| deletion 39 | | | | | |

TABLE 4
The copy number alteration specifications of cluster 3 (genetic subtype 3). All genomic regions
of the biomarker panel of Table 1 are listed together with the frequency that these regions are affected
in cluster 3.

| | | | | | Frequency of region |
|---|---|---|---|---|---|
| | | | | | being affected in |
| Unique Name | Descriptor | Wide Peak Limits | Peak Limits | Region Limits | cluster 3 |
| Amplification | 1p34.2 | chr1: 39144054-44367347 | chr1: 42834925-43114106 | chr1: 42692914-43456374 | 0.06 |
| Peak 1 | | | | | |
| Amplification | 1q22 | chr1: 120494739-199253746 | chr1: 155143717-155177826 | chr1: 120497533-175439985 | 0.42 |
| Peak 2 | | | | | |
| Amplification | 1q43 | chr1: 201615295-249250621 | chr1: 242413158-242638099 | chr1: 242270003-249250621 | 0.39 |
| Peak 3 | | | | | |
| Amplification | 2q33.1 | chr2: 181446594-243199373 | chr2: 199603526-199649638 | chr2: 199525178-199824262 | 0.29 |
| Peak 4 | | | | | |
| Amplification | 3q29 | chr3: 174745992-198022430 | chr3: 195922177-195991311 | chr3: 174746836-198022430 | 0.31 |
| Peak 5 | | | | | |
| Amplification | 5p15.33 | chr5: 1-6418038 | chr5: 1-949725 | chr5: 1-2807521 | 0.33 |
| Peak 6 | | | | | |
| Amplification | 5p12 | chr5: 33430780-50470114 | chr5: 43822799-43929095 | chr5: 37334807-44266311 | 0.35 |
| Peak 7 | | | | | |
| Amplification | 6p21.1 | chr6: 43545080-44164949 | chr6: 43765716-44163738 | chr6: 36748055-52028937 | 0.45 |
| Peak 8 | | | | | |
| Amplification | 6q23.3 | chr6: 135342981-135632150 | chr6: 135513416-135593890 | chr6: 133418393-138096856 | 0.31 |
| Peak 9 | | | | | |
| Amplification | 7p11.2 | chr7: 54949302-55950640 | chr7: 55248172-55271747 | chr7: 54949902-55955413 | 0.77 |
| Peak 10 | | | | | |
| Amplification | 8p11.23 | chr8: 38162916-38237532 | chr8: 38165117-38236937 | chr8: 34342520-48854434 | 0.29 |
| Peak 11 | | | | | |
| Amplification | 8p11.21 | chr8: 41760295-42054539 | chr8: 41767516-41796226 | chr8: 34342520-48854434 | 0.43 |
| Peak 12 | | | | | |
| Amplification | 8q12.2 | chr8: 60847395-62906825 | chr8: 61658252-61874896 | chr8: 54646329-146364022 | 0.54 |
| Peak 13 | | | | | |
| Amplification | 8q21.13 | chr8: 80636493-82552412 | chr8: 81853752-81935079 | chr8: 54646329-146364022 | 0.56 |
| Peak 14 | | | | | |
| Amplification | 8q22.3 | chr8: 101853171-101878037 | chr8: 101853464-101876083 | chr8: 54646329-146364022 | 0.59 |
| Peak 15 | | | | | |
| Amplification | 8q24.21 | chr8: 128574277-128592142 | chr8: 128574768-128591041 | chr8: 54646329-146364022 | 0.63 |
| Peak 16 | | | | | |
| Amplification | 10q22.3 | chr10: 79956009-83248579 | chr10: 80445049-80732930 | chr10: 77952482-83583616 | 0.07 |
| Peak 17 | | | | | |
| Amplification | 11p15.5 | chr11: 2091739-2302637 | chr11: 2095340-2301396 | chr11: 2095340-2305324 | 0.30 |
| Peak 18 | | | | | |
| Amplification | 11q13.3 | chr11: 68746750-69827851 | chr11: 69311828-69824706 | chr11: 68748468-70760456 | 0.19 |
| Peak 19 | | | | | |
| Amplification | 12p13.32 | chr12: 3941807-4552801 | chr12: 4257035-4414967 | chr12: 1-8970181 | 0.31 |
| Peak 20 | | | | | |
| Amplification | 12p11.22 | chr12: 24880798-28477058 | chr12: 27800441-27813180 | chr12: 25955987-28156868 | 0.27 |
| Peak 21 | | | | | |
| Amplification | 13q12.2 | chr13: 28164386-28564331 | chr13: 28174614-28222659 | chr13: 1-115169878 | 0.85 |
| Peak 22 | | | | | |
| Amplification | 13q22.1 | chr13: 73775176-74007122 | chr13: 73906681-74004816 | chr13: 1-115169878 | 0.84 |
| Peak 23 | | | | | |
| Amplification | 13q34 | chr13: 110097815-111753132 | chr13: 110614601-110852082 | chr13: 1-115169878 | 0.81 |
| Peak 24 | | | | | |
| Amplification | 15q26.1 | chr15: 84648780-102531392 | chr15: 90811423-90958538 | chr15: 90086447-91895585 | 0.11 |
| Peak 25 | | | | | |
| Amplification | 16p11.2 | chr16: 30548665-30684980 | chr16: 30549334-30682578 | chr16: 28556705-46663588 | 0.57 |
| Peak 26 | | | | | |
| Amplification | 16q12.1 | chr16: 52243935-52652774 | chr16: 52383686-52652198 | chr16: 51078632-53117566 | 0.48 |
| Peak 27 | | | | | |
| Amplification | 17q11.2 | chr17: 27339261-27528113 | chr17: 27339592-27419750 | chr17: 20042949-40168023 | 0.30 |
| Peak 28 | | | | | |
| Amplification | 17q12 | chr17: 37747533-38052599 | chr17: 37950674-38031986 | chr17: 20042949-40168023 | 0.30 |
| Peak 29 | | | | | |
| Amplification | 17q23.2 | chr17: 58400463-58674819 | chr17: 58400997-58532661 | chr17: 57045336-62714320 | 0.28 |
| Peak 30 | | | | | |
| Amplification | 17q24.2 | chr17: 65638523-65873408 | chr17: 65649475-65769071 | chr17: 63042470-68655121 | 0.29 |
| Peak 31 | | | | | |
| Amplification | 17q25.3 | chr17: 77601682-77857881 | chr17: 77766436-77856811 | chr17: 75845339-81195210 | 0.29 |
| Peak 32 | | | | | |
| Amplification | 18q11.2 | chr18: 19517253-20155059 | chr18: 19525020-20154578 | chr18: 19243919-21268418 | 0.07 |
| Peak 33 | | | | | |
| Amplification | 18q21.1 | chr18: 46041609-46930382 | chr18: 46474762-46604295 | chr18: 46043163-46624631 | 0.04 |
| Peak 34 | | | | | |
| Amplification | 19q13.11 | chr19: 32439834-33021877 | chr19: 32966965-32988344 | chr19: 19939350-41312227 | 0.35 |
| Peak 35 | | | | | |
| Amplification | 19q13.2 | chr19: 39844313-40357920 | chr19: 39848086-40009185 | chr19: 19939350-41312227 | 0.34 |
| Peak 36 | | | | | |
| Amplification | 20p12.3 | chr20: 5376354-5742957 | chr20: 5379141-5737687 | chr20: 5156552-5961498 | 0.59 |
| Peak 37 | | | | | |
| Amplification | 20p12.1 | chr20: 16140291-16967195 | chr20: 16488303-16962609 | chr20: 16146669-16970141 | 0.65 |
| Peak 38 | | | | | |
| Amplification | 20p11.22 | chr20: 22058560-22353663 | chr20: 22063756-22350551 | chr20: 20938975-24286806 | 0.71 |
| Peak 39 | | | | | |
| Amplification | 20q11.21 | chr20: 29981165-30284236 | chr20: 30158807-30231055 | chr20: 24375411-63025520 | 0.95 |
| Peak 40 | | | | | |
| Amplification | 20q13.12 | chr20: 42534268-43281676 | chr20: 42537604-42773258 | chr20: 24375411-63025520 | 0.94 |
| Peak 41 | | | | | |
| Amplification | 20q13.2 | chr20: 52240629-52852159 | chr20: 52246659-52447690 | chr20: 24375411-63025520 | 0.95 |
| Peak 42 | | | | | |
| Amplification | 20q13.32 | chr20: 56110288-57258354 | chr20: 57000076-57085518 | chr20: 24375411-63025520 | 0.95 |
| Peak 43 | | | | | |
| Deletion Peak 1 | 1p36.31 | chr1: 4843384-6053964 | chr1: 5646446-6050774 | chr1: 1-37080378 | 0.62 |
| Deletion Peak 2 | 1p36.11 | chr1: 26898389-27219375 | chr1: 27139248-27185402 | chr1: 1-37080378 | 0.68 |
| Deletion Peak 3 | 1p33 | chr1: 49187432-50544677 | chr1: 48847112-50490440 | chr1: 49196205-51396500 | 0.59 |
| Deletion Peak 4 | 1p31.1 | chr1: 68959091-82269682 | chr1: 79308665-79566142 | chr1: 54944113-149961894 | 0.63 |
| Deletion Peak 5 | 1p21.1 | chr1: 102460262-107618224 | chr1: 104041213-104355050 | chr1: 54944113-149961894 | 0.66 |
| Deletion Peak 6 | 2p25.3 | chr2: 1-4752017 | chr2: 1-488785 | chr2: 1-5260278 | 0.22 |
| Deletion Peak 7 | 2p21 | chr2: 42587649-44009118 | chr2: 43440855-43455807 | chr2: 43159857-43470966 | 0.09 |
| Deletion Peak 8 | 2q23.1 | chr2: 147341956-149499455 | chr2: 148437688-148750846 | chr2: 148444810-148756213 | 0.08 |
| Deletion Peak 9 | 2q37.3 | chr2: 240321205-243199373 | chr2: 240940975-241060875 | chr2: 240589192-241997730 | 0.11 |
| Deletion Peak | 3p26.3 | chr3: 1-8350135 | chr3: 1-2251569 | chr3: 1-9456413 | 0.33 |
| 10 | | | | | |
| Deletion Peak | 3p14.2 | chr3: 58946448-61555632 | chr3: 59692189-61457375 | chr3: 59692189-61460946 | 0.28 |
| 11 | | | | | |
| Deletion Peak | 3p13 | chr3: 70014100-71250049 | chr3: 70976616-71182883 | chr3: 70977475-71315576 | 0.32 |
| 12 | | | | | |
| Deletion Peak | 3q26.31 | chr3: 173995229-175766885 | chr3: 174575326-175766885 | chr3: 174646449-174998725 | 0.10 |
| 13 | | | | | |
| Deletion Peak | 4p16.2 | chr4: 1-9787265 | chr4: 5915497-5925651 | chr4: 1-11455571 | 0.72 |
| 14 | | | | | |
| Deletion Peak | 4q22.1 | chr4: 91143530-93226628 | chr4: 91046982-92557520 | chr4: 91103554-92561908 | 0.74 |
| 15 | | | | | |
| Deletion Peak | 4q25 | chr4: 109046573-109544048 | chr4: 109451604-109544048 | chr4: 109489936-109535239 | 0.74 |
| 16 | | | | | |
| Deletion Peak | 4q31.3 | chr4: 152679194-153694102 | chr4: 153232273-153473069 | chr4: 152233433-191154276 | 0.74 |
| 17 | | | | | |
| Deletion Peak | 4q32.1 | chr4: 156135292-162306004 | chr4: 159683040-159830757 | chr4: 152233433-191154276 | 0.76 |
| 18 | | | | | |
| Deletion Peak | 4q35.1 | chr4: 184432080-185262191 | chr4: 184545375-185154488 | chr4: 152233433-191154276 | 0.78 |
| 19 | | | | | |
| Deletion Peak | 5p15.33 | chr5: 1-2750686 | chr5: 1368344-2252637 | chr5: 1-3864653 | 0.29 |
| 20 | | | | | |
| Deletion Peak | 5q12.1 | chr5: 58263825-59784640 | chr5: 58263825-59784640 | chr5: 50968806-135478060 | 0.45 |
| 21 | | | | | |
| Deletion Peak | 5q22.2 | chr5: 111312546-112362638 | chr5: 111747052-111761475 | chr5: 50968806-135478060 | 0.50 |
| 22 | | | | | |
| Deletion Peak | 6p25.3 | chr6: 1613114-2256643 | chr6: 1621843-2256643 | chr6: 1626114-2752758 | 0.18 |
| 23 | | | | | |
| Deletion Peak | 6p22.2 | chr6: 25926929-26025173 | chr6: 25932794-26025173 | chr6: 25933563-26346501 | 0.18 |
| 24 | | | | | |
| Deletion Peak | 6q21 | chr6: 91006553-129206367 | chr6: 111544567-112706964 | chr6: 108785353-116683959 | 0.25 |
| 25 | | | | | |
| Deletion Peak | 6q26 | chr6: 161540781-163179430 | chr6: 161540781-163179430 | chr6: 160448048-171115067 | 0.31 |
| 26 | | | | | |
| Deletion Peak | 7q31.1 | chr7: 109590674-111367757 | chr7: 110232248-111358792 | chr7: 110595801-110618403 | 0.05 |
| 27 | | | | | |
| Deletion Peak | 8p23.3 | chr8: 1-1449850 | chr8: 1-623109 | chr8: 1-10801317 | 0.79 |
| 28 | | | | | |
| Deletion Peak | 8p21.3 | chr8: 13423967-26607015 | chr8: 21547484-21647267 | chr8: 11646511-25738962 | 0.76 |
| 29 | | | | | |
| Deletion Peak | 8p12 | chr8: 33445578-37452445 | chr8: 34944675-36494184 | chr8: 35186331-35207233 | 0.57 |
| 30 | | | | | |
| Deletion Peak | 8q11.1 | chr8: 42874387-48101823 | chr8: 42931769-48061147 | chr8: 42932439-48062091 | 0.13 |
| 31 | | | | | |
| Deletion Peak | 9p21.3 | chr9: 21558582-22452906 | chr9: 21995318-22021004 | chr9: 21711940-22331169 | 0.20 |
| 32 | | | | | |
| Deletion Peak | 10p15.3 | chr10: 1-855610 | chr10: 1-418075 | chr10: 1-2055670 | 0.32 |
| 33 | | | | | |
| Deletion Peak | 10q21.1 | chr10: 51561927-53481136 | chr10: 51561927-54065263 | chr10: 50763189-54253314 | 0.47 |
| 34 | | | | | |
| Deletion Peak | 10q23.31 | chr10: 89502327-90051809 | chr10: 89574482-89607380 | chr10: 83248858-135534747 | 0.53 |
| 35 | | | | | |
| Deletion Peak | 10q25.2 | chr10: 114197471-115353755 | chr10: 114708532-114929210 | chr10: 83248858-135534747 | 0.49 |
| 36 | | | | | |
| Deletion Peak | 10q26.3 | chr10: 133107199-135534747 | chr10: 135221096-135534747 | chr10: 83248858-135534747 | 0.56 |
| 37 | | | | | |
| Deletion Peak | 11q22.3 | chr11: 102958343-135006516 | chr11: 108251085-108350263 | chr11: 108179920-108470348 | 0.37 |
| 38 | | | | | |
| Deletion Peak | 12p13.2 | chr12: 12412186-13039757 | chr12: 12525464-12721981 | chr12: 11739866-13828006 | 0.22 |
| 39 | | | | | |
| Deletion Peak | 12q21.2 | chr12: 75602135-79819184 | chr12: 76423769-76523220 | chr12: 75648890-78593389 | 0.25 |
| 40 | | | | | |
| Deletion Peak | 12q24.33 | chr12: 125048064-133851895 | chr12: 131256822-131362341 | chr12: 125246979-133851895 | 0.33 |
| 41 | | | | | |
| Deletion Peak | 14q24.1 | chr14: 68280014-69351476 | chr14: 68284712-68948164 | chr14: 68343659-68985929 | 0.58 |
| 42 | | | | | |
| Deletion Peak | 14q32.11 | chr14: 78347564-107349540 | chr14: 90999841-91286309 | chr14: 90170502-92315681 | 0.55 |
| 43 | | | | | |
| Deletion Peak | 15q11.2 | chr15: 25436434-25466900 | chr15: 25438412-25459423 | chr15: 1-58781038 | 0.70 |
| 44 | | | | | |
| Deletion Peak | 15q21.1 | chr15: 44850582-45321541 | chr15: 44851811-45052539 | chr15: 1-58781038 | 0.66 |
| 45 | | | | | |
| Deletion Peak | 15q22.33 | chr15: 67053712-67697647 | chr15: 67337017-67551787 | chr15: 67245914-67753643 | 0.67 |
| 46 | | | | | |
| Deletion Peak | 16p13.3 | chr16: 5141600-8053542 | chr16: 6056420-8053542 | chr16: 5279421-7736962 | 0.13 |
| 47 | | | | | |
| Deletion Peak | 16q23.1 | chr16: 78131135-79628242 | chr16: 78131135-79286336 | chr16: 78424695-79295468 | 0.14 |
| 48 | | | | | |
| Deletion Peak | 17p12 | chr17: 11466949-12461211 | chr17: 11872374-11906034 | chr17: 10231123-12752622 | 0.83 |
| 49 | | | | | |
| Deletion Peak | 17q24.3 | chr17: 68174484-70599305 | chr17: 70337175-70590424 | chr17: 70549664-70552757 | 0.30 |
| 50 | | | | | |
| Deletion Peak | 18p11.31 | chr18: 3277394-4265401 | chr18: 3441391-3481373 | chr18: 3398771-3729936 | 0.83 |
| 51 | | | | | |
| Deletion Peak | 18q12.2 | chr18: 35136370-39061915 | chr18: 36781295-37333625 | chr18: 35827844-46846953 | 0.93 |
| 52 | | | | | |
| Deletion Peak | 18q21.2 | chr18: 48472034-48707815 | chr18: 48547928-48660122 | chr18: 47049746-78077248 | 0.94 |
| 53 | | | | | |
| Deletion Peak | 18q21.33 | chr18: 60645473-61013467 | chr18: 60788090-61013467 | chr18: 47049746-78077248 | 0.95 |
| 54 | | | | | |
| Deletion Peak | 19p13.3 | chr19: 1488247-1660256 | chr19: 1488455-1660256 | chr19: 1-5957502 | 0.24 |
| 55 | | | | | |
| Deletion Peak | 20p12.1 | chr20: 13955189-16350354 | chr20: 14416445-15462745 | chr20: 14096517-16064189 | 0.21 |
| 56 | | | | | |
| Deletion Peak | 21q11.2 | chr21: 15555708-16334057 | chr21: 15854887-15919066 | chr21: 1-31258004 | 0.53 |
| 57 | | | | | |
| Deletion Peak | 21q21.1 | chr21: 23106546-26219369 | chr21: 23248652-23494410 | chr21: 1-31258004 | 0.54 |
| 58 | | | | | |
| Deletion Peak | 22q13.32 | chr22: 48649199-49178363 | chr22: 48906309-49158314 | chr22: 47983027-51304566 | 0.78 |
| 59 | | | | | |
| Whole arm | 10p | NA | NA | chr10: 1-39254935 | 0.15 |
| amplification 1 | | | | | |
| Whole arm | 10q | NA | NA | chr16: 1-35335801 | 0.04 |
| amplification 2 | | | | | |
| Whole arm | 11p | NA | NA | chr16: 38335801-90354753 | 0.23 |
| amplification 3 | | | | | |
| Whole arm | 11q | NA | NA | chr17: 1-22263006 | 0.16 |
| amplification 4 | | | | | |
| Whole arm | 12p | NA | NA | chr17: 25263006-81195210 | 0.23 |
| amplification 5 | | | | | |
| Whole arm | 12q | NA | NA | chr18: 1-15460898 | 0.15 |
| amplification 6 | | | | | |
| Whole arm | 13q | NA | NA | chr18: 18460898-78077248 | 0.80 |
| amplification 7 | | | | | |
| Whole arm | 14q | NA | NA | chr19: 1-24681782 | 0.06 |
| amplification 8 | | | | | |
| Whole arm | 15q | NA | NA | chr19: 27681782-59128983 | 0.03 |
| amplification 9 | | | | | |
| Whole arm | 16p | NA | NA | chr1: 1-121535434 | 0.51 |
| amplification | | | | | |
| 10 | | | | | |
| Whole arm | 16q | NA | NA | chr1: 124535434-249250621 | 0.45 |
| amplification | | | | | |
| 11 | | | | | |
| Whole arm | 17p | NA | NA | chr10: 42254935-135534747 | 0.04 |
| amplification | | | | | |
| 12 | | | | | |
| Whole arm | 17q | NA | NA | chr20: 1-26369569 | 0.23 |
| amplification | | | | | |
| 13 | | | | | |
| Whole arm | 18p | NA | NA | chr20: 29369569-63025520 | 0.04 |
| amplification | | | | | |
| 14 | | | | | |
| Whole arm | 18q | NA | NA | chr21: 14288129-48129895 | 0.02 |
| amplification | | | | | |
| 15 | | | | | |
| Whole arm | 19p | NA | NA | chr22: 16000000-51304566 | 0.32 |
| amplification | | | | | |
| 16 | | | | | |
| Whole arm | 19q | NA | NA | chr2: 1-92326171 | 0.32 |
| amplification | | | | | |
| 17 | | | | | |
| Whole arm | 1p | NA | NA | chr2: 95326171-243199373 | 0.07 |
| amplification | | | | | |
| 18 | | | | | |
| Whole arm | 1q | NA | NA | chr3: 1-90504854 | 0.35 |
| amplification | | | | | |
| 19 | | | | | |
| Whole arm | 20p | NA | NA | chr3: 93504854-198022430 | 0.64 |
| amplification | | | | | |
| 20 | | | | | |
| Whole arm | 20q | NA | NA | chr4: 1-49660117 | 0.93 |
| amplification | | | | | |
| 21 | | | | | |
| Whole arm | 21q | NA | NA | chr4: 52660117-191154276 | 0.08 |
| amplification | | | | | |
| 22 | | | | | |
| Whole arm | 22q | NA | NA | chr11: 1-51644205 | 0.01 |
| amplification | | | | | |
| 23 | | | | | |
| Whole arm | 2p | NA | NA | chr5: 1-46405641 | 0.23 |
| amplification | | | | | |
| 24 | | | | | |
| Whole arm | 2q | NA | NA | chr5: 49405641-180915260 | 0.21 |
| amplification | | | | | |
| 25 | | | | | |
| Whole arm | 3p | NA | NA | chr6: 1-58830166 | 0.12 |
| amplification | | | | | |
| 26 | | | | | |
| Whole arm | 3q | NA | NA | chr6: 61830166-171115067 | 0.20 |
| amplification | | | | | |
| 27 | | | | | |
| Whole arm | 4p | NA | NA | chr7: 1-58054331 | 0.02 |
| amplification | | | | | |
| 28 | | | | | |
| Whole arm | 4q | NA | NA | chr7: 61054331-159138663 | 0.02 |
| amplification | | | | | |
| 29 | | | | | |
| Whole arm | 5p | NA | NA | chr8: 1-43838887 | 0.29 |
| amplification | | | | | |
| 30 | | | | | |
| Whole arm | 5q | NA | NA | chr8: 46838887-146364022 | 0.11 |
| amplification | | | | | |
| 31 | | | | | |
| Whole arm | 6p | NA | NA | chr9: 1-47367679 | 0.34 |
| amplification | | | | | |
| 32 | | | | | |
| Whole arm | 6q | NA | NA | chr9: 50367679-141213431 | 0.28 |
| amplification | | | | | |
| 33 | | | | | |
| Whole arm | 7p | NA | NA | chr11: 54644205-135006516 | 0.77 |
| amplification | | | | | |
| 34 | | | | | |
| Whole arm | 7q | NA | NA | chr12: 1-34856694 | 0.65 |
| amplification | | | | | |
| 35 | | | | | |
| Whole arm | 8p | NA | NA | chr12: 37856694-133851895 | 0.13 |
| amplification | | | | | |
| 36 | | | | | |
| Whole arm | 8q | NA | NA | chr13: 19000000-115169878 | 0.51 |
| amplification | | | | | |
| 37 | | | | | |
| Whole arm | 9p | NA | NA | chr14: 19000000-107349540 | 0.25 |
| amplification | | | | | |
| 38 | | | | | |
| Whole arm | 9q | NA | NA | chr15: 20000000-102531392 | 0.17 |
| amplification | | | | | |
| 39 | | | | | |
| Whole arm | 10p | NA | NA | chr10: 1-39254935 | 0.28 |
| deletion 1 | | | | | |
| Whole arm | 10q | NA | NA | chr16: 1-35335801 | 0.38 |
| deletion 2 | | | | | |
| Whole arm | 11p | NA | NA | chr16: 38335801-90354753 | 0.25 |
| deletion 3 | | | | | |
| Whole arm | 11q | NA | NA | chr17: 1-22263006 | 0.31 |
| deletion 4 | | | | | |
| Whole arm | 12p | NA | NA | chr17: 25263006-81195210 | 0.15 |
| deletion 5 | | | | | |
| Whole arm | 12q | NA | NA | chr18: 1-15460898 | 0.16 |
| deletion 6 | | | | | |
| Whole arm | 13q | NA | NA | chr18: 18460898-78077248 | 0.04 |
| deletion 7 | | | | | |
| Whole arm | 14q | NA | NA | chr19: 1-24681782 | 0.52 |
| deletion 8 | | | | | |
| Whole arm | 15q | NA | NA | chr19: 27681782-59128983 | 0.65 |
| deletion 9 | | | | | |
| Whole arm | 16p | NA | NA | chr1: 1-121535434 | 0.09 |
| deletion 10 | | | | | |
| Whole arm | 16q | NA | NA | chr1: 124535434-249250621 | 0.09 |
| deletion 11 | | | | | |
| Whole arm | 17p | NA | NA | chr10: 42254935-135534747 | 0.67 |
| deletion 12 | | | | | |
| Whole arm | 17q | NA | NA | chr20: 1-26369569 | 0.30 |
| deletion 13 | | | | | |
| Whole arm | 18p | NA | NA | chr20: 29369569-63025520 | 0.84 |
| deletion 14 | | | | | |
| Whole arm | 18q | NA | NA | chr21: 14288129-48129895 | 0.94 |
| deletion 15 | | | | | |
| Whole arm | 19p | NA | NA | chr22: 16000000-51304566 | 0.14 |
| deletion 16 | | | | | |
| Whole arm | 19q | NA | NA | chr2: 1-92326171 | 0.11 |
| deletion 17 | | | | | |
| Whole arm | 1p | NA | NA | chr2: 95326171-243199373 | 0.41 |
| deletion 18 | | | | | |
| Whole arm | 1q | NA | NA | chr3: 1-90504854 | 0.15 |
| deletion 19 | | | | | |
| Whole arm | 20p | NA | NA | chr3: 93504854-198022430 | 0.18 |
| deletion 20 | | | | | |
| Whole arm | 20q | NA | NA | chr4: 1-49660117 | 0.00 |
| deletion 21 | | | | | |
| Whole arm | 21q | NA | NA | chr4: 52660117-191154276 | 0.50 |
| deletion 22 | | | | | |
| Whole arm | 22q | NA | NA | chr11: 1-51644205 | 0.72 |
| deletion 23 | | | | | |
| Whole arm | 2p | NA | NA | chr5: 1-46405641 | 0.04 |
| deletion 24 | | | | | |
| Whole arm | 2q | NA | NA | chr5: 49405641-180915260 | 0.04 |
| deletion 25 | | | | | |
| Whole arm | 3p | NA | NA | chr6: 1-58830166 | 0.21 |
| deletion 26 | | | | | |
| Whole arm | 3q | NA | NA | chr6: 61830166-171115067 | 0.09 |
| deletion 27 | | | | | |
| Whole arm | 4p | NA | NA | chr7: 1-58054331 | 0.71 |
| deletion 28 | | | | | |
| Whole arm | 4q | NA | NA | chr7: 61054331-159138663 | 0.68 |
| deletion 29 | | | | | |
| Whole arm | 5p | NA | NA | chr8: 1-43838887 | 0.14 |
| deletion 30 | | | | | |
| Whole arm | 5q | NA | NA | chr8: 46838887-146364022 | 0.26 |
| deletion 31 | | | | | |
| Whole arm | 6p | NA | NA | chr9: 1-47367679 | 0.13 |
| deletion 32 | | | | | |
| Whole arm | 6q | NA | NA | chr9: 50367679-141213431 | 0.16 |
| deletion 33 | | | | | |
| Whole arm | 7p | NA | NA | chr11: 54644205-135006516 | 0.01 |
| deletion 34 | | | | | |
| Whole arm | 7q | NA | NA | chr12: 1-34856694 | 0.03 |
| deletion 35 | | | | | |
| Whole arm | 8p | NA | NA | chr12: 37856694-133851895 | 0.59 |
| deletion 36 | | | | | |
| Whole arm | 8q | NA | NA | chr13: 19000000-115169878 | 0.06 |
| deletion 37 | | | | | |
| Whole arm | 9p | NA | NA | chr14: 19000000-107349540 | 0.17 |
| deletion 38 | | | | | |
| Whole arm | 9q | NA | NA | chr15: 20000000-102531392 | 0.20 |
| deletion 39 | | | | | |

TABLE 5
Biomarker panel listing the 102 focal genomic regions used to cluster the studied CRC
samples in 3 genomic subtypes. The panel of genomic regions consists of 43 focal
amplifications, 59 focal deletions.

| Unique Name | Descriptor | Wide Peak Limits | Peak Limits | Region Limits |
|---|---|---|---|---|
| Amplification | 1p34.2 | chr1: 39144054-44367347 | chr1: 42834925-43114106 | chr1: 42692914-43456374 |
| Peak 1 | | (probes 8327:9928) | (probes 9441:9533) | (probes 9397:9635) |
| Amplification | 1q22 | chr1: 120494739-199253746 | chr1: 155143717-155177826 | chr1: 120497533-175439985 |
| Peak 2 | | (probes 33660:51419) | (probes 35410:35412) | (probes 33661:42337) |
| Amplification | 1q43 | chr1: 201615295-249250621 | chr1: 242413158-242638099 | chr1: 242270003-249250621 |
| Peak 3 | | (probes 52203:66664) | (probes 65295:65368) | (probes 65251:66664) |
| Amplification | 2q33.1 | chr2: 181446594-243199373 | chr2: 199603526-199649638 | chr2: 199525178-199824262 |
| Peak 4 | | (probes 121383:140439) | (probes 127082:127089) | (probes 127052:127139) |
| Amplification | 3q29 | chr3: 174745992-198022430 | chr3: 195922177-195991311 | chr3: 174746836-198022430 |
| Peak 5 | | (probes 195590:202834) | (probes 202552:202570) | (probes 195591:202834) |
| Amplification | 5p15.33 | chr5: 1-6418038 | chr5: 1-949725 | chr5: 1-2807521 |
| Peak 6 | | (probes 260156:261588) | (probes 260156:260170) | (probes 260156:260460) |
| Amplification | 5p12 | chr5: 33430780-50470114 | chr5: 43822799-43929095 | chr5: 37334807-44266311 |
| Peak 7 | | (probes 269602:272992) | (probes 272538:272572) | (probes 270584:272613) |
| Amplification | 6p21.1 | chr6: 43545080-44164949 | chr6: 43765716-44163738 | chr6: 36748055-52028937 |
| Peak 8 | | (probes 323466:323553) | (probes 323497:323552) | (probes 321584:325929) |
| Amplification | 6q23.3 | chr6: 135342981-135632150 | chr6: 135513416-135593890 | chr6: 133418393-138096856 |
| Peak 9 | | (probes 350621:350776) | (probes 350670:350763) | (probes 350049:351529) |
| Amplification | 7p11.2 | chr7: 54949302-55950640 | chr7: 55248172-55271747 | chr7: 54949902-55955413 |
| Peak 10 | | (probes 380333:380688) | (probes 380546:380547) | (probes 380334:380690) |
| Amplification | 8p11.23 | chr8: 38162916-38237532 | chr8: 38165117-38236937 | chr8: 34342520-48854434 |
| Peak 11 | | (probes 417907:417912) | (probes 417908:417911) | (probes 417738:418611) |
| Amplification | 8p11.21 | chr8: 41760295-42054539 | chr8: 41767516-41796226 | chr8: 34342520-48854434 |
| Peak 12 | | (probes 418156:418183) | (probes 418157:418159) | (probes 417738:418611) |
| Amplification | 8q12.2 | chr8: 60847395-62906825 | chr8: 61658252-61874896 | chr8: 54646329-146364022 |
| Peak 13 | | (probes 421733:422344) | (probes 421947:422037) | (probes 420141:444693) |
| Amplification | 8q21.13 | chr8: 80636493-82552412 | chr8: 81853752-81935079 | chr8: 54646329-146364022 |
| Peak 14 | | (probes 427272:427752) | (probes 427579:427595) | (probes 420141:444693) |
| Amplification | 8q22.3 | chr8: 101853171-101878037 | chr8: 101853464-101876083 | chr8: 54646329-146364022 |
| Peak 15 | | (probes 432850:432855) | (probes 432851:432854) | (probes 420141:444693) |
| Amplification | 8q24.21 | chr8: 128574277-128592142 | chr8: 128574768-128591041 | chr8: 54646329-146364022 |
| Peak 16 | | (probes 440457:440461) | (probes 440458:440460) | (probes 420141:444693) |
| Amplification | 10q22.3 | chr10: 79956009-83248579 | chr10: 80445049-80732930 | chr10: 77952482-83583616 |
| Peak 17 | | (probes 504622:505397) | (probes 504807:504859) | (probes 503919:505484) |
| Amplification | 11p15.5 | chr11: 2091739-2302637 | chr11: 2095340-2301396 | chr11: 2095340-2305324 |
| Peak 18 | | (probes 520960:520971) | (probes 520961:520970) | (probes 520961:520973) |
| Amplification | 11q13.3 | chr11: 68746750-69827851 | chr11: 69311828-69824706 | chr11: 68748468-70760456 |
| Peak 19 | | (probes 539709:539869) | (probes 539796:539868) | (probes 539710:540034) |
| Amplification | 12p13.32 | chr12: 3941807-4552801 | chr12: 4257035-4414967 | chr12: 1-8970181 |
| Peak 20 | | (probes 562075:562238) | (probes 562162:562219) | (probes 561076:563266) |
| Amplification | 12p11.22 | chr12: 24880798-28477058 | chr12: 27800441-27813180 | chr12: 25955987-28156868 |
| Peak 21 | | (probes 567897:568917) | (probes 568722:568722) | (probes 568181:568838) |
| Amplification | 13q12.2 | chr13: 28164386-28564331 | chr13: 28174614-28222659 | chr13: 1-115169878 |
| Peak 22 | | (probes 600542:600653) | (probes 600543:600550) | (probes 599000:624834) |
| Amplification | 13q22.1 | chr13: 73775176-74007122 | chr13: 73906681-74004816 | chr13: 1-115169878 |
| Peak 23 | | (probes 612853:612922) | (probes 612891:612921) | (probes 599000:624834) |
| Amplification | 13q34 | chr13: 110097815-111753132 | chr13: 110614601-110852082 | chr13: 1-115169878 |
| Peak 24 | | (probes 624037:624356) | (probes 624207:624235) | (probes 599000:624834) |
| Amplification | 15q26.1 | chr15: 84648780-102531392 | chr15: 90811423-90958538 | chr15: 90086447-91895585 |
| Peak 25 | | (probes 669559:675028) | (probes 671504:671507) | (probes 671145:671795) |
| Amplification | 16p11.2 | chr16: 30548665-30684980 | chr16: 30549334-30682578 | chr16: 28556705-46663588 |
| Peak 26 | | (probes 681497:681499) | (probes 681498:681498) | (probes 681456:681707) |
| Amplification | 16q12.1 | chr16: 52243935-52652774 | chr16: 52383686-52652198 | chr16: 51078632-53117566 |
| Peak 27 | | (probes 683896:684007) | (probes 683905:684006) | (probes 683397:684118) |
| Amplification | 17q11.2 | chr17: 27339261-27528113 | chr17: 27339592-27419750 | chr17: 20042949-40168023 |
| Peak 28 | | (probes 701849:701907) | (probes 701850:701866) | (probes 701549:704344) |
| Amplification | 17q12 | chr17: 37747533-38052599 | chr17: 37950674-38031986 | chr17: 20042949-40168023 |
| Peak 29 | | (probes 703891:703930) | (probes 703917:703929) | (probes 701549:704344) |
| Amplification | 17q23.2 | chr17: 58400463-58674819 | chr17: 58400997-58532661 | chr17: 57045336-62714320 |
| Peak 30 | | (probes 708991:709072) | (probes 708992:709031) | (probes 708646:710280) |
| Amplification | 17q24.2 | chr17: 65638523-65873408 | chr17: 65649475-65769071 | chr17: 63042470-68655121 |
| Peak 31 | | (probes 710976:711074) | (probes 710978:711037) | (probes 710378:712109) |
| Amplification | 17q25.3 | chr17: 77601682-77857881 | chr17: 77766436-77856811 | chr17: 75845339-81195210 |
| Peak 32 | | (probes 714127:714155) | (probes 714137:714154) | (probes 713778:714413) |
| Amplification | 18q11.2 | chr18: 19517253-20155059 | chr18: 19525020-20154578 | chr18: 19243919-21268418 |
| Peak 33 | | (probes 718275:718278) | (probes 718276:718277) | (probes 718239:718447) |
| Amplification | 18q21.1 | chr18: 46041609-46930382 | chr18: 46474762-46604295 | chr18: 46043163-46624631 |
| Peak 34 | | (probes 725325:725688) | (probes 725462:725478) | (probes 725326:725481) |
| Amplification | 19q13.11 | chr19: 32439834-33021877 | chr19: 32966965-32988344 | chr19: 19939350-41312227 |
| Peak 35 | | (probes 739770:739825) | (probes 739799:739805) | (probes 739769:742785) |
| Amplification | 19q13.2 | chr19: 39844313-40357920 | chr19: 39848086-40009185 | chr19: 19939350-41312227 |
| Peak 36 | | (probes 742339:742488) | (probes 742340:742365) | (probes 739769:742785) |
| Amplification | 20p12.3 | chr20: 5376354-5742957 | chr20: 5379141-5737687 | chr20: 5156552-5961498 |
| Peak 37 | | (probes 749386:749394) | (probes 749387:749393) | (probes 749320:749400) |
| Amplification | 20p12.1 | chr20: 16140291-16967195 | chr20: 16488303-16962609 | chr20: 16146669-16970141 |
| Peak 38 | | (probes 751931:752121) | (probes 751982:752120) | (probes 751932:752123) |
| Amplification | 20p11.22 | chr20: 22058560-22353663 | chr20: 22063756-22350551 | chr20: 20938975-24286806 |
| Peak 39 | | (probes 753204:753212) | (probes 753205:753211) | (probes 753103:753480) |
| Amplification | 20q11.21 | chr20: 29981165-30284236 | chr20: 30158807-30231055 | chr20: 24375411-63025520 |
| Peak 40 | | (probes 753711:753769) | (probes 753729:753748) | (probes 753519:762719) |
| Amplification | 20q13.12 | chr20: 42534268-43281676 | chr20: 42537604-42773258 | chr20: 24375411-63025520 |
| Peak 41 | | (probes 756743:756930) | (probes 756744:756753) | (probes 753519:762719) |
| Amplification | 20q13.2 | chr20: 52240629-52852159 | chr20: 52246659-52447690 | chr20: 24375411-63025520 |
| Peak 42 | | (probes 759885:759989) | (probes 759886:759947) | (probes 753519:762719) |
| Amplification | 20q13.32 | chr20: 56110288-57258354 | chr20: 57000076-57085518 | chr20: 24375411-63025520 |
| Peak 43 | | (probes 760955:761384) | (probes 761307:761325) | (probes 753519:762719) |
| Deletion Peak 1 | 1p36.31 | chr1: 4843384-6053964 | chr1: 5646446-6050774 | chr1: 1-37080378 |
| | | (probes 343:539) | (probes 537:538) | (probes 1:7694) |
| Deletion Peak 2 | 1p36.11 | chr1: 26898389-27219375 | chr1: 27139248-27185402 | chr1: 1-37080378 |
| | | (probes 5577:5639) | (probes 5629:5630) | (probes 1:7694) |
| Deletion Peak 3 | 1p33 | chr1: 49187432-50544677 | chr1: 48847112-50490440 | chr1: 49196205-51396500 |
| | | (probes 11383:11605) | (probes 11319:11598) | (probes 11385:11801) |
| Deletion Peak 4 | 1p31.1 | chr1: 68959091-82269682 | chr1: 79308665-79566142 | chr1: 54944113-149961894 |
| | | (probes 17617:22019) | (probes 20993:20994) | (probes 12746:33663) |
| Deletion Peak 5 | 1p21.1 | chr1: 102460262-107618224 | chr1: 104041213-104355050 | chr1: 54944113-149961894 |
| | | (probes 28724:30035) | (probes 29059:29060) | (probes 12746:33663) |
| Deletion Peak 6 | 2p25.3 | chr2: 1-4752017 | chr2: 1-488785 | chr2: 1-5260278 |
| | | (probes 66665:67509) | (probes 66665:66665) | (probes 66665:67543) |
| Deletion Peak 7 | 2p21 | chr2: 42587649-44009118 | chr2: 43440855-43455807 | chr2: 43159857-43470966 |
| | | (probes 79800:80265) | (probes 80094:80097) | (probes 79991:80104) |
| Deletion Peak 8 | 2q23.1 | chr2: 147341956-149499455 | chr2: 148437688-148750846 | chr2: 148444810-148756213 |
| | | (probes 110935:111344) | (probes 111211:111212) | (probes 111212:111213) |
| Deletion Peak 9 | 2q37.3 | chr2: 240321205-243199373 | chr2: 240940975-241060875 | chr2: 240589192-241997730 |
| | | (probes 139897:140439) | (probes 140116:140117) | (probes 139997:140352) |
| Deletion Peak 10 | 3p26.3 | chr3: 1-8350135 | chr3: 1-2251569 | chr3: 1-9456413 |
| | | (probes 140440:142376) | (probes 140440:140440) | (probes 140440:142660) |
| Deletion Peak 11 | 3p14.2 | chr3: 58946448-61555632 | chr3: 59692189-61457375 | chr3: 59692189-61460946 |
| | | (probes 159123:159363) | (probes 159317:159339) | (probes 159317:159341) |
| Deletion Peak 12 | 3p13 | chr3: 70014100-71250049 | chr3: 70976616-71182883 | chr3: 70977475-71315576 |
| | | (probes 162276:162774) | (probes 162708:162756) | (probes 162709:162793) |
| Deletion Peak 13 | 3q26.31 | chr3: 173995229-175766885 | chr3: 174575326-175766885 | chr3: 174646449-174998725 |
| | | (probes 195434:195620) | (probes 195555:195620) | (probes 195574:195593) |
| Deletion Peak 14 | 4p16.2 | chr4: 1-9787265 | chr4: 5915497-5925651 | chr4: 1-11455571 |
| | | (probes 202835:205184) | (probes 204117:204118) | (probes 202835:205810) |
| Deletion Peak 15 | 4q22.1 | chr4: 91143530-93226628 | chr4: 91046982-92557520 | chr4: 91103554-92561908 |
| | | (probes 229840:230295) | (probes 229814:230049) | (probes 229834:230051) |
| Deletion Peak 16 | 4q25 | chr4: 109046573-109544048 | chr4: 109451604-109544048 | chr4: 109489936-109535239 |
| | | (probes 235677:235829) | (probes 235801:235829) | (probes 235811:235826) |
| Deletion Peak 17 | 4q31.3 | chr4: 152679194-153694102 | chr4: 153232273-153473069 | chr4: 152233433-191154276 |
| | | (probes 249686:250016) | (probes 249909:249977) | (probes 249594:260155) |
| Deletion Peak 18 | 4q32.1 | chr4: 156135292-162306004 | chr4: 159683040-159830757 | chr4: 152233433-191154276 |
| | | (probes 250770:252941) | (probes 252055:252100) | (probes 249594:260155) |
| Deletion Peak 19 | 4q35.1 | chr4: 184432080-185262191 | chr4: 184545375-185154488 | chr4: 152233433-191154276 |
| | | (probes 259152:259216) | (probes 259180:259181) | (probes 249594:260155) |
| Deletion Peak 20 | 5p15.33 | chr5: 1-2750686 | chr5: 1368344-2252637 | chr5: 1-3864653 |
| | | (probes 260156:260443) | (probes 260268:260279) | (probes 260156:260838) |
| Deletion Peak 21 | 5q12.1 | chr5: 58263825-59784640 | chr5: 58263825-59784640 | chr5: 50968806-135478060 |
| | | (probes 275334:275642) | (probes 275334:275642) | (probes 273162:297537) |
| Deletion Peak 22 | 5q22.2 | chr5: 111312546-112362638 | chr5: 111747052-111761475 | chr5: 50968806-135478060 |
| | | (probes 290226:290685) | (probes 290365:290367) | (probes 273162:297537) |
| Deletion Peak 23 | 6p25.3 | chr6: 1613114-2256643 | chr6: 1621843-2256643 | chr6: 1626114-2752758 |
| | | (probes 311779:311876) | (probes 311784:311876) | (probes 311786:312043) |
| Deletion Peak 24 | 6p22.2 | chr6: 25926929-26025173 | chr6: 25932794-26025173 | chr6: 25933563-26346501 |
| | | (probes 319619:319631) | (probes 319620:319631) | (probes 319621:319690) |
| Deletion Peak 25 | 6q21 | chr6: 91006553-129206367 | chr6: 111544567-112706964 | chr6: 108785353-116683959 |
| | | (probes 337170:348684) | (probes 343210:343577) | (probes 342315:344882) |
| Deletion Peak 26 | 6q26 | chr6: 161540781-163179430 | chr6: 161540781-163179430 | chr6: 160448048-171115067 |
| | | (probes 359016:359155) | (probes 359016:359155) | (probes 358753:361110) |
| Deletion Peak 27 | 7q31.1 | chr7: 109590674-111367757 | chr7: 110232248-111358792 | chr7: 110595801-110618403 |
| | | (probes 392698:393070) | (probes 392932:393069) | (probes 393001:393002) |
| Deletion Peak 28 | 8p23.3 | chr8: 1-1449850 | chr8: 1-623109 | chr8: 1-10801317 |
| | | (probes 408036:408314) | (probes 408036:408036) | (probes 408036:410838) |
| Deletion Peak 29 | 8p21.3 | chr8: 13423967-26607015 | chr8: 21547484-21647267 | chr8: 11646511-25738962 |
| | | (probes 411497:416107) | (probes 414395:414425) | (probes 411142:415781) |
| Deletion Peak 30 | 8p12 | chr8: 33445578-37452445 | chr8: 34944675-36494184 | chr8: 35186331-35207233 |
| | | (probes 417581:417886) | (probes 417838:417861) | (probes 417850:417851) |
| Deletion Peak 31 | 8q11.1 | chr8: 42874387-48101823 | chr8: 42931769-48061147 | chr8: 42932439-48062091 |
| | | (probes 418403:418427) | (probes 418413:418414) | (probes 418414:418415) |
| Deletion Peak 32 | 9p21.3 | chr9: 21558582-22452906 | chr9: 21995318-22021004 | chr9: 21711940-22331169 |
| | | (probes 452306:452602) | (probes 452487:452491) | (probes 452378:452557) |
| Deletion Peak 33 | 10p15.3 | chr10: 1-855610 | chr10: 1-418075 | chr10: 1-2055670 |
| | | (probes 482107:482163) | (probes 482107:482107) | (probes 482107:482391) |
| Deletion Peak 34 | 10q21.1 | chr10: 51561927-53481136 | chr10: 51561927-54065263 | chr10: 50763189-54253314 |
| | | (probes 495717:495904) | (probes 495717:496053) | (probes 495699:496079) |
| Deletion Peak 35 | 10q23.31 | chr10: 89502327-90051809 | chr10: 89574482-89607380 | chr10: 83248858-135534747 |
| | | (probes 507191:507378) | (probes 507201:507204) | (probes 505400:520735) |
| Deletion Peak 36 | 10q25.2 | chr10: 114197471-115353755 | chr10: 114708532-114929210 | chr10: 83248858-135534747 |
| | | (probes 515003:515382) | (probes 515200:515269) | (probes 505400:520735) |
| Deletion Peak 37 | 10q26.3 | chr10: 133107199-135534747 | chr10: 135221096-135534747 | chr10: 83248858-135534747 |
| | | (probes 520190:520735) | (probes 520735:520735) | (probes 505400:520735) |
| Deletion Peak 38 | 11q22.3 | chr11: 102958343-135006516 | chr11: 108251085-108350263 | chr11: 108179920-108470348 |
| | | (probes 551128:561075) | (probes 552798:552832) | (probes 552783:552882) |
| Deletion Peak 39 | 12p13.2 | chr12: 12412186-13039757 | chr12: 12525464-12721981 | chr12: 11739866-13828006 |
| | | (probes 564161:564352) | (probes 564230:564257) | (probes 563986:564574) |
| Deletion Peak 40 | 12q21.2 | chr12: 75602135-79819184 | chr12: 76423769-76523220 | chr12: 75648890-78593389 |
| | | (probes 581594:583083) | (probes 581862:581874) | (probes 581614:582689) |
| Deletion Peak 41 | 12q24.33 | chr12: 125048064-133851895 | chr12: 131256822-131362341 | chr12: 125246979-133851895 |
| | | (probes 597584:598999) | (probes 598784:598790) | (probes 597646:598999) |
| Deletion Peak 42 | 14q24.1 | chr14: 68280014-69351476 | chr14: 68284712-68948164 | chr14: 68343659-68985929 |
| | | (probes 640423:640650) | (probes 640425:640565) | (probes 640446:640573) |
| Deletion Peak 43 | 14q32.11 | chr14: 78347564-107349540 | chr14: 90999841-91286309 | chr14: 90170502-92315681 |
| | | (probes 643482:652331) | (probes 647605:647693) | (probes 647318:648035) |
| Deletion Peak 44 | 15q11.2 | chr15: 25436434-25466900 | chr15: 25438412-25459423 | chr15: 1-58781038 |
| | | (probes 652975:652987) | (probes 652978:652984) | (probes 652332:661726) |
| Deletion Peak 45 | 15q21.1 | chr15: 44850582-45321541 | chr15: 44851811-45052539 | chr15: 1-58781038 |
| | | (probes 657156:657216) | (probes 657157:657198) | (probes 652332:661726) |
| Deletion Peak 46 | 15q22.33 | chr15: 67053712-67697647 | chr15: 67337017-67551787 | chr15: 67245914-67753643 |
| | | (probes 664346:664503) | (probes 664432:664448) | (probes 664397:664514) |
| Deletion Peak 47 | 16p13.3 | chr16: 5141600-8053542 | chr16: 6056420-8053542 | chr16: 5279421-7736962 |
| | | (probes 676035:676388) | (probes 676266:676388) | (probes 676069:676383) |
| Deletion Peak 48 | 16q23.1 | chr16: 78131135-79628242 | chr16: 78131135-79286336 | chr16: 78424695-79295468 |
| | | (probes 692486:692917) | (probes 692486:692739) | (probes 692585:692741) |
| Deletion Peak 49 | 17p12 | chr17: 11466949-12461211 | chr17: 11872374-11906034 | chr17: 10231123-12752622 |
| | | (probes 699252:699560) | (probes 699350:699356) | (probes 698870:699686) |
| Deletion Peak 50 | 17q24.3 | chr17: 68174484-70599305 | chr17: 70337175-70590424 | chr17: 70549664-70552757 |
| | | (probes 711906:712713) | (probes 712684:712712) | (probes 712699:712700) |
| Deletion Peak 51 | 18p11.31 | chr18: 3277394-4265401 | chr18: 3441391-3481373 | chr18: 3398771-3729936 |
| | | (probes 715148:715497) | (probes 715189:715190) | (probes 715178:715261) |
| Deletion Peak 52 | 18q12.2 | chr18: 35136370-39061915 | chr18: 36781295-37333625 | chr18: 35827844-46846953 |
| | | (probes 722194:723278) | (probes 722627:722752) | (probes 722377:725639) |
| Deletion Peak 53 | 18q21.2 | chr18: 48472034-48707815 | chr18: 48547928-48660122 | chr18: 47049746-78077248 |
| | | (probes 725965:726002) | (probes 725987:725988) | (probes 725727:734875) |
| Deletion Peak 54 | 18q21.33 | chr18: 60645473-61013467 | chr18: 60788090-61013467 | chr18: 47049746-78077248 |
| | | (probes 730126:730222) | (probes 730167:730222) | (probes 725727:734875) |
| Deletion Peak 55 | 19p13.3 | chr19: 1488247-1660256 | chr19: 1488455-1660256 | chr19: 1-5957502 |
| | | (probes 735045:735049) | (probes 735046:735049) | (probes 734876:735790) |
| Deletion Peak 56 | 20p12.1 | chr20: 13955189-16350354 | chr20: 14416445-15462745 | chr20: 14096517-16064189 |
| | | (probes 751741:751931) | (probes 751773:751774) | (probes 751766:751890) |
| Deletion Peak 57 | 21q11.2 | chr21: 15555708-16334057 | chr21: 15854887-15919066 | chr21: 1-31258004 |
| | | (probes 762725:762981) | (probes 762833:762869) | (probes 762720:767383) |
| Deletion Peak 58 | 21q21.1 | chr21: 23106546-26219369 | chr21: 23248652-23494410 | chr21: 1-31258004 |
| | | (probes 765049:765801) | (probes 765109:765118) | (probes 762720:767383) |
| Deletion Peak 59 | 22q13.32 | chr22: 48649199-49178363 | chr22: 48906309-49158314 | chr22: 47983027-51304566 |
| | | (probes 781080:781144) | (probes 781137:781140) | (probes 780856:781177) |

TABLE 6
Tier 1 selection of genomic markers used in recursive partitioning from all 180 regions.

| Unique Name | Descriptor | Wide Peak Limits | Peak Limits | Region Limits |
|---|---|---|---|---|
| Deletion Peak 56 | 20p12.1 | chr20: 13955189-16350354 | chr20: 14416445-15462745 | chr20: 14096517-16064189 |
| Deletion Peak 11 | 3p14.2 | chr3: 58946448-61555632 | chr3: 59692189-61457375 | chr3: 59692189-61460946 |
| Deletion Peak 26 | 6q26 | chr6: 161540781-163179430 | chr6: 161540781-163179430 | chr6: 160448048-171115067 |
| Amplification Peak | 20p12.3 | chr20: 5376354-5742957 | chr20: 5379141-5737687 | chr20: 5156552-5961498 |
| 37 | | | | |
| Deletion Peak 38 | 11q22.3 | chr11: 102958343-135006516 | chr11: 108251085-108350263 | chr11: 108179920-108470348 |
| Deletion Peak 52 | 18q12.2 | chr18: 35136370-39061915 | chr18: 36781295-37333625 | chr18: 35827844-46846953 |
| Amplification Peak | 20q13.12 | chr20: 42534268-43281676 | chr20: 42537604-42773258 | chr20: 24375411-63025520 |
| 41 | | | | |
| Deletion Peak 34 | 10q21.1 | chr10: 51561927-53481136 | chr10: 51561927-54065263 | chr10: 50763189-54253314 |
| Whole arm | 20p | NA | NA | chr20: 1-26369569 |
| amplification 20 | | | | |
| Deletion Peak 25 | 6q21 | chr6: 91006553-129206367 | chr6: 111544567-112706964 | chr6: 108785353-116683959 |
| Amplification Peak | 8p11.21 | chr8: 41760295-42054539 | chr8: 41767516-41796226 | chr8: 34342520-48854434 |
| 12 | | | | |
| Deletion Peak 40 | 12q21.2 | chr12: 75602135-79819184 | chr12: 76423769-76523220 | chr12: 75648890-78593389 |
| Deletion Peak 28 | 8p23.3 | chr8: 1-1449850 | chr8: 1-623109 | chr8: 1-10801317 |
| Deletion Peak 15 | 4q22.1 | chr4: 91143530-93226628 | chr4: 91046982-92557520 | chr4: 91103554-92561908 |
| Amplification Peak | 17q11.2 | chr17: 27339261-27528113 | chr17: 27339592-27419750 | chr17: 20042949-40168023 |
| 28 | | | | |
| Whole arm | 10q | NA | NA | chr10: 42254935-135534747 |
| deletion 2 | | | | |
| Amplification Peak | 20p12.1 | chr20: 16140291-16967195 | chr20: 16488303-16962609 | chr20: 16146669-16970141 |
| 38 | | | | |
| Whole arm | 5q | NA | NA | chr5: 49405641-180915260 |
| deletion 31 | | | | |
| Deletion Peak 36 | 10q25.2 | chr10: 114197471-115353755 | chr10: 114708532-114929210 | chr10: 83248858-135534747 |
| Deletion Peak 23 | 6p25.3 | chr6: 1613114-2256643 | chr6: 1621843-2256643 | chr6: 1626114-2752758 |
| Deletion Peak 59 | 22q13.32 | chr22: 48649199-49178363 | chr22: 48906309-49158314 | chr22: 47983027-51304566 |
| Amplification Peak | 17q12 | chr17: 37747533-38052599 | chr17: 37950674-38031986 | chr17: 20042949-40168023 |
| 29 | | | | |
| Deletion Peak 24 | 6p22.2 | chr6: 25926929-26025173 | chr6: 25932794-26025173 | chr6: 25933563-26346501 |
| Amplification Peak | 20q13.32 | chr20: 56110288-57258354 | chr20: 57000076-57085518 | chr20: 24375411-63025520 |
| 43 | | | | |
| Deletion Peak 22 | 5q22.2 | chr5: 111312546-112362638 | chr5: 111747052-111761475 | chr5: 50968806-135478060 |
| Deletion Peak 35 | 10q23.31 | chr10: 89502327-90051809 | chr10: 89574482-89607380 | chr10: 83248858-135534747 |
| Deletion Peak 58 | 21q21.1 | chr21: 23106546-26219369 | chr21: 23248652-23494410 | chr21: 1-31258004 |
| Deletion Peak 57 | 21q11.2 | chr21: 15555708-16334057 | chr21: 15854887-15919066 | chr21: 1-31258004 |
| Whole arm | 18q | NA | NA | chr18: 18460898-78077248 |
| deletion 15 | | | | |
| Deletion Peak 17 | 4q31.3 | chr4: 152679194-153694102 | chr4: 153232273-153473069 | chr4: 152233433-191154276 |
| Deletion Peak 18 | 4q32.1 | chr4: 156135292-162306004 | chr4: 159683040-159830757 | chr4: 152233433-191154276 |
| Deletion Peak 53 | 18q21.2 | chr18: 48472034-48707815 | chr18: 48547928-48660122 | chr18: 47049746-78077248 |
| Whole arm | 21q | NA | NA | chr21: 14288129-48129895 |
| deletion 22 | | | | |
| Whole arm | 9p | NA | NA | chr9: 1-47367679 |
| deletion 38 | | | | |
| Amplification Peak | 20q13.2 | chr20: 52240629-52852159 | chr20: 52246659-52447690 | chr20: 24375411-63025520 |
| 42 | | | | |
| Whole arm | 22q | NA | NA | chr22: 16000000-51304566 |
| deletion 23 | | | | |

TABLE 7
Tier 2 selection of genomic markers used in recursive partitioning from all 180 regions.

| Unique Name | Descriptor | Wide Peak Limits | Peak Limits | Region Limits |
|---|---|---|---|---|
| Amplification Peak | 8q24.21 | chr8: 128574277-128592142 | chr8: 128574768-128591041 | chr8: 54646329-146364022 |
| 16 | | | | |
| Amplification Peak | 17q24.2 | chr17: 65638523-65873408 | chr17: 65649475-65769071 | chr17: 63042470-68655121 |
| 31 | | | | |
| Deletion Peak 39 | 12p13.2 | chr12: 12412186-13039757 | chr12: 12525464-12721981 | chr12: 11739866-13828006 |
| Deletion Peak 10 | 3p26.3 | chr3: 1-8350135 | chr3: 1-2251569 | chr3: 1-9456413 |
| Whole arm | 17p | NA | NA | chr17: 1-22263006 |
| deletion 12 | | | | |
| Deletion Peak 55 | 19p13.3 | chr19: 1488247-1660256 | chr19: 1488455-1660256 | chr19: 1-5957502 |
| Amplification Peak 3 | 1q43 | chr1: 201615295-249250621 | chr1: 242413158-242638099 | chr1: 242270003-249250621 |
| Whole arm | 14q | NA | NA | chr14: 19000000-107349540 |
| deletion 8 | | | | |
| Deletion Peak 50 | 17q24.3 | chr17: 68174484-70599305 | chr17: 70337175-70590424 | chr17: 70549664-70552757 |
| Whole arm | 18p | NA | NA | chr18: 1-15460898 |
| deletion 14 | | | | |
| Amplification Peak 5 | 3q29 | chr3: 174745992-198022430 | chr3: 195922177-195991311 | chr3: 174746836-198022430 |
| Whole arm | 7p | NA | NA | chr7: 1-58054331 |
| amplification 34 | | | | |
| Amplification Peak | 13q12.2 | chr13: 28164386-28564331 | chr13: 28174614-28222659 | chr13: 1-115169878 |
| 22 | | | | |
| Deletion Peak 43 | 14q32.11 | chr14: 78347564-107349540 | chr14: 90999841-91286309 | chr14: 90170502-92315681 |
| Amplification Peak | 20p11.22 | chr20: 22058560-22353663 | chr20: 22063756-22350551 | chr20: 20938975-24286806 |
| 39 | | | | |
| Deletion Peak 4 | 1p31.1 | chr1: 68959091-82269682 | chr1: 79308665-79566142 | chr1: 54944113-149961894 |
| Deletion Peak 42 | 14q24.1 | chr14: 68280014-69351476 | chr14: 68284712-68948164 | chr14: 68343659-68985929 |
| Deletion Peak 51 | 18p11.31 | chr18: 3277394-4265401 | chr18: 3441391-3481373 | chr18: 3398771-3729936 |
| Deletion Peak 19 | 4q35.1 | chr4: 184432080-185262191 | chr4: 184545375-185154488 | chr4: 152233433-191154276 |
| Deletion Peak 31 | 8q11.1 | chr8: 42874387-48101823 | chr8: 42931769-48061147 | chr8: 42932439-48062091 |
| Whole arm | 11q | NA | NA | chr11: 54644205-135006516 |
| deletion 4 | | | | |
| Deletion Peak 30 | 8p12 | chr8: 33445578-37452445 | chr8: 34944675-36494184 | chr8: 35186331-35207233 |
| Amplification Peak | 17q23.2 | chr17: 58400463-58674819 | chr17: 58400997-58532661 | chr17: 57045336-62714320 |
| 30 | | | | |
| Whole arm | 4p | NA | NA | chr4: 1-49660117 |
| deletion 28 | | | | |
| Amplification Peak | 8p11.23 | chr8: 38162916-38237532 | chr8: 38165117-38236937 | chr8: 34342520-48854434 |
| 11 | | | | |
| Whole arm | 6p | NA | NA | chr6: 1-58830166 |
| deletion 32 | | | | |
| Amplification Peak | 8q22.3 | chr8: 101853171-101878037 | chr8: 101853464-101876083 | chr8: 54646329-146364022 |
| 15 | | | | |
| Whole arm | 6q | NA | NA | chr6: 61830166-171115067 |
| deletion 33 | | | | |
| Whole arm | 15q | NA | NA | chr15: 20000000-102531392 |
| deletion 9 | | | | |
| Deletion Peak 12 | 3p13 | chr3: 70014100-71250049 | chr3: 70976616-71182883 | chr3: 70977475-71315576 |
| Amplification Peak | 8q12.2 | chr8: 60847395-62906825 | chr8: 61658252-61874896 | chr8: 54646329-146364022 |
| 13 | | | | |
| Deletion Peak 21 | 5q12.1 | chr5: 58263825-59784640 | chr5: 58263825-59784640 | chr5: 50968806-135478060 |
| Deletion Peak 5 | 1p21.1 | chr1: 102460262-107618224 | chr1: 104041213-104355050 | chr1: 54944113-149961894 |
| Deletion Peak 37 | 10q26.3 | chr10: 133107199-135534747 | chr10: 135221096-135534747 | chr10: 83248858-135534747 |
| Whole arm | 20q | NA | NA | chr20: 29369569-63025520 |
| amplification 21 | | | | |
| Amplification Peak 8 | 6p21.1 | chr6: 43545080-44164949 | chr6: 43765716-44163738 | chr6: 36748055-52028937 |
| Whole arm | 10p | NA | NA | chr10: 1-39254935 |
| deletion 1 | | | | |
| Whole arm | 9q | NA | NA | chr9: 50367679-141213431 |
| amplification 39 | | | | |
| Deletion Peak 3 | 1p33 | chr1: 49187432-50544677 | chr1: 48847112-50490440 | chr1: 49196205-51396500 |
| Deletion Peak 49 | 17p12 | chr17: 11466949-12461211 | chr17: 11872374-11906034 | chr17: 10231123-12752622 |
| Deletion Peak 46 | 15q22.33 | chr15: 67053712-67697647 | chr15: 67337017-67551787 | chr15: 67245914-67753643 |
| Whole arm | 8q | NA | NA | chr8: 46838887-146364022 |
| amplification 37 | | | | |
| Deletion Peak 16 | 4q25 | chr4: 109046573-109544048 | chr4: 109451604-109544048 | chr4: 109489936-109535239 |
| Whole arm | 17q | NA | NA | chr17: 25263006-81195210 |
| deletion 13 | | | | |
| Amplification Peak | 8q21.13 | chr8: 80636493-82552412 | chr8: 81853752-81935079 | chr8: 54646329-146364022 |
| 14 | | | | |
| Whole arm | 4q | NA | NA | chr4: 52660117-191154276 |
| deletion 29 | | | | |
| Deletion Peak 54 | 18q21.33 | chr18: 60645473-61013467 | chr18: 60788090-61013467 | chr18: 47049746-78077248 |
| Amplification Peak | 20q11.21 | chr20: 29981165-30284236 | chr20: 30158807-30231055 | chr20: 24375411-63025520 |
| 40 | | | | |

TABLE 8
Tier 3 selection of genomic markers used in recursive partitioning from all 180 regions.

| Unique Name | Descriptor | Wide Peak Limits | Peak Limits | Region Limits |
|---|---|---|---|---|
| Amplification Peak | 11q13.3 | chr11: 68746750-69827851 | chr11: 69311828-69824706 | chr11: 68748468-70760456 |
| 19 | | | | |
| Whole arm | 11q | NA | NA | chr11: 54644205-135006516 |
| amplification 4 | | | | |
| Whole arm deletion | 3q | NA | NA | chr3: 93504854-198022430 |
| 27 | | | | |
| Whole arm deletion | 9q | NA | NA | chr9: 50367679-141213431 |
| 39 | | | | |
| Whole arm | 4q | NA | NA | chr4: 52660117-191154276 |
| amplification 29 | | | | |
| Amplification Peak | 11p15.5 | chr11: 2091739-2302637 | chr11: 2095340-2301396 | chr11: 2095340-2305324 |
| 18 | | | | |
| Deletion Peak 48 | 16q23.1 | chr16: 78131135-79628242 | chr16: 78131135-79286336 | chr16: 78424695-79295468 |
| Whole arm deletion | 8q | NA | NA | chr8: 46838887-146364022 |
| 37 | | | | |
| Amplification Peak | 16q12.1 | chr16: 52243935-52652774 | chr16: 52383686-52652198 | chr16: 51078632-53117566 |
| 27 | | | | |
| Amplification Peak | 19q13.2 | chr19: 39844313-40357920 | chr19: 39848086-40009185 | chr19: 19939350-41312227 |
| 36 | | | | |
| Amplification Peak 4 | 2q33.1 | chr2: 181446594-243199373 | chr2: 199603526-199649638 | chr2: 199525178-199824262 |
| Amplification Peak | 13q34 | chr13: 110097815-111753132 | chr13: 110614601-110852082 | chr13: 1-115169878 |
| 24 | | | | |
| Whole arm | 16q | NA | NA | chr16: 38335801-90354753 |
| amplification 11 | | | | |
| Whole arm | 11p | NA | NA | chr11: 1-51644205 |
| amplification 3 | | | | |
| Whole arm deletion | 3p | NA | NA | chr3: 1-90504854 |
| 26 | | | | |
| Deletion Peak 32 | 9p21.3 | chr9: 21558582-22452906 | chr9: 21995318-22021004 | chr9: 21711940-22331169 |
| Deletion Peak 47 | 16p13.3 | chr16: 5141600-8053542 | chr16: 6056420-8053542 | chr16: 5279421-7736962 |
| Whole arm | 6p | NA | NA | chr6: 1-58830166 |
| amplification 32 | | | | |
| Amplification Peak | 19q13.11 | chr19: 32439834-33021877 | chr19: 32966965-32988344 | chr19: 19939350-41312227 |
| 35 | | | | |
| Whole arm deletion | 20p | NA | NA | chr20: 1-26369569 |
| 20 | | | | |
| Deletion Peak 20 | 5p15.33 | chr5: 1-2750686 | chr5: 1368344-2252637 | chr5: 1-3864653 |
| Deletion Peak 1 | 1p36.31 | chr1: 4843384-6053964 | chr1: 5646446-6050774 | chr1: 1-37080378 |
| Whole arm | 5p | NA | NA | chr5: 1-46405641 |
| amplification 30 | | | | |
| Amplification Peak | 12p11.22 | chr12: 24880798-28477058 | chr12: 27800441-27813180 | chr12: 25955987-28156868 |
| 21 | | | | |
| Whole arm | 19p | NA | NA | chr19: 1-24681782 |
| amplification 16 | | | | |
| Amplification Peak 2 | 1q22 | chr1: 120494739-199253746 | chr1: 155143717-155177826 | chr1: 120497533-175439985 |
| Whole arm deletion | 1q | NA | NA | chr1: 124535434-249250621 |
| 19 | | | | |
| Deletion Peak 41 | 12q24.33 | chr12: 125048064-133851895 | chr12: 131256822-131362341 | chr12: 125246979-133851895 |
| Deletion Peak 13 | 3q26.31 | chr3: 173995229-175766885 | chr3: 174575326-175766885 | chr3: 174646449-174998725 |
| Whole arm deletion 6 | 12q | NA | NA | chr12: 37856694-133851895 |
| Whole arm | 3q | NA | NA | chr3: 93504854-198022430 |
| amplification 27 | | | | |
| Whole arm deletion | 19q | NA | NA | chr19: 27681782-59128983 |
| 17 | | | | |
| Whole arm | 1q | NA | NA | chr1: 124535434-249250621 |
| amplification 19 | | | | |
| Amplification Peak | 17q25.3 | chr17: 77601682-77857881 | chr17: 77766436-77856811 | chr17: 75845339-81195210 |
| 32 | | | | |
| Whole arm | 8p | NA | NA | chr8: 1-43838887 |
| amplification 36 | | | | |
| Amplification Peak | 16p11.2 | chr16: 30548665-30684980 | chr16: 30549334-30682578 | chr16: 28556705-46663588 |
| 26 | | | | |
| Whole arm deletion | 1p | NA | NA | chr1: 1-121535434 |
| 18 | | | | |
| Whole arm | 13q | NA | NA | chr13: 19000000-115169878 |
| amplification 7 | | | | |
| Whole arm | 17q | NA | NA | chr17: 25263006-81195210 |
| amplification 13 | | | | |
| Deletion Peak 44 | 15q11.2 | chr15: 25436434-25466900 | chr15: 25438412-25459423 | chr15: 1-58781038 |
| Whole arm | 1p | NA | NA | chr1: 1-121535434 |
| amplification 18 | | | | |
| Deletion Peak 33 | 10p15.3 | chr10: 1-855610 | chr10: 1-418075 | chr10: 1-2055670 |
| Deletion Peak 45 | 15q21.1 | chr15: 44850582-45321541 | chr15: 44851811-45052539 | chr15: 1-58781038 |
| Whole arm deletion | 5p | NA | NA | chr5: 1-46405641 |
| 30 | | | | |
| Deletion Peak 7 | 2p21 | chr2: 42587649-44009118 | chr2: 43440855-43455807 | chr2: 43159857-43470966 |
| Whole arm deletion | 8p | NA | NA | chr8: 1-43838887 |
| 36 | | | | |
| Deletion Peak 2 | 1p36.11 | chr1: 26898389-27219375 | chr1: 27139248-27185402 | chr1: 1-37080378 |
| Deletion Peak 14 | 4p16.2 | chr4: 1-9787265 | chr4: 5915497-5925651 | chr4: 1-11455571 |
| Amplification Peak | 13q22.1 | chr13: 73775176-74007122 | chr13: 73906681-74004816 | chr13: 1-115169878 |
| 23 | | | | |
| Deletion Peak 29 | 8p21.3 | chr8: 13423967-26607015 | chr8: 21547484-21647267 | chr8: 11646511-25738962 |
| Whole arm deletion 3 | 11p | NA | NA | chr11: 1-51644205 |

TABLE 9
Tier 4 selection of genomic markers used in recursive partitioning from all 180 regions.

| Unique Name | Descriptor | Wide Peak Limits | Peak Limits | Region Limits |
|---|---|---|---|---|
| Whole arm | 2p | NA | NA | chr2: 1-92326171 |
| amplification 24 | | | | |
| Whole arm | 2q | NA | NA | chr2: 95326171-243199373 |
| amplification 25 | | | | |
| Whole arm | 3p | NA | NA | chr3: 1-90504854 |
| amplification 26 | | | | |
| Whole arm | 4p | NA | NA | chr4: 1-49660117 |
| amplification 28 | | | | |
| Whole arm | 5q | NA | NA | chr5: 49405641-180915260 |
| amplification 31 | | | | |
| Whole arm | 6q | NA | NA | chr6: 61830166-171115067 |
| amplification 33 | | | | |
| Whole arm | 7q | NA | NA | chr7: 61054331-159138663 |
| amplification 35 | | | | |
| Whole arm | 9p | NA | NA | chr9: 1-47367679 |
| amplification 38 | | | | |
| Whole arm | 10p | NA | NA | chr10: 1-39254935 |
| amplification 1 | | | | |
| Whole arm | 10q | NA | NA | chr10: 42254935-135534747 |
| amplification 2 | | | | |
| Whole arm | 12p | NA | NA | chr12: 1-34856694 |
| amplification 5 | | | | |
| Whole arm | 12q | NA | NA | chr12: 37856694-133851895 |
| amplification 6 | | | | |
| Whole arm | 14q | NA | NA | chr14: 19000000-107349540 |
| amplification 8 | | | | |
| Whole arm | 15q | NA | NA | chr15: 20000000-102531392 |
| amplification 9 | | | | |
| Whole arm | 16p | NA | NA | chr16: 1-35335801 |
| amplification 10 | | | | |
| Whole arm | 17p | NA | NA | chr17: 1-22263006 |
| amplification 12 | | | | |
| Whole arm | 18p | NA | NA | chr18: 1-15460898 |
| amplification 14 | | | | |
| Whole arm | 18q | NA | NA | chr18: 18460898-78077248 |
| amplification 15 | | | | |
| Whole arm | 19q | NA | NA | chr19: 27681782-59128983 |
| amplification 17 | | | | |
| Whole arm | 21q | NA | NA | chr21: 14288129-48129895 |
| amplification 22 | | | | |
| Whole arm | 22q | NA | NA | chr22: 16000000-51304566 |
| amplification 23 | | | | |
| Whole arm deletion | 2p | NA | NA | chr2: 1-92326171 |
| 24 | | | | |
| Whole arm deletion | 2q | NA | NA | chr2: 95326171-243199373 |
| 25 | | | | |
| Whole arm deletion | 7p | NA | NA | chr7: 1-58054331 |
| 34 | | | | |
| Whole arm deletion | 7q | NA | NA | chr7: 61054331-159138663 |
| 35 | | | | |
| Whole arm deletion 5 | 12p | NA | NA | chr12: 1-34856694 |
| Whole arm deletion 7 | 13q | NA | NA | chr13: 19000000-115169878 |
| Whole arm deletion | 16p | NA | NA | chr16: 1-35335801 |
| 10 | | | | |
| Whole arm deletion | 16q | NA | NA | chr16: 38335801-90354753 |
| 11 | | | | |
| Whole arm deletion | 19p | NA | NA | chr19: 1-24681782 |
| 16 | | | | |
| Whole arm deletion | 20q | NA | NA | chr20: 29369569-63025520 |
| 21 | | | | |
| Amplification Peak 1 | 1p34.2 | chr1: 39144054-44367347 | chr1: 42834925-43114106 | chr1: 42692914-43456374 |
| Amplification Peak 6 | 5p15.33 | chr5: 1-6418038 | chr5: 1-949725 | chr5: 1-2807521 |
| Amplification Peak 7 | 5p12 | chr5: 33430780-50470114 | chr5: 43822799-43929095 | chr5: 37334807-44266311 |
| Amplification Peak 9 | 6q23.3 | chr6: 135342981-135632150 | chr6: 135513416-135593890 | chr6: 133418393-138096856 |
| Amplification Peak | 7p11.2 | chr7: 54949302-55950640 | chr7: 55248172-55271747 | chr7: 54949902-55955413 |
| 10 | | | | |
| Amplification Peak | 10q22.3 | chr10: 79956009-83248579 | chr10: 80445049-80732930 | chr10: 77952482-83583616 |
| 17 | | | | |
| Amplification Peak | 12p13.32 | chr12: 3941807-4552801 | chr12: 4257035-4414967 | chr12: 1-8970181 |
| 20 | | | | |
| Amplification Peak | 15q26.1 | chr15: 84648780-102531392 | chr15: 90811423-90958538 | chr15: 90086447-91895585 |
| 25 | | | | |
| Amplification Peak | 18q11.2 | chr18: 19517253-20155059 | chr18: 19525020-20154578 | chr18: 19243919-21268418 |
| 33 | | | | |
| Amplification Peak | 18q21.1 | chr18: 46041609-46930382 | chr18: 46474762-46604295 | chr18: 46043163-46624631 |
| 34 | | | | |
| Deletion Peak 6 | 2p25.3 | chr2: 1-4752017 | chr2: 1-488785 | chr2: 1-5260278 |
| Deletion Peak 8 | 2q23.1 | chr2: 147341956-149499455 | chr2: 148437688-148750846 | chr2: 148444810-148756213 |
| Deletion Peak 9 | 2q37.3 | chr2: 240321205-243199373 | chr2: 240940975-241060875 | chr2: 240589192-241997730 |
| Deletion Peak 27 | 7q31.1 | chr7: 109590674-111367757 | chr7: 110232248-111358792 | chr7: 110595801-110618403 |

TABLE 10
Tier 1 selection of genomic markers used in recursive partitioning from the 102 focal regions.

| Unique Name | Descriptor | Wide Peak Limits | Peak Limits | Region Limits |
|---|---|---|---|---|
| Amplification Peak 2 | 1q22 | chr1: 120494739-199253746 | chr1: 155143717-155177826 | chr1: 120497533-175439985 |
| Deletion Peak 43 | 14q32.11 | chr14: 78347564-107349540 | chr14: 90999841-91286309 | chr14: 90170502-92315681 |
| Amplification Peak | 20p11.22 | chr20: 22058560-22353663 | chr20: 22063756-22350551 | chr20: 20938975-24286806 |
| 39 | | | | |
| Amplification Peak | 20p12.3 | chr20: 5376354-5742957 | chr20: 5379141-5737687 | chr20: 5156552-5961498 |
| 37 | | | | |
| Deletion Peak 12 | 3p13 | chr3: 70014100-71250049 | chr3: 70976616-71182883 | chr3: 70977475-71315576 |
| Amplification Peak | 20q13.12 | chr20: 42534268-43281676 | chr20: 42537604-42773258 | chr20: 24375411-63025520 |
| 41 | | | | |
| Deletion Peak 25 | 6q21 | chr6: 91006553-129206367 | chr6: 111544567-112706964 | chr6: 108785353-116683959 |
| Amplification Peak | 8q22.3 | chr8: 101853171-101878037 | chr8: 101853464-101876083 | chr8: 54646329-146364022 |
| 15 | | | | |
| Amplification Peak | 20p12.1 | chr20: 16140291-16967195 | chr20: 16488303-16962609 | chr20: 16146669-16970141 |
| 38 | | | | |
| Deletion Peak 28 | 8p23.3 | chr8: 1-1449850 | chr8: 1-623109 | chr8: 1-10801317 |
| Amplification Peak | 8p11.21 | chr8: 41760295-42054539 | chr8: 41767516-41796226 | chr8: 34342520-48854434 |
| 12 | | | | |
| Deletion Peak 40 | 12q21.2 | chr12: 75602135-79819184 | chr12: 76423769-76523220 | chr12: 75648890-78593389 |
| Deletion Peak 26 | 6q26 | chr6: 161540781-163179430 | chr6: 161540781-163179430 | chr6: 160448048-171115067 |
| Deletion Peak 34 | 10q21.1 | chr10: 51561927-53481136 | chr10: 51561927-54065263 | chr10: 50763189-54253314 |
| Deletion Peak 46 | 15q22.33 | chr15: 67053712-67697647 | chr15: 67337017-67551787 | chr15: 67245914-67753643 |
| Deletion Peak 35 | 10q23.31 | chr10: 89502327-90051809 | chr10: 89574482-89607380 | chr10: 83248858-135534747 |
| Deletion Peak 23 | 6p25.3 | chr6: 1613114-2256643 | chr6: 1621843-2256643 | chr6: 1626114-2752758 |
| Deletion Peak 36 | 10q25.2 | chr10: 114197471-115353755 | chr10: 114708532-114929210 | chr10: 83248858-135534747 |
| Deletion Peak 38 | 11q22.3 | chr11: 102958343-135006516 | chr11: 108251085-108350263 | chr11: 108179920-108470348 |
| Deletion Peak 52 | 18q12.2 | chr18: 35136370-39061915 | chr18: 36781295-37333625 | chr18: 35827844-46846953 |
| Amplification Peak | 8q21.13 | chr8: 80636493-82552412 | chr8: 81853752-81935079 | chr8: 54646329-146364022 |
| 14 | | | | |
| Amplification Peak | 20q13.32 | chr20: 56110288-57258354 | chr20: 57000076-57085518 | chr20: 24375411-63025520 |
| 43 | | | | |
| Amplification Peak | 17q11.2 | chr17: 27339261-27528113 | chr17: 27339592-27419750 | chr17: 20042949-40168023 |
| 28 | | | | |
| Deletion Peak 24 | 6p22.2 | chr6: 25926929-26025173 | chr6: 25932794-26025173 | chr6: 25933563-26346501 |
| Deletion Peak 15 | 4q22.1 | chr4: 91143530-93226628 | chr4: 91046982-92557520 | chr4: 91103554-92561908 |
| Deletion Peak 22 | 5q22.2 | chr5: 111312546-112362638 | chr5: 111747052-111761475 | chr5: 50968806-135478060 |
| Deletion Peak 59 | 22q13.32 | chr22: 48649199-49178363 | chr22: 48906309-49158314 | chr22: 47983027-51304566 |
| Deletion Peak 18 | 4q32.1 | chr4: 156135292-162306004 | chr4: 159683040-159830757 | chr4: 152233433-191154276 |
| Deletion Peak 17 | 4q31.3 | chr4: 152679194-153694102 | chr4: 153232273-153473069 | chr4: 152233433-191154276 |
| Deletion Peak 58 | 21q21.1 | chr21: 23106546-26219369 | chr21: 23248652-23494410 | chr21: 1-31258004 |
| Deletion Peak 53 | 18q21.2 | chr18: 48472034-48707815 | chr18: 48547928-48660122 | chr18: 47049746-78077248 |
| Amplification Peak | 20q13.2 | chr20: 52240629-52852159 | chr20: 52246659-52447690 | chr20: 24375411-63025520 |
| 42 | | | | |
| Deletion Peak 57 | 21q11.2 | chr21: 15555708-16334057 | chr21: 15854887-15919066 | chr21: 1-31258004 |

TABLE 11
Tier 2 selection of genomic markers used in recursive partitioning from the 102 focal regions.

| Unique Name | Descriptor | Wide Peak Limits | Peak Limits | Region Limits |
|---|---|---|---|---|
| Amplification Peak | 11p15.5 | chr11: 2091739-2302637 | chr11: 2095340-2301396 | chr11: 2095340-2305324 |
| 18 | | | | |
| Amplification Peak | 15q26.1 | chr15: 84648780-102531392 | chr15: 90811423-90958538 | chr15: 90086447-91895585 |
| 25 | | | | |
| Amplification Peak | 16p11.2 | chr16: 30548665-30684980 | chr16: 30549334-30682578 | chr16: 28556705-46663588 |
| 26 | | | | |
| Deletion Peak 48 | 16q23.1 | chr16: 78131135-79628242 | chr16: 78131135-79286336 | chr16: 78424695-79295468 |
| Deletion Peak 10 | 3p26.3 | chr3: 1-8350135 | chr3: 1-2251569 | chr3: 1-9456413 |
| Deletion Peak 30 | 8p12 | chr8: 33445578-37452445 | chr8: 34944675-36494184 | chr8: 35186331-35207233 |
| Amplification Peak | 17q24.2 | chr17: 65638523-65873408 | chr17: 65649475-65769071 | chr17: 63042470-68655121 |
| 31 | | | | |
| Amplification Peak | 13q22.1 | chr13: 73775176-74007122 | chr13: 73906681-74004816 | chr13: 1-115169878 |
| 23 | | | | |
| Amplification Peak 7 | 5p12 | chr5: 33430780-50470114 | chr5: 43822799-43929095 | chr5: 37334807-44266311 |
| Deletion Peak 50 | 17q24.3 | chr17: 68174484-70599305 | chr17: 70337175-70590424 | chr17: 70549664-70552757 |
| Deletion Peak 45 | 15q21.1 | chr15: 44850582-45321541 | chr15: 44851811-45052539 | chr15: 1-58781038 |
| Amplification Peak | 13q34 | chr13: 110097815-111753132 | chr13: 110614601-110852082 | chr13: 1-115169878 |
| 24 | | | | |
| Deletion Peak 29 | 8p21.3 | chr8: 13423967-26607015 | chr8: 21547484-21647267 | chr8: 11646511-25738962 |
| Amplification Peak 8 | 6p21.1 | chr6: 43545080-44164949 | chr6: 43765716-44163738 | chr6: 36748055-52028937 |
| Deletion Peak 3 | 1p33 | chr1: 49187432-50544677 | chr1: 48847112-50490440 | chr1: 49196205-51396500 |
| Deletion Peak 56 | 20p12.1 | chr20: 13955189-16350354 | chr20: 14416445-15462745 | chr20: 14096517-16064189 |
| Amplification Peak | 7p11.2 | chr7: 54949302-55950640 | chr7: 55248172-55271747 | chr7: 54949902-55955413 |
| 10 | | | | |
| Deletion Peak 51 | 18p11.31 | chr18: 3277394-4265401 | chr18: 3441391-3481373 | chr18: 3398771-3729936 |
| Deletion Peak 33 | 10p15.3 | chr10: 1-855610 | chr10: 1-418075 | chr10: 1-2055670 |
| Amplification Peak 3 | 1q43 | chr1: 201615295-249250621 | chr1: 242413158-242638099 | chr1: 242270003-249250621 |
| Deletion Peak 21 | 5q12.1 | chr5: 58263825-59784640 | chr5: 58263825-59784640 | chr5: 50968806-135478060 |
| Amplification Peak | 17q12 | chr17: 37747533-38052599 | chr17: 37950674-38031986 | chr17: 20042949-40168023 |
| 29 | | | | |
| Amplification Peak 4 | 2q33.1 | chr2: 181446594-243199373 | chr2: 199603526-199649638 | chr2: 199525178-199824262 |
| Amplification Peak 9 | 6q23.3 | chr6: 135342981-135632150 | chr6: 135513416-135593890 | chr6: 133418393-138096856 |
| Deletion Peak 39 | 12p13.2 | chr12: 12412186-13039757 | chr12: 12525464-12721981 | chr12: 11739866-13828006 |
| Deletion Peak 55 | 19p13.3 | chr19: 1488247-1660256 | chr19: 1488455-1660256 | chr19: 1-5957502 |
| Amplification Peak | 8p11.23 | chr8: 38162916-38237532 | chr8: 38165117-38236937 | chr8: 34342520-48854434 |
| 11 | | | | |
| Amplification Peak | 8q24.21 | chr8: 128574277-128592142 | chr8: 128574768-128591041 | chr8: 54646329-146364022 |
| 16 | | | | |
| Deletion Peak 13 | 3q26.31 | chr3: 173995229-175766885 | chr3: 174575326-175766885 | chr3: 174646449-174998725 |
| Deletion Peak 4 | 1p31.1 | chr1: 68959091-82269682 | chr1: 79308665-79566142 | chr1: 54944113-149961894 |
| Deletion Peak 49 | 17p12 | chr17: 11466949-12461211 | chr17: 11872374-11906034 | chr17: 10231123-12752622 |
| Deletion Peak 44 | 15q11.2 | chr15: 25436434-25466900 | chr15: 25438412-25459423 | chr15: 1-58781038 |
| Deletion Peak 37 | 10q26.3 | chr10: 133107199-135534747 | chr10: 135221096-135534747 | chr10: 83248858-135534747 |
| Amplification Peak | 13q12.2 | chr13: 28164386-28564331 | chr13: 28174614-28222659 | chr13: 1-115169878 |
| 22 | | | | |
| Deletion Peak 19 | 4q35.1 | chr4: 184432080-185262191 | chr4: 184545375-185154488 | chr4: 152233433-191154276 |
| Deletion Peak 14 | 4p16.2 | chr4: 1-9787265 | chr4: 5915497-5925651 | chr4: 1-11455571 |
| Amplification Peak | 8q12.2 | chr8: 60847395-62906825 | chr8: 61658252-61874896 | chr8: 54646329-146364022 |
| 13 | | | | |
| Deletion Peak 42 | 14q24.1 | chr14: 68280014-69351476 | chr14: 68284712-68948164 | chr14: 68343659-68985929 |
| Deletion Peak 5 | 1p21.1 | chr1: 102460262-107618224 | chr1: 104041213-104355050 | chr1: 54944113-149961894 |
| Deletion Peak 16 | 4q25 | chr4: 109046573-109544048 | chr4: 109451604-109544048 | chr4: 109489936-109535239 |
| Amplification Peak | 20q11.21 | chr20: 29981165-30284236 | chr20: 30158807-30231055 | chr20: 24375411-63025520 |
| 40 | | | | |
| Deletion Peak 54 | 18q21.33 | chr18: 60645473-61013467 | chr18: 60788090-61013467 | chr18: 47049746-78077248 |

TABLE 12
Tier 3 selection of genomic markers used in recursive partitioning from the 102 focal regions.

| Unique Name | Descriptor | Wide Peak Limits | Peak Limits | Region Limits |
|---|---|---|---|---|
| Amplification Peak 1 | 1p34.2 | chr1: 39144054-44367347 | chr1: 42834925-43114106 | chr1: 42692914-43456374 |
| Amplification Peak 5 | 3q29 | chr3: 174745992-198022430 | chr3: 195922177-195991311 | chr3: 174746836-198022430 |
| Amplification Peak 6 | 5p15.33 | chr5: 1-6418038 | chr5: 1-949725 | chr5: 1-2807521 |
| Amplification Peak | 10q22.3 | chr10: 79956009-83248579 | chr10: 80445049-80732930 | chr10: 77952482-83583616 |
| 17 | | | | |
| Amplification Peak | 11q13.3 | chr11: 68746750-69827851 | chr11: 69311828-69824706 | chr11: 68748468-70760456 |
| 19 | | | | |
| Amplification Peak | 12p13.32 | chr12: 3941807-4552801 | chr12: 4257035-4414967 | chr12: 1-8970181 |
| 20 | | | | |
| Amplification Peak | 12p11.22 | chr12: 24880798-28477058 | chr12: 27800441-27813180 | chr12: 25955987-28156868 |
| 21 | | | | |
| Amplification Peak | 16q12.1 | chr16: 52243935-52652774 | chr16: 52383686-52652198 | chr16: 51078632-53117566 |
| 27 | | | | |
| Amplification Peak | 17q23.2 | chr17: 58400463-58674819 | chr17: 58400997-58532661 | chr17: 57045336-62714320 |
| 30 | | | | |
| Amplification Peak | 17q25.3 | chr17: 77601682-77857881 | chr17: 77766436-77856811 | chr17: 75845339-81195210 |
| 32 | | | | |
| Amplification Peak | 18q11.2 | chr18: 19517253-20155059 | chr18: 19525020-20154578 | chr18: 19243919-21268418 |
| 33 | | | | |
| Amplification Peak | 18q21.1 | chr18: 46041609-46930382 | chr18: 46474762-46604295 | chr18: 46043163-46624631 |
| 34 | | | | |
| Amplification Peak | 19q13.11 | chr19: 32439834-33021877 | chr19: 32966965-32988344 | chr19: 19939350-41312227 |
| 35 | | | | |
| Amplification Peak | 19q13.2 | chr19: 39844313-40357920 | chr19: 39848086-40009185 | chr19: 19939350-41312227 |
| 36 | | | | |
| Deletion Peak 1 | 1p36.31 | chr1: 4843384-6053964 | chr1: 5646446-6050774 | chr1: 1-37080378 |
| Deletion Peak 2 | 1p36.11 | chr1: 26898389-27219375 | chr1: 27139248-27185402 | chr1: 1-37080378 |
| Deletion Peak 6 | 2p25.3 | chr2: 1-4752017 | chr2: 1-488785 | chr2: 1-5260278 |
| Deletion Peak 7 | 2p21 | chr2: 42587649-44009118 | chr2: 43440855-43455807 | chr2: 43159857-43470966 |
| Deletion Peak 8 | 2q23.1 | chr2: 147341956-149499455 | chr2: 148437688-148750846 | chr2: 148444810-148756213 |
| Deletion Peak 9 | 2q37.3 | chr2: 240321205-243199373 | chr2: 240940975-241060875 | chr2: 240589192-241997730 |
| Deletion Peak 11 | 3p14.2 | chr3: 58946448-61555632 | chr3: 59692189-61457375 | chr3: 59692189-61460946 |
| Deletion Peak 20 | 5p15.33 | chr5: 1-2750686 | chr5: 1368344-2252637 | chr5: 1-3864653 |
| Deletion Peak 27 | 7q31.1 | chr7: 109590674-111367757 | chr7: 110232248-111358792 | chr7: 110595801-110618403 |
| Deletion Peak 31 | 8q11.1 | chr8: 42874387-48101823 | chr8: 42931769-48061147 | chr8: 42932439-48062091 |
| Deletion Peak 32 | 9p21.3 | chr9: 21558582-22452906 | chr9: 21995318-22021004 | chr9: 21711940-22331169 |
| Deletion Peak 41 | 12q24.33 | chr12: 125048064-133851895 | chr12: 131256822-131362341 | chr12: 125246979-133851895 |
| Deletion Peak 47 | 16p13.3 | chr16: 5141600-8053542 | chr16: 6056420-8053542 | chr16: 5279421-7736962 |

TABLE 13
Contribution of each of the 180 regions to the random forest classification model.

| Unique Name | Descriptor | Wide Peak Limits | Peak Limits | Region Limits | Contribution value |
|---|---|---|---|---|---|
| Deletion Peak 17 | 4q31.3 | chr4: 152679194-153694102 | chr4: 153232273-153473069 | chr4: 152233433-191154276 | 5.69900368 |
| Deletion Peak 19 | 4q35.1 | chr4: 184432080-185262191 | chr4: 184545375-185154488 | chr4: 152233433-191154276 | 5.4602327 |
| Deletion Peak 15 | 4q22.1 | chr4: 91143530-93226628 | chr4: 91046982-92557520 | chr4: 91103554-92561908 | 5.40079966 |
| Deletion Peak 18 | 4q32.1 | chr4: 156135292-162306004 | chr4: 159683040-159830757 | chr4: 152233433-191154276 | 5.22218115 |
| Whole arm deletion 29 | 4q | NA | NA | chr4: 52660117-191154276 | 5.14074254 |
| Deletion Peak 16 | 4q25 | chr4: 109046573-109544048 | chr4: 109451604-109544048 | chr4: 109489936-109535239 | 5.12389105 |
| Whole arm | 4p | NA | NA | chr4: 1-49660117 | 4.6643299 |
| deletion 28 | | | | | |
| Amplification Peak 41 | 20q13.12 | chr20: 42534268-43281676 | chr20: 42537604-42773258 | chr20: 24375411-63025520 | 3.29423738 |
| Deletion Peak 4 | 1p31.1 | chr1: 68959091-82269682 | chr1: 79308665-79566142 | chr1: 54944113-149961894 | 2.92406308 |
| Deletion Peak 14 | 4p16.2 | chr4: 1-9787265 | chr4: 5915497-5925651 | chr4: 1-11455571 | 2.77180108 |
| Whole arm | 22q | NA | NA | chr22: 16000000-51304566 | 2.72591739 |
| deletion 23 | | | | | |
| Deletion Peak 57 | 21q11.2 | chr21: 15555708-16334057 | chr21: 15854887-15919066 | chr21: 1-31258004 | 2.55008085 |
| Amplification | 20q13.2 | chr20: 52240629-52852159 | chr20: 52246659-52447690 | chr20: 24375411-63025520 | 2.53769321 |
| Peak 42 | | | | | |
| Amplification | 20q11.21 | chr20: 29981165-30284236 | chr20: 30158807-30231055 | chr20: 24375411-63025520 | 2.46858084 |
| Peak 40 | | | | | |
| Whole arm | 20q | NA | NA | chr20: 29369569-63025520 | 2.4607282 |
| amplification 21 | | | | | |
| Deletion Peak 58 | 21q21.1 | chr21: 23106546-26219369 | chr21: 23248652-23494410 | chr21: 1-31258004 | 2.46055021 |
| Whole arm | 21q | NA | NA | chr21: 14288129-48129895 | 2.36208909 |
| deletion 22 | | | | | |
| Amplification | 20q13.32 | chr20: 56110288-57258354 | chr20: 57000076-57085518 | chr20: 24375411-63025520 | 2.35785114 |
| Peak 43 | | | | | |
| Deletion Peak 59 | 22q13.32 | chr22: 48649199-49178363 | chr22: 48906309-49158314 | chr22: 47983027-51304566 | 2.27724108 |
| Deletion Peak 34 | 10q21.1 | chr10: 51561927-53481136 | chr10: 51561927-54065263 | chr10: 50763189-54253314 | 1.85840926 |
| Deletion Peak 5 | 1p21.1 | chr1: 102460262-107618224 | chr1: 104041213-104355050 | chr1: 54944113-149961894 | 1.85495579 |
| Deletion Peak 38 | 11q22.3 | chr11: 102958343-135006516 | chr11: 108251085-108350263 | chr11: 108179920-108470348 | 1.79129861 |
| Deletion Peak 3 | 1p33 | chr1: 49187432-50544677 | chr1: 48847112-50490440 | chr1: 49196205-51396500 | 1.75409699 |
| Deletion Peak 37 | 10q26.3 | chr10: 133107199-135534747 | chr10: 135221096-135534747 | chr10: 83248858-135534747 | 1.74689724 |
| Whole arm | 15q | NA | NA | chr15: 20000000-102531392 | 1.74607014 |
| deletion 9 | | | | | |
| Whole arm | 10q | NA | NA | chr10: 42254935-135534747 | 1.74534855 |
| deletion 2 | | | | | |
| Deletion Peak 45 | 15q21.1 | chr15: 44850582-45321541 | chr15: 44851811-45052539 | chr15: 1-58781038 | 1.74523402 |
| Deletion Peak 52 | 18q12.2 | chr18: 35136370-39061915 | chr18: 36781295-37333625 | chr18: 35827844-46846953 | 1.74103677 |
| Deletion Peak 49 | 17p12 | chr17: 11466949-12461211 | chr17: 11872374-11906034 | chr17: 10231123-12752622 | 1.74080473 |
| Deletion Peak 35 | 10q23.31 | chr10: 89502327-90051809 | chr10: 89574482-89607380 | chr10: 83248858-135534747 | 1.73841158 |
| Whole arm | 18p | NA | NA | chr18: 1-15460898 | 1.69686457 |
| deletion 14 | | | | | |
| Whole arm | 18q | NA | NA | chr18: 18460898-78077248 | 1.63024275 |
| deletion 15 | | | | | |
| Deletion Peak 46 | 15q22.33 | chr15: 67053712-67697647 | chr15: 67337017-67551787 | chr15: 67245914-67753643 | 1.61216031 |
| Deletion Peak 53 | 18q21.2 | chr18: 48472034-48707815 | chr18: 48547928-48660122 | chr18: 47049746-78077248 | 1.54801279 |
| Amplification | 20p12.3 | chr20: 5376354-5742957 | chr20: 5379141-5737687 | chr20: 5156552-5961498 | 1.54217323 |
| Peak 37 | | | | | |
| Deletion Peak 36 | 10q25.2 | chr10: 114197471-115353755 | chr10: 114708532-114929210 | chr10: 83248858-135534747 | 1.49414539 |
| Deletion Peak 54 | 18q21.33 | chr18: 60645473-61013467 | chr18: 60788090-61013467 | chr18: 47049746-78077248 | 1.46051102 |
| Deletion Peak 51 | 18p11.31 | chr18: 3277394-4265401 | chr18: 3441391-3481373 | chr18: 3398771-3729936 | 1.44244308 |
| Amplification | 20p12.1 | chr20: 16140291-16967195 | chr20: 16488303-16962609 | chr20: 16146669-16970141 | 1.39784163 |
| Peak 38 | | | | | |
| Deletion Peak 28 | 8p23.3 | chr8: 1-1449850 | chr8: 1-623109 | chr8: 1-10801317 | 1.376404 |
| Whole arm | 11p | NA | NA | chr11: 1-51644205 | 1.36242884 |
| deletion 3 | | | | | |
| Deletion Peak 2 | 1p36.11 | chr1: 26898389-27219375 | chr1: 27139248-27185402 | chr1: 1-37080378 | 1.35158286 |
| Deletion Peak 22 | 5q22.2 | chr5: 111312546-112362638 | chr5: 111747052-111761475 | chr5: 50968806-135478060 | 1.32777145 |
| Whole arm | 17q | NA | NA | chr17: 25263006-81195210 | 1.31992276 |
| deletion 13 | | | | | |
| Deletion Peak 21 | 5q12.1 | chr5: 58263825-59784640 | chr5: 58263825-59784640 | chr5: 50968806-135478060 | 1.31750542 |
| Amplification | 20p11.22 | chr20: 22058560-22353663 | chr20: 22063756-22350551 | chr20: 20938975-24286806 | 1.29653026 |
| Peak 39 | | | | | |
| Amplification | 13q12.2 | chr13: 28164386-28564331 | chr13: 28174614-28222659 | chr13: 1-115169878 | 1.29142193 |
| Peak 22 | | | | | |
| Deletion Peak 44 | 15q11.2 | chr15: 25436434-25466900 | chr15: 25438412-25459423 | chr15: 1-58781038 | 1.27544343 |
| Deletion Peak 43 | 14q32.11 | chr14: 78347564-107349540 | chr14: 90999841-91286309 | chr14: 90170502-92315681 | 1.2470974 |
| Whole arm | 11q | NA | NA | chr11: 54644205-135006516 | 1.22838424 |
| deletion 4 | | | | | |
| Whole arm | 5q | NA | NA | chr5: 49405641-180915260 | 1.21343422 |
| deletion 31 | | | | | |
| Deletion Peak 42 | 14q24.1 | chr14: 68280014-69351476 | chr14: 68284712-68948164 | chr14: 68343659-68985929 | 1.18921641 |
| Amplification | 13q22.1 | chr13: 73775176-74007122 | chr13: 73906681-74004816 | chr13: 1-115169878 | 1.18041391 |
| Peak 23 | | | | | |
| Amplification | 8q22.3 | chr8: 101853171-101878037 | chr8: 101853464-101876083 | chr8: 54646329-146364022 | 1.1639936 |
| Peak 15 | | | | | |
| Whole arm | 20p | NA | NA | chr20: 1-26369569 | 1.11182166 |
| amplification 20 | | | | | |
| Whole arm | 17p | NA | NA | chr17: 1-22263006 | 1.09115055 |
| deletion 12 | | | | | |
| Whole arm | 13q | NA | NA | chr13: 19000000-115169878 | 1.08715413 |
| amplification 7 | | | | | |
| Deletion Peak 40 | 12q21.2 | chr12: 75602135-79819184 | chr12: 76423769-76523220 | chr12: 75648890-78593389 | 1.08195556 |
| Deletion Peak 33 | 10p15.3 | chr10: 1-855610 | chr10: 1-418075 | chr10: 1-2055670 | 1.03741963 |
| Whole arm | 10p | NA | NA | chr10: 1-39254935 | 1.03431515 |
| deletion 1 | | | | | |
| Whole arm | 8p | NA | NA | chr8: 1-43838887 | 1.01165483 |
| deletion 36 | | | | | |
| Amplification | 8p11.21 | chr8: 41760295-42054539 | chr8: 41767516-41796226 | chr8: 34342520-48854434 | 1.00972856 |
| Peak 12 | | | | | |
| Amplification | 8q21.13 | chr8: 80636493-82552412 | chr8: 81853752-81935079 | chr8: 54646329-146364022 | 1.00902364 |
| Peak 14 | | | | | |
| Whole arm | 8q | NA | NA | chr8: 46838887-146364022 | 0.97550139 |
| amplification 37 | | | | | |
| Amplification | 13q34 | chr13: 110097815-111753132 | chr13: 110614601-110852082 | chr13: 1-115169878 | 0.97236703 |
| Peak 24 | | | | | |
| Whole arm | 1p | NA | NA | chr1: 1-121535434 | 0.9682743 |
| deletion 18 | | | | | |
| Deletion Peak 29 | 8p21.3 | chr8: 13423967-26607015 | chr8: 21547484-21647267 | chr8: 11646511-25738962 | 0.94172849 |
| Amplification | 8q24.21 | chr8: 128574277-128592142 | chr8: 128574768-128591041 | chr8: 54646329-146364022 | 0.90669936 |
| Peak 16 | | | | | |
| Deletion Peak 10 | 3p26.3 | chr3: 1-8350135 | chr3: 1-2251569 | chr3: 1-9456413 | 0.90057889 |
| Deletion Peak 56 | 20p12.1 | chr20: 13955189-16350354 | chr20: 14416445-15462745 | chr20: 14096517-16064189 | 0.89553463 |
| Amplification | 17q12 | chr17: 37747533-38052599 | chr17: 37950674-38031986 | chr17: 20042949-40168023 | 0.8947382 |
| Peak 29 | | | | | |
| Deletion Peak 30 | 8p12 | chr8: 33445578-37452445 | chr8: 34944675-36494184 | chr8: 35186331-35207233 | 0.89027155 |
| Whole arm | 14q | NA | NA | chr14: 19000000-107349540 | 0.87610091 |
| deletion 8 | | | | | |
| Deletion Peak 55 | 19p13.3 | chr19: 1488247-1660256 | chr19: 1488455-1660256 | chr19: 1-5957502 | 0.86068674 |
| Deletion Peak 1 | 1p36.31 | chr1: 4843384-6053964 | chr1: 5646446-6050774 | chr1: 1-37080378 | 0.85950877 |
| Whole arm | 9p | NA | NA | chr9: 1-47367679 | 0.84910578 |
| deletion 38 | | | | | |
| Deletion Peak 50 | 17q24.3 | chr17: 68174484-70599305 | chr17: 70337175-70590424 | chr17: 70549664-70552757 | 0.82566352 |
| Amplification | 17q11.2 | chr17: 27339261-27528113 | chr17: 27339592-27419750 | chr17: 20042949-40168023 | 0.80753708 |
| Peak 28 | | | | | |
| Amplification | 8p11.23 | chr8: 38162916-38237532 | chr8: 38165117-38236937 | chr8: 34342520-48854434 | 0.79953951 |
| Peak 11 | | | | | |
| Amplification | 1q43 | chr1: 201615295-249250621 | chr1: 242413158-242638099 | chr1: 242270003-249250621 | 0.78150602 |
| Peak 3 | | | | | |
| Whole arm | 7q | NA | NA | chr7: 61054331-159138663 | 0.77800193 |
| amplification 35 | | | | | |
| Amplification | 8q12.2 | chr8: 60847395-62906825 | chr8: 61658252-61874896 | chr8: 54646329-146364022 | 0.77096113 |
| Peak 13 | | | | | |
| Whole arm | 7p | NA | NA | chr7: 1-58054331 | 0.76807364 |
| amplification 34 | | | | | |
| Amplification | 5p12 | chr5: 33430780-50470114 | chr5: 43822799-43929095 | chr5: 37334807-44266311 | 0.75708381 |
| Peak 7 | | | | | |
| Deletion Peak 12 | 3p13 | chr3: 70014100-71250049 | chr3: 70976616-71182883 | chr3: 70977475-71315576 | 0.75572694 |
| Deletion Peak 32 | 9p21.3 | chr9: 21558582-22452906 | chr9: 21995318-22021004 | chr9: 21711940-22331169 | 0.75281938 |
| Amplification | 5p15.33 | chr5: 1-6418038 | chr5: 1-949725 | chr5: 1-2807521 | 0.75064607 |
| Peak 6 | | | | | |
| Amplification | 16q12.1 | chr16: 52243935-52652774 | chr16: 52383686-52652198 | chr16: 51078632-53117566 | 0.72390332 |
| Peak 27 | | | | | |
| Whole arm | 16p | NA | NA | chr16: 1-35335801 | 0.71183633 |
| amplification 10 | | | | | |
| Amplification | 7p11.2 | chr7: 54949302-55950640 | chr7: 55248172-55271747 | chr7: 54949902-55955413 | 0.71128453 |
| Peak 10 | | | | | |
| Deletion Peak 11 | 3p14.2 | chr3: 58946448-61555632 | chr3: 59692189-61457375 | chr3: 59692189-61460946 | 0.69805324 |
| Whole arm | 16q | NA | NA | chr16: 38335801-90354753 | 0.6977664 |
| amplification 11 | | | | | |
| Amplification | 16p11.2 | chr16: 30548665-30684980 | chr16: 30549334-30682578 | chr16: 28556705-46663588 | 0.68677078 |
| Peak 26 | | | | | |
| Whole arm | 17q | NA | NA | chr17: 25263006-81195210 | 0.68298273 |
| amplification 13 | | | | | |
| Deletion Peak 26 | 6q26 | chr6: 161540781-163179430 | chr6: 161540781-163179430 | chr6: 160448048-171115067 | 0.68131369 |
| Amplification | 6p21.1 | chr6: 43545080-44164949 | chr6: 43765716-44163738 | chr6: 36748055-52028937 | 0.68018519 |
| Peak 8 | | | | | |
| Whole arm | 12q | NA | NA | chr12: 37856694-133851895 | 0.67955302 |
| deletion 6 | | | | | |
| Deletion Peak 25 | 6q21 | chr6: 91006553-129206367 | chr6: 111544567-112706964 | chr6: 108785353-116683959 | 0.67397177 |
| Amplification | | | | | |
| Peak 2 | 1q22 | chr1: 120494739-199253746 | chr1: 155143717-155177826 | chr1: 120497533-175439985 | 0.6692668 |
| Whole arm | 6p | NA | NA | chr6: 1-58830166 | 0.66083392 |
| amplification 32 | | | | | |
| Whole arm | 8p | NA | NA | chr8: 1-43838887 | 0.65986144 |
| amplification 36 | | | | | |
| Amplification | 12p13.32 | chr12: 3941807-4552801 | chr12: 4257035-4414967 | chr12: 1-8970181 | 0.65326501 |
| Peak 20 | | | | | |
| Whole arm | 1q | NA | NA | chr1: 124535434-249250621 | 0.63810089 |
| amplification 19 | | | | | |
| Whole arm | 5p | NA | NA | chr5: 1-46405641 | 0.62977686 |
| deletion 30 | | | | | |
| Deletion Peak 39 | 12p13.2 | chr12: 12412186-13039757 | chr12: 12525464-12721981 | chr12: 11739866-13828006 | 0.61944112 |
| Whole arm | 19p | NA | NA | chr19: 1-24681782 | 0.61447685 |
| deletion 16 | | | | | |
| Amplification | 19q13.2 | chr19: 39844313-40357920 | chr19: 39848086-40009185 | chr19: 19939350-41312227 | 0.61443667 |
| Peak 36 | | | | | |
| Amplification | 12p11.22 | chr12: 24880798-28477058 | chr12: 27800441-27813180 | chr12: 25955987-28156868 | 0.60473038 |
| Peak 21 | | | | | |
| Amplification | 6q23.3 | chr6: 135342981-135632150 | chr6: 135513416-135593890 | chr6: 133418393-138096856 | 0.6007847 |
| Peak 9 | | | | | |
| Whole arm | 1q | NA | NA | chr1: 124535434-249250621 | 0.5932403 |
| deletion 19 | | | | | |
| Deletion Peak 31 | 8q11.1 | chr8: 42874387-48101823 | chr8: 42931769-48061147 | chr8: 42932439-48062091 | 0.59224336 |
| Whole arm | 19q | NA | NA | chr19: 27681782-59128983 | 0.58885616 |
| deletion 17 | | | | | |
| Whole arm | 12p | NA | NA | chr12: 1-34856694 | 0.58033903 |
| deletion 5 | | | | | |
| Whole arm | 5p | NA | NA | chr5: 1-46405641 | 0.58007436 |
| amplification 30 | | | | | |
| Deletion Peak 23 | 6p25.3 | chr6: 1613114-2256643 | chr6: 1621843-2256643 | chr6: 1626114-2752758 | 0.57614983 |
| Amplification | 17q25.3 | chr17: 77601682-77857881 | chr17: 77766436-77856811 | chr17: 75845339-81195210 | 0.57546275 |
| Peak 32 | | | | | |
| Amplification | 2q33.1 | chr2: 181446594-243199373 | chr2: 199603526-199649638 | chr2: 199525178-199824262 | 0.57035447 |
| Peak 4 | | | | | |
| Whole arm | 12p | NA | NA | chr12: 1-34856694 | 0.56509266 |
| amplification 5 | | | | | |
| Whole arm | 6q | NA | NA | chr6: 61830166-171115067 | 0.55451621 |
| deletion 33 | | | | | |
| Whole arm | 9q | NA | NA | chr9: 50367679-141213431 | 0.54581935 |
| deletion 39 | | | | | |
| Amplification | 3q29 | chr3: 174745992-198022430 | chr3: 195922177-195991311 | chr3: 174746836-198022430 | 0.54264532 |
| Peak 5 | | | | | |
| Amplification | 11p15.5 | chr11: 2091739-2302637 | chr11: 2095340-2301396 | chr11: 2095340-2305324 | 0.54017534 |
| Peak 18 | | | | | |
| Amplification | 17q24.2 | chr17: 65638523-65873408 | chr17: 65649475-65769071 | chr17: 63042470-68655121 | 0.53976335 |
| Peak 31 | | | | | |
| Deletion Peak 41 | 12q24.33 | chr12: 125048064-133851895 | chr12: 131256822-131362341 | chr12: 125246979-133851895 | 0.53826885 |
| Whole arm | 2q | NA | NA | chr2: 95326171-243199373 | 0.53771069 |
| amplification 25 | | | | | |
| Whole arm | 6q | NA | NA | chr6: 61830166-171115067 | 0.53538285 |
| amplification 33 | | | | | |
| Deletion Peak 20 | 5p15.33 | chr5: 1-2750686 | chr5: 1368344-2252637 | chr5: 1-3864653 | 0.52968199 |
| Whole arm | 3p | NA | NA | chr3: 1-90504854 | 0.52919712 |
| deletion 26 | | | | | |
| Amplification | 17q23.2 | chr17: 58400463-58674819 | chr17: 58400997-58532661 | chr17: 57045336-62714320 | 0.52759456 |
| Peak 30 | | | | | |
| Whole arm | 9p | NA | NA | chr9: 1-47367679 | 0.52569831 |
| amplification 38 | | | | | |
| Deletion Peak 6 | 2p25.3 | chr2: 1-4752017 | chr2: 1-488785 | chr2: 1-5260278 | 0.52195842 |
| Whole arm | 6p | NA | NA | chr6: 1-58830166 | 0.51638946 |
| deletion 32 | | | | | |
| Amplification | 19q13.11 | chr19: 32439834-33021877 | chr19: 32966965-32988344 | chr19: 19939350-41312227 | 0.51402152 |
| Peak 35 | | | | | |
| Deletion Peak 24 | 6p22.2 | chr6: 25926929-26025173 | chr6: 25932794-26025173 | chr6: 25933563-26346501 | 0.51056665 |
| Whole arm | 2p | NA | NA | chr2: 1-92326171 | 0.50462529 |
| amplification 24 | | | | | |
| Whole arm | 3q | NA | NA | chr3: 93504854-198022430 | 0.48576757 |
| amplification 27 | | | | | |
| Whole arm | 19q | NA | NA | chr19: 27681782-59128983 | 0.48190073 |
| amplification 17 | | | | | |
| Deletion Peak 13 | 3q26.31 | chr3: 173995229-175766885 | chr3: 174575326-175766885 | chr3: 174646449-174998725 | 0.47316393 |
| Whole arm | 12q | NA | NA | chr12: 37856694-133851895 | 0.4694395 |
| amplification 6 | | | | | |
| Whole arm | 20p | NA | NA | chr20: 1-26369569 | 0.46181551 |
| deletion 20 | | | | | |
| Amplification | 11q13.3 | chr11: 68746750-69827851 | chr11: 69311828-69824706 | chr11: 68748468-70760456 | 0.45349829 |
| Peak 19 | | | | | |
| Deletion Peak 48 | 16q23.1 | chr16: 78131135-79628242 | chr16: 78131135-79286336 | chr16: 78424695-79295468 | 0.45142138 |
| Deletion Peak 47 | 16p13.3 | chr16: 5141600-8053542 | chr16: 6056420-8053542 | chr16: 5279421-7736962 | 0.45028421 |
| Whole arm | 9q | NA | NA | chr9: 50367679-141213431 | 0.43896029 |
| amplification 39 | | | | | |
| Whole arm | 10p | NA | NA | chr10: 1-39254935 | 0.43821908 |
| amplification 1 | | | | | |
| Amplification | 15q26.1 | chr15: 84648780-102531392 | chr15: 90811423-90958538 | chr15: 90086447-91895585 | 0.43390108 |
| Peak 25 | | | | | |
| Whole arm | 19p | NA | NA | chr19: 1-24681782 | 0.42292166 |
| amplification 16 | | | | | |
| Whole arm | 11p | NA | NA | chr11: 1-51644205 | 0.42185291 |
| amplification 3 | | | | | |
| Whole arm | 21q | NA | NA | chr21: 14288129-48129895 | 0.38128102 |
| amplification 22 | | | | | |
| Whole arm | 17p | NA | NA | chr17: 1-22263006 | 0.3799839 |
| amplification 12 | | | | | |
| Whole arm | 3q | NA | NA | chr3: 93504854-198022430 | 0.37733862 |
| deletion 27 | | | | | |
| Whole arm | 3p | NA | NA | chr3: 1-90504854 | 0.3694481 |
| amplification 26 | | | | | |
| Whole arm | 11q | NA | NA | chr11: 54644205-135006516 | 0.36247292 |
| amplification 4 | | | | | |
| Whole arm | 16p | NA | NA | chr16: 1-35335801 | 0.36136172 |
| deletion 10 | | | | | |
| Deletion Peak 9 | 2q37.3 | chr2: 240321205-243199373 | chr2: 240940975-241060875 | chr2: 240589192-241997730 | 0.34823854 |
| Whole arm | 1p | NA | NA | chr1: 1-121535434 | 0.34348283 |
| amplification 18 | | | | | |
| Whole arm | 16q | NA | NA | chr16: 38335801-90354753 | 0.32026275 |
| deletion 11 | | | | | |
| Deletion Peak 7 | 2p21 | chr2: 42587649-44009118 | chr2: 43440855-43455807 | chr2: 43159857-43470966 | 0.31250652 |
| Deletion Peak 8 | 2q23.1 | chr2: 147341956-149499455 | chr2: 148437688-148750846 | chr2: 148444810-148756213 | 0.29511207 |
| Whole arm | 5q | NA | NA | chr5: 49405641-180915260 | 0.2792709 |
| amplification 31 | | | | | |
| Amplification | 10q22.3 | chr10: 79956009-83248579 | chr10: 80445049-80732930 | chr10: 77952482-83583616 | 0.27562286 |
| Peak 17 | | | | | |
| Amplification | 18q11.2 | chr18: 19517253-20155059 | chr18: 19525020-20154578 | chr18: 19243919-21268418 | 0.2636361 |
| Peak 33 | | | | | |
| Whole arm | 14q | NA | NA | chr14: 19000000-107349540 | 0.25989981 |
| amplification 8 | | | | | |
| Amplification | 1p34.2 | chr1: 39144054-44367347 | chr1: 42834925-43114106 | chr1: 42692914-43456374 | 0.25159063 |
| Peak 1 | | | | | |
| Whole arm | 8q | NA | NA | chr8: 46838887-146364022 | 0.24836297 |
| deletion 37 | | | | | |
| Whole arm | 10q | NA | NA | chr10: 42254935-135534747 | 0.21366949 |
| amplification 2 | | | | | |
| Whole arm | 18p | NA | NA | chr18: 1-15460898 | 0.20445617 |
| amplification 14 | | | | | |
| Whole arm | 4p | NA | NA | chr4: 1-49660117 | 0.18414829 |
| amplification 28 | | | | | |
| Deletion Peak 27 | 7q31.1 | chr7: 109590674-111367757 | chr7: 110232248-111358792 | chr7: 110595801-110618403 | 0.15773653 |
| Whole arm | 18q | NA | NA | chr18: 18460898-78077248 | 0.14698558 |
| amplification 15 | | | | | |
| Amplification | 18q21.1 | chr18: 46041609-46930382 | chr18: 46474762-46604295 | chr18: 46043163-46624631 | 0.145565 |
| Peak 34 | | | | | |
| Whole arm | 7q | NA | NA | chr7: 61054331-159138663 | 0.13516126 |
| deletion 35 | | | | | |
| Whole arm | 13q | NA | NA | chr13: 19000000-115169878 | 0.13461326 |
| deletion 7 | | | | | |
| Whole arm | 2p | NA | NA | chr2: 1-92326171 | 0.11809222 |
| deletion 24 | | | | | |
| Whole arm | 2q | NA | NA | chr2: 95326171-243199373 | 0.11400227 |
| deletion 25 | | | | | |
| Whole arm | 15q | NA | NA | chr15: 20000000-102531392 | 0.10837076 |
| amplification 9 | | | | | |
| Whole arm | 22q | NA | NA | chr22: 16000000-51304566 | 0.09878651 |
| amplification 23 | | | | | |
| Whole arm | 4q | NA | NA | chr4: 52660117-191154276 | 0.09805239 |
| amplification 29 | | | | | |
| Whole arm | 7p | NA | NA | chr7: 1-58054331 | 0.04804664 |
| deletion 34 | | | | | |
| Whole arm | 20q | NA | NA | chr20: 29369569-63025520 | 0 |
| deletion 21 | | | | | |

TABLE 14
Contribution of each of the 102 focal regions to the random forest classification model.

| | | | | | Contribution |
|---|---|---|---|---|---|
| Unique Name | Descriptor | Wide Peak Limits | Peak Limits | Region Limits | value |
| Deletion Peak 16 | 4q25 | chr4: 109046573-109544048 | chr4: 109451604-109544048 | chr4: 109489936-109535239 | 9.194419 |
| Deletion Peak 18 | 4q32.1 | chr4: 156135292-162306004 | chr4: 159683040-159830757 | chr4: 152233433-191154276 | 8.1782609 |
| Deletion Peak 17 | 4q31.3 | chr4: 152679194-153694102 | chr4: 153232273-153473069 | chr4: 152233433-191154276 | 8.0446448 |
| Deletion Peak 15 | 4q22.1 | chr4: 91143530-93226628 | chr4: 91046982-92557520 | chr4: 91103554-92561908 | 7.8661453 |
| Deletion Peak 19 | 4q35.1 | chr4: 184432080-185262191 | chr4: 184545375-185154488 | chr4: 152233433-191154276 | 7.1401903 |
| Amplification | 20q13.12 | chr20: 42534268-43281676 | chr20: 42537604-42773258 | chr20: 24375411-63025520 | 4.6635259 |
| Peak 41 | | | | | |
| Amplification | 20q11.21 | chr20: 29981165-30284236 | chr20: 30158807-30231055 | chr20: 24375411-63025520 | 4.4202524 |
| Peak 40 | | | | | |
| Deletion Peak 14 | 4p16.2 | chr4: 1-9787265 | chr4: 5915497-5925651 | chr4: 1-11455571 | 4.4096883 |
| Amplification | 20q13.2 | chr20: 52240629-52852159 | chr20: 52246659-52447690 | chr20: 24375411-63025520 | 4.2672955 |
| Peak 42 | | | | | |
| Deletion Peak 59 | 22q13.32 | chr22: 48649199-49178363 | chr22: 48906309-49158314 | chr22: 47983027-51304566 | 4.1267928 |
| Deletion Peak 57 | 21q11.2 | chr21: 15555708-16334057 | chr21: 15854887-15919066 | chr21: 1-31258004 | 4.0776699 |
| Deletion Peak 58 | 21q21.1 | chr21: 23106546-26219369 | chr21: 23248652-23494410 | chr21: 1-31258004 | 3.9182542 |
| Amplification | 20q13.32 | chr20: 56110288-57258354 | chr20: 57000076-57085518 | chr20: 24375411-63025520 | 3.5799093 |
| Peak 43 | | | | | |
| Deletion Peak 46 | 15q22.33 | chr15: 67053712-67697647 | chr15: 67337017-67551787 | chr15: 67245914-67753643 | 3.455251 |
| Deletion Peak 35 | 10q23.31 | chr10: 89502327-90051809 | chr10: 89574482-89607380 | chr10: 83248858-135534747 | 3.369556 |
| Deletion Peak 34 | 10q21.1 | chr10: 51561927-53481136 | chr10: 51561927-54065263 | chr10: 50763189-54253314 | 3.1436983 |
| Deletion Peak 53 | 18q21.2 | chr18: 48472034-48707815 | chr18: 48547928-48660122 | chr18: 47049746-78077248 | 2.905616 |
| Deletion Peak 36 | 10q25.2 | chr10: 114197471-115353755 | chr10: 114708532-114929210 | chr10: 83248858-135534747 | 2.7938966 |
| Deletion Peak 4 | 1p31.1 | chr1: 68959091-82269682 | chr1: 79308665-79566142 | chr1: 54944113-149961894 | 2.7530879 |
| Deletion Peak 5 | 1p21.1 | chr1: 102460262-107618224 | chr1: 104041213-104355050 | chr1: 54944113-149961894 | 2.6974638 |
| Deletion Peak 52 | 18q12.2 | chr18: 35136370-39061915 | chr18: 36781295-37333625 | chr18: 35827844-46846953 | 2.6594014 |
| Deletion Peak 54 | 18q21.33 | chr18: 60645473-61013467 | chr18: 60788090-61013467 | chr18: 47049746-78077248 | 2.5200778 |
| Amplification | 20p12.1 | chr20: 16140291-16967195 | chr20: 16488303-16962609 | chr20: 16146669-16970141 | 2.5116485 |
| Peak 38 | | | | | |
| Deletion Peak 38 | 11q22.3 | chr11: 102958343-135006516 | chr11: 108251085-108350263 | chr11: 108179920-108470348 | 2.4935124 |
| Deletion Peak 3 | 1p33 | chr1: 49187432-50544677 | chr1: 48847112-50490440 | chr1: 49196205-51396500 | 2.4544835 |
| Deletion Peak 44 | 15q11.2 | chr15: 25436434-25466900 | chr15: 25438412-25459423 | chr15: 1-58781038 | 2.3805641 |
| Deletion Peak 37 | 10q26.3 | chr10: 133107199-135534747 | chr10: 135221096-135534747 | chr10: 83248858-135534747 | 2.3715158 |
| Deletion Peak 51 | 18p11.31 | chr18: 3277394-4265401 | chr18: 3441391-3481373 | chr18: 3398771-3729936 | 2.3395225 |
| Amplification | 20p11.22 | chr20: 22058560-22353663 | chr20: 22063756-22350551 | chr20: 20938975-24286806 | 2.3155324 |
| Peak 39 | | | | | |
| Deletion Peak 49 | 17p12 | chr17: 11466949-12461211 | chr17: 11872374-11906034 | chr17: 10231123-12752622 | 2.2734537 |
| Deletion Peak 45 | 15q21.1 | chr15: 44850582-45321541 | chr15: 44851811-45052539 | chr15: 1-58781038 | 2.2056725 |
| Deletion Peak 22 | 5q22.2 | chr5: 111312546-112362638 | chr5: 111747052-111761475 | chr5: 50968806-135478060 | 2.1163858 |
| Amplification | 20p12.3 | chr20: 5376354-5742957 | chr20: 5379141-5737687 | chr20: 5156552-5961498 | 2.064109 |
| Peak 37 | | | | | |
| Deletion Peak 28 | 8p23.3 | chr8: 1-1449850 | chr8: 1-623109 | chr8: 1-10801317 | 2.0464708 |
| Amplification | 13q12.2 | chr13: 28164386-28564331 | chr13: 28174614-28222659 | chr13: 1-115169878 | 2.0138841 |
| Peak 22 | | | | | |
| Deletion Peak 21 | 5q12.1 | chr5: 58263825-59784640 | chr5: 58263825-59784640 | chr5: 50968806-135478060 | 1.9727809 |
| Amplification | 13q22.1 | chr13: 73775176-74007122 | chr13: 73906681-74004816 | chr13: 1-115169878 | 1.9642941 |
| Peak 23 | | | | | |
| Amplification | 8q21.13 | chr8: 80636493-82552412 | chr8: 81853752-81935079 | chr8: 54646329-146364022 | 1.7754935 |
| Peak 14 | | | | | |
| Deletion Peak 33 | 10p15.3 | chr10: 1-855610 | chr10: 1-418075 | chr10: 1-2055670 | 1.7489137 |
| Deletion Peak 42 | 14q24.1 | chr14: 68280014-69351476 | chr14: 68284712-68948164 | chr14: 68343659-68985929 | 1.7375301 |
| Deletion Peak 40 | 12q21.2 | chr12: 75602135-79819184 | chr12: 76423769-76523220 | chr12: 75648890-78593389 | 1.7138062 |
| Deletion Peak 43 | 14q32.11 | chr14: 78347564-107349540 | chr14: 90999841-91286309 | chr14: 90170502-92315681 | 1.7100382 |
| Amplification | 8q22.3 | chr8: 101853171-101878037 | chr8: 101853464-101876083 | chr8: 54646329-146364022 | 1.6977776 |
| Peak 15 | | | | | |
| Deletion Peak 2 | 1p36.11 | chr1: 26898389-27219375 | chr1: 27139248-27185402 | chr1: 1-37080378 | 1.655325 |
| Deletion Peak 1 | 1p36.31 | chr1: 4843384-6053964 | chr1: 5646446-6050774 | chr1: 1-37080378 | 1.6275767 |
| Amplification | 13q34 | chr13: 110097815-111753132 | chr13: 110614601-110852082 | chr13: 1-115169878 | 1.6177405 |
| Peak 24 | | | | | |
| Deletion Peak 56 | 20p12.1 | chr20: 13955189-16350354 | chr20: 14416445-15462745 | chr20: 14096517-16064189 | 1.5555618 |
| Amplification | 8q12.2 | chr8: 60847395-62906825 | chr8: 61658252-61874896 | chr8: 54646329-146364022 | 1.5323055 |
| Peak 13 | | | | | |
| Deletion Peak 29 | 8p21.3 | chr8: 13423967-26607015 | chr8: 21547484-21647267 | chr8: 11646511-25738962 | 1.5316374 |
| Deletion Peak 55 | 19p13.3 | chr19: 1488247-1660256 | chr19: 1488455-1660256 | chr19: 1-5957502 | 1.5195977 |
| Deletion Peak 30 | 8p12 | chr8: 33445578-37452445 | chr8: 34944675-36494184 | chr8: 35186331-35207233 | 1.5162631 |
| Amplification | 8p11.21 | chr8: 41760295-42054539 | chr8: 41767516-41796226 | chr8: 34342520-48854434 | 1.5015065 |
| Peak 12 | | | | | |
| Amplification | 5p12 | chr5: 33430780-50470114 | chr5: 43822799-43929095 | chr5: 37334807-44266311 | 1.4519356 |
| Peak 7 | | | | | |
| Amplification | 8q24.21 | chr8: 128574277-128592142 | chr8: 128574768-128591041 | chr8: 54646329-146364022 | 1.4373339 |
| Peak 16 | | | | | |
| Deletion Peak 12 | 3p13 | chr3: 70014100-71250049 | chr3: 70976616-71182883 | chr3: 70977475-71315576 | 1.4356909 |
| Deletion Peak 10 | 3p26.3 | chr3: 1-8350135 | chr3: 1-2251569 | chr3: 1-9456413 | 1.4256335 |
| Amplification | 7p11.2 | chr7: 54949302-55950640 | chr7: 55248172-55271747 | chr7: 54949902-55955413 | 1.3851619 |
| Peak 10 | | | | | |
| Amplification | 1q43 | chr1: 201615295-249250621 | chr1: 242413158-242638099 | chr1: 242270003-249250621 | 1.3355112 |
| Peak 3 | | | | | |
| Deletion Peak 26 | 6q26 | chr6: 161540781-163179430 | chr6: 161540781-163179430 | chr6: 160448048-171115067 | 1.3326207 |
| Deletion Peak 25 | 6q21 | chr6: 91006553-129206367 | chr6: 111544567-112706964 | chr6: 108785353-116683959 | 1.3147804 |
| Deletion Peak 50 | 17q24.3 | chr17: 68174484-70599305 | chr17: 70337175-70590424 | chr17: 70549664-70552757 | 1.2848234 |
| Amplification | 6p21.1 | chr6: 43545080-44164949 | chr6: 43765716-44163738 | chr6: 36748055-52028937 | 1.2774639 |
| Peak 8 | | | | | |
| Amplification | 5p15.33 | chr5: 1-6418038 | chr5: 1-949725 | chr5: 1-2807521 | 1.2446252 |
| Peak 6 | | | | | |
| Amplification | 8p11.23 | chr8: 38162916-38237532 | chr8: 38165117-38236937 | chr8: 34342520-48854434 | 1.2319335 |
| Peak 11 | | | | | |
| Deletion Peak 32 | 9p21.3 | chr9: 21558582-22452906 | chr9: 21995318-22021004 | chr9: 21711940-22331169 | 1.1988554 |
| Amplification | 16p11.2 | chr16: 30548665-30684980 | chr16: 30549334-30682578 | chr16: 28556705-46663588 | 1.197811 |
| Peak 26 | | | | | |
| Deletion Peak 11 | 3p14.2 | chr3: 58946448-61555632 | chr3: 59692189-61457375 | chr3: 59692189-61460946 | 1.1379309 |
| Amplification | 1q22 | chr1: 120494739-199253746 | chr1: 155143717-155177826 | chr1: 120497533-175439985 | 1.1037294 |
| Peak 2 | | | | | |
| Deletion Peak 41 | 12q24.33 | chr12: 125048064-133851895 | chr12: 131256822-131362341 | chr12: 125246979-133851895 | 1.092504 |
| Amplification | 17q11.2 | chr17: 27339261-27528113 | chr17: 27339592-27419750 | chr17: 20042949-40168023 | 1.0736711 |
| Peak 28 | | | | | |
| Amplification | 17q12 | chr17: 37747533-38052599 | chr17: 37950674-38031986 | chr17: 20042949-40168023 | 1.061722 |
| Peak 29 | | | | | |
| Deletion Peak 20 | 5p15.33 | chr5: 1-2750686 | chr5: 1368344-2252637 | chr5: 1-3864653 | 1.0603831 |
| Amplification | 16q12.1 | chr16: 52243935-52652774 | chr16: 52383686-52652198 | chr16: 51078632-53117566 | 1.0570224 |
| Peak 27 | | | | | |
| Amplification | 12p11.22 | chr12: 24880798-28477058 | chr12: 27800441-27813180 | chr12: 25955987-28156868 | 1.0564938 |
| Peak 21 | | | | | |
| Amplification | 2q33.1 | chr2: 181446594-243199373 | chr2: 199603526-199649638 | chr2: 199525178-199824262 | 1.0392578 |
| Peak 4 | | | | | |
| Amplification | 17q25.3 | chr17: 77601682-77857881 | chr17: 77766436-77856811 | chr17: 75845339-81195210 | 1.0214531 |
| Peak 32 | | | | | |
| Deletion Peak 39 | 12p13.2 | chr12: 12412186-13039757 | chr12: 12525464-12721981 | chr12: 11739866-13828006 | 1.01047 |
| Amplification | 3q29 | chr3: 174745992-198022430 | chr3: 195922177-195991311 | chr3: 174746836-198022430 | 1.0074218 |
| Peak 5 | | | | | |
| Amplification | 6q23.3 | chr6: 135342981-135632150 | chr6: 135513416-135593890 | chr6: 133418393-138096856 | 0.9983965 |
| Peak 9 | | | | | |
| Deletion Peak 24 | 6p22.2 | chr6: 25926929-26025173 | chr6: 25932794-26025173 | chr6: 25933563-26346501 | 0.9867404 |
| Amplification | 17q24.2 | chr17: 65638523-65873408 | chr17: 65649475-65769071 | chr17: 63042470-68655121 | 0.9853368 |
| Peak 31 | | | | | |
| Amplification | 17q23.2 | chr17: 58400463-58674819 | chr17: 58400997-58532661 | chr17: 57045336-62714320 | 0.928049 |
| Peak 30 | | | | | |
| Deletion Peak 31 | 8q11.1 | chr8: 42874387-48101823 | chr8: 42931769-48061147 | chr8: 42932439-48062091 | 0.9098856 |
| Amplification | 11p15.5 | chr11: 2091739-2302637 | chr11: 2095340-2301396 | chr11: 2095340-2305324 | 0.9026687 |
| Peak 18 | | | | | |
| Deletion Peak 23 | 6p25.3 | chr6: 1613114-2256643 | chr6: 1621843-2256643 | chr6: 1626114-2752758 | 0.8969569 |
| Amplification | 12p13.32 | chr12: 3941807-4552801 | chr12: 4257035-4414967 | chr12: 1-8970181 | 0.892304 |
| Peak 20 | | | | | |
| Amplification | 19q13.2 | chr19: 39844313-40357920 | chr19: 39848086-40009185 | chr19: 19939350-41312227 | 0.8844445 |
| Peak 36 | | | | | |
| Deletion Peak 48 | 16q23.1 | chr16: 78131135-79628242 | chr16: 78131135-79286336 | chr16: 78424695-79295468 | 0.8843983 |
| Amplification | 19q13.11 | chr19: 32439834-33021877 | chr19: 32966965-32988344 | chr19: 19939350-41312227 | 0.880044 |
| Peak 35 | | | | | |
| Deletion Peak 6 | 2p25.3 | chr2: 1-4752017 | chr2: 1-488785 | chr2: 1-5260278 | 0.7876138 |
| Deletion Peak 13 | 3q26.31 | chr3: 173995229-175766885 | chr3: 174575326-175766885 | chr3: 174646449-174998725 | 0.7648052 |
| Deletion Peak 47 | 16p13.3 | chr16: 5141600-8053542 | chr16: 6056420-8053542 | chr16: 5279421-7736962 | 0.6998853 |
| Amplification | 11q13.3 | chr11: 68746750-69827851 | chr11: 69311828-69824706 | chr11: 68748468-70760456 | 0.6309478 |
| Peak 19 | | | | | |
| Amplification | 15q26.1 | chr15: 84648780-102531392 | chr15: 90811423-90958538 | chr15: 90086447-91895585 | 0.5231053 |
| Peak 25 | | | | | |
| Deletion Peak 9 | 2q37.3 | chr2: 240321205-243199373 | chr2: 240940975-241060875 | chr2: 240589192-241997730 | 0.5216232 |
| Deletion Peak 8 | 2q23.1 | chr2: 147341956-149499455 | chr2: 148437688-148750846 | chr2: 148444810-148756213 | 0.5056672 |
| Amplification | 18q11.2 | chr18: 19517253-20155059 | chr18: 19525020-20154578 | chr18: 19243919-21268418 | 0.4808792 |
| Peak 33 | | | | | |
| Deletion Peak 7 | 2p21 | chr2: 42587649-44009118 | chr2: 43440855-43455807 | chr2: 43159857-43470966 | 0.4489627 |
| Amplification | 1p34.2 | chr1: 39144054-44367347 | chr1: 42834925-43114106 | chr1: 42692914-43456374 | 0.4346417 |
| Peak 1 | | | | | |
| Amplification | 10q22.3 | chr10: 79956009-83248579 | chr10: 80445049-80732930 | chr10: 77952482-83583616 | 0.3836683 |
| Peak 17 | | | | | |
| Amplification | 18q21.1 | chr18: 46041609-46930382 | chr18: 46474762-46604295 | chr18: 46043163-46624631 | 0.2879049 |
| Peak 34 | | | | | |
| Deletion Peak 27 | 7q31.1 | chr7: 109590674-111367757 | chr7: 110232248-111358792 | chr7: 110595801-110618403 | 0.2331979 |

TABLE 15
Comparison between recursive partitioning tiers and the contribution to the random
forest model for each of the 180 genomic regions. Genomic regions are listed by their
descriptor name. Full information of these markers (e.g. peak limits and region limits) can be
retrieved from Table 1 using the descriptor name.

| Descriptor name | Contribution | Tier 1 | Tier 2 | Tier 3 | Tier 4 |
|---|---|---|---|---|---|
| del_4q31.3 | 5.69900368 | del_4q31.3 | — | — | — |
| del_4q35.1 | 5.4602327 | — | del_4q35.1 | — | — |
| del_4q22.1 | 5.40079966 | del_4q22.1 | — | — | — |
| del_4q32.1 | 5.22218115 | del_4q32.1 | — | — | — |
| wa_del_4q | 5.14074254 | — | wa_del_4q | — | — |
| del_4q25 | 5.12389105 | — | del_4q25 | — | — |
| wa_del_4p | 4.6643299 | — | wa_del_4p | — | — |
| amp_20q13.12 | 3.29423738 | amp_20q13.12 | — | — | — |
| del_1p31.1 | 2.92406308 | — | del_1p31.1 | — | — |
| del_4p16.2 | 2.77180108 | — | — | del_4p16.2 | — |
| wa_del_22q | 2.72591739 | wa_del_22q | — | — | — |
| del_21q11.2 | 2.55008085 | del_21q11.2 | — | — | — |
| amp_20q13.2 | 2.53769321 | amp_20q13.2 | — | — | — |
| amp_20q11.21 | 2.46858084 | — | amp_20q11.21 | — | — |
| wa_amp_20q | 2.4607282 | — | wa_amp_20q | — | — |
| del_21q21.1 | 2.46055021 | del_21q21.1 | — | — | — |
| wa_del_21q | 2.36208909 | wa_del_21q | — | — | — |
| amp_20q13.32 | 2.35785114 | amp_20q13.32 | — | — | — |
| del_22q13.32 | 2.27724108 | del_22q13.32 | — | — | — |
| del_10q21.1 | 1.85840926 | del_10q21.1 | — | — | — |
| del_1p21.1 | 1.85495579 | — | del_1p21.1 | — | — |
| del_11q22.3 | 1.79129861 | del_11q22.3 | — | — | — |
| del_1p33 | 1.75409699 | — | del_1p33 | — | — |
| del_10q26.3 | 1.74689724 | — | del_10q26.3 | — | — |
| wa_del_15q | 1.74607014 | — | wa_del_15q | — | — |
| wa_del_10q | 1.74534855 | wa_del_10q | — | — | — |
| del_15q21.1 | 1.74523402 | — | — | del_15q21.1 | — |
| del_18q12.2 | 1.74103677 | del_18q12.2 | — | — | — |
| del_17p12 | 1.74080473 | — | del_17p12 | — | — |
| del_10q23.31 | 1.73841158 | del_10q23.31 | — | — | — |
| wa_del_18p | 1.69686457 | — | wa_del_18p | — | — |
| wa_del_18q | 1.63024275 | wa_del_18q | — | — | — |
| del_15q22.33 | 1.61216031 | — | del_15q22.33 | — | — |
| del_18q21.2 | 1.54801279 | del_18q21.2 | — | — | — |
| amp_20p12.3 | 1.54217323 | amp_20p12.3 | — | — | — |
| del_10q25.2 | 1.49414539 | del_10q25.2 | — | — | — |
| del_18q21.33 | 1.46051102 | — | del_18q21.33 | — | — |
| del_18p11.31 | 1.44244308 | — | del_18p11.31 | — | — |
| amp_20p12.1 | 1.39784163 | amp_20p12.1 | — | — | — |
| del_8p23.3 | 1.376404 | del_8p23.3 | — | — | — |
| wa_del_11p | 1.36242884 | — | — | wa_del_11p | — |
| del_1p36.11 | 1.35158286 | — | — | del_1p36.11 | — |
| del_5q22.2 | 1.32777145 | del_5q22.2 | — | — | — |
| wa_del_17q | 1.31992276 | — | wa_del_17q | — | — |
| del_5q12.1 | 1.31750542 | — | del_5q12.1 | — | — |
| amp_20p11.22 | 1.29653026 | — | amp_20p11.22 | — | — |
| amp_13q12.2 | 1.29142193 | — | amp_13q12.2 | — | — |
| del_15q11.2 | 1.27544343 | — | — | del_15q11.2 | — |
| del_14q32.11 | 1.2470974 | — | del_14q32.11 | — | — |
| wa_del_11q | 1.22838424 | — | wa_del_11q | — | — |
| wa_del_5q | 1.21343422 | wa_del_5q | — | — | — |
| del_14q24.1 | 1.18921641 | — | del_14q24.1 | — | — |
| amp_13q22.1 | 1.18041391 | — | — | amp_13q22.1 | — |
| amp_8q22.3 | 1.1639936 | — | amp_8q22.3 | — | — |
| wa_amp_20p | 1.11182166 | wa_amp_20p | — | — | — |
| wa_del_17p | 1.09115055 | — | wa_del_17p | — | — |
| wa_amp_13q | 1.08715413 | — | — | wa_amp_13q | — |
| del_12q21.2 | 1.08195556 | del_12q21.2 | — | — | — |
| del_10p15.3 | 1.03741963 | — | — | del_10p15.3 | — |
| wa_del_10p | 1.03431515 | — | wa_del_10p | — | — |
| wa_del_8p | 1.01165483 | — | — | wa_del_8p | — |
| amp_8p11.21 | 1.00972856 | amp_8p11.21 | — | — | — |
| amp_8q21.13 | 1.00902364 | — | amp_8q21.13 | — | — |
| wa_amp_8q | 0.97550139 | — | wa_amp_8q | — | — |
| amp_13q34 | 0.97236703 | — | — | amp_13q34 | — |
| wa_del_1p | 0.9682743 | — | — | wa_del_1p | — |
| del_8p21.3 | 0.94172849 | — | — | del_8p21.3 | — |
| amp_8q24.21 | 0.90669936 | — | amp_8q24.21 | — | — |
| del_3p26.3 | 0.90057889 | — | del_3p26.3 | — | — |
| del_20p12.1 | 0.89553463 | del_20p12.1 | — | — | — |
| amp_17q12 | 0.8947382 | amp_17q12 | — | — | — |
| del_8p12 | 0.89027155 | — | del_8p12 | — | — |
| wa_del_14q | 0.87610091 | — | wa_del_14q | — | — |
| del_19p13.3 | 0.86068674 | — | del_19p13.3 | — | — |
| del_1p36.31 | 0.85950877 | — | — | del_1p36.31 | — |
| wa_del_9p | 0.84910578 | wa_del_9p | — | — | — |
| del_17q24.3 | 0.82566352 | — | del_17q24.3 | — | — |
| amp_17q11.2 | 0.80753708 | amp_17q11.2 | — | — | — |
| amp_8p11.23 | 0.79953951 | — | amp_8p11.23 | — | — |
| amp_1q43 | 0.78150602 | — | amp_1q43 | — | — |
| wa_amp_7q | 0.77800193 | — | — | — | wa_amp_7q |
| amp_8q12.2 | 0.77096113 | — | amp_8q12.2 | — | — |
| wa_amp_7p | 0.76807364 | — | wa_amp_7p | — | — |
| amp_5p12 | 0.75708381 | — | — | — | amp_5p12 |
| del_3p13 | 0.75572694 | — | del_3p13 | — | — |
| del_9p21.3 | 0.75281938 | — | — | del_9p21.3 | — |
| amp_5p15.33 | 0.75064607 | — | — | — | amp_5p15.33 |
| amp_16q12.1 | 0.72390332 | — | — | amp_16q12.1 | — |
| wa_amp_16p | 0.71183633 | — | — | — | wa_amp_16p |
| amp_7p11.2 | 0.71128453 | — | — | — | amp_7p11.2 |
| del_3p14.2 | 0.69805324 | del_3p14.2 | — | — | — |
| wa_amp_16q | 0.6977664 | — | — | wa_amp_16q | — |
| amp_16p11.2 | 0.68677078 | — | — | amp_16p11.2 | — |
| wa_amp_17q | 0.68298273 | — | — | wa_amp_17q | — |
| del_6q26 | 0.68131369 | del_6q26 | — | — | — |
| amp_6p21.1 | 0.68018519 | — | amp_6p21.1 | — | — |
| wa_del_12q | 0.67955302 | — | — | wa_del_12q | — |
| del_6q21 | 0.67397177 | del_6q21 | — | — | — |
| amp_1q22 | 0.6692668 | — | — | amp_1q22 | — |
| wa_amp_6p | 0.66083392 | — | — | wa_amp_6p | — |
| wa_amp_8p | 0.65986144 | — | — | wa_amp_8p | — |
| amp_12p13.32 | 0.65326501 | — | — | — | amp_12p13.32 |
| wa_amp_1q | 0.63810089 | — | — | wa_amp_1q | — |
| wa_del_5p | 0.62977686 | — | — | wa_del_5p | — |
| del_12p13.2 | 0.61944112 | — | del_12p13.2 | — | — |
| wa_del_19p | 0.61447685 | — | — | — | wa_del_19p |
| amp_19q13.2 | 0.61443667 | — | — | amp_19q13.2 | — |
| amp_12p11.22 | 0.60473038 | — | — | amp_12p11.22 | — |
| amp_6q23.3 | 0.6007847 | — | — | — | amp_6q23.3 |
| wa_del_1q | 0.5932403 | — | — | wa_del_1q | — |
| del_8q11.1 | 0.59224336 | — | del_8q11.1 | — | — |
| wa_del_19q | 0.58885616 | — | — | wa_del_19q | — |
| wa_del_12p | 0.58033903 | — | — | — | wa_del_12p |
| wa_amp_5p | 0.58007436 | — | — | wa_amp_5p | — |
| del_6p25.3 | 0.57614983 | del_6p25.3 | — | — | — |
| amp_17q25.3 | 0.57546275 | — | — | amp_17q25.3 | — |
| amp_2q33.1 | 0.57035447 | — | — | amp_2q33.1 | — |
| wa_amp_12p | 0.56509266 | — | — | — | wa_amp_12p |
| wa_del_6q | 0.55451621 | — | wa_del_6q | — | — |
| wa_del_9q | 0.54581935 | — | — | wa_del_9q | — |
| amp_3q29 | 0.54264532 | — | amp_3q29 | — | — |
| amp_11p15.5 | 0.54017534 | — | — | amp_11p15.5 | — |
| amp_17q24.2 | 0.53976335 | — | amp_17q24.2 | — | — |
| del_12q24.33 | 0.53826885 | — | — | del_12q24.33 | — |
| wa_amp_2q | 0.53771069 | — | — | — | wa_amp_2q |
| wa_amp_6q | 0.53538285 | — | — | — | wa_amp_6q |
| del_5p15.33 | 0.52968199 | — | — | del_5p15.33 | — |
| wa_del_3p | 0.52919712 | — | — | wa_del_3p | — |
| amp_17q23.2 | 0.52759456 | — | amp_17q23.2 | — | — |
| wa_amp_9p | 0.52569831 | — | — | — | wa_amp_9p |
| del_2p25.3 | 0.52195842 | — | — | — | del_2p25.3 |
| wa_del_6p | 0.51638946 | — | wa_del_6p | — | — |
| amp_19q13.11 | 0.51402152 | — | — | amp_19q13.11 | — |
| del_6p22.2 | 0.51056665 | del_6p22.2 | — | — | — |
| wa_amp_2p | 0.50462529 | — | — | — | wa_amp_2p |
| wa_amp_3q | 0.48576757 | — | — | wa_amp_3q | — |
| wa_amp_19q | 0.48190073 | — | — | — | wa_amp_19q |
| del_3q26.31 | 0.47316393 | — | — | del_3q26.31 | — |
| wa_amp_12q | 0.4694395 | — | — | — | wa_amp_12q |
| wa_del_20p | 0.46181551 | — | — | wa_del_20p | — |
| amp_11q13.3 | 0.45349829 | — | — | amp_11q13.3 | — |
| del_16q23.1 | 0.45142138 | — | — | del_16q23.1 | — |
| del_16p13.3 | 0.45028421 | — | — | del_16p13.3 | — |
| wa_amp_9q | 0.43896029 | — | wa_amp_9q | — | — |
| wa_amp_10p | 0.43821908 | — | — | — | wa_amp_10p |
| amp_15q26.1 | 0.43390108 | — | — | — | amp_15q26.1 |
| wa_amp_19p | 0.42292166 | — | — | wa_amp_19p | — |
| wa_amp_11p | 0.42185291 | — | — | wa_amp_11p | — |
| wa_amp_21q | 0.38128102 | — | — | — | wa_amp_21q |
| wa_amp_17p | 0.3799839 | — | — | — | wa_amp_17p |
| wa_del_3q | 0.37733862 | — | — | wa_del_3q | — |
| wa_amp_3p | 0.3694481 | — | — | — | wa_amp_3p |
| wa_amp_11q | 0.36247292 | — | — | wa_amp_11q | — |
| wa_del_16p | 0.36136172 | — | — | — | wa_del_16p |
| del_2q37.3 | 0.34823854 | — | — | — | del_2q37.3 |
| wa_amp_1p | 0.34348283 | — | — | wa_amp_1p | — |
| wa_del_16q | 0.32026275 | — | — | — | wa_del_16q |
| del_2p21 | 0.31250652 | — | — | del_2p21 | — |
| del_2q23.1 | 0.29511207 | — | — | — | del_2q23.1 |
| wa_amp_5q | 0.2792709 | — | — | — | a_amp_5q |
| amp_10q22.3 | 0.27562286 | — | — | — | amp_10q22.3 |
| amp_18q11.2 | 0.2636361 | — | — | — | amp_18q11.2 |
| wa_amp_14q | 0.25989981 | — | — | — | wa_amp_14q |
| amp_1p34.2 | 0.25159063 | — | — | — | amp_1p34.2 |
| wa_del_8q | 0.24836297 | — | — | wa_del_8q | — |
| wa_amp_10q | 0.21366949 | — | — | — | wa_amp_10q |
| wa_amp_18p | 0.20445617 | — | — | — | wa_amp_18p |
| wa_amp_4p | 0.18414829 | — | — | — | wa_amp_4p |
| del_7q31.1 | 0.15773653 | — | — | — | del_7q31.1 |
| wa_amp_18q | 0.14698558 | — | — | — | wa_amp_18q |
| amp_18q21.1 | 0.145565 | — | — | — | amp_18q21.1 |
| wa_del_7q | 0.13516126 | — | — | — | wa_del_7q |
| wa_del_13q | 0.13461326 | — | — | — | wa_del_13q |
| wa_del_2p | 0.11809222 | — | — | — | wa_del_2p |
| wa_del_2q | 0.11400227 | — | — | — | wa_del_2q |
| wa_amp_15q | 0.10837076 | — | — | — | wa_amp_15q |
| wa_amp_22q | 0.09878651 | — | — | — | wa_amp_22q |
| wa_amp_4q | 0.09805239 | — | — | wa_amp_4q | — |
| wa_del_7p | 0.04804664 | — | — | — | wa_del_7p |
| wa_del_20q | 0 | — | — | — | wa_del_20q |

TABLE 16
Comparison between recursive partitioning tiers and the contribution to the random
forest model for each of the 102 focal genomic regions. Genomic regions are listed by
their descriptor name. Full information of these markers (e.g. peak limits and region
limits) can be retrieved from Table 5 using the descriptor name.

| Descriptor name | Contribution | Tier 1 | Tier 2 | Tier 3 |
|---|---|---|---|---|
| del_4q25 | 9.194419 | — | del_4q25 | — |
| del_4q32.1 | 8.1782609 | del_4q32.1 | — | — |
| del_4q31.3 | 8.0446448 | del_4q31.3 | — | — |
| del_4q22.1 | 7.8661453 | del_4q22.1 | — | — |
| del_4q35.1 | 7.1401903 | — | del_4q35.1 | — |
| amp_20q13.12 | 4.6635259 | amp_20q13.12 | — | — |
| amp_20q11.21 | 4.4202524 | — | amp_20q11.21 | — |
| del_4p16.2 | 4.4096883 | — | del_4p16.2 | — |
| amp_20q13.2 | 4.2672955 | amp_20q13.2 | — | — |
| del_22q13.32 | 4.1267928 | del_22q13.32 | — | — |
| del_21q11.2 | 4.0776699 | del_21q11.2 | — | — |
| del_21q21.1 | 3.9182542 | del_21q21.1 | — | — |
| amp_20q13.32 | 3.5799093 | amp_20q13.32 | — | — |
| del_15q22.33 | 3.455251 | del_15q22.33 | — | — |
| del_10q23.31 | 3.369556 | del_10q23.31 | — | — |
| del_10q21.1 | 3.1436983 | del_10q21.1 | — | — |
| del_18q21.2 | 2.905616 | del_18q21.2 | — | — |
| del_10q25.2 | 2.7938966 | del_10q25.2 | — | — |
| del_1p31.1 | 2.7530879 | — | del_1p31.1 | — |
| del_1p21.1 | 2.6974638 | — | del_1p21.1 | — |
| del_18q12.2 | 2.6594014 | del_18q12.2 | — | — |
| del_18q21.33 | 2.5200778 | — | del_18q21.33 | — |
| amp_20p12.1 | 2.5116485 | amp_20p12.1 | — | — |
| del_11q22.3 | 2.4935124 | del_11q22.3 | — | — |
| del_1p33 | 2.4544835 | — | del_1p33 | — |
| del_15q11.2 | 2.3805641 | — | del_15q11.2 | — |
| del_10q26.3 | 2.3715158 | — | del_10q26.3 | — |
| del_18p11.31 | 2.3395225 | — | del_18p11.31 | — |
| amp_20p11.22 | 2.3155324 | amp_20p11.22 | — | — |
| del_17p12 | 2.2734537 | — | del_17p12 | — |
| del_15q21.1 | 2.2056725 | — | del_15q21.1 | — |
| del_5q22.2 | 2.1163858 | del_5q22.2 | — | — |
| amp_20p12.3 | 2.064109 | amp_20p12.3 | — | — |
| del_8p23.3 | 2.0464708 | del_8p23.3 | — | — |
| amp_13q12.2 | 2.0138841 | — | amp_13q12.2 | — |
| del_5q12.1 | 1.9727809 | — | del_5q12.1 | — |
| amp_13q22.1 | 1.9642941 | — | amp_13q22.1 | — |
| amp_8q21.13 | 1.7754935 | amp_8q21.13 | — | — |
| del_10p15.3 | 1.7489137 | — | del_10p15.3 | — |
| del_14q24.1 | 1.7375301 | — | del_14q24.1 | — |
| del_12q21.2 | 1.7138062 | del_12q21.2 | — | — |
| del_14q32.11 | 1.7100382 | del_14q32.11 | — | — |
| amp_8q22.3 | 1.6977776 | amp_8q22.3 | — | — |
| del_1p36.11 | 1.655325 | — | — | del_1p36.11 |
| del_1p36.31 | 1.6275767 | — | — | del_1p36.31 |
| amp_13q34 | 1.6177405 | — | amp_13q34 | — |
| del_20p12.1 | 1.5555618 | — | del_20p12.1 | — |
| amp_8q12.2 | 1.5323055 | — | amp_8q12.2 | — |
| del_8p21.3 | 1.5316374 | — | del_8p21.3 | — |
| del_19p13.3 | 1.5195977 | — | del_19p13.3 | — |
| del_8p12 | 1.5162631 | — | del_8p12 | — |
| amp_8p11.21 | 1.5015065 | amp_8p11.21 | — | — |
| amp_5p12 | 1.4519356 | — | amp_5p12 | — |
| amp_8q24.21 | 1.4373339 | — | amp_8q24.21 | — |
| del_3p13 | 1.4356909 | del_3p13 | — | — |
| del_3p26.3 | 1.4256335 | — | del_3p26.3 | — |
| amp_7p11.2 | 1.3851619 | — | amp_7p11.2 | — |
| amp_1q43 | 1.3355112 | — | amp_1q43 | — |
| del_6q26 | 1.3326207 | del_6q26 | — | — |
| del_6q21 | 1.3147804 | del_6q21 | — | — |
| del_17q24.3 | 1.2848234 | — | del_17q24.3 | — |
| amp_6p21.1 | 1.2774639 | — | amp_6p21.1 | — |
| amp_5p15.33 | 1.2446252 | — | — | amp_5p15.33 |
| amp_8p11.23 | 1.2319335 | — | amp_8p11.23 | — |
| del_9p21.3 | 1.1988554 | — | — | del_9p21.3 |
| amp_16p11.2 | 1.197811 | — | amp_16p11.2 | — |
| del_3p14.2 | 1.1379309 | — | — | del_3p14.2 |
| amp_1q22 | 1.1037294 | amp_1q22 | — | — |
| del_12q24.33 | 1.092504 | — | — | del_12q24.33 |
| amp_17q11.2 | 1.0736711 | amp_17q11.2 | — | — |
| amp_17q12 | 1.061722 | — | amp_17q12 | — |
| del_5p15.33 | 1.0603831 | — | — | del_5p15.33 |
| amp_16q12.1 | 1.0570224 | — | — | amp_16q12.1 |
| amp_12p11.22 | 1.0564938 | — | — | amp_12p11.22 |
| amp_2q33.1 | 1.0392578 | — | amp_2q33.1 | — |
| amp_17q25.3 | 1.0214531 | — | — | amp_17q25.3 |
| del_12p13.2 | 1.01047 | — | del_12p13.2 | — |
| amp_3q29 | 1.0074218 | — | — | amp_3q29 |
| amp_6q23.3 | 0.9983965 | — | amp_6q23.3 | — |
| del_6p22.2 | 0.9867404 | del_6p22.2 | — | — |
| amp_17q24.2 | 0.9853368 | — | amp_17q24.2 | — |
| amp_17q23.2 | 0.928049 | — | — | amp_17q23.2 |
| del_8q11.1 | 0.9098856 | — | — | del_8q11.1 |
| amp_11p15.5 | 0.9026687 | — | amp_11p15.5 | — |
| del_6p25.3 | 0.8969569 | del_6p25.3 | — | — |
| amp_12p13.32 | 0.892304 | — | — | amp_12p13.32 |
| amp_19q13.2 | 0.8844445 | — | — | amp_19q13.2 |
| del_16q23.1 | 0.8843983 | — | del_16q23.1 | — |
| amp_19q13.11 | 0.880044 | — | — | amp_19q13.11 |
| del_2p25.3 | 0.7876138 | — | — | del_2p25.3 |
| del_3q26.31 | 0.7648052 | — | del_3q26.31 | — |
| del_16p13.3 | 0.6998853 | — | — | del_16p13.3 |
| amp_11q13.3 | 0.6309478 | — | — | amp_11q13.3 |
| amp_15q26.1 | 0.5231053 | — | amp_15q26.1 | — |
| del_2q37.3 | 0.5216232 | — | — | del_2q37.3 |
| del_2q23.1 | 0.5056672 | — | — | del_2q23.1 |
| amp_18q11.2 | 0.4808792 | — | — | amp_18q11.2 |
| del_2p21 | 0.4489627 | — | — | del_2p21 |
| amp_1p34.2 | 0.4346417 | — | — | amp_1p34.2 |
| amp_10q22.3 | 0.3836683 | — | — | amp_10q22.3 |
| amp_18q21.1 | 0.2879049 | — | — | amp_18q21.1 |
| del_7q31.1 | 0.2331979 | — | — | del_7q31.1 |

TABLE 17
Report on random forest classifier and k-nearest neighbor classifiers

| | | | |
|---|---|---|---|
| Statistics by class | Cluster 1 | Cluster 2 | Cluster 3 |

Random forest - Using all 180 regions
Accuracy: 0.9412
95% CI: (0.915, 0.9612)

| | | | |
|---|---|---|---|
| Sensitivity | 1 | 0.881 | 0.9744 |
| Specificity | 0.99751 | 0.9781 | 0.9087 |
| Balanced Accuracy | 0.99876 | 0.9295 | 0.9415 |

Random forest - Using the 102 focal regions
Accuracy: 0.9389
95% CI: (0.9124, 0.9594)

| | | | |
|---|---|---|---|
| Sensitivity | 1 | 0.8929 | 0.9615 |
| Specificity | 1 | 0.9672 | 0.9135 |
| Balanced Accuracy | 1 | 0.93 | 0.9375 |

k-nearest neighbor - Using all 180 regions
Accuracy: 0.8643
95% CI: (0.8287, 0.8948)

| | | | |
|---|---|---|---|
| Sensitivity | 1 | 0.9167 | 0.8034 |
| Specificity | 0.9577 | 0.8577 | 0.9808 |
| Balanced Accuracy | 0.9789 | 0.8872 | 0.8921 |

k-nearest neighbor - Using the 102 focal regions
Accuracy: 0.8733
95% CI: (0.8386, 0.9029)

| | | | |
|---|---|---|---|
| Sensitivity | 1 | 0.9226 | 0.8162 |
| Specificity | 0.9677 | 0.8613 | 0.976 |
| Balanced Accuracy | 0.9838 | 0.892 | 0.8961 |

TABLE 18
The relation of copy number instability in different subsets of regions and the response to Avastin. P stands for predictive
for copy number instability, NP stands for not predictive for copy number instability.
The relative number of regions affected by CNAs can be seen as a measure for copy number instability. Using different
thresholds to define tumors as copy number unstable and stratify the patients accordingly we were able to observe
beneficial responses to Avastin treatment for tumor instabilities ranging from 10% to 40% of regions affected by CNAs.
We performed this analysis on 6 different subsets (1) using only the 102 focal regions, (2) using the top 50 ranked
regions from the random forest classification model built with the 102 focal regions, (3) using the tier 1 and tier 2
regions from the recursive partitioning applied on the 102 focal regions, (4) using all 180 genomic regions (5) using the
tier 1 and tier 2 regions from the recursive partitioning applied all 180 regions and (6) using the top 50 ranked
regions from the random forest classification model built with the 180 focal regions.

| % of regions affected | 1th quartile | 1th quartile | 10% | 15% | 20% | 25% | 30% | 35% | 40% |
|---|---|---|---|---|---|---|---|---|---|
| (1) Regions from Table 5 | 27% | P | P | P | P | P | P | P | NP |
| (2) Top 50 ranked regions from Table 14 | 38% | P | P | P | P | P | P | P | P |
| (3) Regions from Table 10 and 11 | 33% | P | P | P | P | P | P | P | P |
| (4) Regions from Table 1 | 22% | P | P | P | P | P | NP | NP | NP |
| (6) Top 50 ranked regions from Table 15 | 36% | P | P | P | P | P | P | P | P |
| (5) Regions from Table 6 and 7 | 31% | P | P | P | P | P | P | P | P |

Materials and Methods

Sample Collection

Tumor tissue of 278 CRC patients receiving combination bevacizumab treatment or chemotherapeutic agents alone were identified and provided from the tissue bio-banks of the Royal College of Surgeons in Ireland (RCSI) Beaumont Hospital (n=29), The University of Heidelberg (UHEI) in Germany (n=107) and the VU university medical centre (VUMC) in The Netherlands (n=142). A second cohort 106 of combination bevacizumab treated tumors and accompanying normal tissue from the MOMA clinical trial was provided by The University of Pisa in Italy (NCT02271464). Informed consent was obtained from the patient, following the ethical approval of the local ethical committee. After tissue collection, samples were reviewed by qualified pathologists to reconfirm cancer diagnosis and delineate adjacent normal tissue. Only tumor blocks with (1) at least 30% tumor cell content, as judged by a routine hematoxylin and eosin (H&E) staining, (2) sufficient tissue volume in order to allow successful DNA isolation and (3) clinical data available were considered for further processing and analysis. Additionally we downloaded publicly available copy number data of a cohort of 205 patients from the CAIRO trial that were treated with Irinotecon-Capecitabine (CAPIRI) or capecitabine (CAP) only (Agilent oligonucleotide hybridization arrays; GSE36864) (Haan et al 2014).

DNA Isolation

After pathological examination, 1-10 FFPE slides (5-10 μm) were used for DNA extraction. Regions with high tumor content as well as regions containing only normal cells as indicated by the pathologist were macro-dissected from individual slides. Subsequently the FFPE tissue sections are deparaffinised using a series of xylene and ethanol washes. The sections were then subjected to purification and homogenization (by gentle shaking at 400 rpm while incubation in buffer ALT and Proteinase K at 56° C.) to remove fixatives and aid lysis. After deparaffinisation and tissue digestion, DNA was further extracted using the QIAamp DNA FFPE Tissue kit (QIAgen) following the manufacturer's instructions. The resulting DNA was quantified using the Picogreen Assay (Life Technologies) following the manufacturer's instructions. This assay allows to accurately determine the concentration of double-strand DNA needed for further sequencing library preparation. Only samples with a yield of more than 0.5 μg of dsDNA and a concentration >7.5 ng/μl were selected for further library preparation.

Low-Coverage Whole Genome Sequencing

Shot-gun whole genome libraries were prepared using KAPA library preparation kit (KAPA Biosystems). Since the DNA was extracted from FFPE tissue blocks, whole genome DNA libraries from matched normal and tumor tissue samples were created according to the manufacturer's instructions with some modifications to the protocol. Before end repair, a 4 hour incubation step at 65° C. was added to remove as many reversible crosslinks as possible after which excessive single stranded DNA was removed using Mung-Bean nuclease. The concentration double stranded DNA was reassessed using picogreen and the concentration of adapters used in the ligation step of the library construction was altered according to the present DNA. For the library enrichment, 5 to 15 cycles of PCR with intermediate assessment steps were used instead to ensure low adapter dimer content and high library yield. After quantification with qPCR, the resulting libraries were sequenced on a HiSeq2500 (Illumina) at low coverage (±0.1×). Raw sequencing reads were mapped to the human reference genome (NCBI37/hg19) using Burrows-Wheeler Aligner (BWA v0.5.8a) (Li and Durbin 2010). Picard (v1.43) was used to remove PCR duplicates. CNAs were identified by binning the reads in 30 Kb windows, correcting for genomic waves using the PennCNV software package (Wang et al 2007) and the resulting number of reads per 30 Kb window were transformed into log R-values. The ASCAT algorithm version 2.0.1 (Van Loo et al 2010) was used to segment the raw data and estimate tumor percentages and overall ploidy. Subsequently, GISTIC v2.0 (Mermel et al 2011) was used to identify the most frequent and overrepresented chromosomal aberrations in tumors. A region was considered deleted if the log R value was <0.1 and amplified when the log R was >0.1 A cut-off q-value of 0.25 was used to select significantly overrepresented CNAs. CNAs spanning >70% of a chromosomal arm were defined as whole-arm CNAs, while CNAs spanning <70% of a chromosomal arm were considered focal CNAs. Significant amplified or deleted regions were assigned as homozygous deletion, loss, diploid, gain or amplification for each sample based on Log R signal and GISTIC output threshold values (t<−1.3; −1.3<t<−0.1; −0.1<t<0.1; 0.1<t<0.9; t>0.9 respectively).

Whole-Exome Sequencing

After confirmation of successful library construction, whole exome enrichment was performed using the SeqCapV3 exome enrichment kit (Roche) following the manufacturer's instructions. The resulting whole-exome libraries were then sequenced on a HiSeq2500 using a V3 flowcell generating 2×100 bp paired end reads. Raw sequencing reads were mapped to the human reference genome (NCBI37/hg19) using Burrows-Wheeler Aligner (BWA v0.5.8a) (Li and Durbin 2010) and aligned reads were processed and sorted with SAMtools (v0.1.19) (Li et al 2009). Duplicate reads were removed using Picard tools. Base recalibration, local realignment around insertions and deletions and single nucleotide variant calling were performed using the GenomeAnalysisToolKit (GATK) (McKenna et al 2010). Insertions and deletions were called using Dindel (Albers et al 2011). By subtracting variants and indels detected in the matched germline DNA from those found in the tumor DNA, somatic mutations were selected. Low quality mutations were removed based on mapping quality and coverage. ANNOVAR (Wang et al 2010) was used to annotate the remaining mutations and exonic non-synonymous mutations and frame-shift insertions or deletions were selected. Common variants (MAF>1%) were filtered out using the following databases as described previously (Zhao et al 2014): (1) dbSNP version 132, (2) 1000 Genomes Project, (3) Axiom Genotype Data Set, (4) Complete Genomics diversity panel (46 hapmap individuals).

Statistical Analysis

Consensus clustering using unsupervised Hierarchical Ward clustering was performed using the packages ‘ConsensusClusterPlus’ and ‘hclust’ in R on all samples using the recurrent CNAs identified from the GISTIC analysis on all mCRC samples as input using a subsampling size of 80% and 50 repetitions. Multivariate survival analysis between the different clusters was performed using a Cox regression analysis using TNM staging and age as numerical factors while gender and the cluster were used as categorical factors. For each cluster and to compare CNA-high with CNA-low patients, survival of patients receiving combination bevacizumab therapy was compared with patients treated with chemotherapy in a univariate analysis using the Kaplan Meier method and evaluated with a log rank-test. Recursive partitioning was performed using the R-package ‘rpart’ using the ‘class’ method. To determine the different tiers we used all 180 regions to build a first most optimal regression tree. Next, in a stepwise manner we removed one of the regions used in the tree and generated a second tree, after that we reinserted that specific region again and removed another region and generated a third, fourth, fifth etc. . . . tree. By performing this on 4 different levels (each time removing and replacing one of the used regions) we selected the most important regions based on recurrent selection by the recursive partitioning. The CNAs selected after the first analysis completed 4 levels were assigned to tier 1, removed from the list of 180 regions and the process was repeated to generate tier 2, tier 3 and tier 4. A similar approach was used for the 102 focal regions to determine tier 1, tier 2 and tier 3 regions. Random forest classification was performed using the R-package ‘rf’ using. K-nearest neighbors classification was performed using the package ‘knn’. For both the random forest and k-nearest neighbors classifiers, we performed a 10-fold cross-validation on the original dataset to determine the accuracy of the model. Hereto, we divided the 442 mCRC samples used for the original clustering 10 times at random, each time in a training set (90% of the samples) and validation set (10% of the samples) in such a manner that each sample is presented only once in the whole of 10 validation sets. Next a random forest classifier was generated using the training data. We then applied this classifier to the validation data to determine the models accuracy.

### REFERENCES

Albers, C. A. et al. Dindel: accurate indel calls from short-read data. Genome research 21, 961-73 (2011).
Ferrara, N. et al (2004). Discovery and development of bevacizumab, an anti-VEGF antibody for treating cancer. Nature Reviews Drug Discovery 3, 391-400.
Guinney, J. et al. The consensus molecular subtypes of colorectal cancer. Nature Medicine 21, 1350-1356 (2015).
Haan, J. C. et al. Genomic landscape of metastatic colorectal cancer. Nature communications 5, 5457 (2014).
Leary, R. et al. (2008). Integrated analysis of homozygous deletions, focal amplifications, and sequence alterations in breast and colorectal cancers. Proc Natl Acad Sci USA. 105, 16224-16229.
Lengauer C, Kinzler K, Vogelstein B (1998). Genetic instabilities in human cancers. Nature 396, 643-649.
Li, B-Q. et al. Classification of non-small cell lung cancer based on copy number alterations. Plos One 9, e88300 (2014).
Li, H. & Durbin, R. Fast and accurate long-read alignment with Burrows-Wheeler transform. Bioinformatics 26, 589-95 (2010).
Li, H. et al. The Sequence Alignment/Map format and SAMtools. Bioinformatics 25, 2078-9 (2009).
McKenna, A. et al. The Genome Analysis Toolkit: a MapReduce framework for analyzing next-generation DNA sequencing data. Genome research 20, 1297-303 (2010).
Mermel, C. H. et al. GISTIC2.0 facilitates sensitive and confident localization of the targets of focal somatic copy-number alteration in human cancers. Genome biology 12, R41 (2011).
Pino, M. S. and Chung, D. C. (2010). The chromosoma instability pathway in colon cancer. Gastroenterology 138, 2059-2072.
Van Loo, P. et al. Allele-specific copy number analysis of tumors. Proceedings of the National Academy of Sciences of the United States of America 107, 16910-5 (2010).
Wang, K. et al. PennCNV: an integrated hidden Markov model designed for high-resolution copy number variation detection in whole-genome SNP genotyping data. Genome research 17, 1665-74 (2007).
Wang, K., Li, M. & Hakonarson, H. ANNOVAR: functional annotation of genetic variants from high-throughput sequencing data. Nucleic acids research 38, e164 (2010).
Wood, L. D. et al (2007). The genomic landscapes of human breast and colorectal cancers. Science 318, 1108-1113.
Zhao, H. et al. Mismatch repair deficiency endows tumors with a unique mutation signature and sensitivity to DNA double-strand breaks. Elife 3, e02725 (2014).

1. A colorectal cancer biomarker panel comprising at least 5 genomic DNA regions or fragments thereof selected from Table 1.

2. A method of measuring the copy number alteration status of a colorectal cancer sample, the method comprising:
obtaining the colorectal cancer sample; and
measuring, in the colorectal cancer sample, the copy number alteration status of the genomic regions of the colorectal biomarker panel of claim 1.

3. A method of measuring the copy number instability of a colorectal cancer sample the method comprising:
obtaining the colorectal cancer sample; and
measuring the copy number instability of the genomic regions in the colorectal biomarker of claim 1.

4.-9. (canceled)

10. A method for the treatment of a subject suffering from colorectal cancer with anti-VEGF therapy, the method comprising:
measuring, in the colorectal cancer sample, the copy number alteration status of the genomic regions of the colorectal biomarker panel of claim 1
classifying the colorectal cancer sample as being one of genetic subtypes 2 or 3 as depicted in Table 3 or 4; and
treating the subject with anti-VEGF therapy.

11. A method for the treatment of a subject suffering from colorectal cancer with anti-VEGF therapy, the method comprising:
measuring a copy number instability of 15% or more in a colorectal cancer sample from the subject utilizing the biomarker panel of claim 1, and
treating the subject with anti-VEGF therapy.
